(19) United States
(12) Patent Application Publication
(10) Pub. No.: US 2026/0020856 A1
(43) Pub. Date: Jan. 22, 2026

(54) SYSTEMS, DEVICES, AND METHODS FOR CONTROLLING AN IMPLANTABLE FLOW RESTRICTION SYSTEM

(71) Applicant: Relief Cardiovascular, Inc., Costa Mesa, CA (US)

(72) Inventors: Alexander H. Cooper, Costa Mesa, CA (US); Matthew A. Sussman, Laguna Niguel, CA (US); Juliet Laura Schwartz, Tustin, CA (US); Joseph Arthur Passman, Costa Mesa, CA (US); J. Brent Ratz, Winchester, MA (US); Arshad Quadri, West Hartford, CT (US)

(21) Appl. No.: 19/113,157

(22) PCT Filed: Jul. 3, 2024
(86) PCT No.: PCT/US2024/036769

§ 371 Date: Mar. 19, 2025

Related U.S. Application Data
(60) Provisional application No. 63/512,152, filed on Jul. 6, 2023.
(60) Provisional application No. 63/591,304, filed on Oct. 18, 2023.
(60) Provisional application No. 63/667,040, filed on Jul. 2, 2024.

(51) Int. Cl. A61B 17/12; A61B 17/00; A61B 90/00

(52) U.S. Cl. CPC A61B 17/12109; A61B 17/12031; A61B 17/12036; A61B 17/12172; A61B 90/06; A61B 2017/00039; A61B 2017/00221; A61B 2017/00398; A61B 2017/00477; A61B 2017/00734; A61B 2017/00876; A61B 2090/064

(57) ABSTRACT

A flow restriction system may include an implantable controller system for controlling an implantable flow restrictor. The controller system may include: an implantable housing, an actuator at least partially disposed within the implantable housing, the actuator having a first configuration corresponding to the implantable flow restrictor being in the low profile state and a second configuration corresponding to the implantable flow restrictor being in the high profile pressure restricting state, an internal power source configured to supply current to the actuator, and a processor configured to control operation of the actuator using power from the internal power source.

### INCORPORATION BY REFERENCE TO ANY PRIORITY APPLICATIONS

This application claims the benefit of priority under 35 U.S.C. § 119(e) to U.S. Provisional Patent Application No. 63/512,152, filed Jul. 6, 2023, U.S. Provisional Patent Application No. 63/591,304, filed Oct. 18, 2023, and U.S. Provisional Patent Application No. 63/667,040, filed Jul. 2, 2024. All of the above-mentioned applications are hereby incorporated by reference herein in their entireties. Any and all applications for which a foreign or domestic priority claim is identified in the Application Data Sheet as filed with the present application are hereby incorporated by reference under 37 CFR 1.57.

### TECHNICAL FIELD

The present disclosure relates to systems, devices, and methods for treating heart failure, including systems, devices, and methods for controllably and selectively occluding, restricting, and/or diverting flow within a patient's vasculature.

### LIMITED COPYRIGHT AUTHORIZATION

A portion of the disclosure of this patent document includes material which is subject to copyright protection. The copyright owner has no objection to the facsimile reproduction by anyone of the patent document or the patent disclosure as it appears in the Patent and Trademark Office patent file or records, but otherwise reserves all copyrights whatsoever.

### BACKGROUND

An identified issue in heart failure is volume overload, wherein there is an excess of pressure built up in the venous system which can cause the heart to not work as well as a pump. Reducing the total volume of fluid in the body, such as by the administration of diuretics, is one method to reduce volume overload and improve heart function. Another way to improve heart function in heart failure is to shift the distribution of blood in the vascular system. Such a shift in the distribution of blood can affect the preload on the heart and thus the heart's ability to pump effectively. Additionally, shifting venous blood volume away from the renal system and/or lymphatic ducts can enhance diuresis, further reducing volume overload and improving heart function.

### SUMMARY

Current nonpharmacological therapies aimed at reducing volume overload and/or reducing preload lack chronic controllability and/or adjustability. Additionally, current methods to improve and/or control diuresis include systemic application of diuretics, which can significantly affect patient quality of life. A more controllable method of controlling diuresis is desired. To address these and other unmet needs, the present disclosure describes various implementations of a chronic, implantable flow restriction systems, devices, and methods for controllably and selectively occluding, restricting, and/or diverting flow within a patient's vasculature. The chronic, implantable flow restriction systems devices described herein include various controller systems for actuating a blood flow modulator in a variety of ways. Furthermore, the chronic, implantable flow restriction systems and devices described herein can be configured to control the blood flow modulator to provide partial and/or full occlusion of a vessel from within the vessel and/or external to the vessel. Such ability to chronically control the occlusion of a patient's vessel(s) can allow, for example, the control of diuresis without systemic drugs/medication.

In one embodiment, an implantable controller system for controlling a blood flow modulator is provided that includes a housing enclosing a microcontroller, an actuator, and a first power source. The microcontroller can include one or more computer readable storage devices configured to store a plurality of computer-executable instructions, one or more hardware computer processors in communication with the one or more computer readable storage devices, and a first communication module. The actuator can include a control member and a motor. The control member can have a first end coupled with the blood flow modulator and a second end configured to be disposed adjacent to or in the housing. The motor can be coupled to the second end of the control member to actuate the control member. Actuation of the control member can cause the blood flow modulator to move between a low profile state and a high profile pressure modulating state/high profile pressure flow restricting state. The first power source can be disposed in the housing and can be configured to generate current for the actuator.

In another embodiment, a system is provided that includes an implantable controller system and an external device. The implantable controller system can be configured to control an implantable flow restrictor between a low profile state and a high profile flow restricting. The implantable controller system can include an implantable housing, an actuator, an internal power source, and a processor. The actuator can be at least partially disposed within the implantable housing. The actuator can have a first configuration corresponding to the implantable flow restrictor being in the low profile state and a second configuration corresponding to the implantable flow restrictor being in the high profile flow restricting state. The internal power source can be configured to supply current to the actuator. The processor can be configured to control the operation of the actuator using power from the internal power source. The external device can include an external housing enclosing a communication model. The communication module can be configured to communicate with the implantable controller system. The external device can be configured to process information related to user physiology and/or receive user input for activating the actuator within the implantable controller system.

In another embodiment, a system is provided that includes an implant and an implantable controller system. The implant can include a flow restrictor coupled with a distal end of a tubing and coupled with a distal end of a shaft extending through a lumen of the tubing. A proximal portion of the tubing can have an outside surface including and/or surrounding one or more electrical contacts of an array of electrical contacts. A proximal length of the lumen can be disposed through the proximal portion of the tubing. The implantable controller system can be configured for controlling the flow restrictor between a low profile state and a high profile flow restricting state. The implantable controller system can include an implantable housing and an actuator at least partially disposed within the implantable housing. The actuator can have a first configuration corresponding to the flow restrictor being in the low profile state and a second configuration corresponding to the flow restrictor being in the high profile flow restricting state. The actuator can be at least partially located within the proximal length of the lumen of the tubing.

In another embodiment, a system is provided that includes a controller and an implant. The controller can include an implantable housing, an actuator, and a first connector. The implantable housing can include an outside surface configured to be placed under skin of a patient and a connection port being located on the outside surface of the implantable housing. The actuator can be at least partially disposed within the implantable housing. The actuator can have a first configuration corresponding to a flow restrictor being in a low profile state and a second configuration corresponding to the flow restrictor being in a high profile flow restricting state. The first connector can be accessible through the connection port. The implant can include a tubing, the flow restrictor, a shaft, and a second connector. The tubing can include a distal end and a proximal end. The flow restrictor can be coupled with the distal end of the tubing. The shaft can include a proximal end and a distal end coupled with the flow restrictor. The second connector can be disposed on the proximal end of the shaft. The second connector can be configured to be engaged with the first connector in a locked position by insertion of the second connector into the connection port by a distance along a first direction.

In another embodiment, a method of regulating blood pressure and/or flow using a flow restrictor positioned within a first blood vessel of a patient upstream of a second blood vessel branching off of the first blood vessel at an ostium is provided. Treatment instructions can be received via a controller system implanted within the patient from another device, such as an external device. The treatment instructions can include a treatment duration and a target state for the flow restrictor. The flow restrictor can be adjusted via an actuator of the controller system from an original state to the target state. The flow restrictor can provide greater flow restriction within the first blood vessel upstream of the ostium of the second blood vessel in the target state than in the original state. In another example, the treatment instructions can include a set pressure threshold. For example, the flow restrictor can be adjusted to a set duration and/or occlusion state when the pressure is above the set pressure threshold (e.g., 10 mmHg). The occlusion state may be determined by displacement of a portion of the actuator and/or the pressure drop across the flow restrictor, for example.

In another embodiment, a pressure sensor including a cavity plate and a piezo plate is provided. The cavity plate can include a first external side and a first internal side of the pressure sensor. The first internal side can include, for example, at least partially surround, a first cavity. The piezo plate can include a second external side and a second internal side of the pressure sensor. The second internal side can include a piezoresistive diaphragm and a plurality of electrical contacts. The second external side can include a second cavity aligned with the piezoresistive diaphragm. The first internal side can be coupled to the second internal side such that the piezoresistive diaphragm is aligned with the first cavity.

In another embodiment, a pressure sensor that includes a cavity plate and a sensor plate is provided. The cavity plate can include a first cavity and a reflow passage disposed through the cavity plate. The sensor plate can be coupled with the cavity plate. The sensor plate can include a second cavity, a strain sensitive member aligned with the second cavity, and a signal conveyance connected to the strain sensitive member. The reflow passage in the cavity plate can be configured to receive a reflow material to secure the pressure sensor to a catheter body.

In another embodiment, a sensor container assembly is provided. The sensor container can be configured to house a pressure sensor. The pressure sensor can include a base portion, a top portion, at least one wall extending from the base portion to the top portion and defining an internal volume, and a pressure sensor disposed in the internal volume.

In another embodiment, a sensor container assembly is provided. The sensor container can be configured to house a pressure sensor. The sensor container can include a base portion, a top portion, at least one wall extending from the base portion to the top portion and defining an internal volume, and a plurality of electrical contacts coupled to at least one of the base portion, the top portion, and the at least one wall.

In another embodiment, a method of assembling a catheter product is provided. A catheter body is provided. A plurality of electrical wires can be extended through peripheral lumens formed within an outside surface of the catheter body. Distal ends of the electrical wires can be routed through a plurality of securement passages formed through a cavity plate and a diaphragm plate of a pressure sensor. The distal ends of the electrical wires can be coupled with contacts formed on the cavity plate. The electrical wires can be retracted within the peripheral lumens of the catheter body to position an external side of the pressure sensor against the outside surface of the catheter body. The pressure sensor can be coupled to the outside surface of the catheter body through the securement passages.

In another embodiment, a system including an implant, a first connector, a second connector, and an implantable controller system is provided. The implant can include a flow restrictor coupled with a distal end of a tubing and coupled with a distal end of a shaft extending through a lumen of the tubing. A proximal length of the lumen can be disposed through a proximal portion of the tubing. The first connector can be disposed at the proximal portion of the tubing. The second connector can be disposed at a proximal end of the shaft. The second connector can be at least partially disposed within the first connector. The implantable controller system can include an implantable housing and an actuator. The implantable controller system can be configured to control the flow restrictor between a low profile state and a high profile flow restricting state. The actuator can be positioned within the implantable housing. The actuator can be configured to cause the second connector to translate within the first connector. The actuator can have a first configuration corresponding to the flow restrictor being in the low profile state and a second configuration corresponding to the flow restrictor being in the high profile flow restricting state.

In another embodiment, a sensor assembly including a sensor mount and a pressure sensor is provided. The sensor assembly can be configured to be coupled with a housing. The sensor mount can include a top side, a bottom side, and a channel extending between the top side to the bottom side. The pressure sensor can be disposed within the channel and coupled to the top side of the sensor mount.

In another embodiment, a sensor assembly including a sensor container, a pressure sensor, and a circuit is provided. The sensor container can be configured to be coupled with a tubular body. The sensor container can include a base and a cover including a first opening. The cover can be configured to be coupled to the base to define an internal volume. The pressure sensor can be disposed in the internal volume and can extend through the first opening. The circuit can be positioned within the sensor container and electrically connected to the pressure sensor via a first set of leads. The circuit can be configured to process and/or amplify signals received from the pressure sensor.

In another embodiment, a system including a blood flow modulator, an implantable controller system, and a pressure sensor is provided. The blood flow modulator can be configured to move between a low profile state and a high profile flow restricting state. The implantable controller system can be configured for implantation in a patient and can be configured to control the blood flow modulator. The implantable controller system can include a microcontroller. The pressure sensor can be configured to generate a pressure signal to be transmitted to the microcontroller.

In another embodiment, a system including a network interface, one or more data stores, and one or more physical computer processors is provided. The network interface can be configured to communicate with a plurality of network devices. The one or more data stores can be configured to store computer-executable instructions, a first data set, and a second data set. The first data set can include a plurality of pressure measurements collected from a plurality of controller systems implanted in a plurality of patients. The second data set can include a plurality of patient events associated with the plurality of patients. The one or more physical computer processors can be in communication with the one or more data stores. When the computer-executable instructions are executed, they can configure the one or more physical computer processors to access the first data set and the second data set, and generate a predictive model. The first data set and the second data set can be accessed by the network interface from the one or more data stores. The predictive model can be generated by training a machine learning algorithm. Training the machine learning algorithm can include inputting the first data set and the second data set into the machine learning algorithm and comparing the first data set to the second data set. The predictive model can be configured to a determine a likelihood of heart failure events based on the comparison.

For purposes of summarizing the disclosure, certain aspects, advantages and novel features of several implementations have been described herein. It is to be understood that not necessarily all such advantages are achieved in accordance with any particular implementation of the technology disclosed herein. Thus, the implementations disclosed herein can be implemented or carried out in a manner that achieves or optimizes one advantage or group of advantages as taught herein without necessarily achieving other advantages that can be taught or suggested herein.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIG. 1A illustrates a patient's anatomy including a heart with a right atrium, a right ventricle, a left atrium, and a left ventricle, a superior vena cava connected to the right atrium, an inferior vena cava connected to the right atrium as well as to the patient's renal veins, and other vessels and organs of the patient.

FIG. 1B illustrates a patient's anatomy including the connections between ducts of the patient's lymphatic system, such as the thoracic duct and right lymphatic duct, and veins of the patient.

FIG. 2 illustrates a chronic, implantable flow restriction system that is implanted within the patient in accordance with some aspects of this disclosure.

FIGS. 3A-3D illustrate an implementation of an implant of a chronic, implantable flow restriction system in accordance with some aspects of this disclosure.

FIGS. 4A-4C illustrate various end views of the implant of FIGS. 3A-3D in accordance with some aspects of this disclosure.

FIG. 4D illustrates an end view of another implementation of the implant of FIGS. 3A-3D in accordance with some aspects of this disclosure.

FIG. 5 illustrates a flat pattern of view of an expandable body of the implant of FIGS. 3A-3D in accordance with some aspects of this disclosure.

FIGS. 6A-6B illustrate various views of components of a chronic, implantable flow restriction system in accordance with some aspects of this disclosure.

FIGS. 7A-7D illustrate interaction of various components of a flow restriction system to actuate the implant of FIGS. 3A-3D in accordance with some aspects of this disclosure.

FIGS. 8A-8B illustrate an implementation of a shaft of an implantable flow restriction system in accordance with some aspects of this disclosure.

FIGS. 9A-9C illustrate an implementation of a connector between components of a flow restriction system in accordance with some aspects of this disclosure.

FIGS. 10A-10B illustrate an implementation of a connector between components of a flow restriction system in accordance with some aspects of this disclosure.

FIGS. 11A-11D illustrate an implementation of a connector between components of a flow restriction system in accordance with some aspects of this disclosure.

FIGS. 11E-11H illustrate an implementation of a connector between components of a flow restriction system in accordance with some aspects of this disclosure.

FIG. 12A-12B illustrate schematic circuit diagrams of certain features of an implantable controller of an implantable flow restriction system in accordance with some aspects of this disclosure.

FIG. 13 illustrates a schematic diagram of an implantable controller of an implantable flow restriction system in accordance with some aspects of this disclosure.

FIGS. 14A-14D illustrate various views of an implantation of an implantable controller of an implantable flow restriction system in accordance with some aspects of this disclosure.

FIGS. 14E-14J illustrate various views of another implantation of an implantable controller of an implantable flow restriction system in accordance with some aspects of this disclosure.

FIGS. 15A-15D illustrate various views of another implantation of an implantable controller of an implantable flow restriction system in accordance with some aspects of this disclosure.

FIGS. 16A-16D illustrate various views of another implantation of an implantable controller of an implantable flow restriction system in accordance with some aspects of this disclosure.

FIGS. 17A-17C illustrate various views of another implantation of an implantable controller of an implantable flow restriction system in accordance with some aspects of this disclosure.

FIGS. 18A-18B illustrate various views of an implementation of an ingress protection system of the implantable controller of FIGS. 15A-15D in accordance with some aspects of this disclosure.

FIGS. 19A-19D illustrate various views of another implementation of an implantable controller of an implantable flow restriction system in accordance with some aspects of this disclosure.

FIGS. 20A-20B illustrate various views of an implementation of a sensor system for an implantable controller of an implantable flow restriction system in accordance with some aspects of this disclosure.

FIG. 20C illustrates voltage measurements of the sensor system of FIGS. 20A-20B in accordance with some aspects of this disclosure.

FIGS. 21A-21G illustrate various views of another implementation of an implantable controller of an implantable flow restriction system in accordance with some aspects of this disclosure.

FIG. 21H illustrates a cross-sectional view of an ingress protection system of the implantable controller of FIGS. 21A-21G.

FIG. 21I illustrates a cross-sectional view of the implantable controller of FIGS. 21A-21G along the line I-I in FIG. 21A.

FIGS. 22A-22D illustrate various views of a connector assembly of an implantable flow restriction system in accordance with some aspects of this disclosure.

FIGS. 23A-23C illustrate various view of an outer connector of the connector assembly of FIGS. 22A-22D in accordance with some aspects of this disclosure.

FIGS. 24A and 24B illustrate a perspective view of a proximal end of a shaft and connector sleeves of an implantable flow restriction system and a perspective view of the shaft coupled via the connector sleeves to an inner connector of the connector assembly of FIGS. 22A-22D in accordance with some aspects of this disclosure.

FIG. 25A illustrates a guideline for treatment of a patient using an implantable flow restriction system in accordance with some aspects of this disclosure.

FIG. 25B illustrates a manual method of using an implantable flow restriction system in accordance with some aspects of this disclosure.

FIG. 25C illustrates a semi-automatic method of using an implantable flow restriction system in accordance with some aspects of this disclosure.

FIG. 25D illustrates an automatic method of using an implantable flow restriction system in accordance with some aspects of this disclosure.

FIG. 25E illustrates a swim-lane flow diagram of an example method of a patient using an implantable flow restriction system in accordance with some aspects of this disclosure.

FIG. 25F illustrates a method of generating a predictive model by training a machine learning algorithm in accordance with some aspects of this disclosure.

FIGS. 26A-26C illustrate an implementation of delivering therapy using an implantable flow restriction system in accordance with some aspects of this disclosure.

FIGS. 27A-27C illustrate various implementations of an implant of a flow restriction system with a sensor located in various positions relative to a flow restrictor portion of the implant in accordance with some aspects of this disclosure.

FIGS. 28A-28B illustrate various view of an implementation of a sensor of an implantable flow restriction system in accordance with some aspects of this disclosure.

FIGS. 29A-29B illustrate implementations of a piezoresistive pattern for a sensor of an implantable flow restriction system in accordance with some aspects of this disclosure.

FIG. 30 illustrates a schematic diagram of a Wheatstone bridge circuit.

FIG. 31A-31F illustrate various view of another implementation of a sensor of an implantable flow restriction system in accordance with some aspects of this disclosure.

FIGS. 32A-32C illustrate various view of another implementation of a sensor of an implantable flow restriction system in accordance with some aspects of this disclosure.

FIG. 33 illustrates a top view of another implementation of a sensor of an implantable flow restriction system in accordance with some aspects of this disclosure.

FIG. 34 illustrates a contact connector that can be used with a sensor of an implantable flow restriction system in accordance with some aspects of this disclosure.

FIGS. 35A-35D illustrate an implementation of the sensor of FIG. 33.

FIG. 36 illustrates a sensor case that can be used with a sensor of an implantable flow restriction system in accordance with some aspects of this disclosure.

FIGS. 37A-37C illustrate various views of an implementation of a sensor assembly in accordance with some aspects of this disclosure.

FIGS. 38A-38C illustrate various views of another implementation of a sensor assembly in accordance with some aspects of this disclosure.

FIGS. 39A-39R illustrate graphical user interfaces presented on a user device and associated with an implantable controller system in accordance with some aspects of this disclosure.

FIGS. 40A-40P illustrate graphical user interfaces presented on a user device and associated with an implantable controller system in accordance with some aspects of this disclosure.

FIG. 41 illustrates an embodiment of a computing system which may implement example embodiments of one or more components of a controller of an implantable flow restriction system, an external device, and/or affiliated systems.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes or which is essential to practicing the systems, devices, and/or methods disclosed herein.

Parts, components, features, and/or elements of the chronic, implantable flow restriction systems and devices described herein that can function the same or similarly across various implementations are identified using the same reference numerals with a different letter added after the reference numerals. Differences between the various implementations are discussed herein.

The present disclosure describes various implementations of chronic, implantable flow restriction systems, devices, and methods for controllably and selectively occluding, restricting, and/or diverting flow within a patient's vasculature. Such systems, devices, and methods can be used to redirect flow and/or enhance perfusion within the patient's vasculature and/or one or more of the patient's organs. In some circumstances, it can be advantageous to controllably and selectively occlude, restrict, and/or divert flow within a patient's vasculature to reduce renal congestion (or promote renal decongestion), to reduce hepatic congestion (or promote hepatic decongestion), to reduce cardiac preload, and/or to reduce lymphatic/interstitial congestion. For example, a chronically implantable flow restriction system adapted to controllably and selectively occlude and/or restrict a patient's superior vena cava upstream of where the superior vena cava enters the patient's right atrium can be used to reduce cardiac preload. Such a chronically implantable flow restriction system can also be adapted to controllably and selectively reduce central venous pressure and/or pressure of other veins disclosed herein and/or improve cardiac output. As another example, a chronically implantable flow restriction system adapted to controllably and selectively occlude and/or restrict a patient's inferior vena cava upstream of where the patient's renal veins connect with the inferior vena cava (e.g., below where the renal veins connect with the inferior vena cava) can be used to reduce renal congestion. Such a chronically implanted system can also be adapted to controllably and selectively enhance renal circulation, enhance and/or control diuresis, and/or reduce volume overload. The various implementations of chronic, implantable flow restriction systems and devices described herein can be configured to be implanted within a patient for months, a year, or years. Furthermore, the various implementations of chronic, implantable flow restriction systems and devices can be configured to controllably and selectively occlude, restrict, and/or divert flow within a patient's vasculature without an assist device or a pump.

The chronic, implantable flow restriction systems, devices, and methods described herein can be adapted for percutaneous delivery. As such, the systems and devices described herein can be configured to be delivered via a catheter or a similar delivery device and can have a collapsed configuration for delivery into a patient and can expand from the collapsed configuration to an expanded configuration for implantation within the patient. Additionally, the systems and devices or components thereof described herein can be adapted to be retrievable after deployment, such as for repositioning and/or for removal from the body (e.g., by including a hook or other feature for retrieval). In some implementations, the systems and devices described herein can be configured to be delivered and implanted within the patient's vasculature. For example, a portion of a chronic, implantable flow restriction system as described herein can be percutaneously implanted within a superior vena cava of a patient upstream of a right atrium of the patient. Such an implantable flow restriction system can be controlled by a controller system to selectively occlude, restrict, and/or divert flow within the patient's superior vena cava (e.g., to reduce cardiac preload). As another example, a portion of a chronic, implantable flow restriction system as described herein can be percutaneously implanted within an inferior vena cava of a patient upstream of where renal veins of the patient connect with the inferior vena cava. Such an implantable flow restriction system can be controlled by a controller system to selectively occlude, restrict, and/or divert flow within the patient's inferior vena cava (e.g., to reduce renal congestion). In some implementations, the systems and devices described herein can be configured to be delivered extravenously to at least partially surround or be positioned adjacent to the patient's vasculature. For example, a portion of a chronic, implantable flow restriction system as described herein can be percutaneously implanted external of an inferior vena cava of a patient and at least partially surround or be positioned adjacent to the patient's superior vena cava. Such an implantable flow restriction system can be controlled by a controller system to selectively occlude, restrict, and/or divert flow within the patient's inferior vena cava (e.g., to reduce renal congestion).

The chronic, implantable flow restriction systems and devices described herein can be configured to partially occlude and/or fully occlude a target vessel. Additionally, the systems and devices described herein can be configured to not occlude or substantially not occlude a target vessel until actuated. In other words, the systems and devices of the present disclosure can be controlled to substantially occlude all flow through a vessel, occlude partial flow through the vessel, and/or allow substantially all flow through the vessel unimpeded. For example, a flow restriction system and/or device can be configured to adjustably occlude blood flow in a vessel in a range of 0 to 100 percent. In some implementations of the systems and devices described herein, an implantable flow restriction system and/or device can be configured to not substantially occlude flow through a vessel unless actuated to close partially and/or fully. In some cases, the systems and devices described herein can be configured to substantially occlude all and/or partial flow through a vessel unless actuated to open. Furthermore, in some implementations, the systems and devices described herein can have a bias to be partially closed, however once implanted they can open fully due to the flow of blood in the target vessel. It should be understood that the chronic, implantable flow restriction systems and devices of the present disclosure can be configured to be controllable so as provide between and including substantially no occlusion of flow to substantially full occlusion to flow within a vessel. In some cases, such control can be binary (e.g., open or closed) or graded (e.g., open, various degrees of partially closed, or closed).

The chronic, implantable flow restriction systems and devices described herein can be sized and configured for implantation within a target vessel of interest of a patient, such as a superior vena cava (SVC), an inferior vena cava (IVC), and others. A flow restriction device, which can also be referred to herein as an implant, an occluder, a blood flow modulator, and/or a prosthetic, can have an expanded (e.g., implanted) diameter in the range of about 5 mm to about 50 mm, about 10 mm to about 40 mm, about 15 mm to about 30 mm, or it can have a diameter greater than about 50 mm or less than about 5 mm depending on the application. In some implementations, an implant as described herein can be oversized for the vessel of interest and thus impart an outward force on the vessel in which it is implanted (e.g., to improve anchoring within the vessel). A flow restriction device can have an expanded (e.g., implanted) length in the range of about 0.5 cm to about 5 cm, about 0.75 cm to about 4 cm, about 1 cm to about 3 cm, or it can have a length greater than about 5 cm or less than about 0.5 cm depending on the application.

The chronic, implantable flow restriction devices described herein configured for implantation within a vessel of a patient can generally include an expandable body (configured for percutaneous delivery as described herein) and a flow restrictor configured to controlled by a controller system to controllably and selectively occlude, restrict, and/or divert flow within the patient's vasculature. The expandable body can have a proximal end, a distal end, and a lumen extending from the proximal end to the distal end. The expandable body can generally comprise a frame (which can also be referred to as a stent) having an open cell and/or a closed cell structure. Furthermore, the expandable body can include features to aid in anchoring and/or maintaining its placement within the body, such as free apices, barbs, and/or anchors, which can extend in any direction relative to the implant. In some cases, such barbs and/or anchors can comprise a partial hook, hook, and/or straight configuration. The expandable body can be made of a material configured to expand upon delivery, and as such can comprise a shape memory material such as nitinol. In some implementations, the expandable body can be configured to radially collapse/crimp. Alternatively, or in addition, the expandable body can be configured to collapse/crimp sideways upon being pushed or pulled. In some variations, the expandable body can comprise a material without or with little shape memory, and a balloon can be used to expand the expandable body for implantation. The expandable body can include one or more material layers, such as an inner material layer (e.g., within its lumen) and/or an outer material layer (e.g., external to its lumen). Such inner and/or outer material layers can comprise ePTFE, PTFE, PET cloth, polyurethane, and/or the like. Additionally, any of such layers can include an anti-thrombotic coating, a drug-eluting coating, or the like. In some implementations, it is desirable to utilize a material and/or coating to prevent ingrowth within the implant to aid in later implant retrieval and/or removal. Conversely, in some cases it is desirable to utilize a material and/or coating to allow and/or promote ingrowth within the implant. Expandable bodies as described herein for one implementation with a particular type of flow restrictor are not limited to only being utilized with that particular flow restrictor, and may be used in other implementations with other types of flow restrictors. In some implementations, a flow restrictor can be integrally formed with an expandable body.

A flow restrictor of an implant as described herein can be sized and/or oriented in a number of ways relative to the expandable body it connects to or is formed with. For example, a flow restrictor can be sized to fully or partially occlude the lumen of the expandable body it connects to or is formed with upon full actuation. Regarding orientation, a flow restrictor can be configured to span the entire length of the expandable body it connects to or is formed with or configured to span a part of the length of the expandable body. In the latter scenario, the flow restrictor can be oriented at the proximal end, the distal end, or anywhere in between (e.g., the middle or near the middle) of the expandable body. In some instances, the flow restrictor can be positioned adjacent to the distal or proximal end of the expandable body, extend beyond the distal or proximal end of the expandable body, or the like.

The implants described herein or portions thereof (e.g., a flow restrictor of an implant) can be configured to secure within a vessel of the patient's vasculature. In some implementations, activating a flow restrictor implanted in a vessel of the patient's vasculature via a controller system causes the flow restrictor to pull in a wall of the vessel to at least partially restrict flow through the vessel and/or a lumen of the implant comprising the flow restrictor. To pull in a wall of the vessel, a flow restrictor or portions thereof can attach or secure to the wall of the vessel (e.g., an inner wall of the vessel). Such attachment/securement can include a mechanical attachment. For example, a flow restrictor can include one or more anchors configured to attach/secure at least a portion of the flow restrictor with at least a portion of a wall of a vessel (e.g., an inner wall of the vessel). As another example, a flow restrictor or a portion thereof can be configured to ingrow at least partially into the wall of the vessel. In such an example, the flow restrictor or a portion thereof can have a structure, material, and/or coating that promotes ingrowth. Further to this example, such flow restrictor can include a structure having struts, a structure having struts with a mesh spanning the struts, or a structure having struts with a material (e.g., a porous or a non-porous material) spanning the struts.

Vascular access for the delivery of a chronic, implantable flow restriction device as described herein can include an internal jugular vein, a subclavian vein, a femoral vein, and/or others. From such access points, a flow restriction device can be advanced within the patient's vasculature by a delivery device (e.g., a delivery catheter) until the desired location of implantation is reached, thereupon the flow restriction device can be delivered and expanded for chronic implantation. A guidewire, introducers, etc. can be utilized for delivery, as well as standard imaging methods. Furthermore, the flow restriction devices herein can include radiopaque features to aid in delivery and implantation. Additionally, the flow restriction devices can include features for indexing to its delivery device to help enable precise orientation of the flow restriction device within the patient. For example, an implant can be indexed to a feature of its delivery device that remains external to the patient (e.g., a logo or other marking). A chronic, implantable flow restriction system can comprise a flow restriction device, a source for actuating the flow restriction device, a controller device, and a delivery device.

The chronic, implantable flow restriction systems and devices described herein can be configured for open-loop and/or closed-loop control. For example, the flow restriction systems and devices described herein can be actuated manually, semi-automatically, and/or fully automatically. In some cases, therapy provided by the flow restriction systems and devices described herein can be digitally actuated, such as by interaction with a smart phone, an external terminal/device, or the like. For example, if a patient desires to enhance diuresis, they can activate such therapy via a press of a button or touchscreen of their smart phone (e.g., therapy can be digitally actuated). In some implementations, the flow restriction devices described herein can comprise and/or work with sensors attached to or located remote from the flow restriction device that can provide physiological parameters of interest useful for control of the flow restriction device. Such physiological parameters of interest can include pressure, flow rate, etc. As an example, a flow restriction device can have a MEMS pressure sensor attached to its proximal end, its distal end, or both of its ends. The pressure sensor can be configured to measure the pressure at such location relative to the flow restriction device (e.g., upstream, downstream, both upstream and downstream, etc.). As another example, MEMS pressure sensors can be located within vessels and/or organs remote from the flow restriction device and provide a measure of the pressure at such locations for the control of the flow restriction device. Sensors can be utilized to allow for fully-automatic, real-time control of the flow restriction devices described herein. Furthermore, absolute values of sensor data and/or differentials of sensor data can be utilized.

Utilization of the chronic, implantable flow restriction systems and devices described herein can be standardized across patients or preferably customized to an individual patient, such as via a prescription provided by a care provider. Treatment protocols can vary depending on the type of flow restriction device implanted, its type of actuation, and/or the location in which it is implanted. The flow restriction systems and devices described herein can be utilized continuously, hourly, multiple times a day, once a day, overnight, once every other day, once every few days, once a week, once a month, or with any frequency as needed or prescribed. Additionally, therapy provided by the flow restriction systems and devices described herein can be based on an amount of time per day, the time of day, a number of days per week, specific days of the week, and the like. Furthermore, instances of treatment can have a duration of seconds, minutes, hours, days, etc. For example, treatment using a flow restriction device described herein can have a duration of 15 minutes, 30 minutes, 1 hour, 1 hour and 30 minutes, 2 hours, 5 hours, 12 hours, or any duration of time necessary or required for the intended use and desired outcome. Additionally, treatment times can vary in their duration, or they can be standardized. In some cases, treatment can be determined via an algorithm, with such algorithm providing a duration and amount of flow restriction to be utilized. Such output from an algorithm can be implemented manually, semi-automatically, or fully-automatically. In some implementations, therapy provided by the flow restriction systems and devices described herein can be based on venous pressure, such as inferior vena cava pressure, renal venous pressure, femoral venous pressure, and/or pressure of other veins disclosed herein. For example, treatment using a flow restriction device described herein can be applied until a pressure threshold is met (e.g., treatment can be applied until a pressure of interest reaches or falls below a pressure threshold). Such threshold can be, for example, about 8 mmHg or in some cases between 6 mmHg and 12 mmHg, between 7 mmHg and 11 mmHg, between 8 mmHg and 10 mmHg, or can be values between the foregoing, etc. for the inferior vena cava. In some implementations, therapy provided by the flow restriction systems and devices described herein can be based on a combination of a duration and a venous pressure. For example, treatment using a flow restriction device described herein can be applied for a duration of time after a pressure threshold is met (e.g., once inferior vena cava pressure gets below 8 mmHg, turn off after 4 hours).

One or more chronic, implantable flow restriction devices as described herein can be implanted within a patient. In some cases, it can be beneficial to have only one flow restriction device implanted within a patient, or it can be beneficial to have multiple flow restriction devices implanted within a patient. If multiple flow restriction devices are implanted within a patient, such devices can work together as needed to achieve the treatment outcome desired. Furthermore, flow restriction devices that utilize the same or different forms of actuation can be implanted within the same patient.

Although the chronic, implantable flow restriction systems, devices, and methods disclosed herein are described in a particular manner which can provide certain advantages, such description is not intended to be limiting. The chronic, implantable flow restriction systems and devices can be implanted in various vessels and/or passageways of a patient, including vessels (e.g., veins, arteries) of the patient's vascular system, the patient's lymphatic system, the patient's reproductive system, etc.

Any and/or all of the implementations and/or features of the chronic, implantable flow restriction systems, devices, and methods described and/or illustrated herein can be applied to the various systems, devices, and methods described and/or illustrated in U.S. Pat. No. 11,883,030, filed Apr. 13, 2023, titled “SYSTEMS, DEVICES, AND METHODS FOR CONTROLLABLY AND SELECTIVELY OCCLUDING, RESTRICTING, AND DIVERTING FLOW WITHIN A PATIENT'S VASCULATURE,” in U.S. patent application Ser. No. 18/300,293, filed Apr. 13, 2023, titled “SYSTEMS AND METHODS FOR TREATING HEART FAILURE BY REDIRECTING BLOOD FLOW IN THE AZYGOS VEIN,” in U.S. Provisional Patent Application No. 63/582,723 filed Sep. 14, 2023, titled “LEADLESS SYSTEMS, DEVICES, AND METHODS FOR CONTROLLABLE ENHANCEMENT OF DIURESIS,” in U.S. Provisional Patent Application No. 63/582,726 filed Sep. 14, 2023, titled “SYSTEMS, DEVICES, AND METHODS FOR IMPLANTING AND/OR RETRIEVING DEVICES FOR CONTROLLABLE ENHANCEMENT OF DIURESIS,” and in U.S. Provisional Patent Application No. 63/591,345, filed Oct. 18, 2023, titled “SYSTEMS, DEVICES, AND METHODS FOR CONTROLLABLY AND SELECTIVELY OCCLUDING, RESTRICTING, AND DIVERTING FLOW WITHIN A PATIENT'S VASCULATURE,” the entire contents of which are hereby incorporated by reference in their entirety, and vice versa. For example, any and/or all of the implementations and/or features of the chronic, implantable flow restriction systems, devices, and methods described and/or illustrated herein, such as a controller system to actuate a flow restrictor, can be applied in a pulmonary artery to azygos vein shunt as described in the above-referenced applications. As another example, any and/or all of the implementations and/or features of a shunt between a pulmonary artery and an azygos vein as described and/or illustrated in the above-referenced applications, such as an adjustable shunt including a rotatable disk that can rotate relative to a stationary frame to control the size of an opening through the shunt, can be applied to the chronic, implantable flow restriction systems, devices, and methods described and/or illustrated herein. Additionally, any and/or all of the implementations and/or features of the chronic, implantable flow restriction systems, devices, and methods described and/or illustrated herein can be applied to and/or used in atrial-septal shunts and/or pulmonary artery-to-superior vena cava shunts.

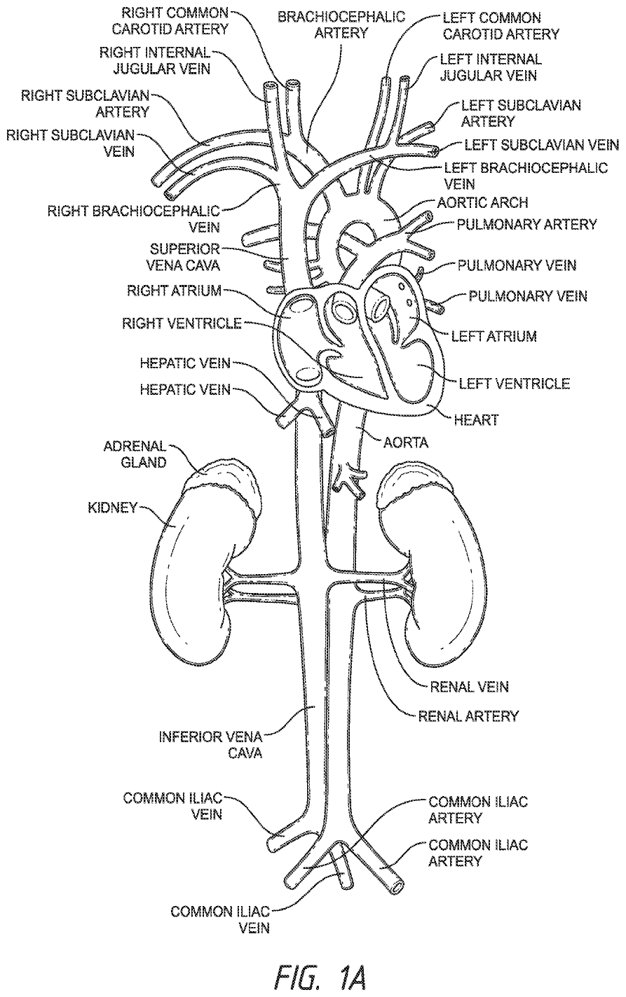

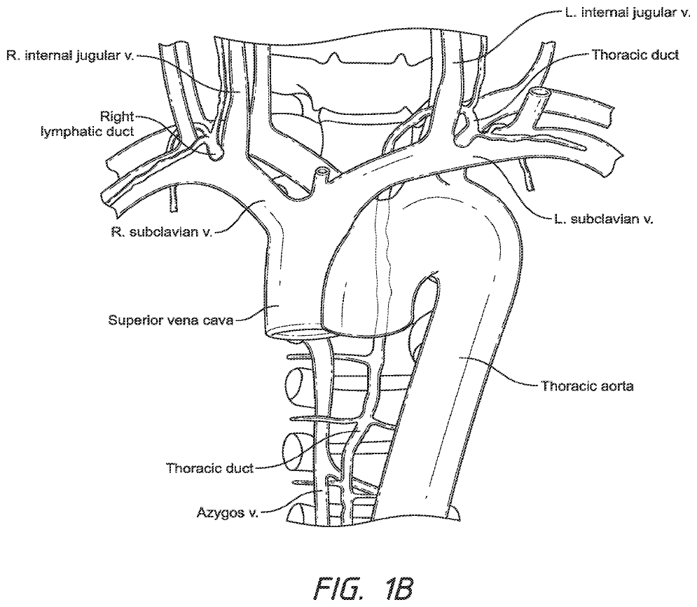

FIG. 1A illustrates a simplified representation of a patient's anatomy including a heart with a right atrium, a right ventricle, a left atrium, and a left ventricle, a superior vena cava connected to the right atrium, an inferior vena cava connected to the right atrium as well as to the patient's renal veins and hepatic veins, and other vessels and organs of the patient.

FIG. 1B illustrates a simplified representation of a patient's anatomy including the connections between ducts of the patient's lymphatic system, such as the thoracic duct and right lymphatic duct, and veins of the patient. As shown, the thoracic duct connects and empties into the left subclavian vein near its confluence with the left internal jugular vein. Also shown, the right lymphatic duct connects and drains into the right subclavian vein.

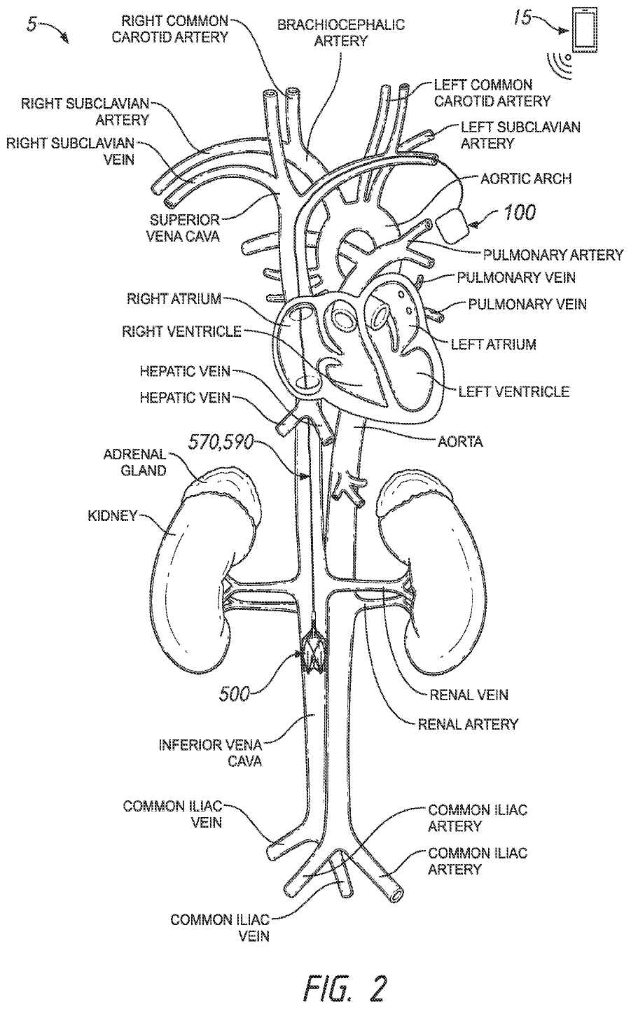

FIG. 2 illustrates a potential location for implantation and placement of an implantable flow restriction system 5 (also referred to herein at the “system 5”). The implantable flow restriction system 5 can be a mechanically-actuated, chronic implantable flow restriction system 5. While certain implementations of the implantable flow restriction system 5 may be described as a chronic system, components of the implantable flow restriction system could be used in an acute system. Moreover, certain implementations of the implant 500 of the implantable flow system 5 are described as being mechanically actuated. This may include electromechanically-actuated implants. Other actuation methods are also possible, for example a fluid or gas driven system.

An implantable flow restriction system 5 can include an implant 500 (which can also be referred to herein as a “blood flow modulator”) connected to an implantable control unit 100 (which can also be referred to herein as a “controller”), for example via tubing 570 and shaft 590 (which can all be implanted), and an external device 15 for operating the system 5. Reference to the implantable control unit 100 throughout this disclosure as part of the implantable flow restriction system 5 is understood to include any of the control units described herein (e.g., the implantable control unit 100, the implantable control unit 100A, the implantable control unit 100B, the implantable control unit 100C, the implantable control unit 100D, the implantable control unit 100E, and the implantable control unit 100F). In some implementations, the tubing 570 may be a catheter. In some implementations, the implantable flow restriction system 5 includes the implant 500, the controller 100, the tubing 570, and the shaft 590. Shown in FIG. 2 is one implant 500 implanted within the patient's IVC upstream of its connection to the renal veins (e.g., below the renal veins). An implant 500 placed in the IVC upstream of the renal veins can controllably and selectively occlude, restrict, and/or divert flow within the patient's IVC and connected vasculature and/or organs, such as to reduce renal congestion (or promote renal decongestion), enhance renal circulation, and/or to control diuresis (e.g., to increase diuresis). For this, the implant 500 can have a flow restrictor portion 550 and/or a flow restrictor 560. Such flow restrictor portion 550 and/or flow restrictor 560 can be actuated by the controller 100 via shaft 590 and tubing 570 as described further herein.

The implant 500 can be implanted such that the flow restrictor portion 550 is upstream of the other portions of the implant 500 (e.g., the flow restrictor portion 550 is the first portion of the implant 500 to receive blood flow therethrough). In such position, the shaft 590 and tubing 570 can extend proximally from the implant 500 up the IVC, through the right atrium, into the superior vena cava (SVC), through a subclavian vein (left subclavian as shown), and out the subclavian vein to connect with the controller 100 that can be implanted in an infraclavicular subcutaneous pocket (e.g., similar to placement of a pacemaker). In some implementations, the implant 500 can be implanted in other positions, such as those shown and described in U.S. Pat. No. 11,883,030. Furthermore, in some implementations more than one implant 500 can be implanted within the patient. In the case of multiple implants 500 being implanted within the patient, each can connect to a single controller 100 via separate tubing 570 and shaft 590, or each can connect to their own controller 100 via separate tubing 570 and shaft 590.

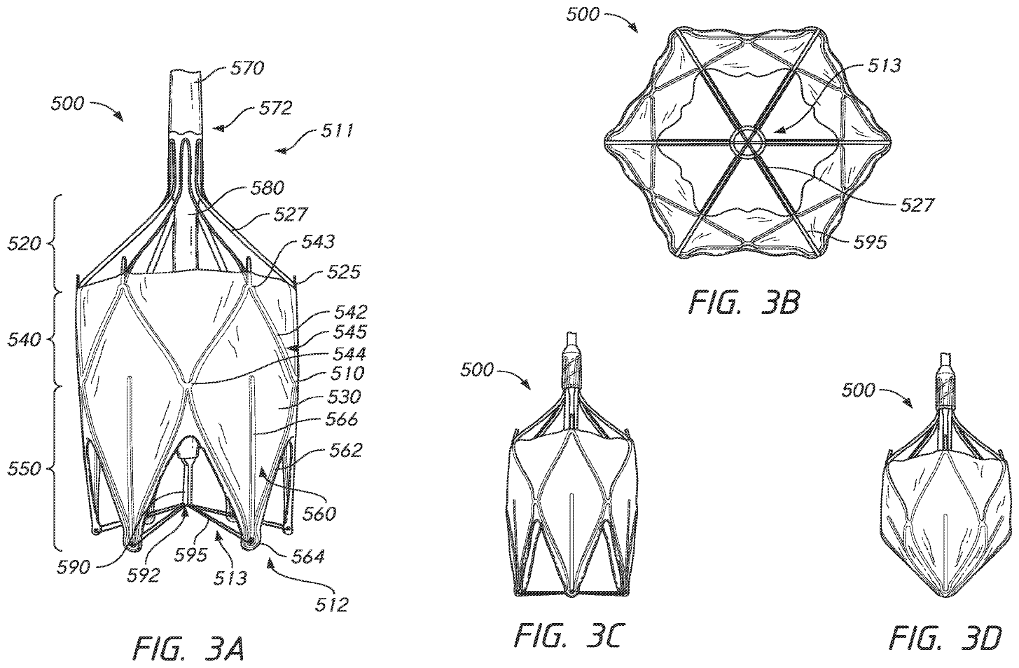

FIGS. 3A-3D illustrate various views of an implementation of the implant 500. FIGS. 3A and 3C show side views, FIG. 3B shows an end view of the implant 500 in a non-occluding (e.g., open, unactuated) state, and FIG. 3D shows a side view of the implant 500 in a fully occluding (e.g., closed, actuated) state. FIGS. 3A-3D also illustrate how the implant 500 can connect with the tubing 570 and the shaft 590 for operation thereof. The implant 500 can comprise an expandable body 510 having a proximal end 511, a distal end 512, and a lumen 513 for receiving blood flow therethrough. The implant 500 can connect to a first or distal end 572 of the tubing 570 and can include a filter portion 520, a radial support portion 540 (which can also be referred to herein as a “scaling portion” or “scaling zone”), and a flow restrictor portion 550.

As shown in FIGS. 3A-3D, the filter portion 520 can be positioned adjacent the proximal end 511, the radial support portion 540 can connect to and be positioned distal of the filter portion 520, and the flow restrictor portion 550 can connect to and be positioned distal of the radial support portion 540. The filter portion 520 can be configured to capture thrombus that may pass through the lumen 513 of the implant 500 and can include a plurality of struts 527 that extend distally and radially outward from the connection between the tubing 570 and the implant 500.

The radial support portion 540 can be configured to fluidically seal against the inner wall of the IVC and can include a ring 545 that extends along a circumference of the implant 500 in a chevron pattern. As shown, the ring 545 can include a plurality of ring struts 542, wherein adjacent pairs of ring struts 542 join at a plurality of proximal apexes 543 and a plurality of distal apexes 544. Further as shown, each of the plurality of proximal apexes 543 of the ring 545 of the radial support portion 540 can be connected to a strut 527 of the filter portion 520.

The flow restrictor portion 550 can include a plurality of petals 560 configured to restrict/occlude flow through the lumen 513 of the implant 500 when actuated. As shown, each of the petals 560 can be formed by a pair of struts 562 that extend distally from adjacent pairs of distal apexes 544 of the ring 545 of the radial support portion 540 and that join at a distal apex 564. Each of the plurality of petals 560 can also include a strut 566 that extends proximally from their respective distal apex 564, which can aid in the ability of the petals to restrict flow when in use. Further as shown, the flow restrictor 550 can include a material 530 that spans each of the plurality of petals 560. The material 530 can comprise ePTFE, PTFE, PET cloth, polyurethane, and/or the like as described herein. Regions between the plurality of petals 560 can be free of the material 530. In some implementations, the material 530 can span regions between the plurality of petals 560. The material 530 can also span the radial support portion 540 to aid in the ability of the implant 500 to fluidically seal against the inner wall of the IVC (or an inner wall of any other lumen/vessel in which it is placed) and restrict/block blood flow when in use. In some implementations and as shown, the implant 500 can include a plurality of anchors 525 configured to anchor the implant 500 within the IVC (or any other lumen/vessel in which it is placed). Such anchors 525 can extend in a generally proximal direction from each of the plurality of proximal apexes 543 of the ring 545.

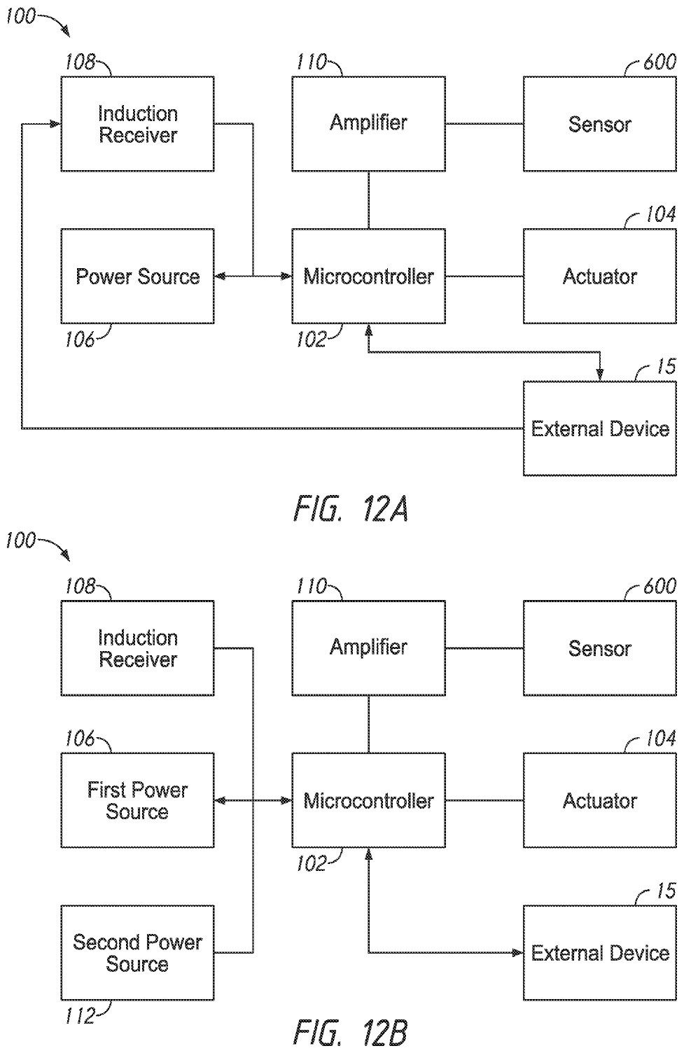

With continued reference to FIGS. 3A-3D, each of the petals 560 can connect to the shaft 590 by a connector. In this arrangement, the shaft 590 functions as a control member for the implant 500. Various connectors are described herein, for example, a suture, wire, strut, or otherwise. For example, each of the distal apexes 564 of the petals 560 can connect to a suture or wire 595 at one end of the suture or wire 595, and the other end of the suture or wire 595 can connect to a first or distal end 592 of the shaft 590 that extends generally centrally through the lumen 513 of the implant 500. As such, the petals 560 may function as moveable elements of the implant 500. Further as shown, the distal end 592 of the shaft 590 can substantially longitudinally align with the distal apexes 564 of the petals 560 in the unactuated/open state of the implant 500. With such relative position, the sutures or wires 595 can extend in a substantially radially outward direction from the distal end 592 of the shaft 590 to connect to the distal apexes 564 of the petals 560. The shaft 590 can slidably move through a lumen of the tubing 570 and extend out the distal end 572 thereof as shown, and a collapsible and extendible coupling 580 can fluidically seal the lumen of the tubing 570 with the shaft 590. A second or proximal end 571 of the tubing 570 (not shown) can connect with the controller 100, and a second or proximal end 591 of the shaft 590 can extend out such proximal end 571 and operably connect with an actuator 104 (see e.g., FIGS. 12A and 12B) of the controller 100. To operate the flow restrictor portion 550 of the implant 500 and at least partially occlude/restrict flow therethrough, the actuator of the controller 100 can be actuated to cause the shaft 590 to move proximally relative to the tubing 570 and implant 500, causing the distal apexes 564 of the plurality of petals 560 to move radially inward towards one another via the connection of the distal apexes 564 to the suture or wire 595 and to the distal end 592 of the shaft 590. In other words, proximal movement of the shaft 590 can cause the petals 560 to come together and at least partially restrict flow through the lumen 513 of the implant 500, such as shown in FIG. 3D. For example, the petals 560 can fold radially inward (e.g., hinge relative to the expandable body) with the distal apexes 564 of the petals 560 forming the distal-most tip of implant 500. In use, blood flows toward and is occluded by exterior surfaces of the petals 560. In some implementations, the flow restrictor portion 550 (e.g., the petals 560) can be configured to attach or secure to a wall of the vessel in which the implant 500 is implanted, and when actuated can pull in the wall of the vessel to at least partially restrict flow through the vessel and/or lumen 513. For this, and as described herein, the flow restrictor portion 550 (e.g., the petals 560) can include one or more anchors and/or be configured to ingrow at least partially into the wall of the vessel.

Tubing 570 can comprise a unitary or a composite structure. For example, tubing 570 can include a tubing portion, a braided portion, and/or a liner. The tubing 570 can comprise, for example, PEBAX. A liner, if included, can comprise PTFE, HDPE, or a silicone blend and can facilitate sliding motion of the shaft 590 within the tubing 570 (e.g., the liner can reduce friction within the tubing 570 and force required to slide the shaft 590 within the tubing 570). Connections between components of the system 5, such as the tubing 570, implant 500, collapsible and extendible coupling 580, and shaft 590, can be made via reflow (e.g., with PEBAX), heat shrink, or the like.

With reference to the end view of the implant 500 shown in FIG. 3B, the implant 500 can be configured such that the sutures or wires 595 substantially align with the struts 527 of the filter portion 520. Such substantial alignment can advantageously allow other interventional devices to pass through the implant 500 if needed. For example, such substantial alignment can allow for a 28 French interventional device to pass through the implant 500.

While the implant 500 of FIGS. 3A-3D is shown as having 6 petals 560, 6 sutures or wires 595 connecting each of the 6 petals 560 to the shaft 590, and a filter portion 520 having 6 struts 527, the implant 500 can be configured to have less than or greater than these numbers of each.

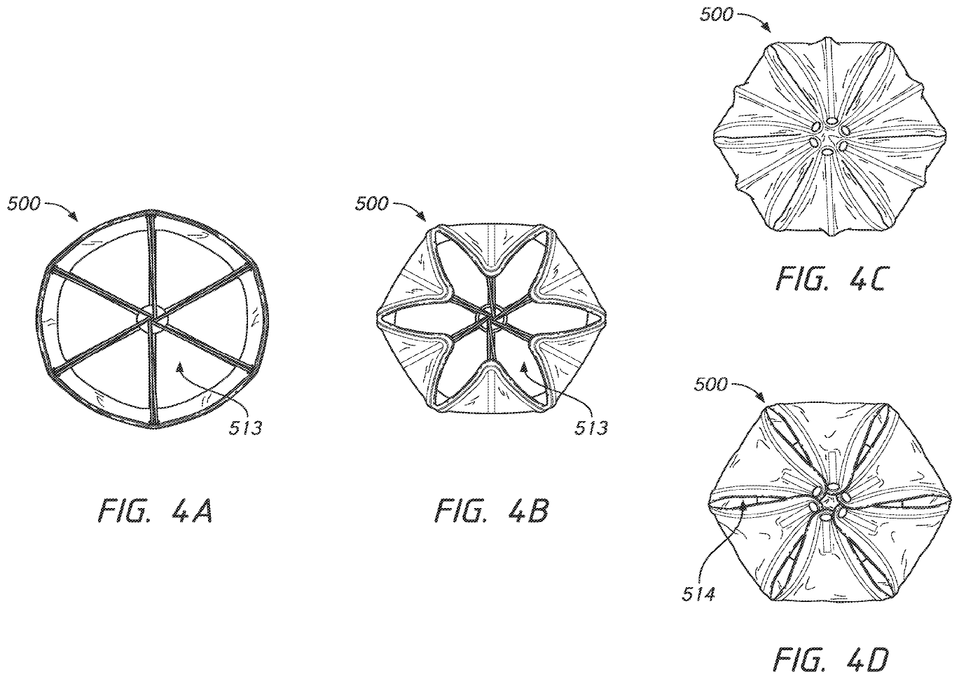

FIGS. 4A-4C illustrate end views of the implant 500 of FIGS. 3A-3D in various states of actuation and restriction/occlusion of flow therethrough. FIG. 4A shows the implant 500 in its unactuated, non-restricting/non-occluding/low profile state, FIG. 4B shows the implant 500 in a partially actuated, partially restricting/occluding/medium profile state, and FIG. 4C shows the implant 500 in a fully actuated, fully restricting/occluding/high profile state. As shown through FIGS. 4A-4C, the lumen 513 of the implant 500 (e.g., the lumen or opening of the flow restrictor portion 550) can change from a generally circular shape (FIG. 4A) when unactuated, to a generally star/stellate shape when at least partially actuated (FIG. 4B), to a substantially blocked lumen when fully actuated (FIG. 4C). In some implementations, the lumen 513 of the implant 500 (e.g., the lumen or opening of the flow restrictor portion 550) can have a generally circular shape when unactuated, a generally circular shape when partially actuated, and a substantially blocked lumen when fully actuated. When folded radially inward, an exterior surface of the each of the plurality of petals 560 can block blood flow via material 530. In some implementations, such as shown in FIG. 4D, the implant 500 can be configured such that its lumen 513 can remain at least partially open even when the flow restrictor portion 550 is fully actuated, such as by the formation of elongate gaps 514 between each of or between at least some of the petals 560.

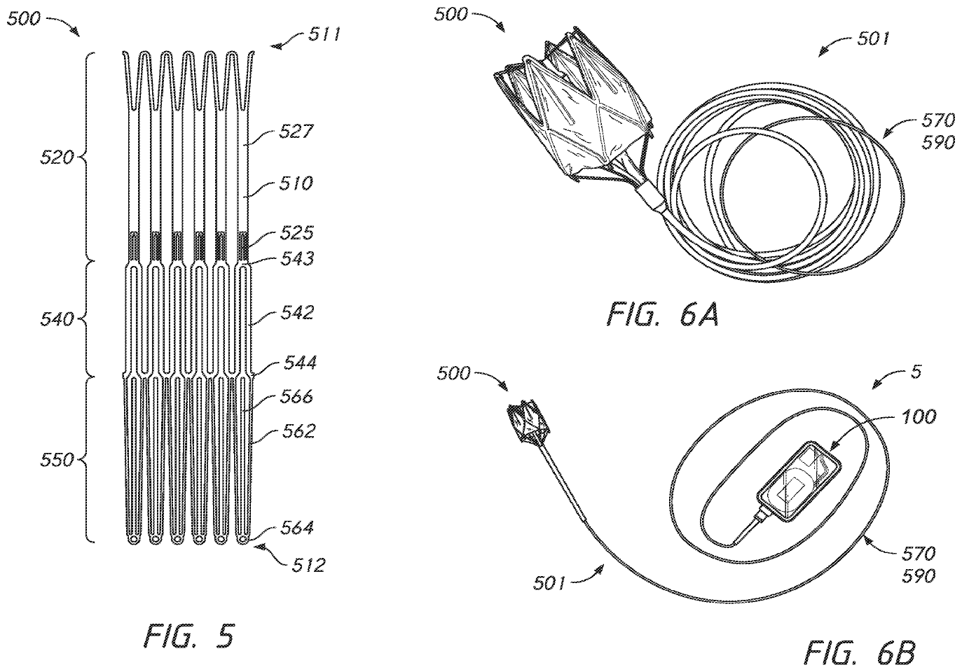

FIG. 5 illustrates a flat pattern of view of the expandable body 510 of the implant 500 of FIGS. 3A-3D with aspects previously discussed identified.

FIGS. 6A-6B illustrate various views of components of the implantable flow restriction system 5, which can be implanted as shown above with respect to FIG. 2. FIG. 6A shows the implant 500 connected to the shaft 590 and tubing 570. The implant 500, the shaft 590, and the tubing 570 can be referred to herein as an implant assembly 501. FIG. 6B shows the implant 500 connected to the shaft 590 and tubing 570, which are in turn connected to the controller 100. In other words, FIG. 6B shows the implant assembly 501 connected to the controller 100.

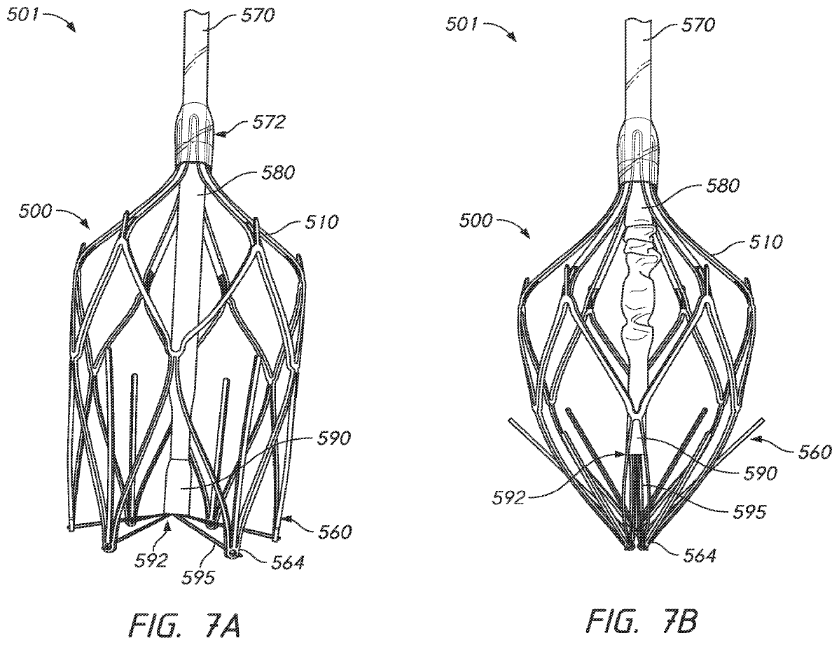

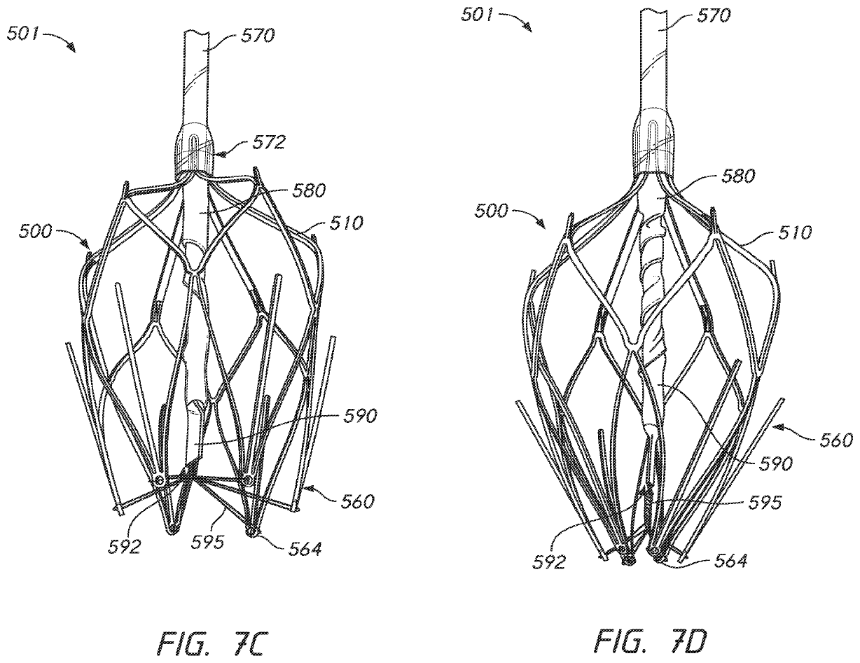

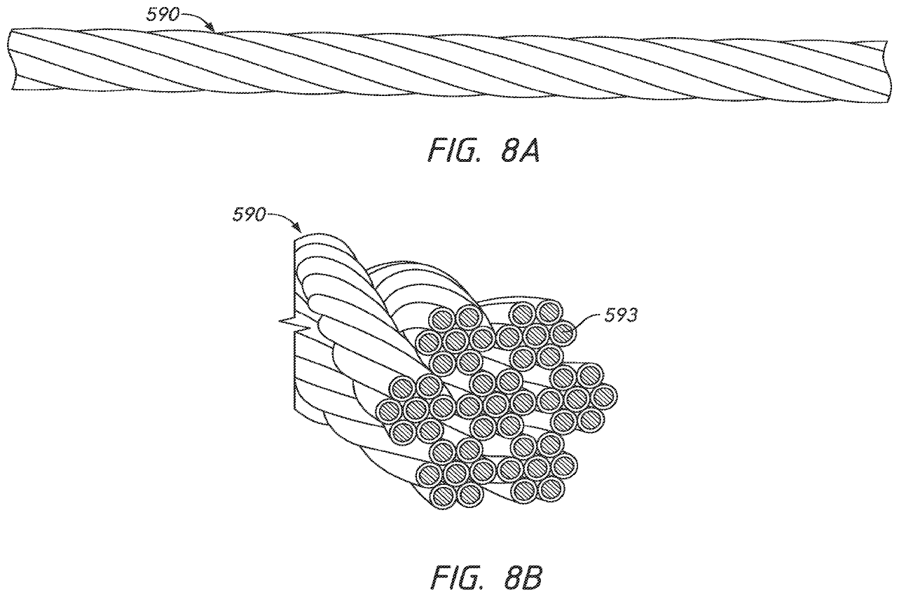

FIGS. 7A-7D illustrate interaction of various components of the implant assembly 501 to actuate the implant 500 of FIGS. 3A-3D, with the material 530 of the implant 500 removed for clarity. As described with respect to FIGS. 3A-3D, each of the distal apexes 564 of the petals 560 can connect to the suture or wire 595 at one end of the suture or wire 595, and the other end of the suture or wire 595 can connect to the distal end 592 of the shaft 590. As an example, the sutures or wires 595 can connect to the distal apexes 564 via eyelets at the distal apexes 564 as shown. Further to this example, the sutures or wires 595 can connect to the distal end 592 of the shaft 590 via a crimp as shown, although other forms of connection are possible and are considered within the scope of this disclosure (e.g., via a set screw, press fit component, adhesive, and/or threaded end). In some implementations, the suture or wire 595 can extend from the distal end 592 of the shaft 590, pass through an eyelet at the distal apex 564 of a petal 560, and double back and connect to the distal end 592 of the shaft 590. In some implementations, the suture or wire 595 can be integrally formed or a part of the shaft 590. For example, in implementations in which the shaft 590 has a braided structure comprising a plurality of individual wires 593 as described with respect to FIGS. 8A-8B, the suture or wire 595 can be one or more of such individual wires 593.

With continued reference to FIGS. 7A-7D, with the material 530 removed from view, the collapsible and extendible coupling 580 configured to fluidically seal the tubing 590 with the shaft 590 can be seen. In some implementations and as shown, the collapsible and extendible coupling 580 can extend around the shaft 590 such that no portion of the shaft 590 is exposed except for where the shaft 590 is connected to the sutures or wires 595. Alternatively, in some implementations the collapsible and extendible coupling 580 can extend around the shaft 590 such that no portion of the shaft 590 is exposed, which can include covering where the shaft 590 is connected to the sutures or wires 595. As shown, the collapsible and extendible coupling 580 can connect at its proximal end to the distal end 572 of the tubing 570, and it can connect at its distal end to the shaft 590 adjacent its distal end 592, which can allow for sliding and/or rotational movement of the shaft 590 therewithin.

In some implementations and as shown in FIGS. 7A-7B, the implant 500 (e.g., the flow restrictor portion 550) can be actuated by longitudinal movement (e.g., proximal and distal movement) of the shaft 590 relative to the implant 500. Such longitudinal movement can include a sliding of the shaft 590 within the tubing 570. In the unactuated, non-restricting/non-occluding state shown in FIG. 7A, the shaft 590 is in its distal-most position relative to the implant 500 and the collapsible and extendible coupling 580 is in its extended state. In its extended state, the collapsible and extendible coupling 580 can have a generally straight configuration as shown. Upon proximal movement of the shaft 590 within the tubing 570, as shown in FIG. 7B (e.g., upon proximal movement of the shaft 590 relative to the tubing 570 and implant 500), the shaft 590 pulls the petals 560 radially inward towards one another via the sutures or wires 595 to occlude/restrict flow through the implant 500. Such proximal movement of the shaft 590 also causes the collapsible and extendible coupling 580 to collapse into its collapsed state. Furthermore, and as shown in FIG. 7B, when the shaft 590 is in its proximal-most position relative to the implant 500, the sutures or wires 595 can be oriented substantially longitudinally.

In some implementations and as shown in FIGS. 7C-7D, the implant 500 (e.g., the flow restrictor portion 550) can be actuated by rotating the shaft 590 (e.g., clockwise or counterclockwise) relative to the implant 500. Such rotation of the shaft 590 can cause the sutures or wires 595 to spool about the shaft 590 or twist and at least partially close the flow restrictor portion 550. In other words, such rotation of the shaft 590 can cause the sutures or wires 595 to spool about the shaft 590 or twist and cause the petals 560a to at least partially fold radially inward. For this, the implantable controller 100 can be configured to rotate the shaft 590. Furthermore, in such implementations the tubing 570 can be configured for rotational movement of the shaft 590 therewithin. Additionally, in such implementations the collapsible and extendable coupling 580 can be configured for such rotational movement. FIG. 7C-7D each show the petals 560 of the implant 500 pulled at least partially radially inward as a result of the shaft 590 being rotated, with FIG. 7D showing the shaft 590 in a more rotated state than as shown in FIG. 7C.

Referring back to FIG. 2, the implant 500 can be positioned within the IVC below the renal veins such that the distal apexes 564 of the petals 560 are aimed towards the incoming flow of blood. In other words, the implant 500 can be implanted such that the flow restrictor portion 550 is upstream of the other portions of the implant 500 (e.g., the flow restrictor portion 550 is the first portion of the implant 500 to receive blood flow therethrough). In such position, the shaft 590 and tubing 570 can extend proximally from the implant 500 up the IVC, through the right atrium, into the superior vena cava (SVC), through a subclavian vein (left subclavian as shown), and out the subclavian vein to connect with the controller 100 that can be implanted in an infraclavicular subcutaneous pocket (e.g., similar to placement of a pacemaker). Furthermore, such positioning of the flow restrictor portion 550 comprising the petals 560 can provide for a functional benefit of pushing any thrombi that may form or otherwise be gathered at the outer surface of the petals 560 when the implant 500 is actuated/closed towards the wall of the IVC upon opening of the petals 560 rather than allowing such thrombi to pass through the implant 500 upon opening of the petals 560 (such as may occur if the petals 560 were not aimed towards the incoming flow of blood). In this way, any thrombi are directed towards the sealing area around the implant 500 with the wall of the IVC and not through the implant 500 and towards the heart.

FIGS. 8A-8B illustrate an implementation of the shaft 590 of the implantable flow restriction systems 5 described herein. FIG. 8A shows a side view and FIG. 8B shows a perspective cross-sectional view of the shaft 590. As shown, the shaft 590 (which can also be referred to as a “wire” or “cable” herein) can have a braided structure comprising a plurality of individual wires 593. In some implementations, a plurality of individual wires 593 can be twisted upon one another to form a bundle, and the shaft 590 can be made of a plurality of such bundles twisted upon one another. In some implementations, the shaft 590 can be made of a single wire, a rod, a hypotube, or a laser cut hypotube depending on the application. For example, for flow restrictions systems that actuate via the shaft pulling on a portion of an implant to actuate a flow restrictor thereof, the shaft can be configured for tension and may have a form the same as or similar to that shown in FIGS. 8A-8B. As another example, for flow restrictions systems that actuate via the shaft pushing on a portion of an implant to actuate a flow restrictor thereof, the shaft can be configured for compression. In another example, for flow restriction systems that actuate via the shaft rotating, the shaft can be configured for rotation. In some implementations, one or more wires 593 of the shaft 590 can be configured to transmit power and/or signals to and/or from one more sensors 600 of a flow restriction system 5. The shaft 590 can be flexible and in some implementations can have a lubricious coating or a lubricious surface to facilitate sliding movement within tubing 570 as described herein. Furthermore, the shaft 590 can be made of biocompatible material (e.g., stainless steel).

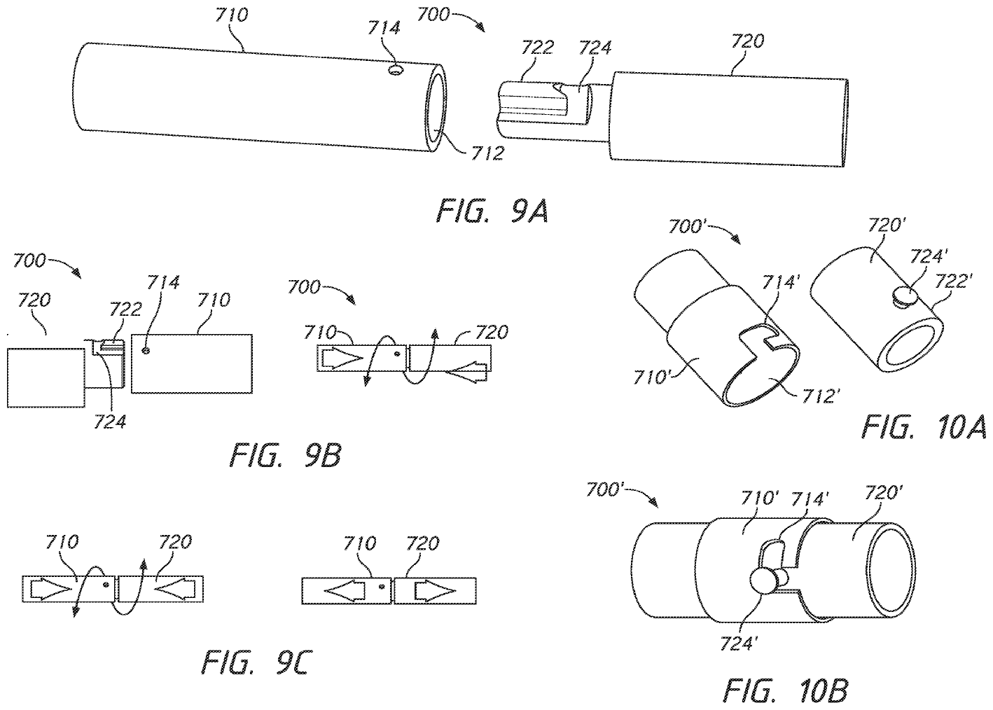

FIGS. 9A-9C illustrate an implementation of a connector 700 between components of a flow restriction system 5. The connector 700 can be configured, for example, to releasably connect a proximal end 591 of the shaft 590 to the actuator 51 of the controller 100. As shown in FIG. 9A, the connector 700 can include a first component 710 and a second component 720 configured to releasably connect with one another via complementary features. Such first component 710 and second component 720 can be secured to the proximal end 591 of the shaft 590 and the actuator 51, respectively, or vice versa. The first component 710 can be configured as a cylinder and can have a circular recess 712 at one of its ends and a protrusion extending radially inward into the recess 712 that is marked visually by point 714 located on an external surface of the first component 710. The second component 720 can be configured as a cylinder and can have a circular rod-like protrusion 722 extending from one of its ends sized to fit within the recess 712 of the first component 710. Furthermore, the protrusion 722 can have a slot 724 configured to receive the protrusion extending radially inward into the recess 712 of the first component 710. As shown in FIGS. 9A-9B, the slot 724 can extend in a longitudinal direction from the end of the protrusion 722 then turn about 90 degrees or more so that, upon alignment of the slot 724 with the point 714 and upon full insertion of the protrusion 722 into recess 712, the first component 710 and the second component 720 can be rotated in a first direction relative to one another to secure the first component 710 and the second component 720 together (e.g., the first and second components can stay connected via interaction between the protrusion of the first component 710 and the slot 724 of the second component 720). FIG. 9C shows how to release the first component 710 from the second component 720, which can include pressing the first component 710 and the second component 720 together, rotating the first component 710 and the second component 720 relative to one another in a second direction that is opposite the first direction, and then separating the first component 710 and the second component 720 from one another.

FIGS. 10A-10B illustrate a variant 700′ of the connector 700. Like the connector 700, the connector 700′ can be configured to releasably connect a proximal end 591 of the shaft 590 to the actuator 51 of the controller 100. As shown in FIG. 10A, the connector 700′ can include a first component 710′ and a second component 720′ configured to releasably connect with one another via complementary features. The first component 710′ can be configured as a cylinder and can have a circular recess 712 at one of its ends similar to the first component 710. Instead of a protrusion and point 714 marking the location of such protrusion, the first component 710′ can include a slot 714′ through a wall of the first component that extends longitudinally from the end of the first component 710′ having the recess 712′ then turns about 90 degrees or more. The second component 720′ can be configured as a cylinder 722′ sized to fit within the recess 712′ of the first component 710′ and can have a circular rod-like protrusion 724′ extending radially outward from its external surface configured to fit within the slot 714′. To connect the first component 710′ and the second component 720′ to one another, the cylinder 722′ can be inserted fully into the recess 712′ with the protrusion 724′ aligned with the slot 714′ and the first component 710′ and the second component 720′ can be rotated in a first direction relative to one another (e.g., the first and second components can stay connected via interaction between the slot 714′ of the first component 710′ and the protrusion 724′ of the second component 720′). To release the first component 710′ from the second component 720′, the first component 710′ and the second component 720′ can be pressed together and rotated relative to one another in a second direction that is opposite the first direction, then the first component 710 and the second component 720 can be separated from one another.

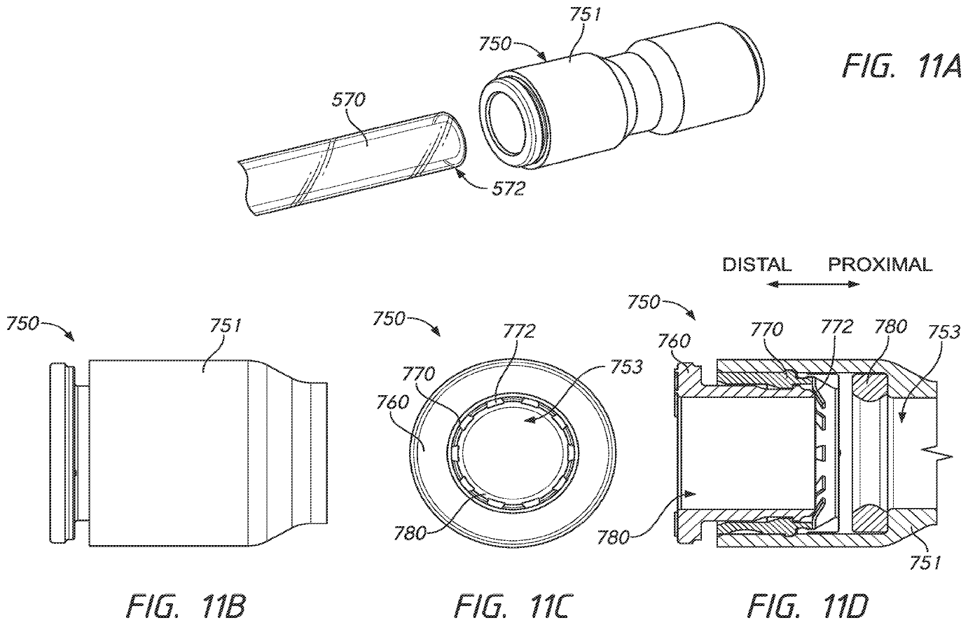

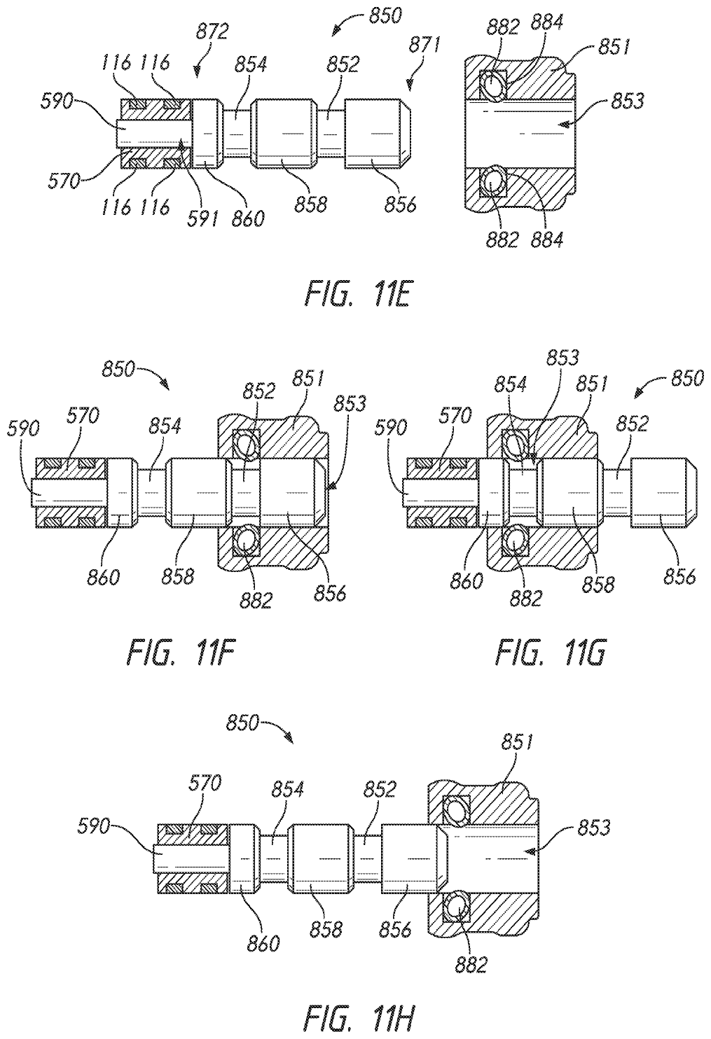

FIGS. 11A-11D illustrate an implementation of a connector 750 between components of an implantable flow restriction system 5. The connector 750 can be configured, for example, to releasably connect and/or fluidically seal a proximal end 571 of the tubing 570 to the controller 100 (e.g., to a housing 200 of the controller 100). For this, the connector 750 can extend from the controller 100 (e.g., extend from the housing of the controller 100). FIG. 11A shows the tubing 570 separated from the connector 750 but in a position for connecting thereto, FIG. 11B shows a side view of a portion of the connector 750, FIG. 11C shows an end view of the connector 750, and FIG. 11D shows a cross-sectional side view of the connector 750. The connector 750 can have a main body 751 having a generally cylindrical shape with a lumen 753 extending therethrough. The connector 750 can include a first component 760 having a longitudinal through hole 762 configured to receive the proximal end 571 of tubing 570 and a second component 770 configured to receive the first component 760 (e.g., shown in FIG. 11D). Both the first and second components 760, 770 can be received by the main body 751. A proximal end of the second component 770 can include a plurality of radially inward extending arms 772 configured to bias the first component 760 in a direction distally away from the second component 770. Such arms 772 can also be configured to grab onto an external surface of the tubing 570 when the tubing 570 is inserted into the connector 750. To connect and fluidically seal the tubing 570 with the connector 750, the proximal end 571 of tubing 570 can be inserted fully into the connector 750 via the through hole 762 of the first component 760 until it cannot be inserted any further. A fluidic seal can be made between the tubing 570 and the connector 750 via a third component 780 configured as a circumferential ring within the main body 751 located proximal to the first and second components 760, 770. In such fully inserted position, the arms 772 of the second component 770 can grab onto the external surface of the tubing 570 and prevent it from releasing from the connector 750. To release the connection between the tubing 570 and the connector 750, the first component 760 can be pressed inward into the connector 750 (e.g., pressed proximally into the connector 750), causing a proximal end of the first component 760 to radially expand the arms 772 and release them from the tubing 570, while the tubing 570 is pulled out of the connector 750 (e.g., moved distally relative to the connector 750).

FIGS. 11E-11H illustrate an implementation of a connector system between components of an implantable flow restriction system 5. The connector system can include a first connector 851 and a second connector 850. The first connector 851 can form a portion of an external housing 200 of an implantable control unit 100. The first connector 851 can be disposed within a portion of an external housing 200 of an implantable control unit 100. The first connector 851 can form and be disposed within a portion of an external housing 200 of an implantable control unit 100. The second connector 850 can be configured, for example, to releasably connect the proximal end 571 of the tubing 570 to the controller 100 (e.g., to the housing 200 of the controller 100). The second connector 850 can be configured, for example, to fluidically seal the proximal end 571 of the tubing 570 to the controller 100 (e.g., to the housing 200 of the controller 100). The second connector 850 can be configured, for example, to releasably connect and fluidically seal the proximal end 571 of the tubing 570 to the controller 100 (e.g., to the housing 200 of the controller 100). For this, the second connector 850 can extend from the controller 100 (e.g., extend from the housing 200 of the controller 100). The second connector 850 can be configured to engage with the first connector 851 in a locked position by insertion of the second connector 850 into the first connector 851 by a distance along a first direction. The second connector 850 can be configured to disengage the locked position with the first connector 851 by being retracted from the controller 100 (e.g., from the housing 200 of the controller 100). The second connector 850 can be configured to disengage the locked position with the first connector 851 by insertion of the second connector 850 into the first connector 851 by a further distance in the first direction. The second connector 850 can then be removed from the first connector 851 by retraction of the second connector 850 out of the first connector 851 by a distance in a second direction opposite the first direction, for example, after being inserted the further distance in the first direction.

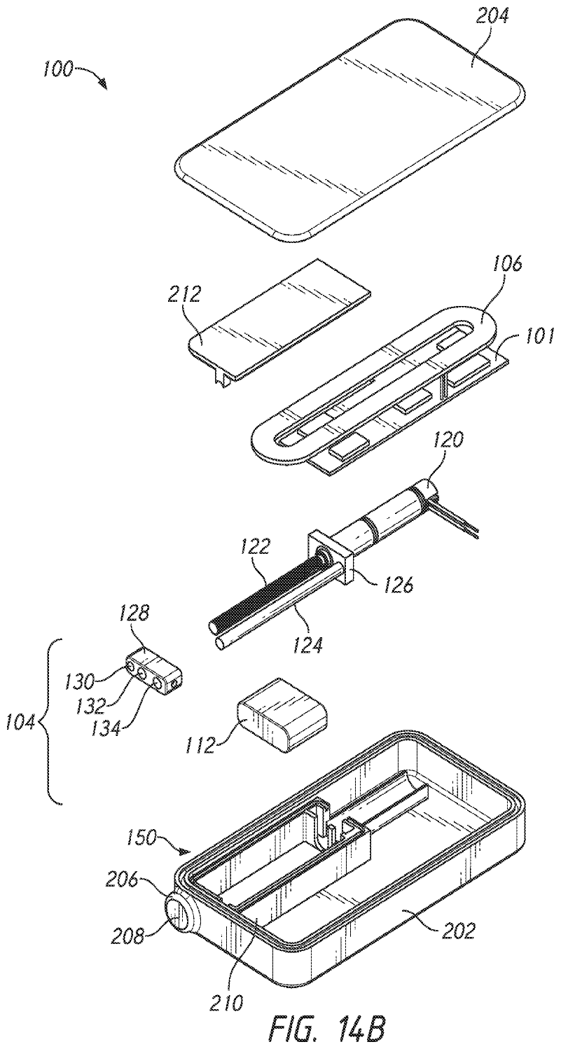

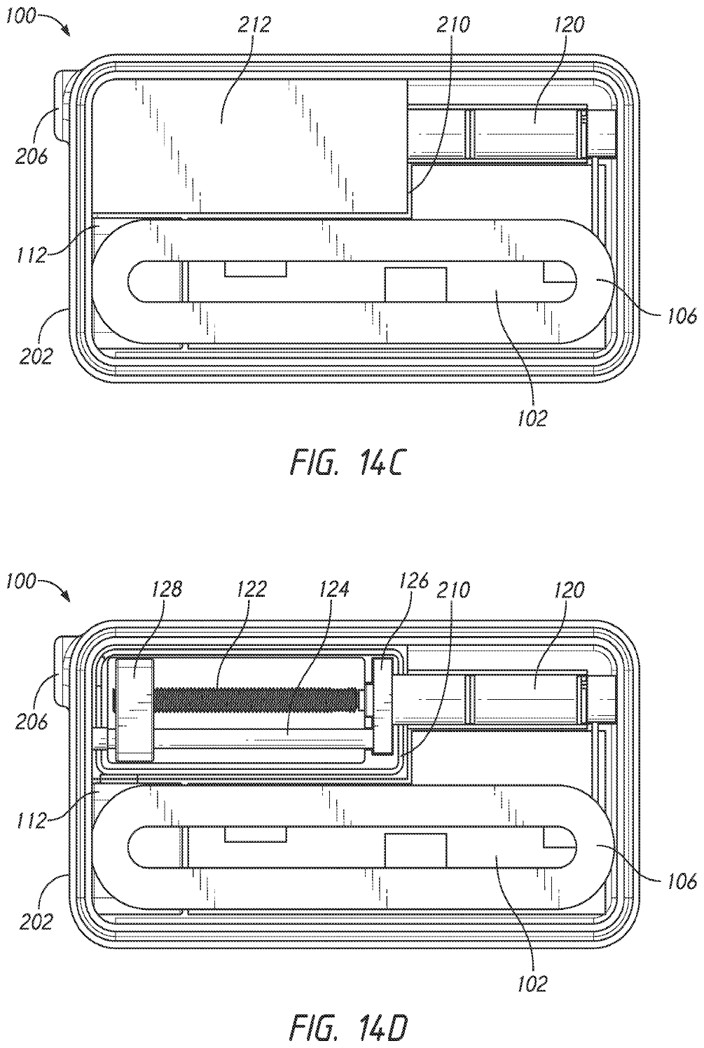

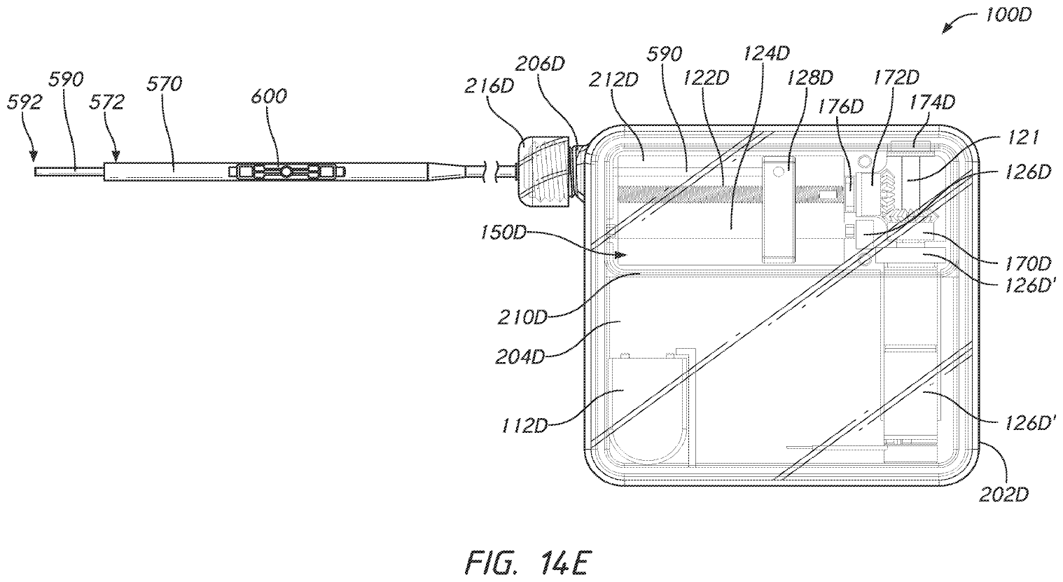

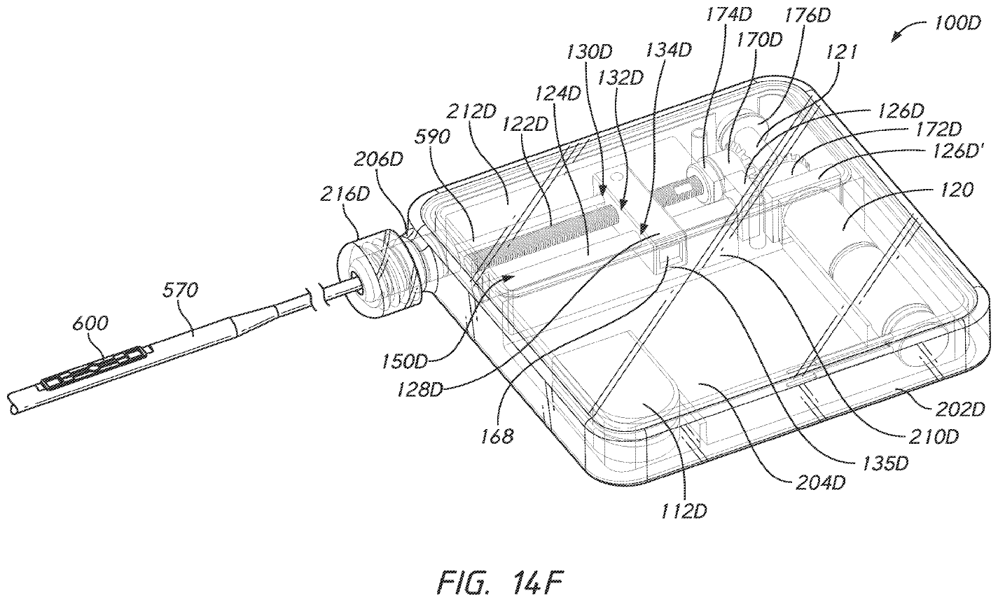

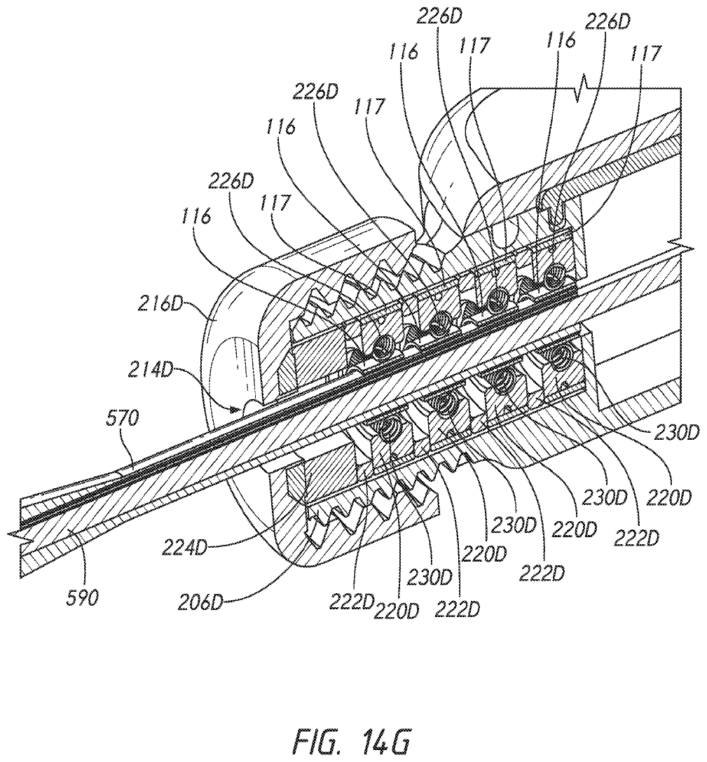

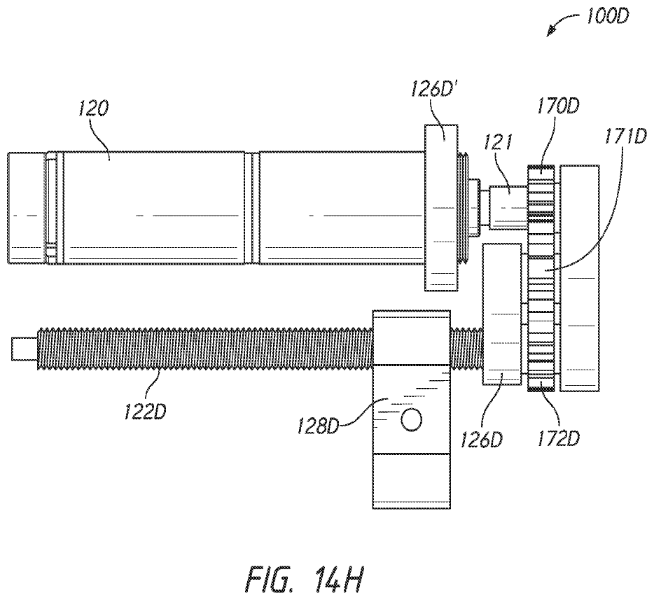

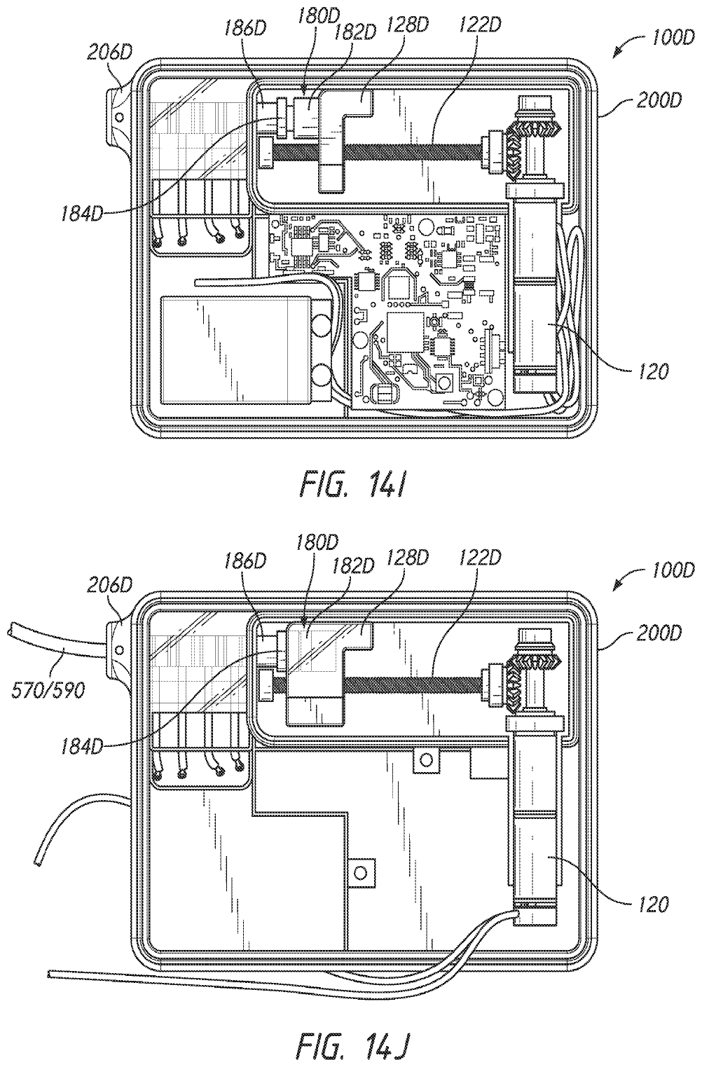

FIG. 11E shows the first connector 851 and the second connector 850 isolated from some components of the controller and the tubing 570 and shaft 590. The first connector 851 can include a bore or lumen 853 and one or more resilient members 882. The one or more resilient members 882 can include a retention spring 882. The retention spring 882 can be secured within a channel 884 of the first connector 851. The channel 884 can be adjacent to the lumen 853 such that the retention spring 882 extends at least partially into the lumen 853 in a relaxed or first configuration. The lumen 853 can be configured to receive the second connector 850. The lumen 853 can be configured to provide access to the internal body of the external housing 200. The retention spring 882 can be configured to selectively retain and/or lock the second connector 850 to the external housing 200. The first connector 851 can be located within a connection port (e.g., a connector sleeve 206, shown in FIG. 14A) of the external housing 200.

The second connector 850 can include a proximal end 871 (also referred to herein as the free end 871) and a distal end 872. The distal end 872 of the second connector 850 can be coupled to the proximal end 591 of the shaft 590. The tubing 570 can abut the distal end 872 and can include electrical contacts 116 described further herein. The second connector 850 can include a first groove 852 and a second groove 854. The grooves 852, 854 can be configured to receive the retention spring 882 to lock or temporarily retain the second connector 850 to the first connector 851. The groove 852 can be configured to receive the retention spring 882 to lock the second connector 850 to the first connector 851 during extended use. The groove 854 can be configured to receive the retention spring 882 to temporarily retain the second connector 850 to the first connector 851 during removal of the second connector 850 from the first connector 851. The first groove 852 can be located closest to or adjacent to the proximal end 871. The first and second grooves 852, 854 can be formed between compression surfaces of the second connector 850. For example, the second connector 850 can include a first compression surface 856, a second compression surface 858, and/or a third compression surface 860. The compression surfaces 856, 858 can be raised portions of the second connector 850 defining the groove 852. The compression surfaces 858, 860 can be raised portions of the second connector 850 defining the groove 854. One or more of the compression surfaces 856, 858, 860 can be configured to compress the retention spring 882 upon engagement to a compressed or second configuration in which the retention spring 882 extends into the channel 884.

The compression surfaces 856, 858, 860 can include one or more square edges and/or one or more angled edges/features. The first groove 852 can be located between the first compression surface 856 and the second compression surface 858. The second groove 854 can be located between the second compression surface 858 and the third compression surface 860. The first compression surface 856 can include, or be disposed adjacent to or between, an angled edge on the proximal end 871 of the second connector 850 and a square edge on the side of the first groove 852. The second compression surface 858 can include, or be disposed adjacent to or between, two angled edges connecting the second compression surface to the first and second grooves 852, 854. The third compression surface 860 can include, or be disposed adjacent to, an angled edge extending to the second groove 854. As such, the first groove 852 can have a square edge closest to the free end 871. The first groove 852 can be located between an angled edge and the square edge. The first groove 852 can be located farther from the free end 871 than is the square edge. The second groove 854 can include an angled edge between the second groove 854 and the first groove 852. The second groove 854 can have two angled edges.

The angled edges can be configured to allow the retention spring 882 to move between a relaxed configuration locking the second connector 850 to the first connector 851 and a compressed configuration when the second connector 850 is being inserted into the first connector 851. The square edge can prevent the retention spring 882 from moving from the relaxed configuration to the compressed configuration. To lock the second connector 850 to the first connector 851, the second connector 850 can be inserted a first distance into the lumen 853 while moving in a first direction. The angled edge of the first compression surface 856 can cause the profile of the free end 871 of the second connector 850 in the direction of the first groove 852 to gradually enlarge such that the retention spring 882 can be compressed to a profile radially outward of the square edge of the first groove 852. Continued travel of the second connector 850 to the first distance causes the retention spring 882 to move off of the first compression surface 856 and into the first groove 852, as shown in FIG. 11F. When the retention spring 882 is in the first groove 852, the square edge between the first groove 852 and the first compression surface 856 restricts, minimizes, or prevents movement of the second connector 850 in a second direction opposite the first direction. In this position, the second connector 850 is engaged with, for example, locked to the first connector 851. To disengage, for example, unlock the second connector 850, the second connector 850 can be further inserted into the lumen 853 to a second distance in the first direction. The angled edge of the first groove 852 (or between the groove 852 and the surface 858) allows the retention spring 882 to move to a compressed configuration for travel along the second compression surface 858 and into the second groove 854, as shown in FIG. 11G. When the retention spring 882 is in the second groove 854, the angled edges of or adjacent to the second groove 854 do not inhibit or provide a much lower inhibition of movement in the first or second direction, such that the second connector 850 is in a partially unlocked state. To fully unlock or disconnect the second connector 850 from the first connector 851, the second connector 850 can be retracted in the second direction. When the second connector 850 moves in the second direction from the second groove 854, the second compression surface 858 alone or in combination with the tapered surface between the compression surface 858 and the second groove 854 compresses the retention spring 882 to allow the second connector 850 to be removed, as shown in FIG. 11H. In some implementations, the second connector 850 can be rapidly removed to prevent the retention spring 882 from expanding fully into the first groove 852, which could cause the second connector 850 to return to the engaged or locked configuration of FIG. 11F.

FIGS. 12A and 12B illustrate schematic circuit diagrams for an implantable control unit 100 (also referred to herein as a “controller” and a “control system”) in a first configuration and a second configuration respectively. The implantable control unit 100 can be incorporated in the implantable flow restriction system 5 as well as any other implementations of the implantable flow restriction systems described herein or in U.S. Pat. No. 11,883,030. The implantable control unit 100 can be patient-controlled and/or patient monitored, for example, wirelessly through a software application (e.g., a mobile device application or an “app”) on an external device 15 as shown. The implantable control unit 100 can be configured to be implantable within the patient and can including a source of implant actuation, a mother board comprising a processor, a memory, and in some implementations a communications module, and a source of power. The implantable control unit 100 can be configured to receive wireless or wired signals from sensor(s) 600 (e.g., pressure sensors) positioned in various locations within or around the heart or at other locations in the body, for example to measure pressure in the right ventricle, right atrial pressure, central venous pressure, aortic pressure, left atrial pressure, left ventricular pressure, aortic pressure, SVC pressure, IVC pressure, hepatic vein pressure, renal vein pressure, femoral vein pressure, and/or the pressure of any of the veins or portions thereof disclosed herein. Based on these readings, the implantable control unit 100 can appropriately actuate the implant 500 to control the adjustable occlusion of the implant in order to control the amount of blood flowing through the implant. The implantable control unit 100 can provide for closed-loop, fully autonomous, and/or real-time adjustability and control of the implant. The implantable control unit 100 can implement a treatment protocol/algorithm prescribed by a physician and/or the control logic can be optimized to treat heart failure patients, for example by reducing cardiac preload, reducing central venous pressure and/or pressure of other veins disclosed herein, increasing cardiac output, reducing renal congestion (or promoting renal decongestion), enhancing renal circulation, and/or enhancing or controlling diuresis (e.g., to increase diuresis). In some implementations, the implantable control unit 100 can receive data from sensor(s) connected to the implant 500 as described herein for the control of actuation of the implant 500. The sensor(s) can include any of the sensors described herein (e.g., sensor 600, sensor 600A, capacitive sensor 600B, sensor 600C, sensor 600D, and/or the like). Reference to the sensor 600 throughout this disclosure as part of the implantable flow restriction system 5 is understood to include any of the sensors described herein.

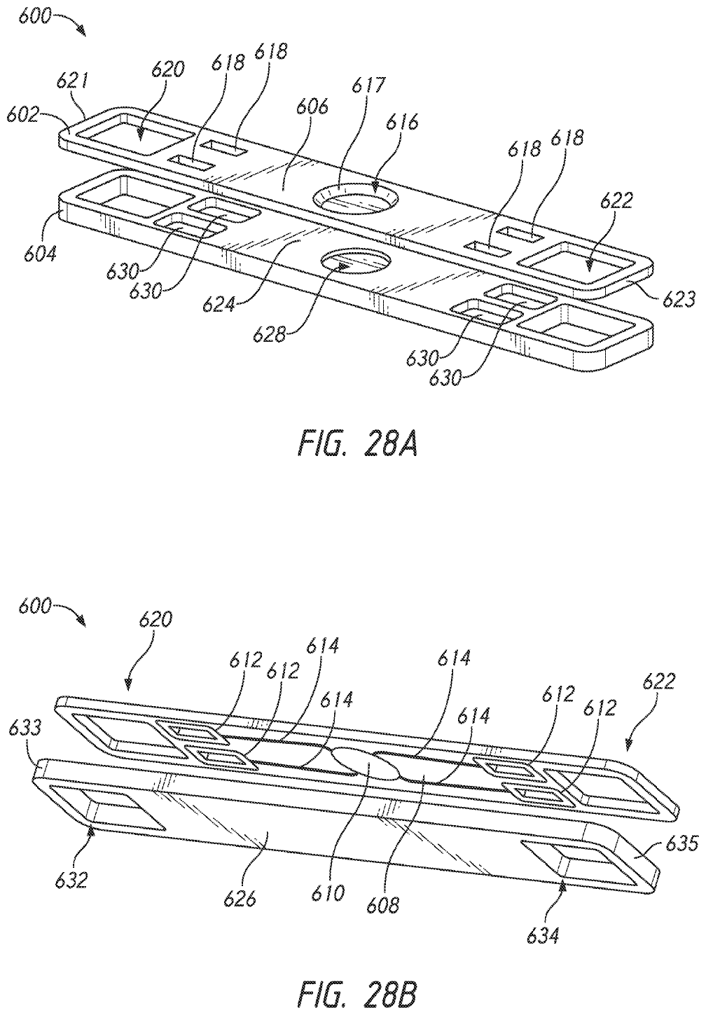

The implantable control unit 100 of the implantable flow restriction system 5 can include a microcontroller 102, an actuator 104, and a first power source 106. In some configurations, the implantable control unit 100 can include an induction receiver 108, an amplifier 110, and/or a second power source 112. FIGS. 12A and 12B also illustrate an external device 15 that can be used to operate the implantable flow restriction system 5 and a sensor 600 that may be electrically coupled to and/or in communication with the implantable control unit 100. The external device 15 can include a processor, a user interface, a storage device, a device power source, and/or a communication module. In some implementations, the external device 15 can be a mobile phone, a smart phone, a tablet, a handheld or mobile device, or otherwise. In some implementations, the external device 15 can include a wireless charger for powering the implantable control unit 100. In some implementations, the external device 15 can be a smart phone or other computing device used in combination with a wireless charger. In this example, the combination of the smart phone and wireless charger may be referred to herein as the “external device 15”. In some cases, the wireless charger may include an atmospheric pressure sensor. As described herein, in some cases, pressure measurements from sensors of the system 5 may be compared to local atmospheric pressure readings for improved therapy decisions. In this example, the wireless charger may be configured to communicate with the smart phone, and the smart phone can be used to communicate with the implantable control unit 100. For example, the smart phone may receive atmospheric pressure readings from the wireless charger and may transmit these atmospheric pressure readings to the implantable control unit 100. In other implementations, the smart phone or another dedicated pressure sensing device may include an atmospheric pressure sensor and/or a wireless charger, and a separate wireless charging device may not be required. The sensor(s) 600 may work and/or be used in conjunction with any of the flow restriction devices described herein and the implants described in U.S. Pat. No. 11,883,030 to provide physiological parameters of interest useful for control of the flow restriction device. The sensor(s) 600 are described in further detail with reference to at least FIGS. 28A-38E.

In some implementations, the implantable control unit 100 and any of the flow restriction devices described herein (e.g., the implant 500) may work with and/or be used in conjunction with sensors that are located remote from the flow restriction device that can provide physiological parameters of interest useful for control of the implant 500. These sensors may be used in addition to or alternatively to the sensor(s) 600. Such physiological parameters of interest can include pressure, flow rate, heart rate, and/or the like. As an example, a flow restriction device can be used with a pressure sensor located within vessels and/or organs remote from the flow restriction device and provide a measure of the pressure at such locations for the control of the flow restriction device. One example of an implantable sensor is a MEMS pressure sensor. The MEMS or other implantable pressure sensor may be a remote component of the flow restriction device or may be an independent sensor with a separate control system. In one example, the MEMS pressure sensor may be located in the pulmonary artery and may measure the pressure of blood flowing through the pulmonary artery. The MEMS or other pressure sensor may include a separate electronics system that is configured to receive readings (e.g., data indicative of pressure) from the MEMS pressure sensor. These readings may be used by the patient, the patient's physician, etc. to determine when the patient should receive treatment via the flow restriction device. In one example, the MEMS pressure sensor may comprise a capacitive sensor. In another example, the MEMS pressure sensor may include a barometer and may be powered by an external antenna (e.g., in the form of radiofrequency signals). For example, the external antenna may be contained within an antenna device and a pressure reading may be taken and transmitted to the electronics system when the patient holds the antenna device near and/or against their body. Additionally or alternatively, the MEMS pressure sensor may include an inductor that can be used to create a circuit that creates a frequency, e.g., an LC circuit or LC tank circuit. The frequency may then be used to determine the pressure.

In some implementations, the MEMS pressure sensor described above may be coupled to a portion of the flow restriction devices described herein. As an example, a flow restriction device can have the MEMS pressure sensor attached to its proximal end, its distal end, both of its ends, its shaft, and/or the like. In the example of the implant 500, the MEMS pressure sensor may be coupled to the shaft 590. The MEMS pressure sensor may be tied to/coupled to the flow restriction device with suture, a reflow process, and/or the like. In this example, the MEMS pressure sensor would be configured to measure the pressure at such location relative to the flow restriction device (e.g., upstream, downstream, both upstream and downstream, etc.). As noted above, the MEMS pressure sensor may transmit the pressure readings to a separate electronics system. Additionally or alternatively, the MEMS pressure sensor may transmit readings to a control system of the flow restriction device (e.g., the controller 100).

The microcontroller 102 may include one or more processors, storage devices (e.g., computer readable storage devices storing computer-executable instructions), and/or communication modules. The microcontroller 102 can be mounted on a printed circuit board 101 (see e.g., printed circuit board 101 of FIG. 14B) to facilitate the integration of the electrical components of the implantable control unit 100. The processors can be configured, among other things, to process data, execute instructions to perform one or more functions, and/or control the operation of the implant 500, the sensor(s) 600, and the external device 15, respectively. For example, the processor can control operation of the actuator 104 and the sensor(s) 600 of the chronic, implantable flow restriction system 5. As another example, the processor can process signals and/or data received and/or obtained from the sensor(s) 600 of the implantable flow restriction system 5. Further, the processor can execute instructions to perform functions related to storing and/or transmitting such signals and/or data received and/or obtained from the sensor(s) 600 of the implantable flow restriction system 5 (e.g., such as transmitting such signals and/or data to external device 15). The processor can execute instructions to perform functions related to storing and/or transmitting any or all of such received data. As shown in FIGS. 12A and 12B, the implantable control unit 100 may include the amplifier 110. The amplifier 110 may amplify the signals from the sensor(s) 600. The amplifier 110 may be a transistor based circuit, in one example.

The storage devices can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed data obtained from the implantable flow restriction system 5, for example.

The communication modules can facilitate communication (e.g., via wireless connection) between the implantable flow restriction system 5 (and/or components thereof, such as the implantable control unit 100), the external device 15, as well as other separate devices, such as separate monitoring, computing, electrical, and/or mobile devices. For example, the communication module can be configured to allow the implantable flow restriction system 5 to wirelessly communicate with external device 15 and/or other devices, systems, and/or networks over any of a variety of communication protocols. The communication modules can be configured to use any of a variety of wireless communication protocols, such as Wi-Fi (802.11x), Bluetooth®, ZigBee®, Z-Wave®, cellular telephony, infrared, near-field communications (NFC), RFID, satellite transmission, proprietary protocols, combinations of the same, and the like. The communication module can allow data and/or instructions to be transmitted and/or received to and/or from the implantable flow restriction system 5 and separate computing devices, such as the external device 15. The communication module can be configured to transmit (for example, wirelessly) processed and/or unprocessed data (e.g., data from sensor(s) 600) and/or other information to one or more separate computing devices, which can include, among others, external device 15, a patient monitor, a mobile device (for example, an iOS or Android enabled smartphone, tablet, laptop), a desktop computer, a server or other computing or processing device for display and/or further processing, among other things. Such separate computing devices can be configured to store and/or further process the received data and/or other information, to display information indicative of or derived from the received data and/or information, and/or to transmit information, including displays, alarms, alerts, and notifications, to various other types of computing devices and/or systems that can be associated with a hospital, a caregiver (for example, a primary care provider), and/or a user (for example, an employer, a school, friends, family, etc.) that have permission to access the patient's data. As another example, the communication module of the implantable control unit 100 of the implantable flow restriction system 5 can be configured to wirelessly transmit processed and/or unprocessed obtained data, information and/or other information (for example, a status of actuation of an implant 500) to a mobile phone which can include one or more hardware processors configured to execute an application that generates a graphical user interface displaying information representative of the processed or unprocessed data, and/or other information obtained from the implantable flow restriction system 5. The communication modules can be and/or include a wireless transceiver.

The power sources 106, 112 can provide power for hardware components of the implantable flow restriction system 5 described herein. For example, one or both the first power source 106 and the second power source 112 of the implantable control unit 100 can provide power to the sensor(s) 600, the microcontroller 102, and/or the actuator 104. In some implementations, the power sources 106, 112 can comprise a battery, a receiver/rectifier (e.g., induction), or both. In the first configuration shown in FIG. 12A, the implantable control unit 100 includes only one power source 106 that is configured to be implanted and to store energy for delivering current to the electrical components of the control unit 100. In some implementations, the power source 106 can be a battery (e.g., a 1 mAh battery) or a capacitor. The power source 106 is electrically coupled to the induction receiver 108 and the microcontroller 102. Generally, the power source 106 is not configured to store a large amount of energy. As such, the implantable control unit 100 may use the external device 15 to power the implantable flow restriction system 5. For example, the external device 15 can include an induction transmitter to wirelessly transmit power to the power source 106 via the induction receiver 108. As such, the power source 106 may generate a current (e.g., directed towards the microcontroller 102 and/or the motor 120) when exposed to or in close proximity to the external device 15. Exposure, as the term is used herein, may refer to the patient placing the external device 15 near or on their skin over or around the location of the implantable control unit 100. In some implementations, the patient may use a separate external device for inductive coupling and the external device 15 for communicating with the implantable control unit 100.

In the second configuration shown in FIG. 12B, the implantable control unit 100 includes the first power source 106 and the second power source 112, both of which are configured to store energy for delivering current to the electrical components of the control unit 100. The second power source 112 can be a battery (e.g., a 100 mAh battery). The second power source 112 can be electrically coupled to the microcontroller. In some implementations, the second power source 112 may be electrically coupled to the induction receiver 108 such that the second power source 112 can be charged via induction. Because the implantable control unit 100 includes two power sources 106, 112 in the second configuration, the power sources 106, 112 can serve to provide power to different components of the implantable flow restriction system 5. In some implementations the first power source 106 is used for high energy use functions, such as actuating the implant 500, while the second power source 112 is used for low energy use functions, such as powering the microcontroller 102 for communications. For example, the first power source 106 may provide power to the actuator 104 for the actuation of the implant 500. As such, the implant 500 may be actuated at set times when the external device 15 is at a close proximity to the implantable control unit 100. When the first power source 106 has the capacity to store energy, the first power source 106 may be used to actuate the implant 500 after a treatment time has expired. For example, a user may place the external device 15 in close proximity of the implantable control unit 100 to power the first power source 106 and move the implant 500 into a treatment state (e.g., where the implant 500 at least partially occludes a vessel). After a set duration, the first power source 106 may provide power to the implantable control unit 100 to return the implant 500 to an original state (e.g., where the implant 500 is not occluding the vessel). The second power source 112 may be configured to provide power to the microcontroller 102 to allow the microcontroller 102 to communicate with the sensor(s) 600 and/or the external device. The second power source 112 may ensure that the microcontroller 102 has sufficient power to always be monitoring the implantable flow restriction system 5. The second power source 112 and/or the first power source 106 can be rechargeable batteries. For example, such batteries can be a lithium, a lithium polymer, a lithium-ion, a lithium-ion polymer, a lead-acid, a nickel-cadmium, or a nickel-metal hydride battery. In some implementations, such batteries can be non-rechargeable. In some implementations, the implantable control unit 100 may only include the second power source 112 and may not include the first power source 106.

The actuator 104 of the implantable control unit 100 of the implantable flow restriction system 5 can be configured to move the shaft 590 within tubing 570 for actuation of flow restrictor 560 and/or flow restrictor portion 550 of implant 500 (which can include any of the implants described herein). For example, the actuator 104 can be configured to slidingly move the shaft 590 proximally and/or distally relative to the tubing 570 and the implant 500. As another example, the actuator 104 can be configured to rotationally move the shaft 590 relative to the tubing 570 and the implant 500. Furthermore, the actuator 104 can be configured to cause the flow restrictor 560 and/or the flow restrictor portion 550 of the implant 500 to occlude/restrict flow through the implant 500 in a range of from and including about 0% to about 100%. Here, 0% is the original low profile state of the implant 500 and 100% is the high profile state. The actuator 104 may include a motor to perform these functions, as described further herein.

As noted above, the external device may include a user interface. The user interface can be configured to allow a patient or their care provider to interact with the external device 15 for control of the implantable flow restriction system 5. The user interface can include button(s), a touch screen, and/or a microphone to accept physical touch and/or verbal input/commands.

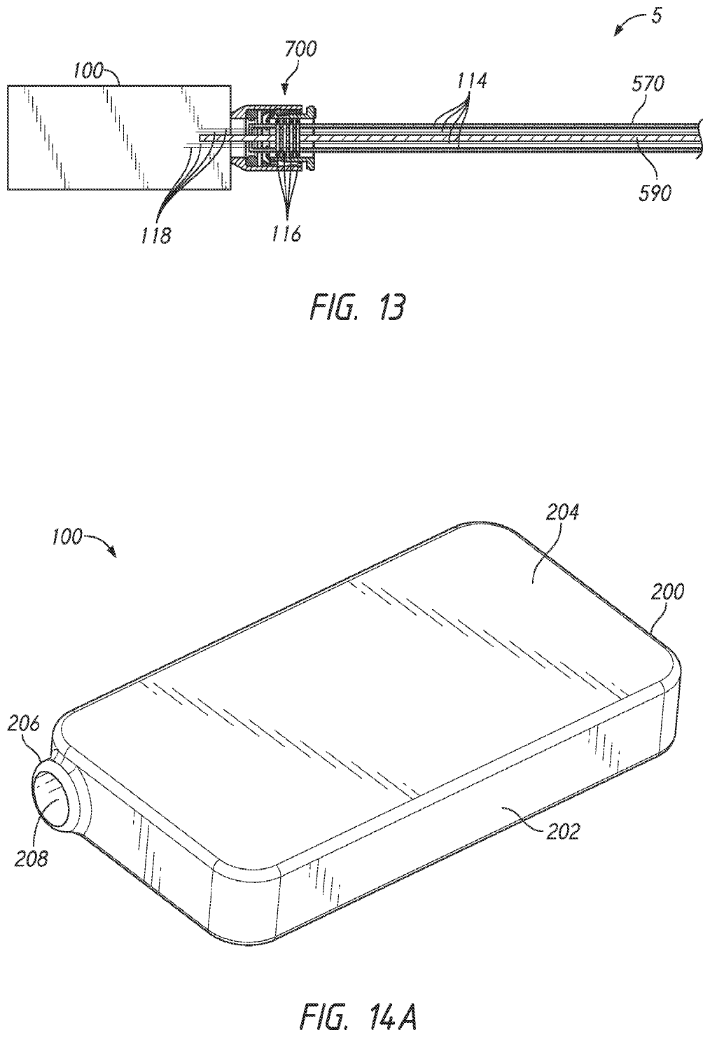

FIG. 13 illustrates a schematic diagram of a portion of an implementation of the implantable flow restriction system 5. As shown, the implantable control unit 100 may be coupled to the tubing 570 and shaft 590 of the implant 500 via the connector 700. The shaft 590 functions as the control member for the implant 500. The connector 700 is shown for illustrative purposes, but it is recognized that any of the connectors described herein can be used with the implantable control unit 100. The tubing 570 may house a plurality of wires 114 that extend from one or more sensors 600 to electrically connect the implantable control unit 100 to the one or more sensors 600. For example, the wires 114 may be positioned between the outer diameter of the shaft 590 and the inner diameter of the tubing 570. In another example, the wires 114 may be positioned between the outer diameter and inner diameter of the tubing 114. For example, the wires 114 may be disposed within the walls of the tubing 570. In some implementations, there may be four or more wires 114. In some implementations, the wires 114 may be electrically coupled to a plurality of electrical contacts 116. The electrical contacts 116 may be positioned within the connector 700, disposed on the tubing 570, coupled to a separate connector portion, and/or the like. The plurality of electrical contacts 116 may be electrically coupled to wires housed in the connector 700, a separate controller portion, and/or controller wires 118, which can be connected to the microcontroller 102.

FIGS. 14A-14D illustrate an implementation of the implantable control unit 100 of the implantable flow restriction system 5. FIG. 14A shows a perspective view of an external housing 200 of the implantable control unit 100, FIG. 14B shows an exploded view of the implantable control unit 100 and external housing 200, and FIGS. 14C and 14D show top views of the implantable control unit 100.

With reference first to FIG. 14A, the external housing 200 can include a base 202 and a cover 204. The external housing 200 may be any suitable shape, such as square, rectangular, circular, and/or the like. As shown in FIG. 14A, the external housing 200 can be generally rectangularly shaped and may include rounded edges. The external housing 200 may be any suitable material such as a metal, a plastic, a ceramic, or a combination of two or more materials. In some configurations, a plastic external housing 200 may provide certain benefits of reducing interference for inductive coupling and communication (e.g., via Bluetooth®) between the implantable control unit 100 and the external device 15. The components of the implantable control unit 100 may be enclosed within the base 202. The cover 204 is configured to mate with the base 202. The external housing 200 protects the components of the implantable control unit 100 when the cover 204 is fixed to the base 202. The external housing 200 may also include a connector sleeve 206. The connector sleeve 206 includes a hole 208 that extends through the base 202 to provides access to the internal portion of the external housing 200. Other components of the implantable flow restriction system 5, such as the shaft 590, may extend through hole 208 into the external housing 200. Generally, the hole 208 is scaled during use, to protect the implantable control unit 100. The connector sleeve 206 may be a circular projection. In some implementations, the connector sleeve 206 may be threaded.

As shown in FIG. 14B, the external housing 200 may include an ingress protection system 150. The ingress protection system 150 may include an internal housing 210 and an internal cover 212. The ingress protection system 150 can isolate the mechanical portions of the implantable control unit 100 from the electrical portions, as described further below. The ingress protection system 150 may be hermetically sealed relative to the external housing 200. In some implementations, the internal housing 210 and the cover 212 may be a different material than the external housing 200. For example, the internal housing and cover 210, 212 may be made of metal, such as titanium.

The actuator 104 of the implantable control unit 100 may be a linear actuator. The actuator 104 may include a motor 120, a rod 122, and a traveler 128. In some implementations, the actuator 104 may further include a linear guide 124. The motor 120 can be controlled by the microcontroller 102 (not shown) via a printed circuit board 101 and can be powered by one or both of the first power source 106 and second power source 112. For example, the first power source 106 can be used to charge the second power source 112, which can power the actuator 104, when inductively coupled to the external device 15. A shaft of the motor 120 may be coupled to the rod 122. As such, rotation of the shaft causes corresponding rotation of the rod 122. The rod 122 may include external threads. The motor shaft and the rod 122 may extend through a divider 126. The divider 126 may isolate the rod 122 from the motor 120 when the internal housing 210 is sealed with the cover 212. The linear guide 124 may include a rod. The linear guide 124 may extend from the divider 126. A central axis of the linear guide 124 may be parallel to a central axis of the rod 122. The divider 126 may be configured to prevent rotation of the linear guide 124.

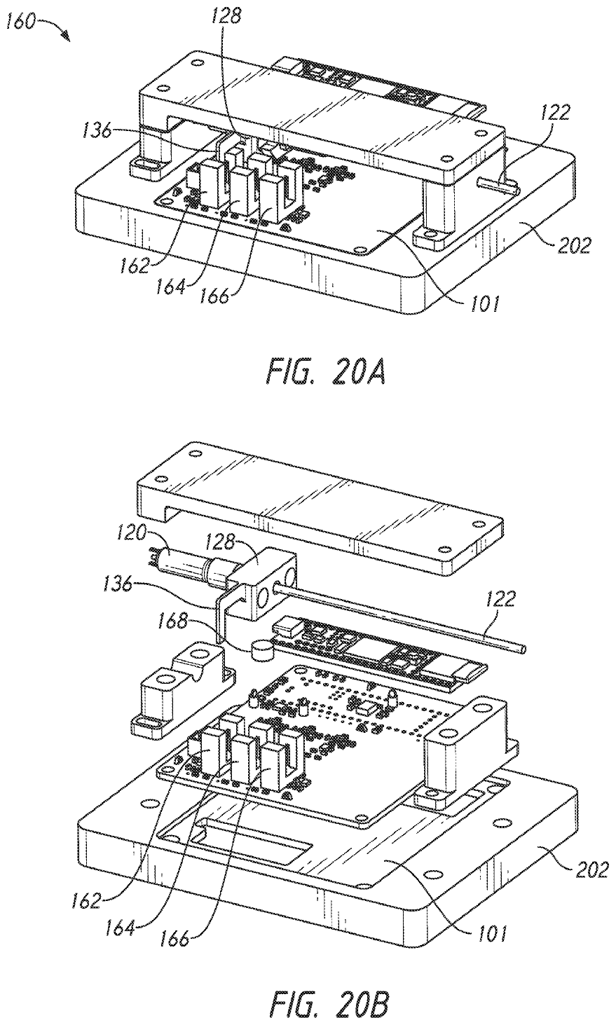

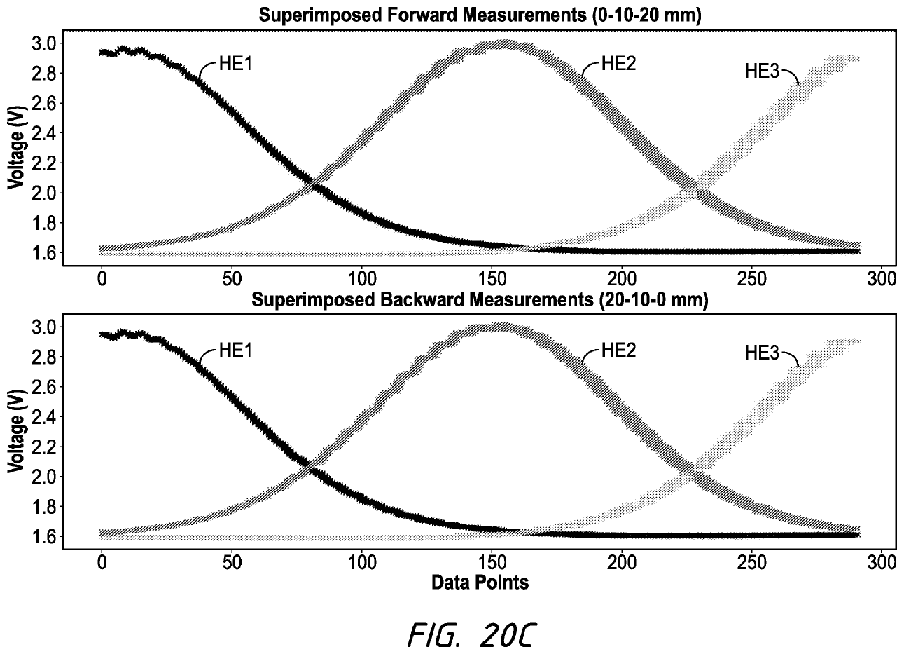

The traveler 128 is configured to translate along the rod 122. The traveler 128 may include one or more holes. For example, in the implementation illustrated in FIG. 14B, the traveler 128 includes a first hole 130, a second hole 132, and a third hole 134. The implant 500 may be coupled to the traveler 128 via the shaft 590. For example, the first/distal end 592 of the shaft 590 may be coupled to the implant 500 and the second/proximal end 591 end of the shaft 590 may extend into the first hole 130 of the traveler 128. As such, the shaft 590 is configured to move when the traveler 128 is translated along the rod 122. The second hole 132 may be threaded and is configured to receive the rod 122. As the rod 122 is rotated, the threads of the rod 122 engage the threads of the second hole 132 such that the traveler 128 translates along the length of the rod 122. The position of the traveler 128 along the length of the rod 122 may define the actuation of the implant 500. For example, the traveler 128 may move between a first end position, which corresponds to a minimum-occlusive state of the implant 500, and a second end position, which corresponds to a maximum occlusive state of the implant 500. The amount of occlusion provided by the implant 500 can be defined by the position of the traveler 128 between the first end position and the second end position. For example, the implant 500 can be configured to provide a 50% restriction of flow when the traveler 128 is equal distance from the first end position and the second end position of the rod 122. Movement of the traveler 128 toward the hole 208 can allow the implant 500 to move toward a low profile state corresponding to less restriction of flow. Movement of the traveler 128 away from the hole 208 can apply a load on the distal apices 564 of the implant 500 to move the petals 560 toward a flow restricting state. The percent occlusion provided by the implant 500 may not be linearly correlated with the position of the traveler 128. In one embodiment, a control method is implemented by the microcontroller 102 whereby a known percent occlusion as a function of linear position of the traveler 128 is provided. The third hole 134 may be configured to receive the linear guide 124. As the traveler 128 translates along the rod 122, the traveler 128 also translates along the linear guide 124. The linear guide 124 may reduce, minimize or prevent rotation of the traveler 128 about the axis of the rod 122 during translation along the rod 122 and may also help guide the traveler 128. In some implementations, the traveler 128 may include one or more additional holes. For example, the traveler 128 may include a fourth hole (not shown) positioned on a bottom side of the traveler 128 that is configured to receive a magnet (not shown) for position detection (e.g., see magnet 168 of FIGS. 20A and 20B). A fourth hole can be placed on other sides of the traveler 128 in other embodiments. The traveler 128 can be molded around a magnet in another approach such that no holes are required for the magnet to be disposed in or on the traveler.

FIG. 14C illustrates a top view of the implantable control unit 100 with the cover 204 removed. FIG. 14D illustrates a top view of the implantable control unit 100 with the cover 204 and the cover 212 removed. As shown, the rod 122, linear guide 124, and the traveler 128 are positioned within the internal housing 210 and isolated from the internal portion of the external housing 200. In some implementations, the divider 126 provides a seal between a wall of the internal housing 210 and the base 202. In some implementations, when the cover 212 is coupled to the internal housing 210, the internal housing 210 is hermetically sealed from the external housing 200. As such, if there is a leak in the connector 700 or at the connector sleeve 206, any fluid (e.g., blood), that enters the implantable control unit 100 would remain in the internal housing 210, protecting the electrical components of the external housing 200. This arrangement may provide a benefit of enabling the implantable control unit 100 to maintain communications during a mechanical failure. The mechanical components within the internal housing 210 can continue to operate even in the presence of some fluid flowing therein due to leakage. In some implementations, the main internal portion (e.g., not the internal housing 210) may enclose or be at least partially filled with an insulating material/isolation layer, e.g., a resin, such as epoxy (e.g., an insulating/isolating epoxy), to block flow of any fluid from leakage into the internal housing 210. The epoxy may protect the electrical components of the implantable control unit 100 from damage if any fluid enters the internal housing 210 and would otherwise flow into other parts of the external housing 200. In some implementations, either the internal housing 210 or the cover 212 may include a slot or hole for routing the controller wires 118 to the microcontroller 102. The slot/hole may then be sealed (e.g., with an epoxy or other isolation layer).

FIGS. 14E-14H illustrate an implementation of an implantable control unit 100D of the implantable flow restriction system 5. Some of the features of the implantable control unit 100D are similar to features of the implantable control unit 100 in at least FIGS. 12A-14D. Thus, reference numerals used to designate the various features or components of the implantable control unit 100 are identical to those used for identifying the corresponding features of components of implantable control unit 100D in FIGS. 14E-14F, except that an “D” has been added to the numerical identifier for the implantable control unit 100D. Therefore, the structure and description for the various features of the implantable control unit 100 and the operation thereof as described in at least FIGS. 12A-14D are understood to also apply to the corresponding features of the implantable control unit 100D in FIGS. 14E-14F, except as described differently below.

FIG. 14E illustrates a top view of the external housing 200D of the implantable control unit 100D and FIG. 14F illustrates a perspective view of the external housing 200D of the implantable control unit 100D. In FIGS. 14E-14G, the implantable control unit 100D is shown coupled to the shaft 590 and the tubing 570 of the implantable flow restriction system 5, with the sensor 600 coupled to tubing 570 near the distal end 572. In FIGS. 14E and 14F, a closure 216D, cover 212D of the ingress protection system 150D, and cover 204D are shown as partially see-through for illustrative purposes.

The implantable control unit 100D differs from the implantable control unit 100 primarily in the relationship between the motor 120 and the rod 122D. In the implantable control unit 100D, a central axis of the motor 120 may be perpendicular or at approximately a 90-degree angle relative to a central axis of the rod 122D. The implantable control unit 100D may include a first gear 170D and a second gear 172D. The first and second gears 170D, 172D may be beveled gears. The first gear 170D may be rotationally coupled to a shaft 121 of the motor 120. The second gear 172D may be rotationally coupled to the rod 122D. The first gear 170D may mesh with the second gear 172D such that rotation of the shaft 121 causes corresponding rotation of the rod 122D. In some implementations, the first and second gears 170D, 172D may have a one-to-one gear ratio. In some implementations, the first and second gears 170D, 172D may have a gear ratio of more or less than one-to-one.

The implantable control unit 100D may include a first bearing 174D and a second bearing 176D. The outer race of the first bearing 174D may be coupled to the base 202D of the external housing 200D and the inner race of the first bearing 174D may be coupled to the shaft 121. In this arrangement, the shaft 121 is supported by and can rotate relative to the base 202D. The outer race of the second bearing 176D may be coupled to the base 202D and the inner race of the first bearing 174D may be coupled to the second gear 172D. In this arrangement, the second gear 172D is supported by and can rotate relative to the base 202D.

The implantable control unit 100D may include a first divider 126D and a second divider 126D′. The first divider 126D can function in a similar manner to the divider 126, except that the first divider 126D only provides support and rotational prevention for the linear guide 124D. Additionally, the first divider 126D may not provide the sealing function described above. The second divider 126D′ may provide support for the motor 120. In some implementations, the second divider 126D′ provides a seal between a wall of the internal housing 210 and the base 202. In some implementations, the first divider 126D and the second divider 126D′ may form a single L-shaped component. One benefit of having separate dividers 126D, 126D′ may be that less space is used inside the external housing 200D. Additionally, because the first divider 126D is positioned within the internal housing 210D, the first divider 126D may not provide a sealing function while the separate second divider 126D′ can provide a seal between a wall of the internal housing 210D and the base 202D. In some arrangements, the separate second divider 126D′ provides a barrier to flow between the internal housing 210D D and an interior of the base 202D outside of the internal housing 210D without fully sealing these internal spaces from each other.

As shown more clearly in FIG. 14E, the shaft 121, the first and second gears 170D, 172D, the bearings 174D, 176D, and the dividers 126D, 126D′ can all be housing in the internal housing 210D. The internal housing 210D and the cover 212D form some or all of the ingress protection system 150D. The ingress protection system 150D isolates the mechanical portions of the implantable control unit 100D from the electrical portions, as described above. As a result of the arrangement between the rod 122D and the motor 120, the internal portion of the base 202D outside of the internal housing 210D may be generally rectangularly shaped, as opposed to L-shaped in the implantable control unit 100.

As shown in FIG. 14F, the traveler 128D can house a magnet 168 and may include an additional hole 135D. The magnet 168 can be used for determining the position of the traveler 128D relative to the external housing 200 using a sensor system, such as the sensor system 160 described below with reference to at least FIGS. 20A-20C. The hole 135D can be included in the traveler 128D to facilitate assembly of the magnet 168 to the traveler 128D. The hole 135D can be included in the traveler 128D to reduce the interference with the magnetic field of the magnet 168.

Use of gears 170D and 172D in the implantable control unit 100D may provide certain benefits. For example, because the rod 122D is perpendicular to the motor 120, the size of the external housing 200D can be reduced. Additionally, the external housing 200D can be generally square shaped and may include rounded edges, which may be desirable.

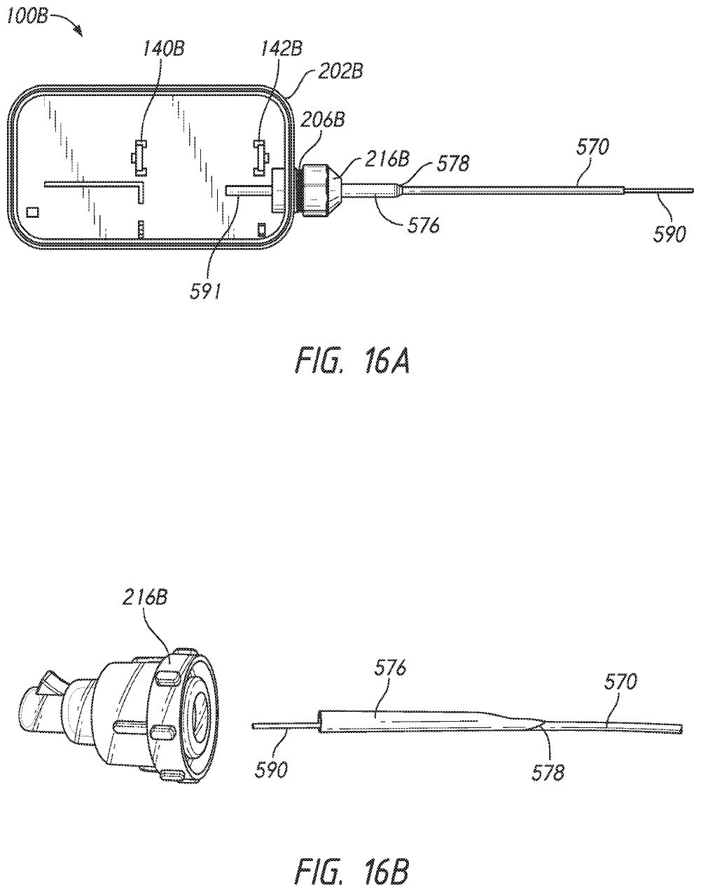

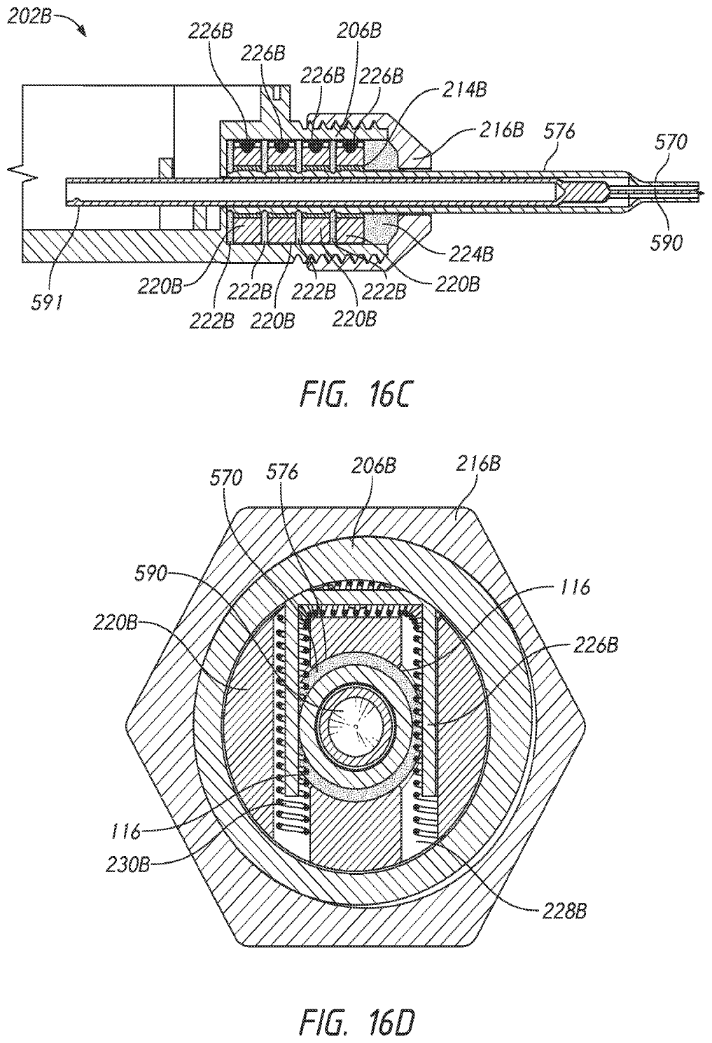

FIG. 14G illustrates a close up perspective view of the connector sleeve 206D and the closure 216D engaged with the tubing 570 and shaft 590 of the implantable flow restriction system 5. The closure 216D and associated components are similar or identical to the closure 216B and associated components described below in FIGS. 16A-16D. Thus, reference numerals used to designate the various features or components of the closure 216B are identical to those used for identifying the corresponding features of components of closure 216D in FIGS. 14G, except that a “D” has been added to the numerical identifier for the closure 216D. Therefore, the structure and description for the various features of the closure 216B and associated component and the operation thereof as described below in at least FIGS. 16A-16D are understood to also apply to the corresponding features of the closure 216D and associated components, except as described differently below.

The wire housings 220D can each include a connector electrode 117 on an external surface thereof. The connector electrodes 117 may be coupled to the controller wires 118, which can connect to the microcontroller 102. The connector electrodes 117 can also be in electrical contact with the connector wires 226D and/or the springs 230B, which are in electrical contact with the electrical contacts 116 of the wires 114. Thus, the sensor 600 can be electrically connected to the microcontroller 102. While the connector electrodes 117 are not shown in FIGS. 16A-16D, it is recognized that the wire housings 220B can include connector electrodes 117 or a similar system.

FIG. 14H illustrates an alternative arrangement of some internal components of the implantable control unit 100D. The arrangement shown in FIG. 14H differs from the arrangement shown in FIG. 14E in the implantable control unit 100D includes an additional third gear 171D. The third gear 171D can be positioned between the first and second gears 170D, 172D, and can mesh with the other two gears 170D, 172D. This arrangement can allow the motor 120 to be parallel to the rod 122D within the external housing 200D. As such, the volume of the external housing 200D can be reduced compared to the arrangement shown in FIG. 14E. In variations, the rod 122D and the shaft 121 could be coupled by more or fewer than three gears (as illustrated) while providing the configuration where the motor 120 and the rod 122D are aligned parallel or overlap in a direction within a housing.

FIGS. 141 and 14J illustrate top views of an implementation of the implantable control unit 100D that includes a connector system 180D. The connector system 180D can be used to releasably connect the proximal end 591 of the shaft 590 to the traveler 128D. The connector system 180D can move between a first configuration and a second configuration. In one embodiment, in the first configuration, the shaft 590 is in a locked position relative to the traveler 128D. In one embodiment, in the second configuration, the shaft 590 is in an unlocked position relative to the traveler 128D and can be removed from the traveler 128D.

The connector system 180D can include push-to-lock connector 182D and a flange actuator 184D. The push-to-lock connector 182D can be configured to allow the shaft 590 to be removably coupled and decoupled from the control unit 100D by applying and removing a force to the push-to-lock connector 182D. The force to couple and decouple can be applied in the same direction when connecting and when disconnecting. The push-to-lock connector 182D can include a first bore (not shown) that can receive the proximal end 591 of the shaft 590. The push-to-lock connector 182D can be coupled to the traveler 128D such that the connector system 180D moves proximally and distally with the traveler 128D. The push-to-lock connector 182D can include one or more push-to-lock mechanisms (not shown) or other locking mechanisms within the first bore that are configured to lock the shaft 590 to the connector system 180D in the locked configuration. For example, push-to-lock mechanisms can include rings, sleeves, barbs, collars, and/or the like. In some examples, the push-to-lock mechanisms can extend inwardly into a center of first bore of the push-to-lock connector 182D in the locked configuration to engage the shaft 590 and can retract outwardly from the center of the bore in the unlocked configuration. The flange actuator 184D can be configured to move the push-to-lock connector 182D between the locked and unlocked configuration. The flange actuator 184D can extend at least partially into the first bore of the push-to-lock connector 182D. The flange actuator 184D can include a second bore (not shown). The second bore can extend into the first bore in a concentric manner. In the locked configuration (shown in FIG. 14I), the flange actuator 184D can be at a maximum extension outside of the push-to-lock connector 182D. In the unlocked configuration (shown in FIG. 14J), the flange actuator 184D can be at a maximum extension inside of the push-to-lock connector 182D. To move from the locked configuration to the unlocked configuration, the motor 120 can drive the traveler 128D distally such that the flange actuator 184D contacts an internal wall 186D of the external housing 200D. Continued engagement between the flange actuator 184D and the internal wall 186D can cause the flange actuator 184D to extend into the first bore of the push-to-lock connector 182D. As the flange actuator 184D moves into the first bore, the push-to-lock mechanisms can release the proximal end 591 of the shaft 590, such that the connector system 180D is in the unlocked configuration. For example, the flange actuator 184D may cause the push-to-lock mechanisms to retract outwardly from the center of the first bore. Stated another way, in the locked configuration (shown in FIG. 14I), a gap is provided between a proximal surface of the flange actuator 184D and a distal surface of the push-to-lock connector 182D. In the unlocked configuration (shown in FIG. 14J), the gap is reduced or eliminated as the proximal surface of the flange actuator 184D moves toward or contacts the distal surface of the push-to-lock connector 182D. In the unlocked configuration, the shaft 590 can be released from the connector system 180D by moving the shaft 590 distally. During normal movement of the traveler 128D within the external housing 200D, the connector system 180D may not contact the internal wall 186D. The implantable control unit 100D may include a sensor system (e.g., the sensor system 160 described in FIGS. 20A-20C, which can be a Hall effect sensor) to track the position of the traveler 128D, such that the implantable control unit 100D can be instructed to move the traveler 128D to a position where the connector system 180D engages the internal wall 186D. When the implantable control unit 100D is not instructed in this manner, the sensor system can ensure that the connector system 180D remains in the locked configuration.

To move the connector system 180D to the locked configuration from the unlocked configuration, the proximal end 591 of the shaft 590 can be inserted through the first bore of the flange actuator 184D and into the second bore of the push-to-lock connector 182D. A proximal force applied to the shaft 590 can cause the flange actuator 184D to retract out of the push-to-lock connector 182D such that the connector system 180D moves to the locked configuration.

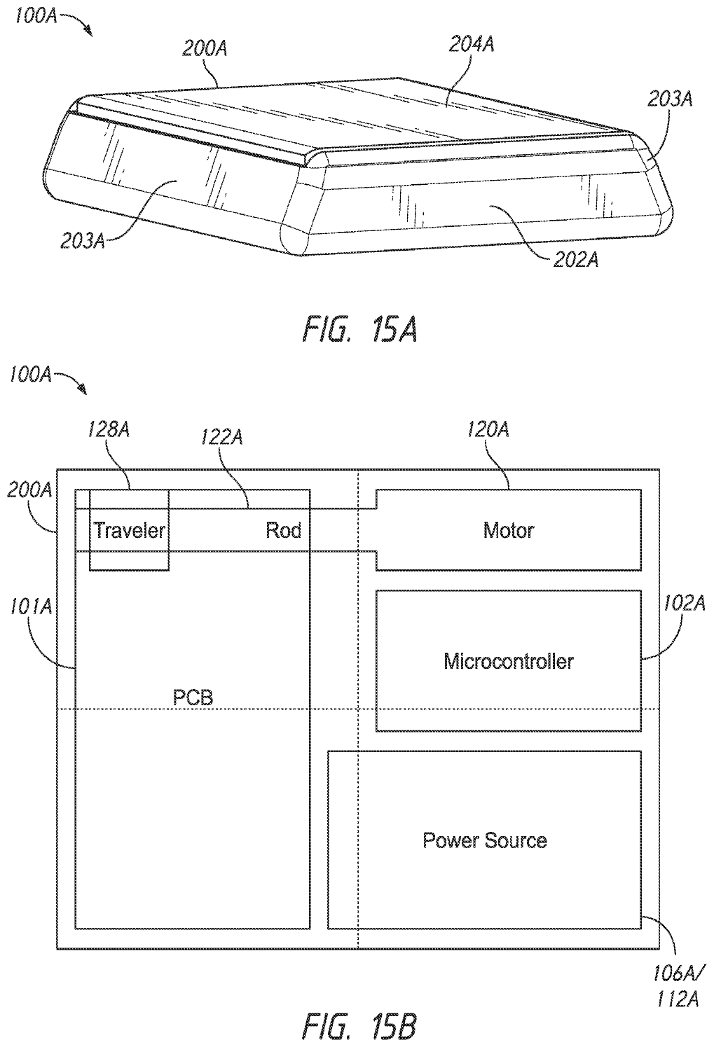

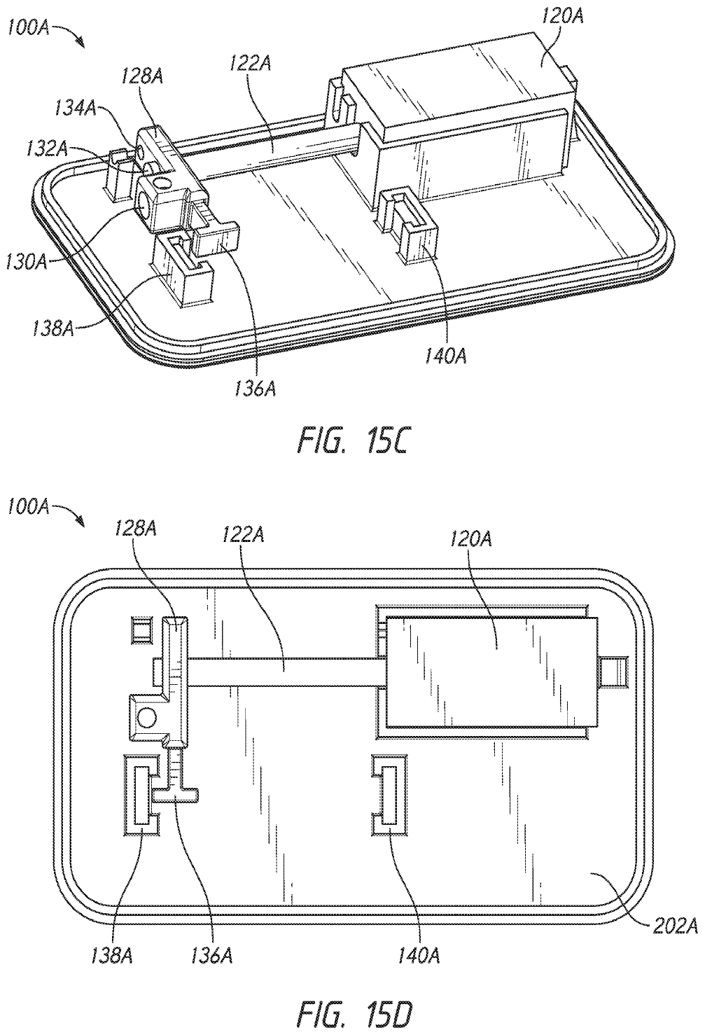

FIGS. 15A-15D illustrate an implementation of an implantable control unit 100A of the implantable flow restriction system 5. Some of the features of the implantable control unit 100A are similar to features of the implantable control unit 100 in at least FIGS. 12A-14D. Thus, reference numerals used to designate the various features or components of the implantable control unit 100 are identical to those used for identifying the corresponding features of components of implantable control unit 100A in FIGS. 15A-15D, except that an “A” has been added to the numerical identifier for the implantable control unit 100A. Therefore, the structure and description for the various features of the implantable control unit 100 and the operation thereof as described in at least FIGS. 12A-14D are understood to also apply to the corresponding features of the implantable control unit 100A in FIGS. 15A-15D, except as described differently below.

FIG. 15A illustrates a perspective view of the external housing 200A of the implantable control unit 100A. The external housing 200A may have tapered side walls 203A. For example, the external housing 200A may be generally trapezoidal shaped (e.g., the outer surfaces of the housing 200A may be seen as a trapezoid when a cross-section is taken through the wall 203A and in a direction perpendicular to a top surface of a cover 204A of the housing 200A). A trapezoidal implantable control unit 100A may provide certain benefits, including increased patient comfort once implanted. The bottom of a base 202A of the housing 200A can be implanted inward of the cover 204A, which is a narrower side of the housing 200A. The cover 204A can be the portion of the housing 200A closest to the skin when the housing 200A is implanted.

FIG. 15B illustrates a schematic diagram of some components of implantable control unit 100A. FIG. 15B shows a possible arrangement for these components relative to each other. For example, one end of the printed circuit board 101A may extend beneath the rod 122A and/or the traveler 128A. The printed circuit board 101A may be on one side of the base 202A and the power source(s) 106A, 112A and the microcontroller 102A may be positioned on an opposite side of the base 202A.

FIGS. 15C and 15D illustrate the internal portion of the external housing 200A with some components of the implantable control unit 100A removed. The implantable control unit 100A may differ from the implantable control unit 100 in that the implantable control unit 100A may not include an internal housing 210. The housing 200A of the control unit 100A can have an undivided interior in which components of the control unit 100A are housed. The traveler 128A may include an extension portion 136A. The extension portion 136A may be coupled to the traveler 128A or may form a single unit with the traveler 128A. In some implementations, the extension portion 136A may be generally T-shaped. The external housing 200A may further include a first end member 138A and a second end member 140A. The first and second end members 138A, 140A may be C-shaped projections extending from the base 202A. As the traveler 128A translates along the rod 122A, the extension portion 136A may move between the first end member 138A and the second end member 140A. In some implementations, the first end member 138A may define a minimum occlusive position for the implant 500 and the second end member 140A may define a maximum occlusive position for the implant 500. In some implementations, position sensors (not shown) may be mounted on the first and second end members 138A, 140A. The position sensors may be configured to generate a signal corresponding to the position of the extension portion 136A, which corresponds to the position of the traveler 128A and the occlusive state of the implant 500. The position sensors may be configured to generate a signal corresponding to the position of the traveler 128A or a portion thereof. The position sensors may be configured to generate a signal corresponding to the occlusive state of the implant 500. The position sensors may communicate the signal to the microcontroller 102A, which can calculate and provide an output indicative of the degree of occlusion or the occlusive state of the implant 500 such that a user can control the behavior of the implant 500, e.g., the percent occlusion provided by the implant 500.

FIGS. 16A-16D illustrate an implementation of select components of an implantable control unit 100B of the implantable flow restriction system 5. Specifically, FIGS. 16A and 16C-16D illustrate a base 202B of an external housing 200B configured to house the components of the implantable control unit 100B. While FIGS. 16A-16D do not illustrate all the components of the implantable control unit 100B, the components described with reference to the implantable control unit 100 or the implantable control unit 100A may be present in the implantable control unit 100B. Some of the features of the implantable control unit 100B are similar to features of the implantable control unit 100A in at least FIGS. 15A-15D and control unit 100 in at least FIGS. 12A-14D. Thus, reference numerals used to designate the various features or components of the implantable control units 100, 100A are identical to those used for identifying the corresponding features of components of implantable control unit 100B in FIGS. 16A-16D, except that a “B” has been added to the numerical identifier for the implantable control unit 100B. Therefore, the structure and description for the various features of the implantable control units 100 and 100A and how such units operate are understood to also apply to the corresponding features of the implantable control unit 100B in FIGS. 16A and 16C, except as described differently below.

FIG. 16A shows a top view of the base 202B of the external housing 200B of the implantable control unit 100B with some components of the implantable control unit 100B removed. The external housing 200B differs from the external housing 200A in that the connector sleeve 206B may be threaded. The connector sleeve 206B may be configured to engage with a closure 216B. The closure 216B may be a configured to clamp down or secure the tubing 570 as the closure 216B is threaded on the connector sleeve 206B. The closure 216B may apply a compressive force on the tubing 570. In some implementations, the applied compressive force increases as the closure 216B is threaded on the connector sleeve 206B. For example, the closure 216B may be or function as a Tuohy Borst device. The closure 216B may be a nut. As shown, the tubing 570 can include step up section 576 with an increased outer diameter relative to the main body of the tubing 570. The step up section 576 may be located at the proximal end 571 of the tubing 570, as shown more clearly in FIG. 16B, which illustrates an example closure 216B, the proximal end 571 of the tubing and the proximal end 591 of the shaft 590. The increase in diameter of the step up section 576 may provide room for the wires 114 to connect to the plurality of electrical contacts 116. The closure 216B may be configured to provide a seal at the seal location 578. The seal location 578 may be the point where the tubing 570 transitions to the step up section 576. In this implementation, the tubing 570 would extend further into the external housing 200 than shown in FIGS. 16A and 16C, such that the closure 216B is positioned over the seal location 578 when threaded on the connector sleeve 206B. The seal location 578 may be located at other locations along the tubing 570.

FIG. 16C shows a schematic section side view of the connector sleeve 206B and the closure 216B engaged with the tubing 570. FIG. 16D shows a schematic section front view of the connector sleeve 206B and the closure 216B engaged with the tubing 570. As discussed with reference to FIG. 13, the wires 114 of the sensor(s) 600 can extend through the tubing 570 to a plurality of electrical contacts 116. In the illustrated configuration, each wire 114 extends to an electrical contact 116 that is located on outer surface of the step up section 576. The electrical contacts 116 may extend completely or partially around the outer surface of the step up section 576 or another portion of the tubing 570. To facilitate the connection of the electrical contacts 116 and the controller wires 118, a plurality of wire housings 220B may be positioned within the connector sleeve 206B. Each wire housing 220B can be configured to house a connector wire 226B. The wire housings 220B can maintain separation between the connector wires 226B. The plurality of wire housings 220B include a central hole 214B and a wire hole 228B. The central hole 214B extends through the center of the wire housing 220B and is configured to allow the tubing 570 and shaft 590 to pass through the wire housing 220B and into the external housing 200B. The wire hole 228B extends vertically at least partially through the wire housing 220B on one or both sides of the central hole 214B. The wire hole 228B at least partially intersects with the central hole 214B such that the connector wire 226B can be in electrical contact with the electrical contact 116 when positioned in the wire housing 220B. As shown in FIG. 16D, the wire hole 228B may extend through the top of the wire housing 220B so that the wire housing 220B includes an open ended top. This top portion allows the connector wire 226B to be inserted into the wire housing 220B and allows the controller wires 118 to electrically coupled with the connector wires 226B. In some implementations, the connector wire 226B may be positioned within a spring 230B. In some implementations one or both the connector wire 226B and the spring 230B may be shaped as a three-sided rectangle (e.g., staple shaped).

With reference to FIG. 16C, each wire 114 (not shown) may be electrically coupled to an individual electrical contact 116. Similarly, each electrical contact 116 may engage an individual connector wire 226B positioned within an individual wire housing 220B. In the example illustrated, there are four wires 114, electrical contacts 116, connector wires 226B, and wire housings 220B. A plurality of seals 222B may be positioned within the connector sleeve 206B. The seals 222B can be Bal seals. In some implementations, a seal 222B is positioned between each pair of adjacent wire housings 220B. A main seal 224B may be positioned between the distal most wire housing 220B and the closure 216B. The seals 222B, 224B may be may of one or more insulating materials, such as silicon rubber. In some implementations, two connector wires 226B may apply a voltage and two connector wires 226B may measure a voltage to provide a pressure reading as explained further with reference to at least FIG. 30.

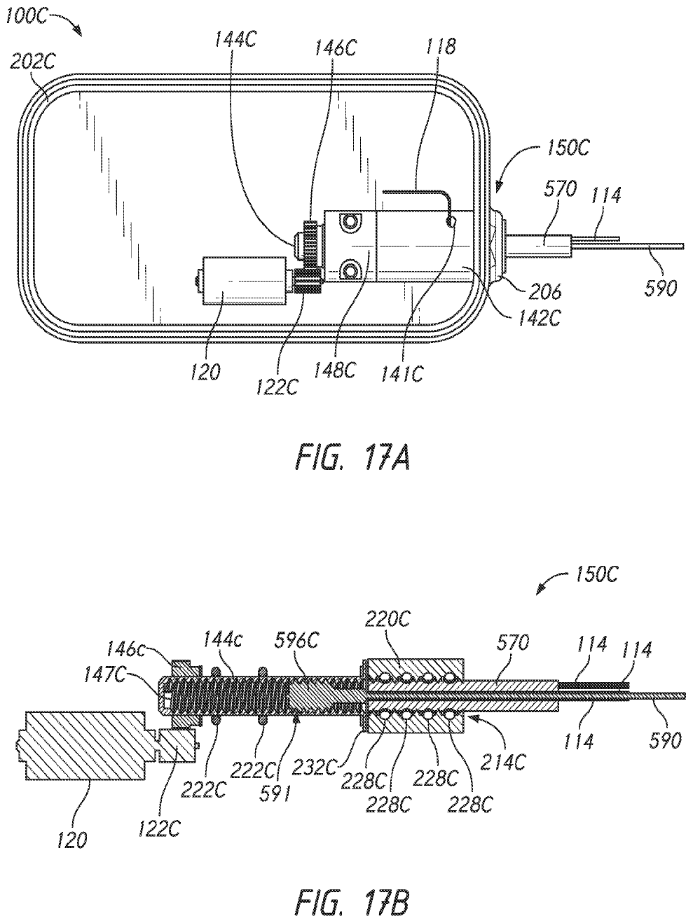

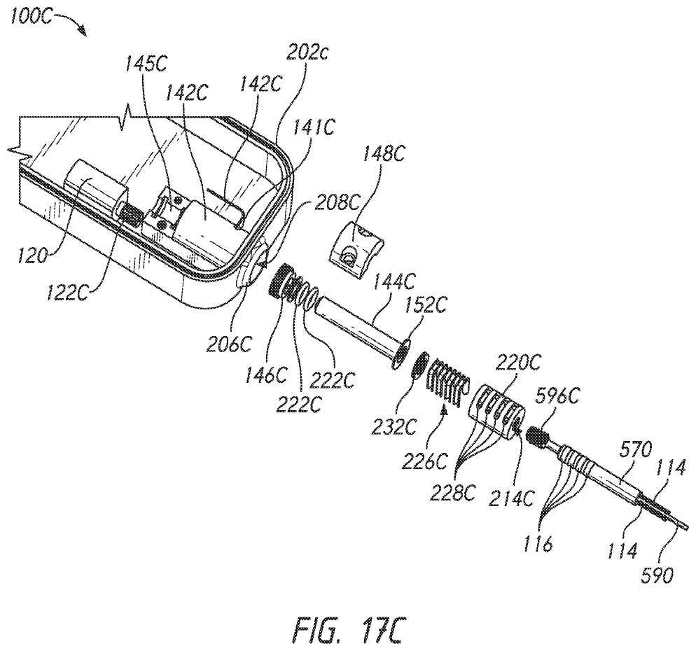

FIGS. 17A-17C illustrate an implementation of select components of an implantable control unit 100C of the implantable flow restriction system 5. Specifically, FIGS. 17A and 17C illustrate a base 202C of an external housing 200C configured to house the components of the implantable control unit 100C, and FIG. 17B illustrates an ingress protection system 150C of the implantable control unit 100C. While FIGS. 17A-17C do not illustrate all the components of the implantable control unit 100C, it is recognized that the components not shown and described with reference to the implantable control unit 100 or the implantable control unit 100A may be present in the implantable control unit 100C. Some of the features of the implantable control unit 100C are similar to features of the implantable control unit 100 in at least FIGS. 14A-14D. Thus, reference numerals used to designate the various features or components of the implantable control unit 100 are identical to those used for identifying the corresponding features of components of the implantable control unit 100C in FIGS. 17A-17C, except that an “C” has been added to the numerical identifier for the implantable control unit 100C. Therefore, the structure and description for the various features of the implantable control unit 100 and how it operates in at least FIGS. 14A-14D are understood to also apply to the corresponding features of the implantable control unit 100C in FIGS. 17A-17C, except as described differently below.

FIG. 17A shows a top view of the base 202C of the external housing 200C with components of the implantable control unit 100C removed. The external housing 200C differs from the external housing 200 primarily in that the external housing 200C include an ingress protection system 150C instead of the ingress protection system 150. The ingress protection system 150C serves a similar function of isolating mechanical components of the implantable control unit 100 from the electrical component to protect the electrical components in the event of a leak. The ingress protection system 150C includes a compartment 142C and a sleeve 144C. The compartment 142C includes an internal slot 145C (see e.g., FIG. 17C) configured to receive the sleeve 144C. The sleeve 144C includes a threaded cavity 147C. The internal slot 145C and the sleeve 144C may be axially aligned with the hole 208C of the connector sleeve 206C. As a result of this alignment, the proximal end 591 of the shaft 590 extends into the threaded cavity 147C of the sleeve 144C when engaged with the external housing 200C. The compartment 142C may include a cover 148C that covers a portion of the length of the compartment 142C. The cover 148C may be removable to permit access to the sleeve 144C within the compartment 142C. The compartment 142C may include one or more wire holes 141C so that the controller wires 118 can connect to the microcontroller 102 and the electrical contacts 116 of the implantable control unit 100C.

As shown more clearly in FIG. 17B, which shows a side section view of the ingress protection system 150C, the proximal end 591 of the shaft 590 may include a threaded tip 596C. The threaded tip 596C can be configured to engage with the threads of the threaded cavity 147C. The motor 120 may include a first gear 122C coupled to the shaft of the motor 120. The first gear 122C may engage a second gear 146C. The second gear 146C may be coupled to the outer surface of the 144C. Rotation of motor 120 causes rotation of the first gear 122C, which causes rotation of the second gear 146C. Because the sleeve 144C is coupled to the second gear 146C, rotation of the motor causes the sleeve 144C to rotate. As the sleeve 144C rotates, the threaded tip 596C moves proximally and distally along the threaded cavity 147C, causing corresponding movement for the shaft 590, which moves the implant 500 between different restrictive or occlusive states.

FIG. 17C shows an exploded view of the external housing 200C and ingress protection system 150C, as well as other components of the implantable flow restriction system 5. With reference to FIGS. 17B and 17C, the external housing 200C may also house a wire housing 220C. The wire housing 220C may be positioned within one or both of the internal slot 145C of the compartment 142C and the hole 208C of the connector sleeve 206C. A connector 232C may be positioned between the sleeve 144C and the wire housing 220C. In some implementations, the wire housing 220C may be generally tubular shaped. A distal end 152C of the sleeve 144C may be slotted in the connector 232C such that the sleeve 144C can rotate relative to the wire housing 220C, connector 232C, and the compartment 142C.

As discussed with reference to FIG. 13, the wires 114 of the sensor(s) 600 can extend through the tubing 570 to a plurality of electrical contacts 116. In the illustrated configuration, each wire 114 extends to an electrical contact 116 that is located on outer surface of the tubing 570. The electrical contacts 116 may extend completely or partially around the outer surface of the tubing 570. The wire housing 220C may facilitate the connection of the electrical contacts 116 and the controller wires 118. The wire housing 220C is configured to house a plurality of electrodes 226C. The wire housing 220C maintains separation between each of the plurality of electrodes 226C. The wire housing 220C includes a central hole 214C and a plurality of electrode holes 228C. The central hole 214C extends through the center of the wire housing 220C and is configured to allow the tubing 570 and shaft 590 to pass through the wire housing 220C and into the external housing 200C. The wire holes 228C can extend vertically at least partially through the wire housing 220C on one or both sides of the central hole 214C. The wire holes 228C at least partially intersect with the central hole 214C such that the plurality of electrodes 226C can contact the electrical contact 116 when positioned in the wire housing 220C. As shown in FIG. 17C, the wire holes 228C may extend through the top of the wire housing 220C so that the wire housing 220C includes a number of openings in a top surface. This top portion allows the plurality of electrodes 226C to be inserted into the wire housing 220C and allows the controller wires 118, which may be arranged as a bus, to electrically couple with the electrodes 226. In some implementations, the plurality of electrodes 226C may be shaped as a three-sided rectangle (e.g., staple shaped). Each wire 114 may be electrically coupled to an individual electrical contact 116. Similarly, each electrical contact 116 may engage an individual electrode 226C positioned within the wire housing 220C. In the example illustrated, there are four wires 114, electrical contacts 116, and electrodes 226C. A plurality of seals 222C may be positioned on the outer surface of the sleeve 144C. The seals 222C may be O-rings.

The ingress protection system 150C may provide certain advantages. For example, the ingress protection system 150C may prevent fluid from entering the main portion of the external housing 200C. For example, even if fluid enters the threaded cavity 147C of the sleeve 144C, the electrical components of the implantable control unit 100C will be isolated from the fluid. The ingress protection system 150C can be combined with other fluid isolation configurations. For example, in some implementations, at least a portion of the external housing 200C may be filled with a further isolation layer, such as an insulating/isolating epoxy, as described above.

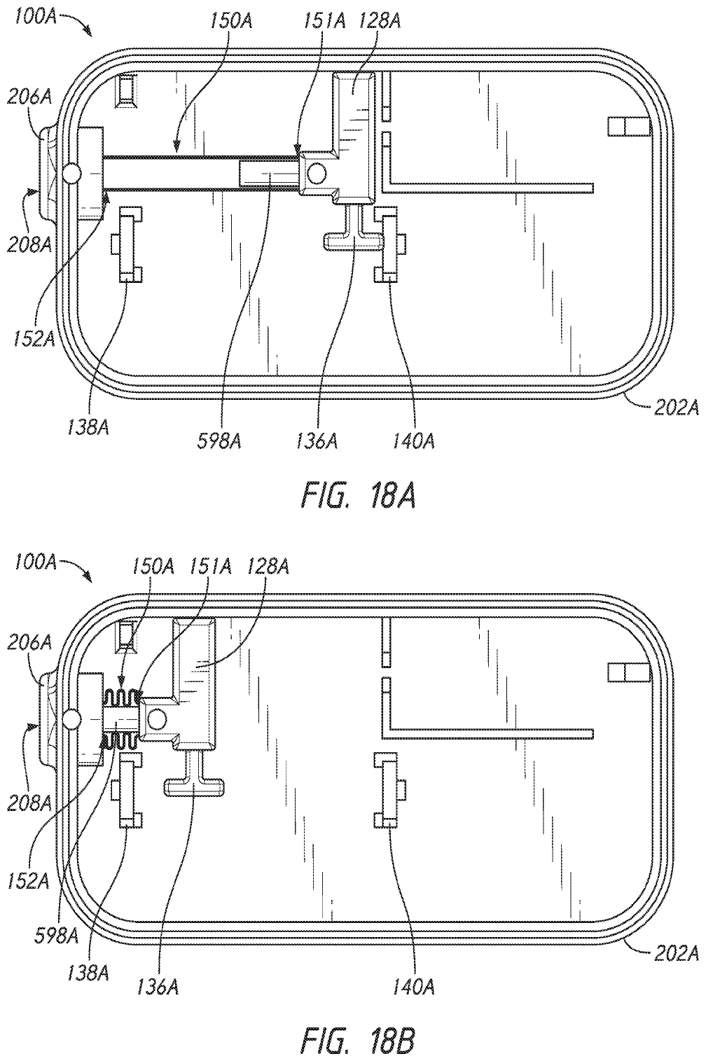

FIGS. 18A and 18B illustrate an ingress protection system 150A that can be used with the implantable control unit 100A or any other variation of an implantable control unit in which the base includes an undivided compartment including mechanical and electrical components. The ingress protection system 150A may comprise a sheath that includes a first end 151A and a second end 152A. The first end 151A may be coupled to the traveler 128A and the second end 152A may be coupled to the connector sleeve 206A on the internal side of the base 202A. The proximal end 591 of the shaft 590 (not shown) is configured to extend through the ingress protection system 150A, such that the shaft 590 is isolated from other components of the implantable control unit 100A within the base of the enclosure thereof. For example, the proximal end 591 may extend through the hole 208A and may be coupled to the traveler 128A via a connector 598A. The ingress protection system 150A may be flexible and may be configured to elastically deform without compromising the seal provided by the ingress protection system 150A. For example, as the traveler 128A moves proximally and distally within the base 202B, the ingress protection system 150A may move between an extended/elongated state, shown in FIG. 18A, and a compressed state, shown in FIG. 18B. In some implementations, the ingress protection system 150A may include set folds or crinkles such that the ingress protection system 150A collapses in an accordion style as the traveler 128A moves distally. The accordion style collapsing can employ less material stretching, which can be a more robust structure less prone to plastic deformation and failure from cycling.

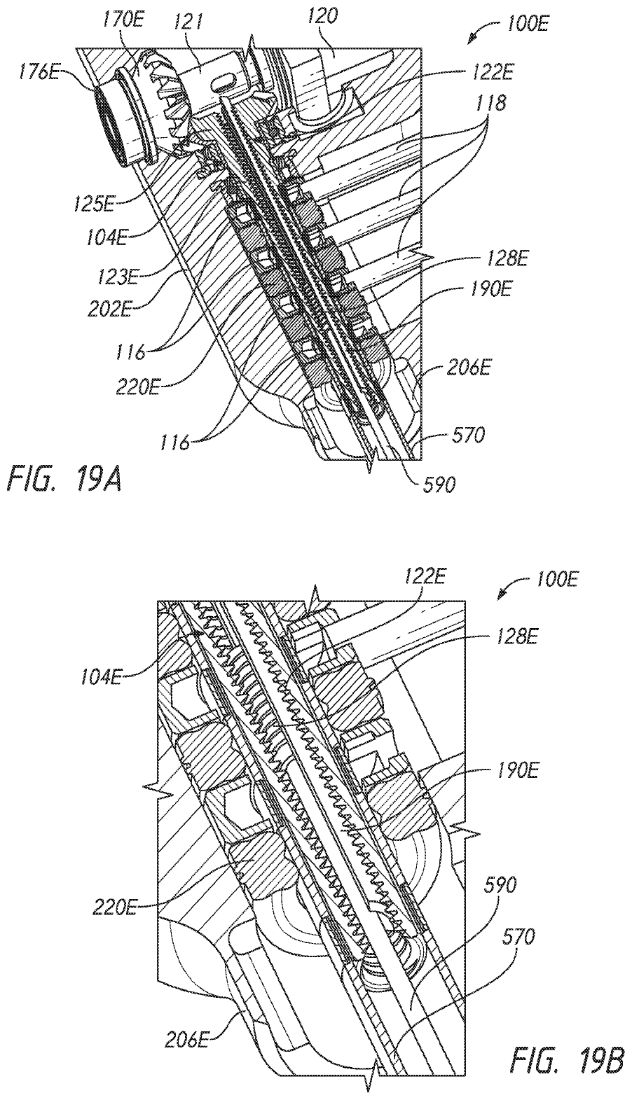

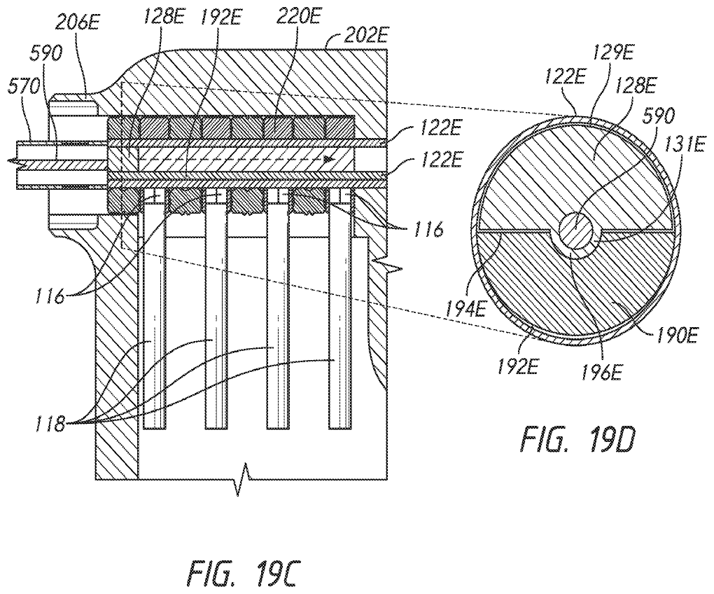

FIGS. 19A-19D illustrate an implementation of select components of an implantable control unit 100E of the implantable flow restriction system 5. Specifically, FIGS. 19A and 19B show an actuator system 104E of the implantable control unit 100E and FIGS. 19C and 19D show schematic section views of the actuator system 104E. While FIGS. 19A-19D do not illustrate all the components of the implantable control unit 100E, the components described with reference to the implantable control unit 100 and/or the implantable control units 100A-100D may be present in the implantable control unit 100E. Some of the features of the implantable control unit 100E are similar to features of the implantable control unit 100D in at least FIGS. 14A-14H, the implantable control unit 100A in at least FIGS. 15A-15D, and control unit 100 in at least FIGS. 12A-14D. Thus, reference numerals used to designate the various features or components of the implantable control units 100, 100A, 100D are identical to those used for identifying the corresponding features of components of implantable control unit 100E in FIGS. 19A-19D, except that an “E” has been added to the numerical identifier for the implantable control unit 100E. Therefore, the structure and description for the various features of the implantable control units 100 and 100A-100D and how such units operate are understood to also apply to the corresponding features of the implantable control unit 100E in FIGS. 19A-19D, except as described differently below.

FIG. 19A shows a perspective isolated section view of a portion of the base 202E of the external housing 200E of the implantable control unit 100E with some components of the implantable control unit 100E not shown or removed. FIG. 19B shows a close up view of FIG. 19A. FIGS. 19C and 19D show schematic section views of the actuator system 104E of the implantable control unit 100E. The implantable control unit 100E differs from the implantable control unit 100D primarily in the actuator system 104E. The actuator system 104E can include an internally threaded tube 122E, a traveler 128E, and/or a support member 190E. The traveler 128E can be coupled to the shaft 590. Rotation of the internally threaded tube 122E can cause the traveler 128E to travel proximally and distally within the internally threaded tube 122E, moving the implant 500 between different states. For example, the actuator system 104E can have a first configuration corresponding to the implant 500 being in the low profile state and a second configuration corresponding to the implant 500 being in the high profile pressure modulating/flow restricting state (referred to herein as the “high profile state”). The actuator system 104E can be at least partially located within a lumen of the tubing 570. The actuator system 104E can extend out of the proximal end 571 of the tubing 570 for engagement with the motor 120. The tubing 570 can include electrical contacts 116 that are configured to connect to controller wires 118 of the implantable control unit 100E. The tubing 570 can include a wire housings 220E that includes similar components and functions in a similar manner to the external housing 200D described with reference to at least FIG. 14G.

The internally threaded tube 122E can include a body portion 123E and a head portion 125E. The head portion 125E can be proximal to and coupled to the body portion 123E. Internal threads can extend between the body portion 123E and the head portion 125E within a lumen of the internally threaded tube 122E. The head portion 125E can be configured to engage with the motor 120 to cause rotation of the internally threaded tube 122E. For example, the head portion 125E can be a gear (e.g., a beveled gear) that meshes with a gear 170E coupled to the shaft 121 of the motor 120. As such, rotation of the shaft 121 causes corresponding rotation of the head portion 125E and the internally threaded tube 122E. The internally threaded tube 122E can be configured to rotate within the lumen of the tubing 570. In the illustrated example, the internally threaded tube 122E and the tubing 570 extend along a first axis and the shaft 121 extends along a second axis perpendicular to the first axis. In some implementations, the first axis can be aligned with the second axis similar to the arrangement shown in FIG. 14B. In some implementations, the first axis and the second axis can be parallel to each other, similar to the arrangement shown in FIG. 15H. In this implementation, a body of the motor 120 can be supported in the external housing 200E along the tubing 570 surrounding the proximal length of the lumen of the tubing 570.

The traveler 128E can be configured to travel proximally and distally within the internally threaded tube 122E. The support member 190E can allow the traveler 128E to travel within the internally threaded tube 122E without rotating. For example, the rotational motion of the internally threaded tube 122E can cause axial movement of the traveler 128E. The traveler 128E can include an externally threaded surface 129E that can mesh with the internal threads of the internally threaded tube 122E. The external threads 129E can extend over a semicircular periphery of the traveler 128E. The traveler 128E can be coupled to a proximal end 591 of the shaft 590. For example, the traveler 128E can include a connecting structure 131E for coupling with the shaft 590. The connecting structure 131E can be opposite the externally threaded surface 129E. The connecting structure 131E can be a surface spanning a portion of a diameter of the semicircular periphery of the externally threaded surface 129E and a portion coupled to the proximal end 591 of the shaft 590. The connecting structure 131E can be a projection with a circular periphery.

The support member 190E can be positioned within the internally threaded tube 122E. The support member 190E may not include external threads. As such, rotation of the internally threaded tube 122E does not cause rotation or movement of the support member 190E. The support member 190E can be a cylindrical portion having a semicircular periphery 192E. The support member 190E can include a support portion 194E and a channel 196E. The support member 190E can be configured to support the connecting structure 131E for linear motion of the traveler 128E. For example, the support portion 194E can be configured to slidably support the traveler 128E on an opposite side of the externally threaded surface 129E. In this arrangement, the connecting structure 131E can be disposed within the channel 192E. As the traveler 128E travels within the internally threaded tube 122E, the connecting structure 131E can travel along the channel 192E. In some implementations, the channel 192E can have a circular periphery for mating with the circular projection that forms the connecting structure 131E.

In operation, the motor 120 drives the shaft 121 causing the gear 170E to rotate. As the head portion 125E of the internally threaded tube 122E meshes with the gear 170E, the internally threaded tube 122E rotates within the lumen of the tubing 570. Rotation of the internally threaded tube 122E can cause the traveler 128E to move axially along a length of the tubing 570 in a first direction. Because the support member 190E is not threaded, the support member 190E does not rotate and prevents the traveler 128E from rotating within the internally threaded tube 122E. Movement of the traveler 128E in the first direction causes corresponding movement of the shaft 590 in the first direction, moving the implant 500 into a high profile state. Opposite rotation of the motor 120 causes the traveler 128E to move axially along a length of the tubing 570 in a second direction opposite the first direction, returning the implant 500 to the low profile state. In some implementations, the proximal end 591 of the shaft 590 is located within the proximal length of the tubing 570 (e.g., within the external housing 200E) when the implant 500 is in both the high profile state and low profile state.

FIG. 20A illustrates a sensor system 160 that can be utilized in any of the implantable control units described herein (e.g., the implantable control units 100, 100A, 100B, 100C, 100D, 100E, and/or 100F). FIG. 20B illustrates an exploded view of the sensor system 160. The sensor system 160 may include a plurality of sensors spaced apart from each other along a direction parallel to a length of the rod 122. For example, the sensor system 160 may include a first Hall effect sensor 162, a second Hall effect sensor 164, and a third Hall effect sensor 166. The Hall effect sensors 162, 164, 166 may be equally spaced along a portion of the length of the rod 122. For example, 10 mm spacing between each sensor may be used. However, the size of the spacing may vary based on the size and use of the implantable control unit 100. The Hall effect sensors 162, 164, 166 are configured to detect the presence and strength of a magnetic field. The Hall effect sensors 162, 164, 166 may be electrically coupled to the printed circuit board 101 and the microcontroller 102. The Hall effect sensors 162, 164, 166 may include a strip or plate of conductor or semiconductor material and are configured to generate a voltage difference when subjected to a magnetic field that is perpendicular to the current flow. The traveler 128 may include a magnet 168. The magnet 168 may be positioned within the traveler 128. As the traveler 128 translates along the rod 122, the magnetic field of the magnet 168 changes its position relative to the Hall effect sensors 162, 164, 166. The microcontroller 102 may be configured to receive voltage readings from the Hall effect sensors 162, 164, 166, where the voltage readings are proportional to the strength of the magnetic field of the magnet 168, which varies with the position of the traveler 128. Based in part on this information, the microcontroller 102 may determine the relative position of the traveler 128 within the external housing 200, which corresponds to an occlusive state of the implant 500.

FIG. 20C illustrates example voltage measurements generated by the Hall effect sensors 162, 164, 166 relative to a position of the traveler 128 between 0 mm and 30 mm. Voltage readings from the Hall effect sensors 162, 164, 166 were generated for each position of the traveler 128 at positions spaced 0.1 mm apart. The top graph illustrates the voltage readings as the traveler 128 was advanced proximally, and the bottom graph illustrates the voltage readings as the traveler was retracted distally. As shown, the voltage measured by the microcontroller 102 for each Hall effect sensor 162, 164, 166 varies based on the position of the traveler 128. Using this data, an algorithm can be used to determine the position of the traveler 128 along the rod 122, which can be correlated to the degree of occlusion provided by the implant 500. As such, the microcontroller 102 can be configured to actuate the implant 500 to a set occlusion percentage based on determining a desired position for the rod 122 relative to the Hall effect sensors 162, 164, 166.

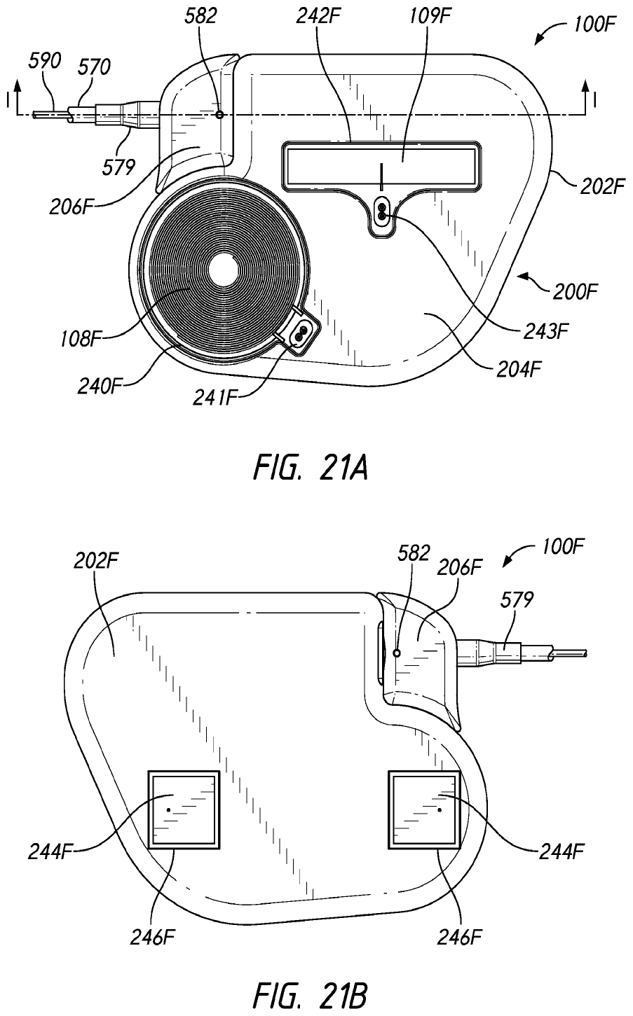

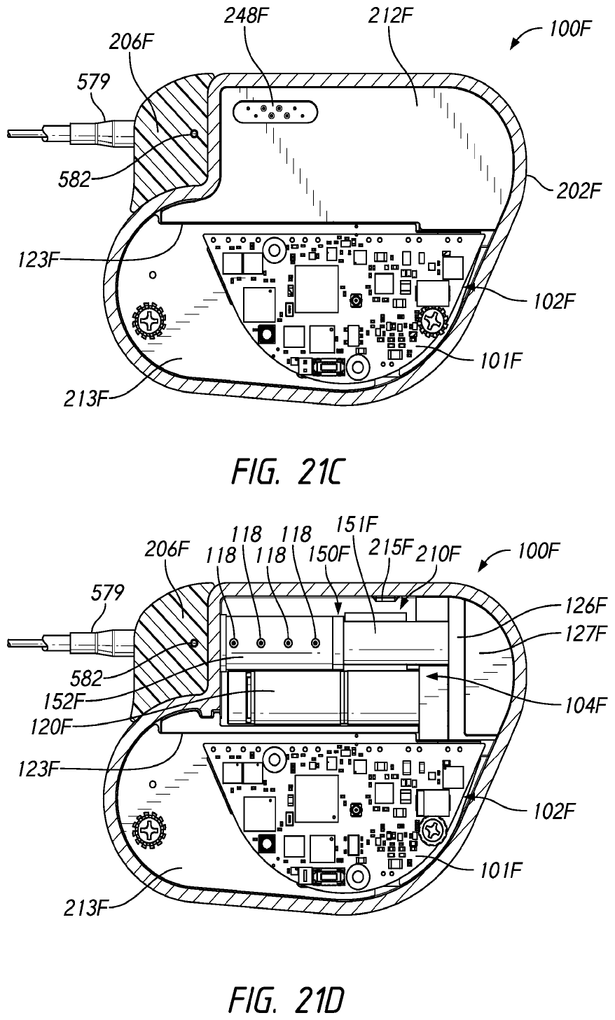

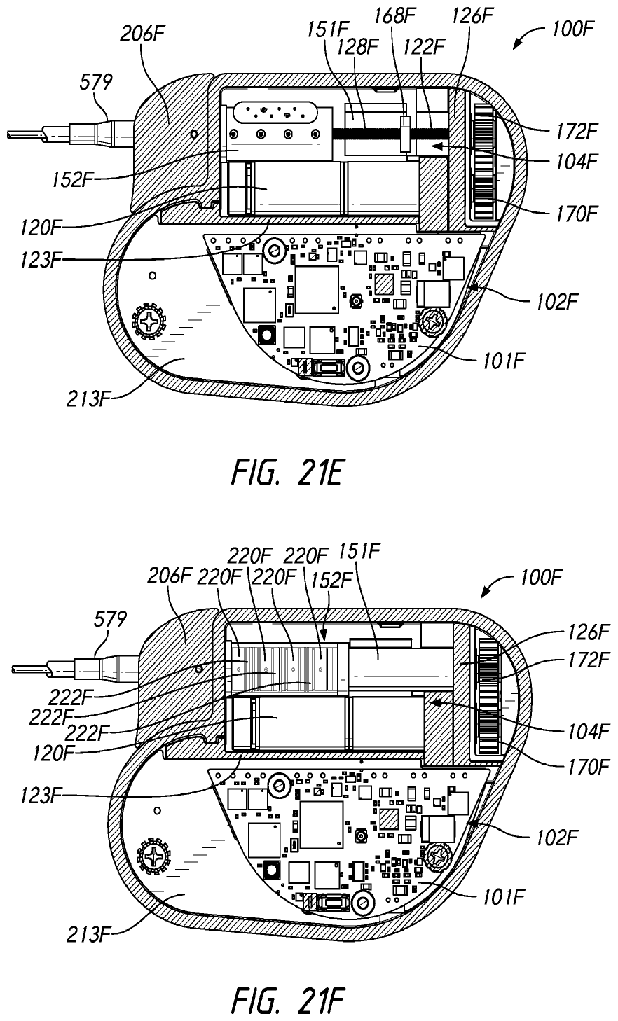

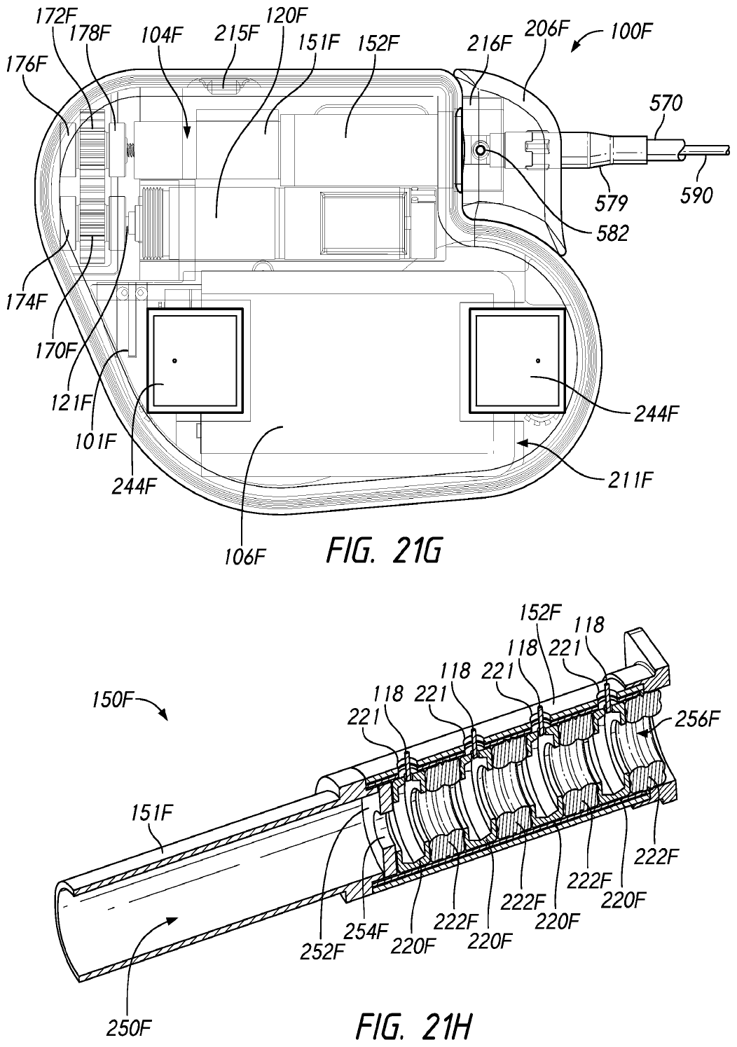

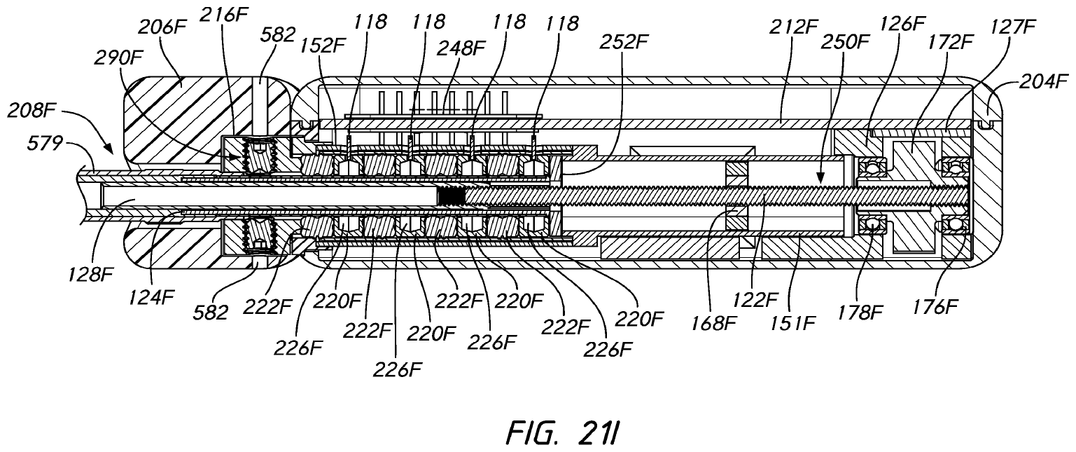

FIGS. 21A-21I illustrate an implementation of an implantable control unit 100F of the implantable flow restriction system 5 and components thereof. While FIGS. 21A-21I do not illustrate all the components of the implantable control unit 100F, the components described with reference to the implantable control unit 100 and/or the implantable control units 100A-100E may be present in the implantable control unit 100F. Some of the features of the implantable control unit 100F are similar to features of the implantable control unit 100 in at least FIGS. 12A-14D, the implantable control unit 100D in at least FIGS. 14E-14J, the implantable control unit 100A in at least FIGS. 15A-15D, the implantable control unit 100B in at least FIGS. 16A-16D, the implantable control unit 100C in at least FIGS. 17A-17C, and the implantable control unit 100E in at least FIGS. 19A-19D. Thus, reference numerals used to designate the various features or components of the implantable control units 100 and 100A-100E are identical to those used for identifying the corresponding features of components of implantable control unit 100F in FIGS. 21A-21I, except that an “F” has been added to the numerical identifier for the implantable control unit 100F. Therefore, the structure and description for the various features of the implantable control units 100 and 100A-100E and how such units operate are understood to also apply to the corresponding features of the implantable control unit 100F in FIGS. 21A-21I, except as described differently below.

The implantable control unit 100F, (also referred to herein as a “controller” and a “control system”) can be incorporated in the implantable flow restriction system 5 as well as any other implementations of the implantable flow restriction systems described herein or in U.S. Pat. No. 11,883,030. In some implementations, the implantable control unit 100F can have a circuit configuration shown in FIG. 12A and/or FIG. 12B. For example, the implantable control unit 100F of the implantable flow restriction system 5 can include a microcontroller 102F, an actuator system 104F, and a first power source 106F. In some configurations, the implantable control unit 100 can include a receiver 108F, an amplifier (not shown), and/or a second power source (not shown).

The implantable control unit 100F can be patient-controlled and/or patient monitored, for example, wirelessly through a software application (e.g., a mobile device application or an “app”) on the external device 15. The implantable control unit 100F can be configured to be implantable within the patient and can include a source of implant actuation, a mother board comprising a processor, a memory, in some implementations a communications module, and a source of power. The implantable control unit 100F can be configured to receive wireless or wired signals from sensor(s) (e.g., pressure sensors, such as any of the pressure sensors 600, 600A, 600B, 600C, 600D) positioned in various locations within or around the heart or at other locations in the body, for example to measure pressure in the right ventricle, right atrial pressure, central venous pressure, aortic pressure, left atrial pressure, left ventricular pressure, aortic pressure, SVC pressure, IVC pressure, hepatic vein pressure, renal vein pressure, femoral vein pressure, and/or the pressure of any of the veins or portions thereof disclosed herein. Based at least in part on these readings, the implantable control unit 100F can appropriately actuate the implant 500 to control the adjustable occlusion of the implant in order to control the amount of blood flowing through the implant. The implantable control unit 100F can provide for closed-loop, fully autonomous, and/or real-time adjustability and control of the implant 500. The implantable control unit 100F can implement a treatment protocol/algorithm prescribed by a physician and/or the control logic can be optimized to treat heart failure patients, for example, by reducing cardiac preload, reducing central venous pressure, and/or pressure of other veins disclosed herein, increasing cardiac output, reducing renal congestion (or promoting renal decongestion), enhancing renal circulation, and/or enhancing or controlling diuresis (e.g., to increase diuresis). In some implementations, the implantable control unit 100F can receive data from sensor(s) connected to the implant 500 as described herein for the control of actuation of the implant 500. The sensor(s) may work and/or be used in conjunction with any of the flow restriction devices described herein and the implants described in U.S. Pat. No. 11,883,030 to provide physiological parameters of interest useful for control of the flow restriction device. The sensor(s) can include any of the sensors described herein (e.g., sensor 600, sensor 600A, capacitive sensor 600B, sensor 600C, sensor 600D, and/or the like). The sensors may also include any conventional pressure sensors that can be implemented into the implantable flow restriction system 5. Various example sensor(s) and sensor assemblies are described in further detail with reference to at least FIGS. 28A-38C.

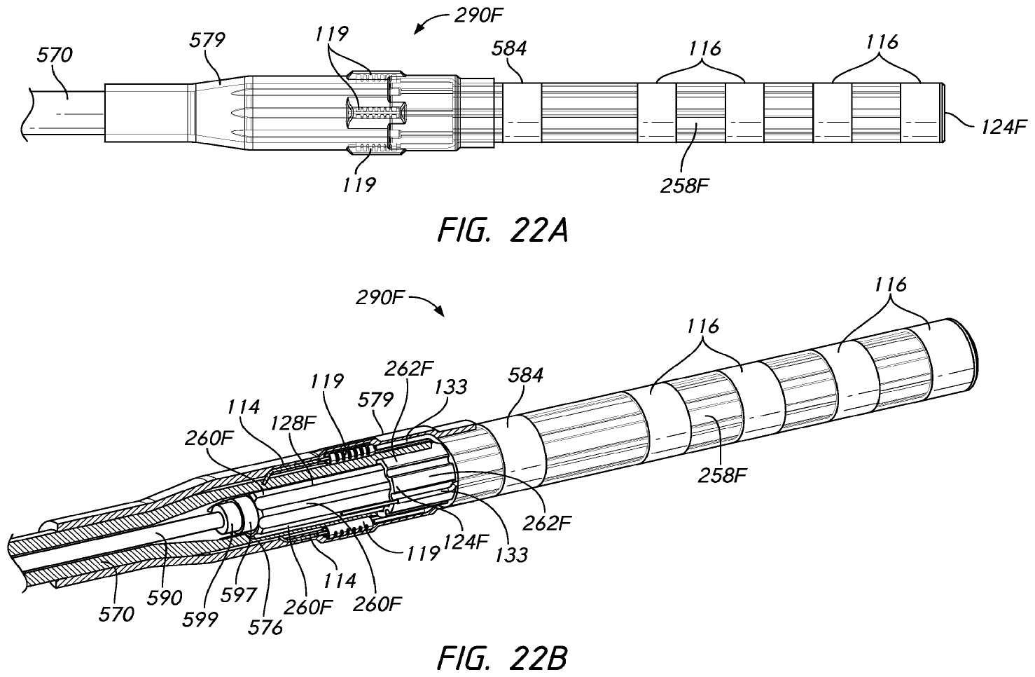

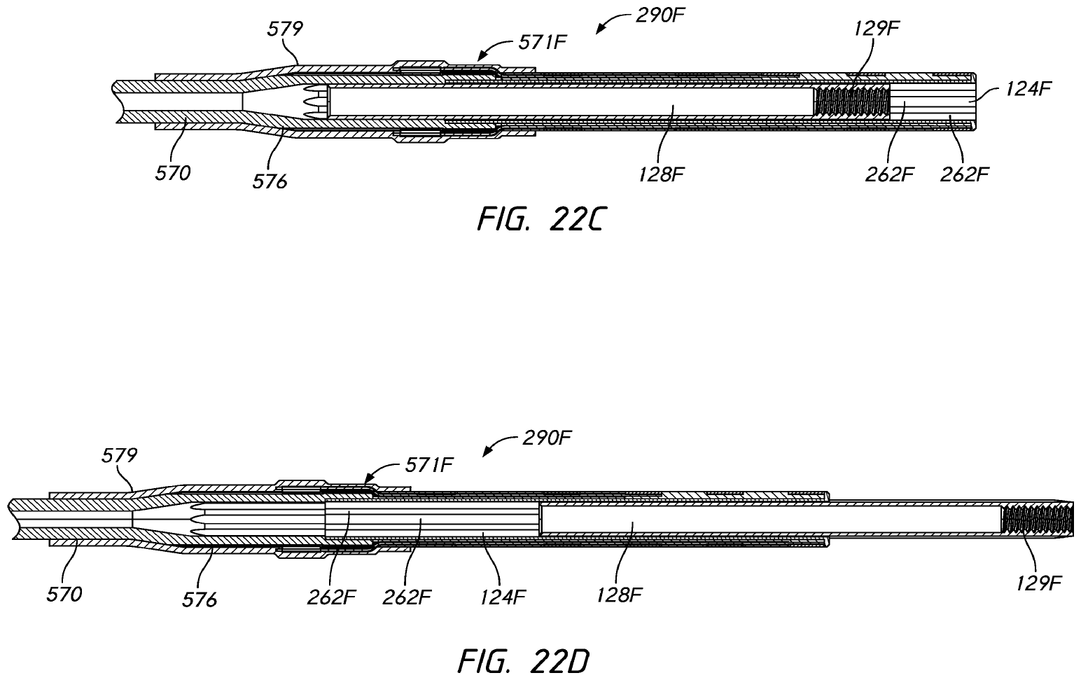

With reference first to FIGS. 21A and 21B, the implantable control unit 100F can be housed within an external housing 200F. FIG. 21A shows a top view of the external housing 200F and FIG. 21B shows a bottom view of the external housing 200F. The external housing 200F may be any suitable shape, such as square, rectangular, circular, and/or the like. As shown in FIG. 21A, the external housing 200F can have a general parallelogram shape and may include rounded edges. The external housing 200F may be any suitable material such as a metal, a plastic, a ceramic, a metal, or a combination of two or more materials. In some configurations, a plastic external housing 200F may provide certain benefits of reducing interference for inductive coupling and communication (e.g., via Bluetooth® wireless communication) between the implantable control unit 100F and the external device 15. In some configurations, the communication and receiver portions of the implantable control unit 100F may be disposed on an outer surface the external housing 200F. One or more of communications and wireless charging components may be disposed in recesses formed on the outer surface to enhance transmission and receipt of communications signals and wireless charging coupling. Accordingly, a material that has the potential to cause interference, such as titanium, may be used for the external housing 200F, with limited or no reduction in the performance of the implantable control unit 100F. A majority of the components of the implantable control unit 100F may be enclosed between a base 202F and an outer cover 204F of the external housing 200F. The outer cover 204F is configured to mate with the base 202F. The external housing 200F protects the components of the implantable control unit 100F when the outer cover 204F is fixed to the base 202F. The external housing 200F may also include a connector portion 206F. The connector portion 206F can include a hole 208F (see e.g., FIG. 21I) that extends through the base 202F and/or a portion of the outer cover 204F to provide access to an internal portion of the external housing 200F. Other components of the implantable flow restriction system 5, such as the shaft 590 and a connector assembly 290F (see e.g., FIGS. 22A-22D), may extend through hole 208F into the external housing 200F. The hole 208F can include one or more sealing mechanisms to protect the implantable control unit 100F.

Turning to FIG. 21C, a top view of the implantable control unit 100F is shown with the cover 204F removed. As shown, the implantable control unit 100F can include a microcontroller 102F. The microcontroller 102F can be similar or identical to the microcontroller 102 described with reference to at least FIGS. 12A and 12B. The microcontroller 102F can be mounted on a printed circuit board 101F to facilitate the integration of the electrical components of the implantable control unit 100F and/or the system 5. The microcontroller 102F can be mounted to one or more inner covers of the external housing 200F. For example, the external housing 200F can include a first inner cover 212F and a second inner cover 213F. In the illustrated example, the microcontroller 102F is positioned on the second inner cover 213F and between the second inner cover 213F and the outer cover 204F. In this arrangement, the microcontroller 102F can be isolated from the other components of the implantable control unit 100F. Isolating the microcontroller 102F can help protect the microcontroller 102F from ingress of foreign matter, for example, body tissue or fluids, when the control unit 100F is implanted. In some implementations, the space between the inner covers 212F, 213F and the outer cover 204F may enclose or be at least partially filled with an insulating material/isolation layer. For example, a resin, such as an epoxy, may be deposited within this internal space to mitigate any damage to the microcontroller 102F should any fluid enter the external housing 200F.

The external housing 200F can include a first internal housing 210F (see e.g., FIG. 21D) and a second internal housing 211F (see e.g., FIG. 21G). The first internal housing 210F can be covered by the cover 212F and the second internal housing 211F can be covered by the cover 213F. An internal wall 123F can separate the first internal housing 210F from the second internal housing 211F. The first internal housing 210F can house the actuator system 104F, as described further herein. The second internal housing 211F can house the first power source 106F. The first power source 106F can be similar or identical to the first power source 106 and/or the second power source 112 described with reference to at least FIGS. 12A and 12B. One or both of the first internal housing 210F and the second internal housing 211F may be filled with an insulating material.

Referring back to FIG. 21A, the external housing 200F can include one or more isolated compartments recessed into the base 202F and/or the outer cover 204F. The one or more isolated compartments can be configured to receive one or more wireless components of the implantable control unit 100F. For example, the implantable control unit 100F can include a receiver 108F. The receiver 108F can be used to receive power from a transmitter external to the implantable control unit 100F (e.g., the external device 15). The receiver 108F can be used to power the first power source 106F and/or a second power source of the microcontroller 102F. In some cases, the receiver 108F can be configured to activate the actuator system 104F. In some examples, the receiver 108F can be an induction receiver, an ultrasound receiver, and/or the like. The receiver 108F may be positioned within a first outer compartment 240F formed in the outer cover 204F. In other implementations, the first outer compartment 240F may be formed in the base 202F. Accordingly, the receiver 108F can be substantially isolated from the other components of the implantable control unit 100F. The receiver 108F can be electrically coupled to the first power source 106F and/or the microcontroller 102F via one or more wires that extend through one or more hermetic passthroughs 241F in the outer cover 204F. In some implementations, the first outer compartment 240F may be filled and the receiver 108F may be covered with an insulating material, such as a resin. The insulating material can prevent the receiver 108F from being exposed to a patient's subcutaneous tissue when the implantable control unit 100F is implanted.

The implantable control unit 100F may include an antenna 109F. The antenna 109F can be electrically connected to the microcontroller 102F. The antenna 109F can enable the implantable control unit 100F to transmit data (e.g., pressure readings, ECG data, other sensor data, device performance metrics, power storage information, and/or the like) wirelessly to external monitoring equipment, such as the external device 15. The antenna 109F may also enable the implantable control unit 100F to be remotely adjusted and/or updated. In one example, the antenna 109F may be a Bluetooth® Low Energy (“BTLE”) antenna configured to provide the implantable control unit 100F with efficient, reliable wireless connectivity for low-power and/or short-range applications. The antenna 109F may be positioned within a second outer compartment 242F formed in the outer cover 204F. In other implementations, the second outer compartment 242F may be formed in the base 202F. Accordingly, the antenna 109F can be substantially isolated from the other components of the implantable control unit 100F. The antenna 109F can be electrically coupled to the microcontroller 102F via one or more wires that extend through one or more hermetic passthroughs 243F in the outer cover 204F. In some implementations, the second outer compartment 242F may be filled and the antenna 109F may be covered with an insulating material, such as a resin. The insulating material can prevent the antenna 109F from being exposed to the patient's subcutaneous tissue when the implantable control unit 100F is implanted.

Referring back to FIG. 21B, the implantable control unit 100F may include one or more electrocardiogram (“ECG”) sensors 244F. The ECG sensors 244F can be configured to detect the electrical activity of the patient's heart to provide real-time data/signals to the microcontroller 102F. The ECG sensors 244F can be positioned in one or more third outer compartments 246F formed in the base 202F. In other implementations, the one or more third outer compartments 246F may be formed in the outer cover 204F. The ECG sensors 244F can be electrically coupled to the microcontroller 102F via one or more wires that extend through hermetic passthroughs (not shown) in the base 202F. Including the ECG sensors 244F in the implantable control unit 100F can allow a physician to customize the therapy setting of the implantable control unit 100F for the patient based on the signals received from the heart.

In some implementations, the implantable control unit 100F can include one or more additional sensors and/or devices configured to provide additional patient information to the microcontroller 102F. For example, the implantable control unit 100F may include an accelerometer (not shown). The accelerometer may be housed in the external housing 200F, for example, mounted on the same printed circuit board 101 upon which the microcontroller 102F is mounted. The accelerometer may be configured to generate an accelerometer signal to be transmitted to the microcontroller 102F and/or the external device 15. The accelerometer can be part of an inertial measurement unit (IMU) that may include other inertial sensors such as gyroscopes and/or magnetometers. In one example, the accelerometer may be electrically connected to the microcontroller 102F. The accelerometer or IMU signal can be used to provide an indication of a patient condition and/or activity level. The patient condition can include whether the patient is lying down, sitting, standing, remaining stationary, moving, and/or the like. The activity level can provide an indication of whether the patient is engaged in a low level activity, medium level activity, high level activity, and/or the like. As explained herein, it may be desirable to base therapy decisions, to be provided by the flow restriction system 5, on when the patient is in a certain patient condition and/or activity level. For example, it may be desirable to take pressure readings when the patient is lying down and at a low activity level (e.g., at night). This is in part due to the changes in patient pressure caused by certain patient conditions and activity levels. By taking pressure readings when the patient is generally inactive, more accurate therapy assessments can generally be made.

Turning now to FIGS. 21D-21F, top views of the implantable control unit 100F with outer cover 204F and the first inner cover 212F removed are shown. With the cover 212F removed, the first internal housing 210F can be seen. The first internal housing 210F can house the actuator system 104F. The actuator system 104F can be configured to move the shaft 590 within tubing 570 for actuation of the flow restrictor 560 and/or flow restrictor portion 550 of the implant 500 (which can include any of the implants described herein). In the illustrated example, the actuator system 104F can be configured to slidingly move the shaft 590 proximally and/or distally relative to the tubing 570 and implant 500. In other examples, the implantable control unit 100F may include an actuator system configured to rotationally move the shaft 590 relative to the tubing 570 and the implant 500. Furthermore, the actuator system 104 can be configured to cause the flow restrictor 560 and/or flow restrictor portion 550 of implant 500 to occlude/restrict flow through the implant 500 in a range of between about 0% to about 100%, for example, from and including about 0% to about 100% or a suitable range of therein. Here, 0% is the original low profile state of the implant 500 and 100% is the high profile state.

The actuator system 104F may include a motor 120F, a rod 122F, and an inner connector 128F. At least a portion of the actuator system 104F, including the rod 122F and the inner connector 128F, can be housed in an ingress protection system 150F. The inner connector 128F can also form a part of the connector assembly 290F, which can be at least partially housed in the ingress protection system 150F. The ingress protection system 150F can be configured to prevent ingress of foreign matter, for example, fluid into the first internal housing 210F. The ingress protection system 150F may comprise titanium or other suitable material for such purpose. The ingress protection system 150F can include a first compartment 151F and a second compartment 152F. The first compartment 151F is shown as transparent in FIG. 21E and the second compartment 152F is shown as transparent in FIG. 21D for illustrative purposes. The ingress protection system 150F is described further with reference to at least FIGS. 21H and 21I.

The motor 120F can be controlled by the microcontroller 102F and can be powered by one or both of the first power source 106F and/or a second power source (not shown). The motor 120F can be configured to cause rotation of the rod 122F, which in turn causes the inner connector 128F to travel axially within the ingress protection system 150F. As explained further herein, movement of the inner connector 128F causes corresponding movement of the shaft 590, such that the implant 500 moves between the low profile state and the high profile state. For example, the inner connector 128F can be coupled to and/or disposed at the proximal end 591 of the shaft 590. An output shaft 121F (see e.g., FIG. 21G) of the motor 120 may be coupled to a first gear 170F and the rod 122F may be coupled to a second gear 172F. The second gear 172F can mesh with the first gear 170F such that rotation of the shaft 121F causes corresponding rotation of the rod 122F. In some implementations, the first and second gears 170F, 172F may have a one-to-one gear ratio. In some implementations, the first and second gears 170F, 172F may have a gear ratio of more or less than one-to-one. Other forms of rotational motion transmission are possible in various alternative embodiments.

The implantable control unit 100F may include one or more bearings to facilitate the rotation of the gears 170F, 172F relative to the external housing 200F. For example, as shown in FIG. 21G, which illustrates a bottom view of the implantable control unit 100F with the external housing 200F shown as transparent for illustrative purposes, the implantable control unit 100F can include a first bearing 174F, a second bearing 176F, and/or a third bearing 178F. The outer race of the first bearing 174F may be coupled to the base 202F of the external housing 200F and the inner race of the first bearing 174F may be coupled to the shaft 121F. In this arrangement, the shaft 121F is supported by and can rotate relative to the base 202F. The outer races of the second and third bearings 176F, 178F may be coupled to the base 202F and the inner races of the bearings 176F, 178F may be coupled to the second gear 172F. In this arrangement, the second gear 172F is supported by and can rotate relative to the base 202F. In some cases, the shaft 121F of the motor 120 may be configured to rotate about a first axis and the rod 122F may be configured to rotate about a second axis parallel to the first axis. This arrangement can reduce the overall size of the external housing 200F. The gears 170F, 172F may be isolated from the ingress protection system 150F and the motor 120F via a divider wall 126F. The divider wall 126F can be positioned within the first internal housing 210F and can be covered by a third internal cover 127F.

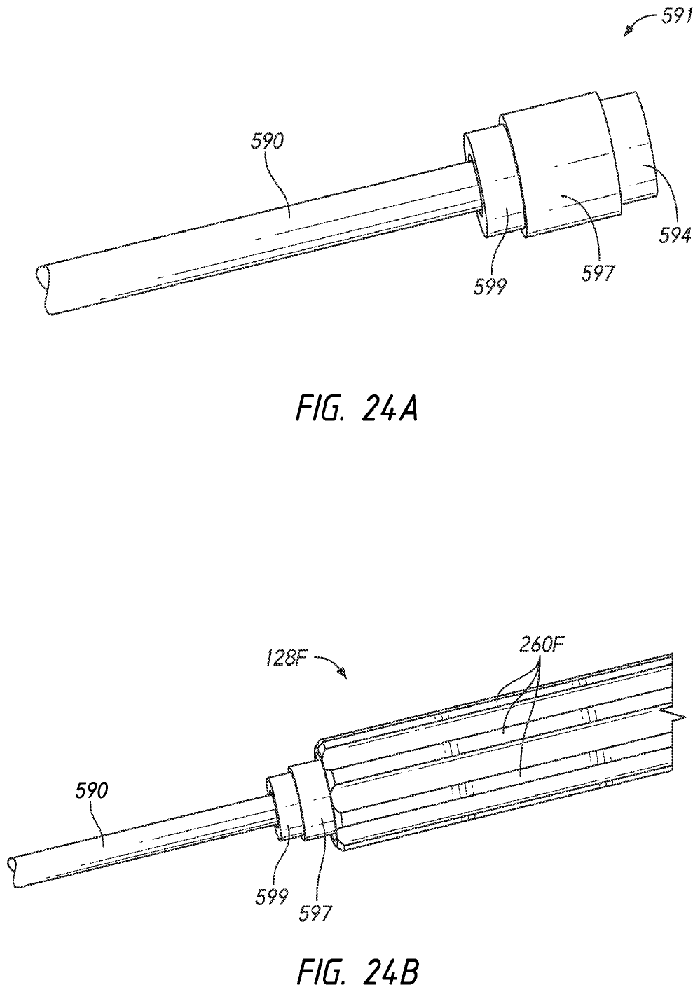

The inner connector 128F can be coupled to the shaft 590 and can be configured to cause the shaft 590 to move relative to the tubing 570. FIG. 24A shows an isolation view of the proximal end 591 of the shaft 590 and FIG. 24B shows an isolation view of the inner connector 128F coupled to the shaft 590. In one example, the inner connector 128F can be coupled to the shaft 590 via one or more connector sleeves. For example, the flow restriction systems 5 can include a first sleeve 594, a second sleeve 597, and/or a third sleeve 599. The first sleeve 594 can be coupled to the shaft 590 at or near its proximal end 591. The third sleeve 599 can be coupled to the shaft 590 distal to the first sleeve 594. The second sleeve 597 can be disposed between the first and third sleeves 594, 599. In some cases, one or both of the first and third sleeves 594, 599 can be welded to the shaft 590. In other cases, one or both of the first and third sleeves 594, 599 can be crimped to the shaft 590. The second sleeve 597 may have a clearance fit with the shaft 590. For example, the second sleeve 597 may not be rotationally fixed to the shaft 590. The sleeves 594, 597, 599 may have inner diameters that are substantially the same size. In some variations, the sleeve 597 has an inner diameter that is larger than the inner diameters of the sleeves 594, 599 such that the sleeve 597 is provided with clearance to the shaft 590. However, the second sleeve 597 may have an outer diameter that is larger than the outer diameters of the first and third sleeves 594, 599. As such, the second sleeve 597 can be prevented from translating along the shaft 590 past the first sleeve 594 or the third sleeve 599 but may still be configured to rotate relative to the shaft 590.

As shown in FIG. 24B, the inner connector 128F can be coupled to the second sleeve 597. As such, axial or linear movement of the inner connector 128F relative to the external housing 200F causes corresponding linear movement of shaft 590 to move the implant 500 between flow restricting states. However, because the second sleeve 597 is not rotationally fixed to the shaft 590, the shaft 590 and the inner connector 128F can rotate independently of each other. As explained further herein, the rotational position of the inner connector 128F is generally locked or fixed relative to the external housing 200F. As such, the shaft 590 can rotate without causing strain on the inner connector 128F. During use and/or implantation of the flow restriction systems 5, the shaft 590 may rotate. By coupling the shaft 590 to the inner connector 128F via the rotationally free second sleeve 597, rotation of the shaft 590 does not cause undue strain or damage to or decoupling of the shaft 590 from the inner connector 128F.

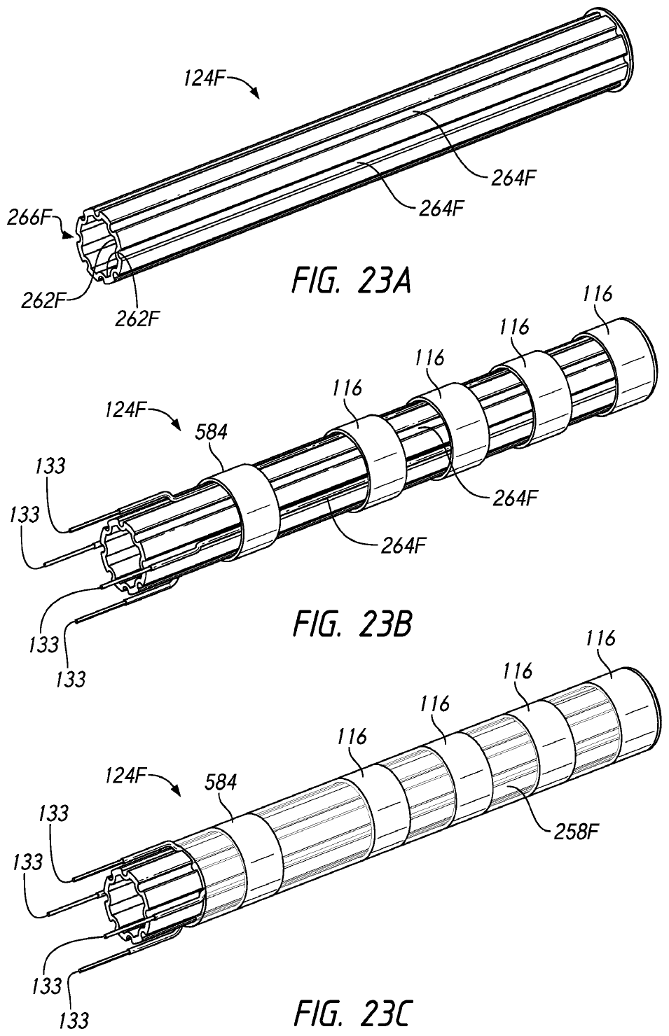

FIG. 21I illustrates a cross-sectional view of the external housing 200F taken along the line I-I in FIG. 21A. As shown, the rod 122F can be coupled to and configured to rotate with the second gear 172F. The rod 122F can have an externally threaded surface. The rod 122F can extend through the first compartment 151F of the ingress protection system 150 and may extend into the second compartment 152F. The threads of the rod 122F can be configured to mate with an internally threaded surface 129F of the inner connector 128F, shown more clearly in FIGS. 22C and 22D. The rod 122F can comprise a tube with an internally threaded surface 129F. The internally threaded surface 129F can extend wholly or partially along the length of the inner connector 128F. In the illustrated example, only a portion of the internal surface of the inner connector 128F is shown as threaded. Additionally, the inner connector 128F is shown at an extreme distal position in FIG. 21I for illustrative purposes. To prevent the inner connector 128F from rotating with the rod 122F, the inner connector 128F can be positioned within an outer connector 124F of the connector assembly 290F. In various embodiments, the connector 124F need not be received in the connector 128F and thus the outer connector 124F may be referred to herein as a “first connector” and the inner connector 128F may be referred to as a “second connector”. The outer connector 124F can be configured to be coupled to the tubing 570, while the inner connector 128F is configured to be coupled to the shaft 590, as shown and described further with reference to at least FIGS. 22A and 22B. The outer connector 124F is shown in isolation in FIG. 23A. To prevent the inner connector 128F from rotating with the rod 122F, the inner connector 128F can include one or more projections that are configured to mate with one or more recesses of the internally threaded surface 129F, or vice-versa. In the illustrated example, the inner connector 128F includes one or more splines 260F formed on its the outer surface. Similarly, the outer connector 124F includes one or more spline guides 262F formed on its inner surface. The spline guides 260F of the inner connector 128F are configured to be positioned in and mate with the spline guides 262F of the outer connector 124F. As such, the inner connector 128F is rotationally fixed relative to the outer connector 124F. Accordingly, when the rod 122F rotates, the engagement of the external threads of the rod 122F and the internally threaded surface 129F of the inner connector 128F causes the inner connector 128F to translate axially along the outer connector 124F. For example, engagement between the one or more splines 260F and the one or more spline guides 262F allows the inner connector 128F to translate within the outer connector 124F when driven by the actuator system 104F.

Because the inner connector 128F can be coupled to the shaft 590, translation of the inner connector 128F within the outer connector 124F driven by the rod 122F causes the implant 500 to move between the low profile state and the high profile state. For example, FIG. 22C shows the inner connector 128F in a first position relative to the outer connector 124F, which may correspond to a first configuration of the actuator system 104F and a low profile state for the implant 500. Similarly, FIG. 22C shows the inner connector 128F in a second position relative to the outer connector 124F, which may correspond to a second configuration of the actuator system 104F and a high profile state for the implant 500. The microcontroller 102F can cause the motor 120F to rotate the rod 122F to move the inner connector 128F from the position shown in FIG. 22C to the position shown in FIG. 22D and vice-versa, causing the implant 500 to move between occlusive states.

In some implementations, the connector assembly 290F can be coupled to the external housing 200F by inserting the connector assembly 290F into the hole 208F of the connector portion 206F. Once the connector assembly 290F is positioned at least partially within the ingress protection system 150F, the proximal end of the inner connector 128F may be disposed against the distal end of the rod 122. To connect the inner connector 128F to the rod 122F, the motor 120F can be instructed (e.g., via the external device 15) to move the implant 500 to a higher profile state. As explained herein, the state of the implant 500 may be determined, for example, detected, based on a position of the magnet 168F within the external housing 200F. This instruction can cause the rod 122F to begin rotating, causing the inner connector 128F to catch on the rod 122F. Once the internally threaded surface 129F of the inner connector 128F is engaged with the external threads of the rod 122F, continued rotation of the rod 122F will cause the inner connector 128F to move proximally (e.g., away from the hole 208F). The rod 122F can continue to rotate until the inner connector 128F reaches the set position (e.g., which may be determined based on the position of the magnet 168F), at which point, the motor 120F may stop rotating the rod 122F. Once the rod 122F is engaged with the inner connector 128F, the connector assembly 290F is coupled with the external housing 200F. In some cases, additional methods of coupling the connector assembly 290F to the external housing 200F can be used, such as the set screw(s) 582, as described herein.

FIGS. 23A-23C show perspective views of the outer connector 124F in isolation in various states of assembly. As shown in FIG. 23A, the outer connector 124F can include a channel 266F that extends along the length of the outer connector 124F. Accordingly, the outer connector 124F can be a first tube of the actuator system 104F. The spline guides 262F can be formed on the inner surface of the channel 266F. The outer connector 124F can also include one or more grooves 264F on its outer surface.

As shown in FIG. 23B, the outer connector 124F can include a plurality of leads or wires 133 that are disposed on the outer surface of the outer connector 124F. In some cases, the wires 133 may be positioned in the grooves 264F. The wires 133 can extend to an array of electrical contacts 116 disposed on the outer surface of the outer connector 124F. As explained herein, the electrical contacts 116 can facilitate an electrical connection between one or more pressure sensors (e.g., sensor 600) of the flow restriction system 5 and the microcontroller 102F. In some implementations, each wire 133 may be welded to a different electrical contact 116. The wires 133 can be electrically connected to the wires 114 of the tubing 570, as explained further with reference to at least FIGS. 22A and 22B. In some implementations, the outer connector 124F can include an outer cover 258F shown in FIG. 23C, that extends over the outer surface of the outer connector 124F and at least a portion of the wires 133. The outer cover 258F can assist in securing the wires 133 to the outer connector 124F. In some cases, the outer cover 258F can be a shrink wrapped polymer or resin. In some cases, the outer cover 258F may comprise a reflow material, a solder, adhesive, conformal coating, and/or the like. The outer cover 258F may be added to the outer connector 124F via a reflow process. In some cases, the electrical contacts 116 may not be covered by the outer cover 258F. In some cases, the outer connector 124F can include a clamping surface 584 configured to be engaged by one or more set screws 582 of the external housing 200F to connect the tubing 570 to the external housing 200F.

FIGS. 22A and 22B show a side view of the connector assembly 290F and the tubing 570 and a partial section view of the connector assembly 290F and the tubing 570 respectively. The connector assembly 290F can be coupled to the tubing 570 and can be used to facilitate a mechanical and/or electrical connection between the various components of the system 5 and the implantable control unit 100F. The outer connector 124F of the connector assembly 290F can be coupled to the tubing 570. In the illustrated example, the outer connector 124F is coupled to the tubing 570 via a jacket 579. The jacket 579 can be a reflow material, such as solder, adhesive, conformal coating, and/or the like, and can be added to the tubing 570 and the outer connector 124F via a reflow process. The jacket 579 can extend over the outer surface of the tubing 570 near the proximal end 571, including the step up section 576, and the outer surface of outer connector 124F. Accordingly, the outer connector 124F can be axially and rotationally fixed to the tubing 570.

As shown in FIG. 22B, in some cases, the outer connector 124F can extend at least partially into the proximal end 571 of the tubing 570. Similarly, the inner connector 128F may extend partially into the tubing 570 when translating within the outer connector 124F. In some cases, the shaft 590 may remain within the lumen of the tubing 570 as the implant 500 moves between occlusive states. In other cases, the proximal end 591 of the shaft 590 may travel outside of the proximal end 571 of the tubing 570 and into the outer connector 124F when the actuator system 104F causes the inner connector 128F to translate. To facilitate an electrical connection between a pressure sensor of the flow restriction system 5 and the microcontroller 102F, the wires 114 of the tubing 570 can be electrically and mechanically coupled to the wires 133 of the outer connector 124F. As explained herein, the leads/wires 114 may be positioned radially outward of the lumen of the tubing 570 and radially inward of the outside surface of the tubing 570. At their distal ends, the wires 114 can be electrically connected to a pressure sensor of the implantable flow restriction system. In one example, crimp sleeves 119 can be used to couple each wire 114 to a corresponding wire 133. In other examples, other conventional electrical coupling methods can be used. When crimp sleeves 119 are used, the crimp sleeves 119, the wires 114, and/or the wires 133 may be positioned in the grooves 264F of the outer connector 124F and secured to the outer surface of the outer connector 124F via the jacket 579. For example, a reflow process (e.g., forming the jacket 579) can be used to couple the crimp sleeves 119 to the outer connector 124F. Arranging the crimp sleeves 119 within the grooves 264F can provide a lower profile outer surface for the connector assembly 290F. Using crimp sleeves can also provide strain relief between the wires 114 and the wires 133.

Referring back to FIG. 21I, the connector assembly 290F can extend entirely or partially into the external housing 200F via the hole 208F in the connector portion 206F. In this arrangement, the tubing 570 extends partially into the external housing 200F via the hole 208F. The tubing 570 and connector assembly 290F can be removably coupled to the external housing 200F via engagement between the one or more set screws 582 and the clamping surface 584 of the outer connector 124F of the connector assembly 290F. When the tubing 570 is coupled to the external housing 200F, the outer connector 124F can extend into the second compartment 152F of the ingress protection system 150F. The inner connector 128F can also extend into the ingress protection system 150F and through the outer connector 124F. The internally threaded surface 129F of the inner connector 128F can be threadedly engaged with the threads of the rod 122F in this arrangement.

During use of the flow restriction system 5, the inner connector 128F may travel at least partially into the first compartment 151F of the ingress protection system 150 through a channel wall 252F that separates the first compartment 151F from the second compartment 152F. As shown more clearly in FIG. 21H, which shows a perspective sectional view of the ingress protection system 150F along its central axis, the channel wall 252F can include an opening 254F. The opening 254F can be sized to prevent the outer connector 124F from extending into a main channel 250F of the first compartment 151F. The opening 254F can be sized to allow the inner connector 128F to translate through the opening 254F between compartments 151F, 152F.

With continued reference to FIGS. 21H and 21I, the second compartment 152F can house a plurality of wire housings 220F. The wire housings 220F can be separated from each other by a plurality of seals 222F. Both the wire housings 220F and the seals 222F can have an annular shape to define a second channel 256F. The outer connector 124F can be positioned within the second channel 256F. The wire housings 220F can be used to facilitate an electrical connection between the electrical contacts 116 of the outer connector 124F and the controller wires 118 that can be electrically connected to the printed circuit board 101F of the microcontroller 102F. Each wire housing 220F can include an inner compartment 226F. The inner compartments 226F can extend around the electrical contacts 116 of the outer connector 124F when the outer connector 124F is disposed in the second compartment 152F. A controller wire 118 can extend into each inner compartment 226F. In some cases, the inner compartments 226F can house one or more wires, springs, and/or other electrically conductive material that can contact both the electrical contacts 116 and the controller wires 118 to facilitate an electrical connection. In this arrangement, separation can be maintained between each electrical contact 116 and between each controller wire 118. The controller wires 118 can extend through the second compartment 152F via openings 221 and into the first internal housing 210F. As shown in FIG. 21C, the cover 212F can include a hermetic passthrough plate 248F configured to allow the controller wires 118 to extend through the cover 212F while maintaining the isolation of the first internal housing 210F. After the controller wires 118 extend through the plate 248F, the controller wires 118 can be electrically connected to the printed circuit board 101F to establish the electrical connection between the pressure sensor(s) (e.g., pressure sensor 600) and the microcontroller 102F.

As explained herein, movement of the inner connector 128F relative to the external housing 200F causes corresponding shaft 590 movement, which causes the implant 500 to move between flow restricting states. Accordingly, the relative position of the inner connector 128F can be used to determine the percent occlusion of the implant. In some implementations, the external housing 200F can include a sensor system (not shown) similar to the sensor system 160 described with reference to at least FIGS. 20A-20C. For example, the sensor system can include one or more Hall effect sensors 215F spaced along a portion of the length of the rod 122F. In the illustrated example of FIG. 21C, only one Hall effect sensor 215F is shown; however, it is recognized the more than one Hall effect sensor 215F can be included in the implantable control unit 100F. The Hall effect sensor(s) 215F can be configured to detect the presence and strength of a magnetic field. The Hall effect sensor(s) 215F may be electrically coupled to the printed circuit board 101F and the microcontroller 102F. The Hall effect sensor(s) 215F may include a strip or plate of conductor or semiconductor material and can be configured to generate a voltage difference when subjected to a magnetic field that is perpendicular to the current flow. As shown in FIGS. 21E and 21I, a magnet 168F can be disposed on the rod 122F. The magnet 168F can have an annular shape. For example, the magnet 168F can include a central hole (not shown) and the rod 122F can extend through the central hole. The magnet 168F can be configured to travel with the inner connector 128F as it translates within the external housing 200F. In one example, the magnet 168F may be coupled to the inner connector 128F. In another example, the magnet 168F may be biased against the inner connector 128F during translation. For example, a spring (not shown) may be positioned between the magnet 168F and the divider wall 126F. For example, the spring may be disposed about the rod 122F. In this example, the spring may be configured to be compressed between the magnet 168F and the divider wall 126F as the inner connector 128F translates proximally (e.g., away from the implant 500), and to extend as the inner connector 128F translates distally (e.g., towards the implant 500). In this arrangement, as the spring extends, the magnet 168F can maintain a position biased against the inner connector 128F. Accordingly, as the inner connector 128F translates along the rod 122F, the magnetic field of the magnet 168F changes its position relative to the Hall effect sensor(s) 215F. As such, the microcontroller 102F may be configured to receive voltage readings from the Hall effect sensor(s) 215F, where the voltage readings are proportional to the strength of the magnetic field of the magnet 168F, which varies with the position of the inner connector 128F. Based in part on this information, the microcontroller 102F can be configured to determine the relative position of the magnet 168F and the inner connector 128F within the external housing 200F, which corresponds to an occlusive state of the implant 500. In some implementations, the microcontroller 102F can be configured to stop the motor 120F when the magnet 168F reaches a position corresponding to the implant's high profile state and/or low profile state.

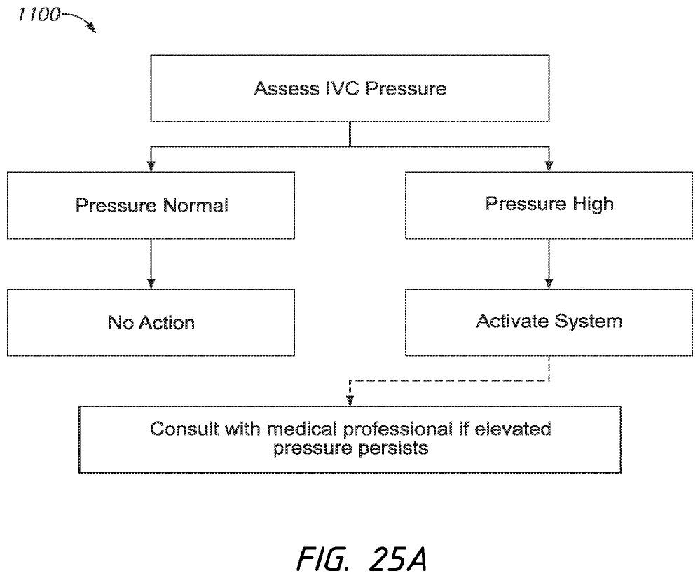

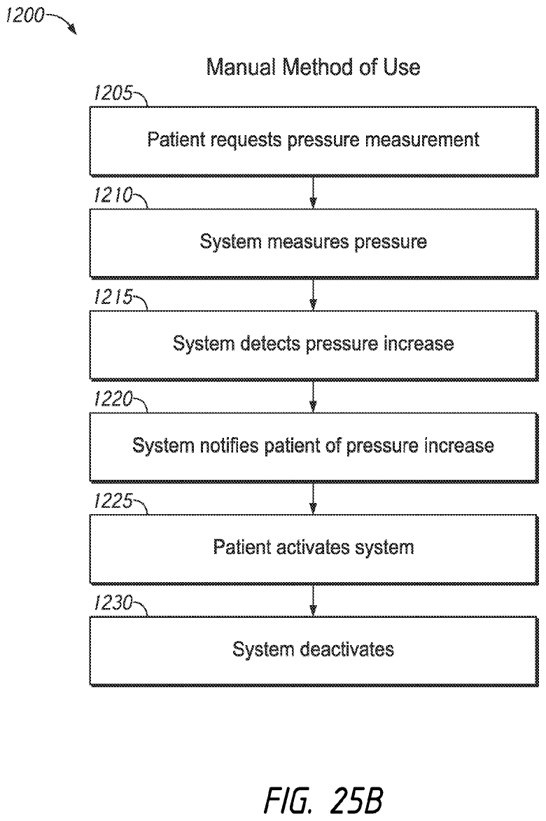

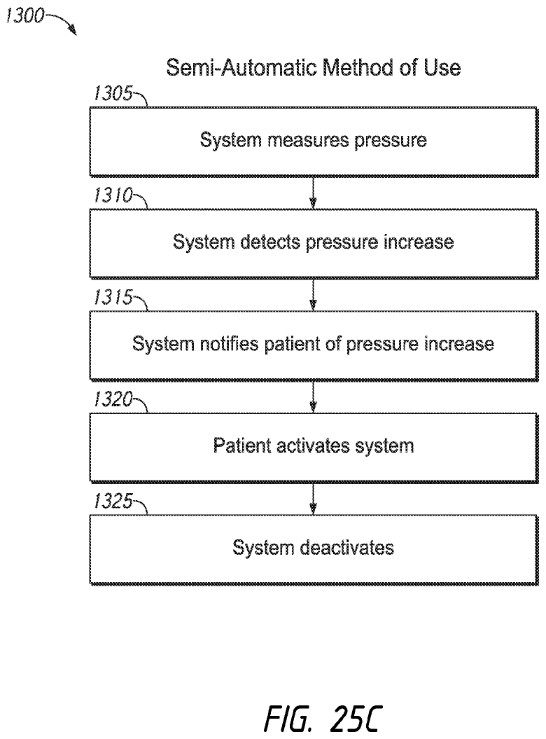

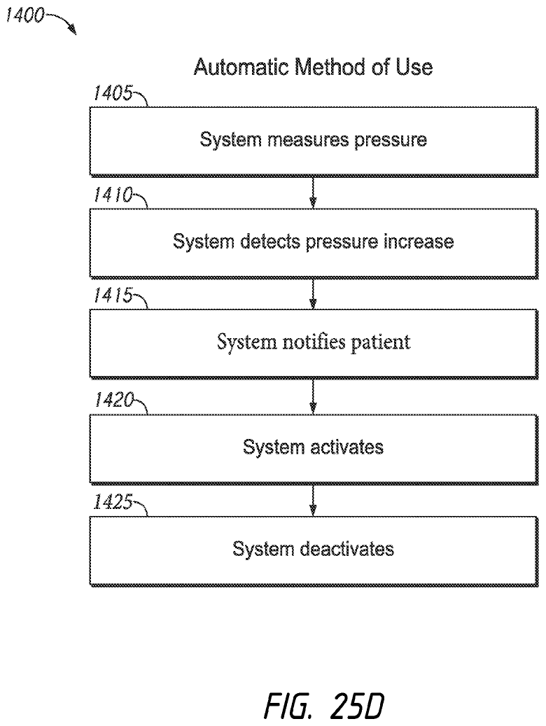

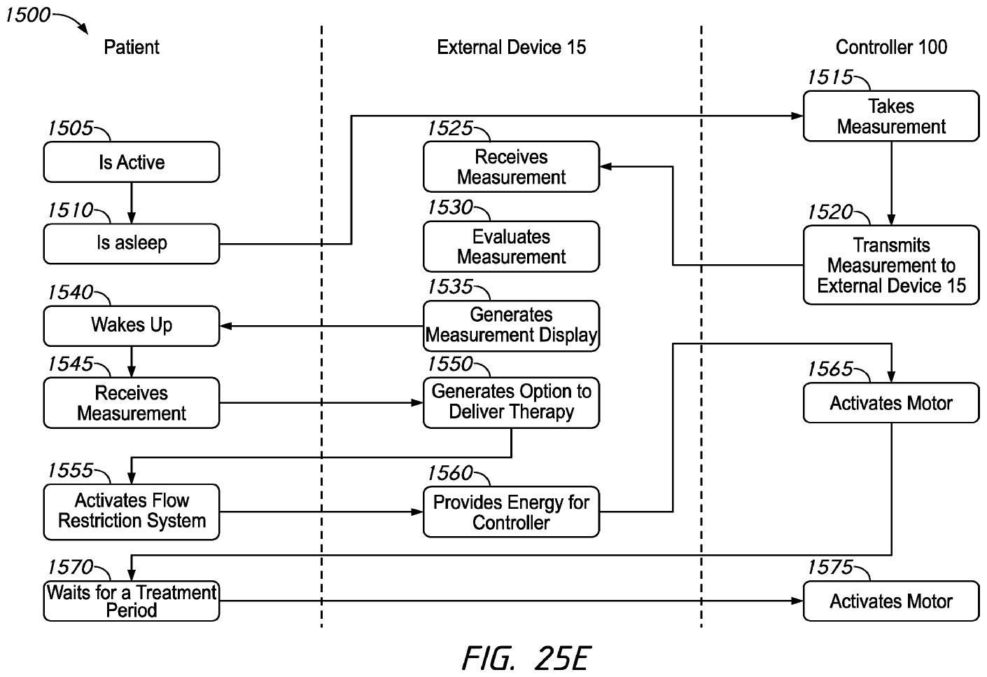

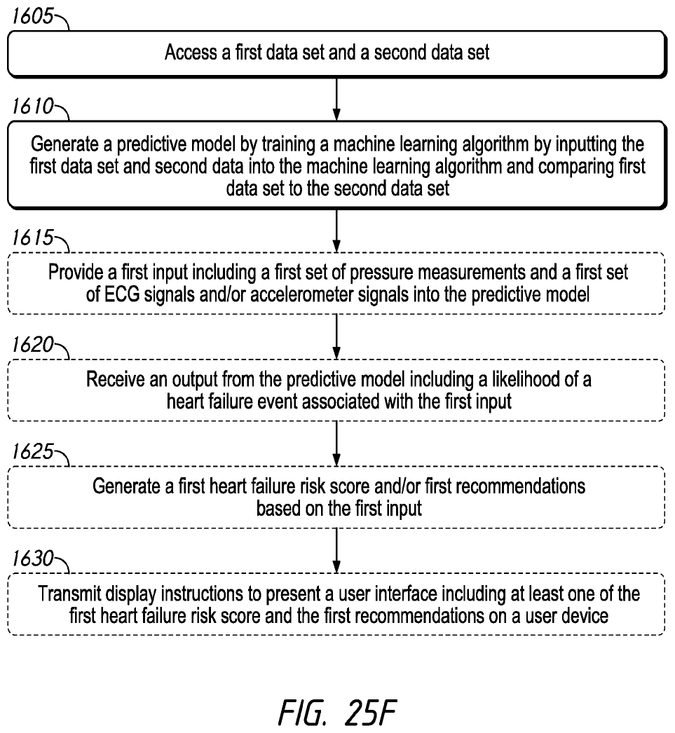

FIG. 25A illustrates a guideline 1100 for enabling a patient to use the implantable flow restriction system 5 described herein. In some implementations, the guideline 1100 can apply to any of the flow restriction systems 5 described herein. The guideline 1100 can include assessing the IVC pressure of the patient. If the IVC pressure is determined to be normal, no action may be required by or recommended by the system 5 (e.g., via a user interface of the external device 15) as shown. Normal IVC pressure can be a pressure of between about 0 mmHg and about 8 mmHg. If the IVC pressure is determined to be high, the system 5 can be activated as shown or activation of the system 5 can be recommended (e.g., via a user interface of the external device 15). High IVC pressure can be a pressure of greater than about 8 mmHg. The IVC pressure can be measured by the system 5 via sensor(s) 600. Furthermore, activation of the system can occur via the external device 15 as described herein (e.g., digital, wireless activation). If the IVC pressure remains high after activation of the system 5, a patient can be recommended to consult with their medical professional/care provider (e.g., via a user interface of the external device 15).

FIGS. 25B-25D illustrate various methods of using the implantable flow restriction system 5 described herein. The methods described with respect to FIGS. 25B-25D can be adapted to any of the flow restriction systems described herein. Furthermore, while the methods described with respect to FIGS. 25B-25D are described using sensor(s) 600 connected to the implant 500, the system 5 can include other sensors proximate the implant 500 and/or remote from the implant 500 for pressure determination(s) and control of the system 5. Similarly, reference to the implantable control unit 100 and its components in the methods of FIGS. 25B-25D is understood to include any of the control units and their respective components described herein (e.g., the implantable control unit 100, the implantable control unit 100A, the implantable control unit 100B, the implantable control unit 100C, the implantable control unit 100D, the implantable control unit 100E, and the implantable control unit 100F). Additionally, while the methods described with respect to FIGS. 25B-25D have been described as being performed by a patient having the system 5 implanted, any steps of such methods can be performed by a medical professional/care provider of the patient or an authorized user.

FIG. 25B illustrates a manual method 1200 (which can also be referred to as a “patient driven method”) of using an implantable flow restriction system 5. Furthermore, the method 1200 can include other steps and/or omit steps.

The manual method 1200 can include a step 1205 of requesting a pressure measurement (e.g., a renal venous pressure measurement or a femoral venous pressure measurement). Such a request can be made by the patient using the external device 15 or other separate electronic device as described herein (e.g., via wireless communication with the system 5).

The manual method 1200 can include a step 1210 of the system 5 measuring the pressure based on the request from step 1205. Such pressure measurement can be measured via the sensor(s) 600 of the system 5. For this, the microcontroller 102 of implantable control unit 100 can be operably connected to the pressure sensor(s) 600 and configured to receive and process a signal from the pressure sensor(s) 600 to determine the pressure (e.g., of the patient's vasculature). For example, an implant 500 that is implanted in the IVC upstream of the renal veins having a sensor 600 connected thereto can be used to measure an IVC pressure, a renal venous pressure, and/or a femoral venous pressure. In other words, an IVC pressure, a renal venous pressure, and/or a femoral venous pressure can be measured from the implant 500.

The manual method 1200 can include a step 1215 of the system 5 detecting a pressure increase. For example, the system 5 can compare the pressure measured in step 1210 to a previously measured pressure and/or to a pressure value in memory (e.g., in a storage device of the implantable control unit 100) to determine if the pressure has increased and/or is elevated/high. Determination of a high pressure can be performed according to the guideline 1100.

The manual method 1200 can include a step 1220 of the system 5 notifying the patient of a pressure increase if detected in step 1215. For this, the system 5 (e.g., the implantable control unit 100) can transmit to the external device 15 an indication that the pressure has increased. Such pressure can include the IVC pressure, the renal venous pressure, and/or the femoral venous pressure. Furthermore, the step 1220 can include notifying the patient, via external device 15, that the pressure has increased and/or is elevated/high. This can include receiving, from the external device 15, an instruction to activate the implant 500.

The manual method 1200 can include a step 1225 of activating the system 5, such as by the patient. For this, the patient can interact with the external device 15 (e.g., via a user interface) to cause actuation of the implant 500. Actuation of the implant 500 can include actuation of flow restrictor 560 and/or flow restrictor portion 550 as described herein, which can at least partially occlude the lumen 513 of the implant 500. Furthermore, actuation of the implant 500 can at least partially occlude the flow of blood through a vessel in the patient's vasculature. For example, for an implant 500 implanted in the IVC below the renal veins of the patient, activating the implant 500 can cause the implant 500 to at least partially occlude blood flow through the IVC.

The manual method 1200 can include a step 1230 of deactivating the system 5. Deactivation of the system 5 can include returning the implant 500 to its un-activated, non-occluding/non-restricting state as described herein. Such deactivation can occur manually, semi-automatically, or automatically. For example, the system 5 can remain activated until deactivated by interaction with external device 15. As another example, the system 5 can notify the patient that therapy is complete and present a notification to deactivate the system 5. Such notification can occur similar to the notification of pressure increase described in step 1220. In another example, the system 5 can remain activated for a duration of time, and the system 5 can deactivate after such duration of time has passed. In yet another example, the system 5 can remain activated for as long as the pressure remains elevated/high, which can include periodic measurements of the pressure for such determination.

FIG. 25C illustrates a semi-automatic method 1300 (which can also be referred to as a “auto-sense with patient activation”) of using an implantable flow restriction system 5. Furthermore, the method 1200 can include other steps and/or omit steps. The method 1300 can be similar to the method 1200 in many respects. For example, the method 1300 can include steps 1305, 1310, 1315, 1320, and 1325 that are the same as the steps 1210, 1215, 1220, 1225, and 1230 of method 1200, respectively. Different than the manual method 1200, the semi-automatic method 1300 can omit the step 1205 of requesting a pressure measurement. In the semi-automatic method 1300, without such a request for a pressure measurement, the system 5 can automatically measure pressure via the system 5. Such automatic pressure measurement can occur based on a predetermined schedule or time interval, which can be the same or different depending on the time of day, the patient, or other factors of the patient. The method 1300 can be referred to as “semi-automatic” in that the system 5 must be activated in step 1320.

FIG. 25D illustrates an automatic method 1400 (which can also be referred to as a “closed loop” or “fully closed loop”) of using an implantable flow restriction system 5. The method 1400 can be similar to the method 1300 in many respects. For example, the method 1400 can include steps 1405, 1410, 1415, and 1425 that are the same as the steps 1305, 1310, 1315, and 1325 of method 1300, respectively. Different than the semi-automatic method 1300, the automatic method 1400 can omit the step 1320 of the system being activated by the patient. In the automatic method 1400, without such a need to be activated by the patient, the system 5 can automatically activate to provide therapy.

The method 1400 can optionally include the step 1415, where the system 5 notifies the patient after detecting the pressure increase at step 1410. The external device 15 may provide a notification to the patient that indicates that therapy will be delivered unless the patient opts-out of the therapy within a certain time period. For example, when the external device 15 determines therapy is required, or would be beneficial due to the pressure increase at step 1410, the external device may generate a notification with a countdown until therapy begins. If the patient takes no action, the method 1400 proceeds to step 1420, where the external device 15 may instruct the controller 100 to proceed with therapy. Conversely, if the patient declines the therapy on the external device 15, the method 1400 may terminate, and the external device 15 may not instruct the controller to proceed with therapy.

FIG. 25E illustrates a swim-lane flow diagram of a method 1500 of a patient using an implantable flow restriction system 5. The method 1500 shows the states of a patient with the implantable flow restriction system 5 implanted, the external device 15, and the controller 100 throughout a patient's day. The method 1500 can be similar to the methods 1200, 1300, and 1400 in many respects. Generally, the method 1500 follows the method 1300. It is recognized that there are other embodiments of method 1500 which may exclude some of the blocks shown and/or may include additional blocks not shown. Additionally, the blocks discussed may be combined, separated into sub-blocks, and/or rearranged to be completed in a different order and/or in parallel.

At block 1505, the patient may be active and going about their day. While the patient is active, the microcontroller 102 may be in a deep sleep with a minimum amount of current being provided to the microcontroller 102. When the implantable control unit 100 includes a second power source 112, the second power source 112 may provide a small amount of power to the microcontroller 102 while the patient is active to maintain the timer provided by the microcontroller 102.

At block 1510, the patient may be asleep. In one example, the implantable control unit 100 may determine whether the patient is asleep or awake based on a continuously running timer. For example, the implantable control unit 100 may treat the patient as “active” from 8 AM-10 PM and “asleep” from 10 PM-8 AM. These time ranges are examples only and the timer may be customizable for individual users. In another example, the implantable control unit 100 may determine the patient is asleep based on an accelerometer or IMU signal used to provide an indication of a patient condition and/or activity level.

At block 1515, while the patient is asleep, the microcontroller 102 wakes up and sends instructions to the sensor(s) 600 to provide one or more pressure measurements. For example, the sensor(s) 600 may provide three to five measurements each night. The number and duration between measurements generated by the sensor(s) 600 can vary between patients and implementations. When the implantable control unit 100 includes a second power source 112, the second power source 112 may provide the power to the microcontroller 102 to communicate with the sensor(s) 600.

At block 1520, while the patient is asleep, the implantable control unit 100 transmits the pressure measurements to the external device 15. For example, the implantable control unit 100 may use the communication module to transmit the pressure measurements to the external device 15 via Bluetooth®. In some implementation, the microcontroller 102 may be configured to receive new instructions from the external device 15. For example, the new instructions may include one or more of: a timer recalibration, a new pressure threshold, and/or the like. Once the implantable control unit 100 transmits the pressure measurements, the microcontroller 102 can return to a deep sleep with a timer set (e.g., the timer may be set for another 24 hours).

At block 1525, the external device 15 receives and stores the one or more pressure measurements from the controller 100. At block 1530, the external device 15 evaluates the pressure measurements. For example, the pressure measurements may be compared to acceptable pressure ranges for the patient. As explained with reference to FIG. 25A, normal IVC pressure can be a pressure of between about 0 mmHg and about 8 mmHg. However, this range may vary from patient to patient.

At block 1535, the external device 15 generates a measurement display/indicator, which may be presented on a user interface of the external device 15. For example, the external device 15 may generate a notification including the measurement display. In some implementations, the external device 15 may display the one or more pressure measurements. In some implementations, the external device 15 may display an average pressure measurement. In some implementations, the external device 15 may display a pressure indicator related to the pressure measurements. For example, the pressure indicator may include a graphical display where a color indicates the pressure status. For example, if the pressure measurements were within a certain tolerance range (e.g., between about 0 mmHg and about 8 mmHg), the external device 15 may display a first color notification (e.g., green). If the pressure measurements were outside of the tolerance, the external device 15 may display a different color notification. In some implementations, the external device 15 tracks the nightly pressure readings and generates a separate notification depending on how many nights the pressure measurements were outside of the tolerance range. For example, when the patient experiences only one night of high pressure (e.g., outside of the tolerance range), the external device 15 may display a second color notification (e.g., yellow). Similarly, when the patient experiences multiple nights of high pressure, the external device 15 may display a third color notification (e.g., red).

At block 1540, the patient wakes up. At block 1545, the patient receives the pressure measurement and/or the measurement indicator/notification generated by the external device 15. If the patient's pressure is within the tolerance range (e.g., the patient receives a green notification), the patient may take no further action and the method 1500 may terminate. If the patient's pressure is outside the tolerance range (e.g., the patient receives a yellow or red notification), the patient may proceed to block 1550. Whether the patient receives treatment for a yellow or red notification may depend on the specific patient. For example, in some cases, if the patient only has one night of high pressure, their physician may not recommend treatment.

At block 1550, the external device may generate an option to deliver therapy. For example, the option may be presented on the user interface of the external device 15. In some implementations, the therapy option may include one or more of a therapy duration and a target state of the implant 500. For example, the target state of the implant 500 may be correlated to one or both of a target occlusion amount for the lumen 513 (as provided by the implant 500) and a target displacement of a portion of the actuator 104 (e.g., a target distance for the traveler 128 to travel).

In some implementations, the patient may be presented with the option to receive therapy. For example, the external device 15 may present a user executable option that allows the patient to select a treatment option and proceed with the therapy. In some implementations, the external device 15 may provide a notification to the patient that indicates therapy will be delivered unless the patient opts-out of the therapy within a certain time period. For example, when the external device determines therapy is required or would be beneficial, the external device 15 may generate a notification with a countdown until therapy begins. If the patient takes no action, the external device 15 may instruct the controller 100 to proceed with the therapy. Conversely, if the patient declines the therapy on the external device 15, the external device 15 may not instruct the controller to proceed with therapy.

At block 1555, the patient may activate the flow restriction system 5. Depending on the implementation, the patient may not need to take any action to activate the flow restriction system 5, as described above. In some implementations, the patient may accept or approve of the therapy option presented on the external device 15. Approving the therapy option may cause the external device 15 to generate and/or transmit treatment instructions for the therapy option (e.g., target displacement and target duration) to the controller 100. In some implementations, activating the flow restriction system 5 may require the patient to place the external device 15 in close proximity to the implantable control unit 100, as described above. In this example, at block 1560, the external device 15 provides the energy to the controller 100 for delivering the therapy (e.g., via induction with the first power source 106). The power provided to the controller 100 may wake the microcontroller 102 from the deep sleep and/or provide the power to the motor 120 to actuate the implant 500.

At block 1565, the controller 100 activates the motor 120 to provide therapy via the implant 500. As noted above, the external device 15 may have transmitted a target duration and/or target displacement for the therapy to the controller 100. The targets may be received by the communication module of the microcontroller 102 and instructions may be executed by the processors of the microcontroller 102. For example, the microcontroller 102 may set a timer for the target duration of the therapy. In another example, the microcontroller 102 may instruct the motor 120 to displace the traveler 128 to a certain position along the rod 122 so that a desired actuation of the implant 500 occurs. As explained herein, actuation of the implant 500 can include actuation of flow restrictor 560 and/or flow restrictor portion 550, which can at least partially occlude the lumen 513 of the implant 500. Furthermore, actuation of the implant 500 can at least partially occlude the flow of blood through a vessel in the patient's vasculature. For example, for an implant 500 implanted in the IVC below the renal veins of the patient, activating the implant 500 can cause the implant 500 to at least partially occlude blood flow through the IVC. In some implementations, the external device 15 may request one or more pressure readings from the controller 100 during the therapy. For example, the external device may transmit a request for a second set of pressure readings during or after the implant 500 is in the target state. This request may cause the microcontroller 102 to instruct the sensor 600 to provide a second set of pressure readings, which may be transmitted back to the external device 15. In some implementations, the patient may be given the option to request pressure readings via the external device 15.

At block 1570, the patient waits for the treatment period to expire. In some implementations, the patient may be able to go about their day as normal during a therapy session. At block 1575, after the target duration has expired (e.g., as determined by the microcontroller 102), the controller 100 reactivates the motor 120 and the implant 500 may return to the minimum occlusive state. In some implementations, the first power source 106 may have stored sufficient power during the inductive coupling with the external device 15 to power the motor 120 to return the implant 500 to its original state. In some implementations, the second power source 112 provides power to the motor 120 to return the implant 500 to its original state. In some implementations, the external device 15 may be used to power the first power source 106 to return the implant 500 to its original state. Although the method 1500 has been described in connection with the controller 100 it can equally be applied to the control units 100A, 100B, 100C, 100D, 100E, and 100F.

In some implementations, the flow restriction system 5 may not include the sensor(s) 600. In this implementation, a patient may choose to deliver therapy via the implant 500 based on symptoms, weight, time prescription (e.g., where a physician instructs the patient on frequency of use), and/or the like. In some implementations, the flow restriction system 5 may be used with an implantable pressure sensor not coupled to the flow restriction system 5, as described above. In some implementations, the external device 15 is configured to communicate with a remote server configured to receive and store data from the pressure sensor and treatment details used by and transmitted from the implantable controller system 100 to the external device 15.

As explained herein, and at least with reference to at least FIGS. 25A-25E above, the implantable flow restriction system 5 is configured to be implanted in a patient to provide therapy to the patient. Therapy can include moving the implant 500 between a low profile state and a high profile state for a duration of time, and then back to a low profile state. Movement back to the low profile state can be to the original low profile state or to a profile state lower than the high profile state to provide an intermediate occlusive effect. Generally, therapy is controlled by an implantable control unit of the implantable flow restriction system 5, such as the implantable control unit 100F. In the following examples, particular reference is made to the implantable control unit 100F and the sensor 600. However, it is recognized that any of the implantable control units described herein may be configured in the same manner as described with reference to the implantable control unit 100F and any of the sensors described herein may be used to generate the relevant pressure signals.

In some implementations, the implantable control unit 100F is configured to communicate with one or more external devices. For example, the implantable control unit 100F may communicate with the external device 15 and/or additional external devices (collectively external device(s) 15). The microcontroller 102F can be configured to cause data to be transmitted to the external device(s) 15 and/or data to be received from the external device(s) 15. In one example, data transmitted from the implantable control unit 100F may include one or more measurements or signals received by the implantable control unit 100F from one or more of the various sensors of the system 5. For example, pressure measurements/signals received from one or more pressure sensors (e.g., sensor 600) of the system 5, accelerometer and/or IMU measurements/signals received from an accelerometer and/or other inertial sensor of the system 5, ECG measurements/signals received from one or more ECG sensors (e.g., ECG sensors 244F) of the system 5, impedance signals received from one or more impedance sensors/instruments of the system 5 or another system, sounds signals received from one or more sound measurement devices of the system 5 or another system, temperature signals received from one or more temperature measurement devices of the system 5 or another system, oxygen signals received from one or more oxygen sensors of the system 5 or another system, and/or the like. In these examples, the sensors and devices of the system 5 can be configured to generate various signals associated with the particular measurements for transmission to the microcontroller 102F. The sensors/devices may record these measurements at scheduled intervals, times, and/or in response to requests from the microcontroller 102F and/or the external device(s) 15. For example, data received by the microcontroller 102F from the external device(s) 15 may include one or more requests for measurements from the sensors and devices of the system 5. In one example, the microcontroller 102F can be configured to provide one or more pressure measurements to the external device(s) 15 in response to a request received from the external device(s) 15. The pressure measurements may have been stored by the microcontroller 102F and can relate to pressures recorded over a past period of time (e.g., the past hour, day, week, and/or the like) or may be real-time pressure readings generated in response to the request. In the second example, the external device(s) 15 may send requests to the microcontroller 102F, which can cause the sensor 600 to generate a first pressure signal in response to the request. The first pressure signal and/or past pressure signals can be transmitted back to the external device(s) 15 via the implantable control unit 100F. In some cases, the sensor 600 can be configured to generate pressure signals at scheduled times, at scheduled intervals, and/or based on instructions received from the external device(s) 15. For example, the instructions can include scheduled times throughout the day where the microcontroller 102F causes the sensor 600 to record pressure signals (e.g., at 8 AM and 10 PM each day). In another example, the instructions can include scheduled intervals throughout one or more days where the microcontroller 102F causes the sensor 600 to record pressure signals (e.g., every 5 minutes, 30 minutes, hour, 4 hours, and/or the like). The instructions can be generated by the external device(s) 15 (e.g., by a physician or at a physician's recommendation) or be preprogrammed into the microcontroller 102F (e.g., in a closed-loop system). In some implementations, the microcontroller 102F is configured to continue executing a set of pressure reading and/or pressure transmission instructions until the set of instructions is modified by the external device(s) 15.

As explained herein, the implantable control unit 100F can be configured to move the implant 500 between low profile states and high profile states (or to occlusive states therebetween) in response to input instructions. In some cases, the input instructions may be programmed into the microcontroller 102F, and the flow restriction system can work in a close-loop environment. In other cases, the input instructions can be received remotely and/or locally from the external device(s) 15. In such cases, the input instructions may be based on a therapy routine provided by a physician. The instructions can cause the implantable control unit 100F to apply the therapy routine for one or more specific duration (e.g., 5 minutes, 15 minutes, 30 minutes, 1 hour, and/or the like), at one or more specific magnitudes of occlusion (e.g., 25%, 50%, 75%, 100% occlusion, and/or the like), and/or at one or more specific times and/or intervals.

In some implementations, the external device(s) 15 can be configured to receive response data from the patient. The response data can indicate one or more patient events and/or one or more patient conditions. Patient events can include changes in the patient's medical condition. For example, a change in a medical condition can be an update in a patient's medical history based on a consultation with a physician, a recent hospital visit or hospitalization, a patient indicated change in health, and/or the like. Patient events may include time and/or date stamps. In some cases, the external device(s) 15 can be configured to receive patient events from the patient, the patient's physician, the hospital, and/or the like. When patient event information is received, the therapy routine applied by the system 5 may be modified. For example, the patient's physician may prescribe a different therapy routine based in part on the patient events, such as, for example, different times, durations, magnitudes of occlusion, and/or the like for the applied therapy. In some implementations, the patient's therapy routines may be generated by a predictive model, which can include patient event information as an input, in generating the patient's therapy routine, as described further with reference to at least FIG. 25F.

As explained herein, in some implementations, an accelerometer can be used with the system 5 to provide additional patient information. In some cases, the accelerometer may be part of the system 5. For example, the accelerometer may be part of the implantable control unit 100F (e.g., positioned in or coupled to the external housing 200F) and/or in wired or wireless communication with the implantable control unit 100F. In other cases, the accelerometer may be in direct communication with the external device(s) 15. The accelerometer can be configured to generate accelerometer signals that can be transmitted to the microcontroller 102F and/or to the external device(s) 15 (e.g., directly or via the implantable control unit 100F). The accelerometer and/or IMU signals can be a measurement of the intensity and frequency of the patient's movements. For example, the accelerometer signal can indicate a patient condition and/or activity level. The patient condition can include an indication of the physical state of the patient at the time the signal is generated. For example, an IMU signal can be processed to determine whether the patient is lying down, sitting, standing, remaining stationary, moving and/or the like. The activity level can be an indication of the intensity of the patient's activities. For example, the activity level can indicate the patient is engaged in a low level activity, medium level activity, and/or high level activity. In some cases, the implantable control unit 100F can be configured to cause the sensor 600 to generate pressure signals when the patient is in a certain patient condition and/or at a certain activity level. For example, it can be desirable to record pressure measurements only when the patient is generally remaining stationary and/or at a low activity level. This is because patient movement and/or medium to high activity can cause the patient's pressure levels to fluctuate in a manner unrelated to their medical conditions for which therapy is being provided. For example, in some implementations, the implantable control unit 100F may only record pressure measurements in the evening and/or morning when it is confirmed, via the accelerometer signal, that the patient is lying down and at a low activity level. In such cases, it may be likely that the patient is sleeping.

In some implementations, when a predictive model is used for generating the patient's therapy routine, the accelerometer signal may provide additional information that the predictive model can use. For example, the accelerometer signal can indicate a patient's general fitness and lifestyle, the patient's response to exercise (e.g., in combination with ECG signals), whether the patient is generally sedentary, whether the patient has suffered a recent fall, increases or decreases in the patient's activity level (e.g., which may indicate an improvement or decline in the patient's health), whether the patient is in compliance with proscribed activity regiments, and/or the like.

In some implementations, it can be desirable to record pressure measurements before, during, and after a therapy routine is performed to provide information about the effects of the therapy. Accordingly, in some implementations, the implantable control unit 100F can be configured to cause the sensor 600 to generate one or more first pressure signals before a therapy routine, one or more second pressure signals during the therapy routine, and one or more third pressure signals after completion of a therapy routine.

The microcontroller 102F can be configured to generate and transmit or to cause a transceiver to transmit a notification when the pressure signals indicates a pressure above a threshold and/or a pressure remaining above the threshold for a first time period. In some cases, the microcontroller 102F may only cause the notification to be sent when the patient's pressure is above the threshold and the microcontroller 102F determines that the patient is resting (e.g., based on the accelerometer data, the time of day, and/or the like). In some implementations, the external device 15 may set the threshold, and the microcontroller 102F may not store the threshold. In this example, regular pressure signals may be transmitted to the external device 15, and the external device may cause a notification to be generated and transmitted/displayed when the pressure signals indicate pressure above the threshold. In some implementations, the implantable control unit 100F may be configured to compare patient pressure measurements generated based on the pressure signal to or to collate such patient pressure measurements with atmospheric pressure measurements. For example, atmospheric pressure measurements, which may be based on the patient's altitude level, can have an impact on the oxygen availability to the patient and the cardiac output, fluid balance, autonomic responses, respiratory patterns, and/or the like of the patient. Accordingly, pressure measurements generated by the sensor 600 depend on a balance of these factors and the patient's individual physiological response to altitude and changes in atmospheric pressure. As such, the sensor 600 may generate a pressure signal that indicates the patient's pressure is above a threshold, but therapy may not be required due to the change in the patient's pressure caused by a lower atmospheric pressure. The microcontroller 102F can compare the patient's pressure measurements to atmospheric pressure in a multitude of different ways. In one example, the microcontroller 102F can cause the pressure readings to be transmitted to the external device(s) 15, which can access an atmospheric pressure measurement for the comparison. For example, the external device(s) 15 may include pressure sensors for generating the atmospheric pressure measurements, may receive the atmospheric pressure measurements from the third party devices (e.g., a wireless charger or a base station for the external device 15 with an atmospheric pressure sensor), may access atmospheric pressure measurements based on the location of the patient and/or the external device 15 itself, and/or the like. In some cases, each patient pressure measurement may be compared to atmospheric pressures on an individual basis and in real-time or substantially real-time. In other cases, pressure measurements taken during an interval (e.g., every hour, half day, day, week, and/or the like) may be compared to atmospheric pressure. For example, the microcontroller 102F can be configured to transmit or cause a transceiver to transmit the patient pressure measurements to the external device 15, which can be configured to compare the patient pressure measurements to or to collate such patient pressure measurements with the atmospheric pressure measurements in a batch process based on time stamps. In some implementations, the microcontroller 102F may be configured to compare patient pressure measurements to atmospheric pressure. For example, the microcontroller 102F may be configured to receive one or more atmospheric pressure measurements from the external device 15 and compare a first patient pressure measurement generated based on the pressure signal to a corresponding atmospheric pressure measurement (e.g., taken at a similar time, in the same location, and/or the like). Based on the comparison, the microcontroller 102F may generate a patient gauge pressure. When the patient gauge pressure exceeds a threshold, the microcontroller 102F may be configured to transmit a notification to the external device 15 and/or to cause the system 5 to perform a therapy routine.

It some cases, it can be desirable to calibrate the pressure sensors of the system 5 to ensure the pressure readings are accurate. For example, in some implementations, the microcontroller 102F can be configured to calibrate the pressure sensor 600 based on a comparison of the pressure signal to a second pressure signal generated by a non-invasive pressure measurement device. Non-invasive pressure measurement devices can include ultrasound imaging devices, doppler ultrasound devices, electrical bioimpedance devices, near-infrared spectroscopy devices, and/or the like. In some implementations, the sensor 600 can be calibrated using an invasive pressure measurement device.

The implantable control unit 100F may be configured to execute a predictive model, communicate with a device, such as the external device 15, which is executing a predictive model, and/or receive instructions based on outputs generated by a predictive model. The predictive model can be generated by training a machine learning algorithm. Machine learning generally refers to automated processes by which received data is analyzed to generate and/or update one or more models. Machine learning may include artificial intelligence such as neural networks, genetic algorithms, clustering, or the like. Machine learning may be performed using a training set of data. The training data may be used to generate the model that best characterizes a feature of interest using the training data. In some implementations, the class of features may be identified before training. In such instances, the model may be trained to provide outputs most closely resembling the target class of features. In some implementations, no prior knowledge may be available for training the data. In such instances, the model may discover new relationships for the provided training data. Such relationships may include similarities between data elements such as sensors signals and heart failure events, as will be described in further detail below.

Predictive models associated with the implantable flow system 5 may be used to determine when the implantable control unit 100F should cause therapy to be performed. For example, the model may be configured to receive pressure measurements from the sensor 600 and/or signals from the other sensors and devices associated with the system 5 and may determine optimal times for therapy to be performed. In another example, predictive models associated with the system 5 may be used to a determine a likelihood of heart failure events for a patient based on signals received from the patient's implantable control unit 100F. The signals can be generated by sensors and devices associated with the implantable control unit 100F, such as pressure sensors (e.g., sensor 600), accelerometers, IMUs, ECG sensors (e.g., ECG sensors 244F), impedance sensors, sound sensors (microphones or auscultation devices), oxygen sensors, temperature sensors, and/or the like. In some cases, the microcontroller 102F may include an AI engine configured to execute a predictive model. The AI engine can be used to improve different aspects of the processes and analytics performed or implemented by the microcontroller 102F. In other cases, the microcontroller 102F may communicate with a third party system, such as the external device 15, that is configured to received sensor/device data from the microcontroller 102F for inputs into the AI engine and generate predictions related to the inputs. The AI engine may include one or more machine learning systems/models, such as, for example, machine learning, artificial intelligence, neural networks, decision trees, and/or the like. For example, the AI engine can implement machine learning algorithms or artificial intelligence algorithms that may, for example, implement models that are executed by one or more processors. Having an artificial intelligence/machine learning model to analyze patient data can provide significant improvements as compared to conventional systems because weighting different factors/inputs may vary in unpredictable or surprising ways that the artificial intelligence/machine learning model can be customized and trained to determine. In some embodiments, the AI engine can use one or more machine learning algorithms to implement one or more models or parameter functions for the analysis performed in heart failure prediction reports.

In some embodiments, a machine learning model can receive inputs it uses to train and/or apply the predictive model to generate an output. In some embodiments, for example, and with respect to a particular patient, inputs can include any and/or all data and signals associated with, generated by, and or in response to requests from the implantable control unit 100F. For example, inputs can include pressure measurements, ECG signals, accelerometer signals, IMU signals, impedance signals, sound signals, oxygen signals, temperature signals, and/or the like. Inputs can additionally include any data associated with the patient, such as patient medical history, patient information (e.g., height, weight, age, sex, gender, lifestyle, activity level, and/or the like), patient responses to medical questionaries, and/or the like. With respect to outputs from the predictive model, for example, the predictive model may output prescribed therapy for the patient, heart failure events predictions, recommendations for the patient, and/or the like, based on weighted inputs, where the weights are determined by the predictive model during training.

In some embodiments, the predictive model can be generated based on training a machine learning algorithm on training data, which may be annotated data, received from or generated by a plurality of implantable control units implanted in a plurality of patients. Training data can also include patient events associated with the plurality of patients. Patient events can include changes in the patient's medical condition, hospital visits, hospitalizations, and/or the like. In some embodiments, the trained predictive model can then be applied, by the implantable control unit 100F and/or a system or device associated with the implantable control unit 100F, to new patient data as part of an output or heart failure prediction report.

A number of different types of algorithms may be used by the machine learning component to generate the models. For example, certain embodiments herein may use a logistical regression model, decision trees, random forests, convolutional neural networks, deep networks, or others. However, other models are possible, such as a linear regression model, a discrete choice model, or a generalized linear model. The machine learning algorithms can be configured to adaptively develop and update the models over time based on new input received by the machine learning component. For example, the models can be regenerated on a periodic basis as newly received data is available to help keep the predictions in the model more accurate as the data is collected over time. Also, for example, the models can be regenerated based on configurations received from external device(s) 15, implantable control units associated with flow restriction systems, and/or the like. Some non-limiting examples of machine learning algorithms that can be used to generate and update the models can include supervised and non-supervised machine learning algorithms, including regression algorithms (such as, for example, Ordinary Least Squares Regression), instance-based algorithms (such as, for example, Learning Vector Quantization), decision tree algorithms (such as, for example, classification and regression trees), Bayesian algorithms (such as, for example, Naive Bayes), clustering algorithms (such as, for example, k-means clustering), association rule learning algorithms (such as, for example, Apriori algorithms), artificial neural network algorithms (such as, for example, Perceptron), deep learning algorithms (such as, for example, Deep Boltzmann Machine), dimensionality reduction algorithms (such as, for example, Principal Component Analysis), ensemble algorithms (such as, for example. Stacked Generalization), and/or other machine learning algorithm. These machine learning algorithms may include any type of machine learning algorithm including hierarchical clustering algorithms and cluster analysis algorithms, such as a k-means algorithm. In some cases, the performing of the machine learning algorithms may include the use of an artificial neural network. By using machine-learning techniques, large amounts (such as terabytes or petabytes) of received data may be analyzed to generate models without manual analysis or review by one or more people.

FIG. 25F shows a flow diagram of an example method of generating a predictive model by training a machine learning algorithm. It is recognized that there are other embodiments of the method of FIG. 25F which may exclude some of the blocks shown and/or may include additional blocks not shown. Additionally, the blocks discussed may be combined, separated into sub-blocks, and/or rearranged to be completed in a different order and/or in parallel. FIG. 25F also shows an example method of applying the predictive model to data associated with an individual patient. The blocks associated with the application of the predictive model to patient data are shown in dotted lines and are not required for the method of FIG. 25F. In some implementations, the method of FIG. 25F may be performed by a system. The system may include a network interface configured to communicate with a plurality of network devices. The system may include or be configured to access one or more data stores. The one or more data stores may store computer-executable instructions configured to perform the method of FIG. 25F. For example, one or more physical computer processors of the system in communication with the one or more data store can be configured to execute the computer-executable instructions to cause the method of FIG. 25F to be performed. The one or more data stores may also store one or more of: a first data set comprising a plurality of pressure measurements collected from a plurality of controller systems (e.g., a plurality of implantable control units 100F) implanted in a plurality of patients, a second data set comprising a plurality of patient events associated with the plurality of patients, and/or a third data set comprising a plurality of measurements collected from the plurality of controller systems and/or one or more additional devices or sensors associated with the plurality of patients.

At block 1605, the system can access, by the network interface and from the one or more data stores. The first data set and the second data set. As noted above, the first data set can include a plurality of pressure measurements collected from a plurality of controller systems. For example, the first data set may include pressure measurement generated by pressure sensors (e.g., sensors 600) implanted in a plurality of patients. The pressure measurements may have been collected over a period of time such as for example, a week, month, year, and/or the like. Similarly, the second data set can include a plurality of patient events associated with the plurality of patients. The patient events can include hospitalization events and hospitalization dates for the plurality of patients. The hospitalization events may be associated with heart failure or heart failure symptoms associated with or exhibited by the plurality of patients.

At block 1610, the system can generate a predictive model by training a machine learning algorithm. Training the machine learning algorithm can include inputting the first data set and the second data set into the machine learning algorithm. Training the machine learning model can further include comparing the first data set to the second data set. For example, the machine learning algorithm may identify features in the patient's pressure measurements that relate to the patient events. Based on the comparison, the predictive model can be configured to determine a likelihood of heart failure events.

In some implementations, training the machine learning model can further include inputting a third data set into the machine learning algorithm. The third data set can include a plurality of signals/measurements collected from the plurality of controller systems or one or more devices/sensors associated with the plurality of controller systems and/or the plurality of patients. For example, the third data set can include one or more of a plurality of accelerometer or IMU signals collected from a plurality of accelerometers or IMUs coupled with the plurality of patients, a plurality ECG signals collected from the plurality of controller systems, a plurality of impedance signals collected from a plurality of impedance measurement instruments associated with the plurality of patients, a plurality of sounds signals collected from a plurality of sound measurement devices associated with the plurality of patients, a plurality of temperature signals collected from a plurality of temperature measurement devices associated with the plurality of patients, a plurality of oxygen signals collected from a plurality oxygen sensors associated with the plurality of patients, and/or the like. In such implementations, the training can further include comparing at least one of the first data set and the second data set to the third data set. For example, the machine learning model may identify features in the patient's accelerometer signals, ECG signals, impedance signals, sound signals, temperature signals, and/or oxygen signals that relate to the patient events. After the predictive model is generated, the predictive model can be applied to specific patient data relating to one or more individual patients.

Impedance signals can be collected from impedance measurement instruments, such as an impedance measurement circuit. In one example, the impedance measurement instruments may be configured to monitor impedance in the patients, which can help in assessing the quality of electrode-tissue interface, detecting electrode dislodgement or damage, ensuring efficient electrical signal transmission, and/or the like. Sound signals can be collected from sound measurement devices. In one example, the sound measurement devices may be configured to monitor the patient's lung sounds, which may assist in early detection of pulmonary congestion. Temperature signals can be collected from temperature measurement devices, such as infrared sensors, contact thermistors, and/or the like. The temperature measurement devices can be configured to detect changes in the patient's temperature. In one example, an increase in patient temperature may indicate a fever, which can be an early sign of inflammation. Oxygen signals can be collected from oxygen sensors and may indicate a patient's oxygen concentration/levels. In one example oxygen levels can provide information about the patient's respiratory status, tissue oxygenation, and/or the like. Identified decreases in oxygen levels may indicate risk of pulmonary edema, cardiac decompensation, and/or the like. Any of the impedance measurement instruments, sound measurement devices, temperature measurement devices, and oxygen sensors can be part of the implantable control units, in communication with the implantable control units, and/or otherwise associated with the plurality of patients. For example, these measurement devices/sensors can implanted in the plurality of patients or used to measure signals from the plurality of patients.

At block 1615, the method can optionally include providing a first input into the predictive model. The first input can include a first set of pressure measurements collected from a first implantable control unit (e.g., the implantable control unit 100F) associated with a first patient. The first input can further include a first set of ECG signals, accelerometer signals, impedance signals, sound signals, temperature signals, and/or oxygen signals collected from the first implantable control unit and/or one or more additional devices associated with the first patient. The first set of pressure measurements and the first set of additional signals may have been collected over a period of time or may be provided into the predictive model in real-time or substantially real-time.

In some implementations, the system may be configured to provide a second input into the predictive model. The second input can include a patient-reported outcome measure data set associated with the first patient. For example, this data set may include patient responses to a survey or questionnaire presented to the patient. The patient responses can provide the first predictive model with further information about the patient based on the patient's assessment of their own health. In one example, the patient-reported outcome measure data set may include first patient responses to the Kansas City Cardiomyopathy Questionnaire and/or the Minnesota Living with Heart Failure Questionnaire.

At block 1620, the system can receive an output from the predictive model. The output can include a likelihood of a heart failure event associated with the first input and the first patient. For example, the predictive model may determine that the first patient is likely to experience a heart failure event in the near future or that the patient is unlikely to experience a heart failure event based at least in part on the first input.

At block 1625, the system can generate a first heart failure risk score and/or first recommendations for the first patient based on the first input. The first heart failure risk score can provide an indication of how likely the first patient is to experience a heart failure event in a future time period. For example, a high risk score can indicate that the first predictive model determined that a heart failure event is likely, while a low risk score can indicate that the first predictive model determined that a heart failure event is unlikely. The first recommendations can relate to the first predictive model's determinations. For example, where a high risk score is indicated, the recommendations may include prescribing increased or modified therapy routines for the patient, recommendations to consult with a physician, recommendations for the first patient to go to a hospital for evaluation (e.g., check into a hospital), and/or the like. In another example, where a low risk score is indicated, the recommendations may include continuing with the prescribed therapy routines. In some cases, the recommendations may include a recommended therapy routine to be administered to the first patient via a blood flow modulator (e.g., implant 500) associated with the first controller system implanted in the first patient.

At block 1630, based at least in part on the first heart failure risk score, the system may be configured to generate and transmit display instructions to a first user device (e.g., the external device 15) associated with the first patient. The display instructions may be configured to present a user interface comprising at least one of the first heart failure risk score and the first recommendations. In some implementations, the system may transmit the display instructions and/or another type of notification to the first patient and/or the patient's physician.

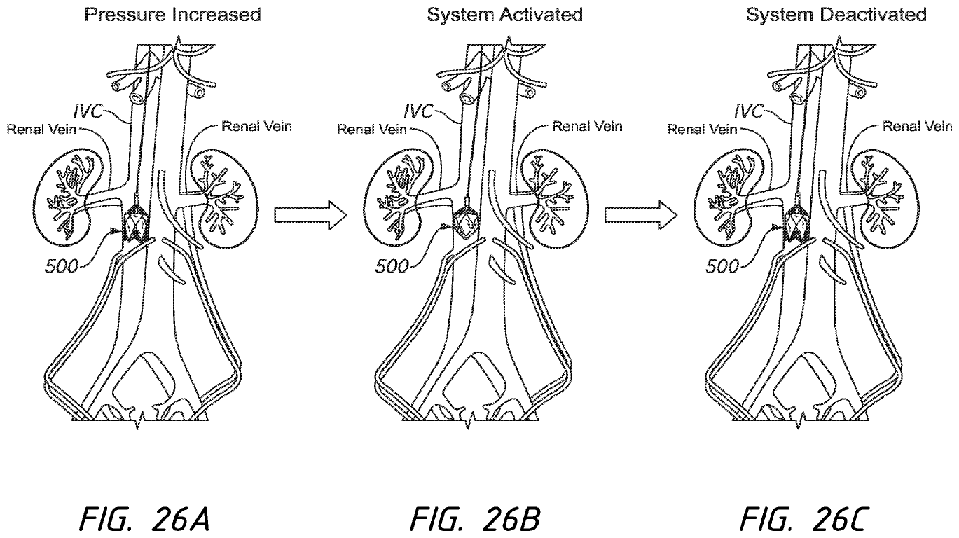

FIGS. 26A-26C illustrate an implementation of delivering therapy using the implantable flow restriction system 5 described herein. The delivery of therapy using the system 5 described with respect to FIGS. 26A-26C can apply to any of the methods described with respect to FIGS. 22-25. FIGS. 26A-26C show the implant 500 of system 5 in the IVC of a patient below the renal veins. As indicated in FIG. 26A, the system 5 has detected an increased or elevated/high IVC pressure, renal venous pressure, and/or femoral venous pressure. Concomitant with the increased or elevated/high renal venous pressure, urine production may be reduced. Depending on the method of use, the system 5 can be activated manually, semi-automatically, or automatically. When activated, the implant 500 can at least partially occlude/restrict blood flow in the IVC as described herein and as shown in FIG. 26B (wherein the implant 500 is shown in an occluding/restricting state). By such placement of the implant 500 in the IVC, when activated the system 5 can reduce renal pressure (e.g., reduce renal venous pressure). Such a reduction in renal pressure can increase urine production of the patient (e.g., enhance/increase diuresis). Also, when activated the system 5 can increase femoral pressure (e.g., femoral venous pressure). The system 5 can be deactivated as shown in FIG. 26C. When deactivated, the implant 500 can assume its substantially non-occluding/non-restricting state and not substantially block/restrict blood flow therethrough. In other words, in the deactivated state the implant 500 may not substantially block/occlude/restrict blood flow in the IVC. Such deactivation can decrease femoral pressure while not substantially affecting renal pressure or urine production (e.g., renal pressure and urine production may normalize upon deactivation of the system 5).

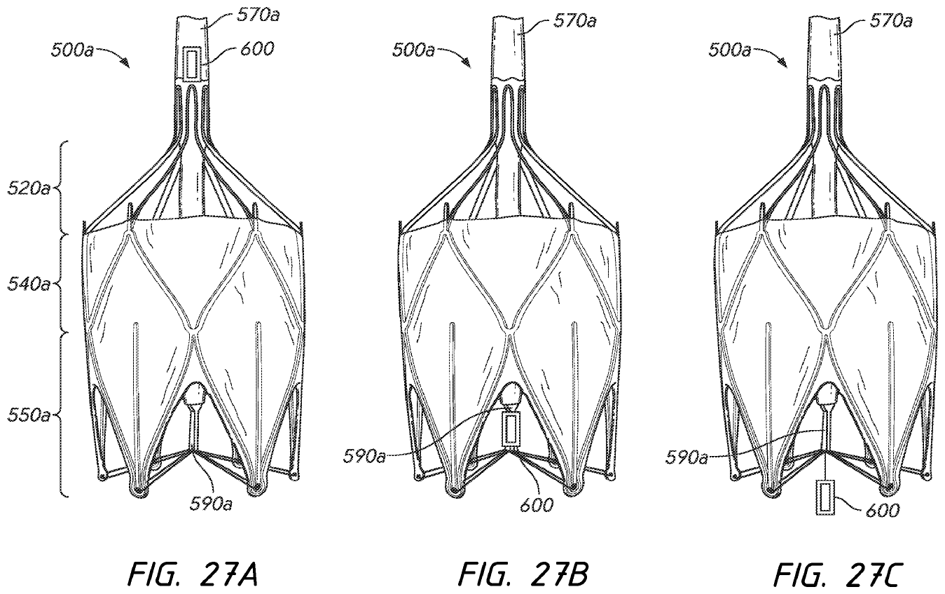

FIGS. 27A-27C show the implant 500 with a sensor 600 located in various positions relative to the flow restrictor portion 550. The sensor 600 can represent any of the sensors described herein. The sensor 600 can be configured to measure the pressure within the vasculature at its location, and as such the placement of the sensor 600 relative to the flow restrictor portion 550 can determine which vascular pressure is being measured depending on the activation state of the flow restrictor portion 550. The sensor 600 can be located adjacent the proximal end of the implant 500 (e.g., such as attached to the tubing 570 proximal to the implant 500) and proximal of the flow restrictor portion 550 if it were to be actuated as shown in FIG. 27A, located adjacent the distal end of the shaft 590 and proximal of the flow restrictor portion 550 if it were to be actuated as shown in FIG. 27B, and/or located on an extension of the shaft 590 and distal of the flow restrictor portion 550 if it were to be actuated as shown in FIG. 27C. When the flow restrictor portion 550 of the implant 500 is unactuated (e.g., the implant is in a non-restricting/non-occluding state), the sensor 600 positioned as shown in any of FIGS. 27A-27C would measure substantially the same pressure. For example, when the flow restrictor portion 550 of the implant 500 is unactuated, the sensor 600 positioned as shown in any of FIGS. 27A-27C would measure substantially the same IVC pressure. When the flow restrictor portion 550 of the implant 500 is actuated, however, the sensor 600 positioned as shown in FIGS. 27A-27B would measure the renal venous pressure (e.g., since it can be positioned proximate the renal veins), whereas the sensor 600 positioned as shown in FIG. 27C would measure the femoral venous pressure. The implant 500 can include the sensor 600 in either of the positions as shown in FIGS. 27A-27B where it would measure the renal venous pressure. In some implementations, the implant 500 can include more than one sensor 600, with a first sensor 600 located as shown in FIGS. 27A-27B to measure renal venous pressure, and a second sensor 600 located as shown in FIG. 27C to measure femoral venous pressure. The sensor(s) 600 of the implant 500 can operably connect with the controller 100, for example, via wires 114 that extend between the sensor(s) 600 and the controller 100 through the tubing 570. In some implementations, the shaft 590 or a portion thereof can operably connect the sensor(s) 600 with the controller 100. Pressures determined from signals generated by the sensor(s) 600 and/or differentials thereof (e.g., differentials between multiple sensors, and/or differentials between pressures determined through time) can be utilized in the control of the implant 500.

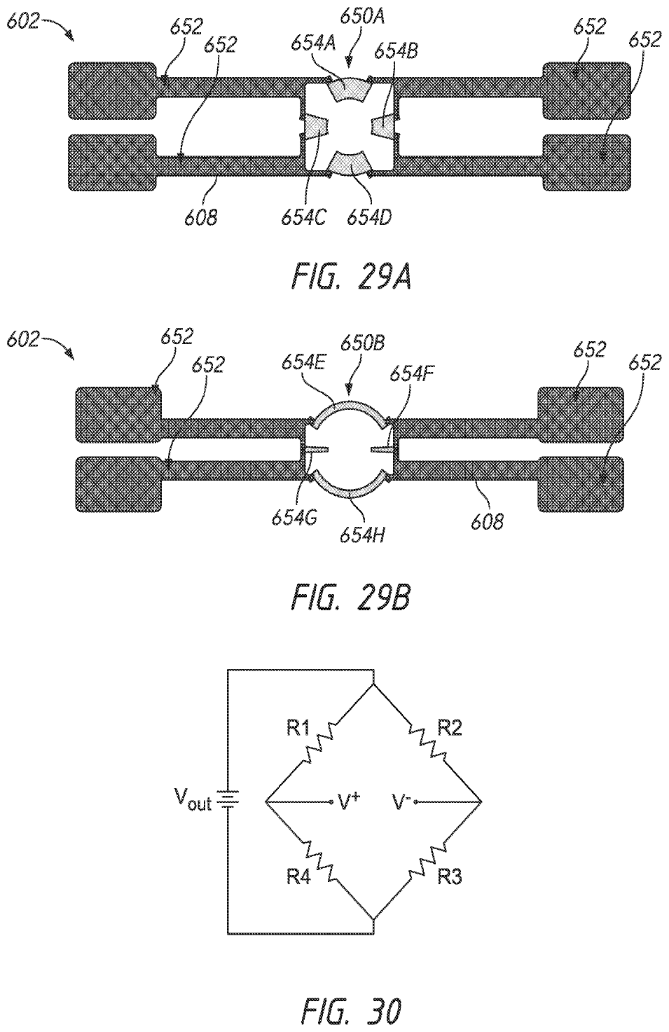

FIGS. 28A and 28B illustrate an implementation of a piezoresistive pressure sensor 600. FIG. 28A shows a top perspective exploded view of the sensor 600 and FIG. 28B shows a bottom perspective exploded view of the sensor 600. The sensor 600 may include a piezo plate 602 and a cavity plate 604. The plates 602, 604 may be generally rectangular. The plates 602, 604 may be generally flat. The plates 602, 604 may be a portion or a segment of one or more wafer plates (e.g., glass wafer plates). In some implementations, the cavity plate 604 may have a greater thickness than the piezo plate 602. The piezo plate 602 may have a first or external side 606 and a second or internal side 608. The piezo plate 602 may include a diaphragm 610 on the internal side 608. The diaphragm 610 may be a piezoresistive diaphragm (e.g., a doped polysilicon). The diaphragm 610 may be a strain-sensitive member. The piezoresistive pattern is not illustrated in FIGS. 28A and 28B. Example piezoresistive patterns 650A and 650B for the diaphragm 610 are illustrated in FIGS. 29A and 29B respectively. The diaphragm 610 can be any suitable shape (e.g., as defined by it periphery), such as a square, circle, rectangle, oval, and/or the like. In the implementation illustrated in FIGS. 28A and 28B, the diaphragm 610 is circular. A circular diaphragm 610 may result in a reduced pressure sensitivity and/or an improvement in durability and reliability as compared to other shaped diaphragms, such as square diaphragms. The diaphragm 610 is configured to deflect, flex, or deform when exposed to pressure, causing strain or stress on the piezoresistive elements (see e.g., resistive elements 654 in FIG. 29A) embedded within the diaphragm 610 or positioned on the diaphragm 610. In some cases, the external side 606 of the piezo plate 602 may include a metallic layer. For example, the metallic layer may be added using a physical or chemical vapor deposition, electroplating, spray coating, sol-gel process, vacuum metallizing, welding process, and/or the like. Including a metallic layer on the piezo plate 602 can allow the sensor 600 to be welded to the inside of a sensor container (e.g., the sensor container 901 of the sensor assembly 900). Any of the other sensors described herein (e.g., the sensor 600A, the capacitive sensor 600B, sensor 600C, sensor 600D, etc.) may also include a metallic layer formed on an outer surface for a similar purpose.

The internal side 608 may include a plurality of electrical contacts 612. The plurality of electrical contacts 612 may be any suitable conductive material (e.g., gold). In the illustrated example, the piezo plate 602 includes four electrical contacts 612, however, the number of contacts may vary between implementations. Two electrical contacts 612 may be positioned between a first end 621 of the piezo plate 602 and the diaphragm 610 and two electrical contacts 612 may be positioned between a second end 623 of the piezo plate 602 and the diaphragm 610. A plurality of leads or conductive lines 614 may extend between the diaphragm 610 and the electrical contacts 612. For example, in the illustrated implementations, the internal side 608 includes four leads 614, one lead 614 extending between each electrical contact 612 and the diaphragm 610, resulting in the electrical contacts 612 being conductively coupled to the diaphragm 610. In some implementations, the electrical contacts 612 can comprise a first layer of a first material (e.g., chrome) and a second layer of a second material (e.g., gold). In some implementations, the electrical contacts 612 can include one or more additional layers, such as a third layer of the first material. In this arrangement, the second material can be sandwiched between two layers of the first material. This arrangement may provide a benefit of allowing the electrical contacts 612 to comprise multiple materials with different properties. For example, the first material may have a strong adherence to the material of the piezo plate 602 (e.g., glass), and the second material may have low resistance.

The piezo plate 602 may include a cavity 616 extending from the external side 606 towards the internal side 608. The cavity 616 is aligned with the diaphragm 610 such that the diaphragm 610 is exposed to an external environment via the cavity 616 when the sensor 600 is assembled. As explained further herein, the cavity 616 can create the diaphragm 610 by removing enough wafer (e.g., within the portion that forms the cavity the cavity 616) such that the diaphragm 610 is a semi-rigid portion of the piezo plate 602. The cavity 616 can be any shape, such as a square, circle, rectangle, oval and/or the like. Generally, the cavity 616 is the same shape as the diaphragm 610 (e.g., circular). In some implementations, the cavity 616 may include a tapered edge 617, such that the internal side of the cavity 616 (e.g., adjacent to the diaphragm 610) has a smaller diameter than the external side of the cavity 616.

In some implementations, the piezo plate 602 may include a plurality of wire bonding locations which can be in the form of wire bonding slots 618. The wire bonding slots 618 may be holes extending through the piezo plate 602. The wire bonding slots 618 can be aligned with the electrical contacts 612, such that each wire bonding slot 618 extends partially through one electrical contact 612. The wire bonding slots 618 are configured to receive wires or leads to power and transmit signals to and from the sensor 600. For example, the wires 114 disposed in the tubing 570 of the implant 500 may be received within the wire bonding slots 618 and electrically coupled to the electrical contacts 612. This arrangement may allow the implantable control unit 100 to power, transmit signals, and/or receive signals from the sensor 600.

In some implementations, the piezo plate 602 may include one or both of a first hole 620 at a first end 621 and a second hole 622 and a second end 623 of the plate 602. The holes 620, 622 may be attachment holes that allow the sensor 600 to be fixed to a portion of the implantable flow restriction system 5. For example, the sensor 600 may be coupled via the holes 620, 622 to the implant 500. For example, the sensor 600 may be coupled to the tubing 570 and/or the shaft 590 as discussed with reference to at least FIGS. 27A-27C. The sensor 600 may be coupled to the implant 500 with an adhesive, reflow technique, and/or the like. The sensor 600 may be tied to the implant 500 with a suture or other tension member.

The cavity plate 604 may have a first or external side 626 and a second or internal side 624. In the assembled configuration, the internal side 624 of the cavity plate 604 may be coupled to the internal side 608 of the piezo plate 602. The cavity plate 604 may include a cavity 628 extending from the internal side 624 towards the external side 626. Generally, the cavity 628 does not extend through the cavity plate 604. The cavity 628 can be any suitable shape, such as a square, circle, rectangle, oval and/or the like. Generally, the cavity 628 is the same shape as the diaphragm 610 (e.g., circular). In some implementations, the diameter of the cavity 628 is around the same size or larger than the diameter of the diaphragm 610. In use, the cavity 628 accommodates the deflection of the diaphragm 610. For example, the cavity 628 allows the diaphragm 610 to deflect without contacting or with minimum contact between the diaphragm 610 and the cavity plate 604.

In some configurations, when the sensor 600 is in the assembled configuration, a vacuum exists within the cavity 628, for example, in the spaced bounded by the walls of the cavity plate 604 around the cavity 628 and by the diaphragm 610. Having a vacuum in the cavity 628 beneath the diaphragm 610 may provide certain benefits, including enabling the sensor 600 to generate a pressure reading that is not impacted by temperature. In some configurations, a partial vacuum (e.g., around 0.2 atm) exists within the spaced bounded by the walls of the cavity plate 604 around the cavity 628 and by the diaphragm 610. This configuration may result in temperature fluctuations within the patient having a small or negligible impact on the pressure readings of the sensor 600. In some configurations, no vacuum exists within the cavity 628. In this configuration, a temperature sensor may be used with the implantable flow restriction system 5. For example, the temperature sensor may relay temperature readings to the implantable control unit 100 such that the microcontroller 102 can account for the fluctuations in pressure readings from the sensor 600 based on variation in the temperature readings.

In some implementations, the cavity plate 604 may include a plurality of wire bonding locations which can be in the form of wire bonding slots 630. The wire bonding slots 630 may be aligned with the electrical contacts 612 and the wire bonding slots 618 of the piezo plate 602. The wire bonding slots 630 may extend from the internal side 624 towards the external side 626. In some implementations, the wire bonding slots 630 do not extend the entire way through the cavity plate 604. Like the wire bonding slots 618, the wire bonding slots 630 are configured to allow wires to connect to the electrical contacts 612.

In some implementations, the cavity plate 604 may include one or both of a first hole 632 at a first end 633 and a second hole 634 and a second end 635. In the assembled configuration, the holes 632, 634 are aligned with the holes 620, 622 to allow the sensor 600 be fixed to a portion of the implantable flow restriction system 5, as described above. When reflow is used to fix the sensor 600 to the implantable flow restriction system 5, the combination of the first holes 620, 632 function as a first reflow passage, and the combination of the second holes 622, 634 function as a second first reflow passage. It is recognized that reflow passages are just one example of ways the sensor 600 can be coupled to the tubing 570 or other structure of the system 5. For example, in other implementations, the sensor 600 can include a brazed/welded metallic component (e.g., titanium) that can be welded onto a portion of the system 5.

In some implementations, the cavity plate 604 can be manufactured from a first wafer plate (e.g., made of glass). Depending on the size of the first wafer plate, a number of the cavity plates 604 can be manufactured at the same time from the same first wafer plate. For ease of explanation, reference will be made to manufacturing a single cavity plate 604, however, it is recognized that a plurality of cavity plates 604 would be manufactured at the same time. The first wafer plate may be approximately 0.4 mm thick. However, other thicknesses can be used instead. First, the cavity 628 may be cut into the first wafer plate. When the cavity 628 is circular, the cavity 628 may have a diameter of approximately 1.5 mm, however, other diameters can be used in other implementations. In some implementations, it may be desirable for the cavity 628 to have a depth of greater than 0.005 mm. However, the depth can vary depending on the desired thickness of the cavity plate 604 and/or the expected deflection of the diaphragm 610. In some implementations, the cavity 628 may be cut using a laser or through chemical etching. Laser cutting may provide a more precise cut in some configurations. Next, the holes 632, 634, and the wire bonding slots 630 can be cut (e.g., using a laser). In some implementations, the wire bonding slots 630 may be through-cut, while in other implementations, the wire bonding slots 630 may not be through-cut. At this point, the first wafer plate includes a plurality of cavity plates 604, each with a cavity 628, holes 632, 634, and wire bonding slots 630. The first wafer plate may remain intact until bonded with a second wafer plate, as described below.

In some implementations, the piezo plate 602 can be manufactured from a second wafer plate (e.g., made of glass). The second wafer plate may be the same size and/or identical to the first wafer plate. Depending on the size of the second wafer plate, a number of the piezo plates 602 can be manufactured at the same time from the same second wafer plate. For case of explanation, reference will be made to manufacturing a single piezo plate 602, however, it is recognized that a plurality of piezo plates 602 would be manufactured at the same time.

The second wafer plate may be approximately 0.4 mm thick. However, other thicknesses can be used instead (e.g., a 0.2 mm thick plate). First, polysilicon can be deposited on a first side the second wafer plate (e.g., the first side corresponds to the internal side 608 of the piezo plate 602). The polysilicon can be deposited using chemical vapor deposition, plasma-enhanced chemical vapor deposition, physical vapor deposition, electrochemical deposition, sputtering, and/or the like. Next, the polysilicon can be doped to enhance the gauge factor. The polysilicon can be doped with boron, phosphorus, arsenic, and/or the like. The polysilicon can be doped using ion implantation, diffusion, spin-on doping, plasma doping, and/or the like. Next, the second wafer plate may be annealed. Next a mask including the desired piezoresistive pattern may be applied to the second wafer (see e.g., patterns 650A and 650B shown in FIGS. 29A and 29B respectively). At this point, the majority of the second wafer plate is covered in polysilicon, so the mask covers and protects the portions of the second wafer plate that should remain covered in polysilicon.

Once the mask is applied, the excess polysilicon can be removed (e.g., the portions not covered by the mask) from the second wafer plate. The excess polysilicon can be removed using chemical etching, ultraviolet rays, a laser, and/or the like. Next, a layer of conductive material (e.g., gold) can be deposited on the first side of the second wafer plate (e.g., the same side as the piezoresistive pattern). In some implementations, the mask used to cover the piezoresistive pattern remains on the second wafer plate while the conductive material is deposited. The conductive material can be deposited using chemical vapor deposition, plasma-enhanced chemical vapor deposition, physical vapor deposition, electrochemical deposition, sputtering, and/or the like. Next, a mask including the desired conductive pattern may be applied to the first side of the second wafer (see e.g., conductive masks 652 shown in FIGS. 29A and 29B). Next, the excess conductive material can be removed (e.g., the portions not covered by the conductive mask and the piezoresistive mask). The excess conductive material can be removed using chemical etching, ultraviolet rays, a laser, and/or the like. At this point, the second wafer plate includes a doped polysilicon piezoresistive pattern (e.g., as used with the diaphragm 610) and conductive material in a desired pattern for the electrical contacts 612 and the conductive lines 614.

When the second wafer plate includes the doped polysilicon piezoresistive pattern and the desired conductive material, the first side of the second wafer plate can be completely masked and a portion of a second side of the second wafer plate (e.g., the second side corresponds to the external side 606 of the piezo plate 602) can be removed until a desired overall thickness of the second wafer plate is obtained (e.g., a 0.2 mm thickness may be desired). The portion of the second side may be removed by grinding, etching, laser removal, and/or the like. Next, a second side of the second wafer plate can be masked with only the portions of the second wafer plate corresponding to the cavity 616 being exposed (e.g., the second side corresponds to the external side 606 of the piezo plate 602). For example, the mask may include an exposed circle corresponding a desired shape of the cavity 616. Next, the second side of the second wafer plate may be chemically etched to obtain the cavity 616. By creating the cavity 616, the diaphragm 610 is created.

Once the manufacturing steps described above are complete, the first wafer plate includes the plurality of cavity plates 604 and the second wafer plate includes the plurality of piezo plates 602 with the majority of the features described in FIGS. 28A and 28B complete. Generally, each wafer plate include one or more alignment features to assist with aligning the two plates. When the alignment features are aligned, each cavity plate 604 in the first wafer plate may be aligned with a corresponding piezo plate 602 in the second wafer plate.

In some implementations, the first wafer plate and the second wafer plate may be coupled using epoxy. For example, a thin layer of epoxy may be applied to one or both wafer plates and the plates may be pressed together using the alignment features. Generally, it may be desirable for the epoxy layer to be at least as thick as the conductive layer and/or the piezoresistive layer (e.g., between one and two micrometers thick or greater). Next, the two wafer plates can be compressed for a set duration (e.g., two hours). Finally, any additional holes such as the holes 620, 622 are cut (e.g., using a laser) for each sensor 600 as required and the sensors 600 are cut from the compressed plates to form individual sensors 600.

In some implementations, the first wafer plate and the second wafer plate may be coupled using glass fusion. For example, the wafer plates may be cleaned to remove any debris and the plates may be pressed together using the alignment features. Next, the two wafer plates can be heated to fuse the wafer plates together. Finally, any additional holes such as the holes 620, 622 are cut (e.g., using a laser) for each sensor 600 as required and the sensors 600 are cut from the compressed plates to form individual sensors 600. In some implementations, the first wafer plate and the second wafer plate may be coupled using laser welding.

In some implementations, the first wafer plate and the second wafer plate may be coupled using glass frit bonding. However, this method may require individual piezo plates 602 and individual cavity plates 604 to be coupled instead of the first wafer plate and second wafer plates. First, any additional holes such as the holes 620, 622 are cut (e.g., using a laser) for each piezo plate 602 as required and the individual piezo plates 602 and cavity plates 604 are cut from the wafer plates. Next, the plates 602, 604 may be cleaned to remove any debris and the plates 602, 604 may be pressed together using the alignment features. Next, hot glass frit may be applied along the seam between the two plates 602, 604 and the assembled sensors 600 are allowed to cool.

In some implementations, one or both of an individual piezo plate 602 and individual cavity plate 604 can be metalized to couple the two components. For example, a metalized ring can be added around the circumference of the piezo plate 602 and/or the cavity plate 604 prior to bonding. The piezo plate 602 and the cavity plate 604 could be brazed together to form the bond. In some implementations, the metalized bonding can be used as an alternative to epoxy, frit, and anodic bonding.

In some implementations, after the coupling or bonding step described above, the coupled first and second wafer plates may undergo an annealing process. For example, the coupled wafer plates may be heated (e.g., to 450C) and before slowly cooling. This process may remove any residual stress in the wafer plates and sensors 600. Additionally, this process may help mitigate drift as the residual stress in the sensor 600 slowly relieves itself as the sensor 600 cycles under pressure.

As noted above, in some implementations, the cavity 628 may include a vacuum or partial vacuum. In order to achieve this feature, the first and second wafer plates may be assembled/fused in a vacuum or partial vacuum. Generally, only the bonding steps described above need to be performed in the vacuum or partial vacuum.

FIGS. 29A and 29B illustrate schematic implementations of the internal side 608 of the piezo plate 602. FIG. 29A shows a first piezoresistive pattern 650A and a plurality of conductive masks 652. The piezoresistive pattern 650A may have been created on the diaphragm 610 of the piezo plate 602 as described above. The piezoresistive pattern 650A includes piezoresistive elements 654A, 654B, 654C, 654D. The piezoresistive elements 654 act as resistors and are configured to exhibit changes in their resistance that is proportional to an applied pressure (e.g., from the blood in the lumen 513). As noted above, the diaphragm 610 is configured to flex/deform/deflect when exposed to pressure, which causes stress or stain on the piezoresistive elements 654 embedded in the diaphragm 610. As the pressure on the diaphragm 610 changes, the resistance of the piezoresistive elements 654 also changes. The piezoresistive elements 654 may be arranged as a full bridge circuit, such as the Wheatstone bridge configuration illustrated in FIG. 30. For example, the piezoresistive elements 654A, 654B, 654C, 654D correspond to R1, R2, R3, and R4 respectively. The change in the resistance can be measured (e.g., by the microcontroller 102) using the bridge configuration, which converts the resistance variations into an electrical output signal. For example, two diagonal electrical contacts 612 (e.g., the top left and bottom right electrical contacts 612 in FIG. 28B) can be used to provide power to the piezoresistive elements 654 and the other two electrical contacts 612 (e.g., the top right and bottom left electrical contacts 612 in FIG. 28B) can be used to measure the voltage, which the microcontroller 102 can use to determine the pressure in the lumen 513. In some implementations, the gauge factor of the piezoresistive pattern 650A may be greater than 100. The gauge factor of the piezoresistive pattern 650A may vary with temperature, so the microcontroller 102 may factor in the temperature of the patient when determining the pressure.

FIG. 29B shows a second piezoresistive pattern 650B and the plurality of conductive masks 652. The second piezoresistive pattern 650B includes piezoresistive elements 654E, 654F, 654G, and 654H. The second piezoresistive pattern 650B differs from the piezoresistive pattern 650A in the shape of the pattern, as defined by the piezoresistive elements 654E, 654F, 654G, and 654H. For example, the shape of the pattern in FIG. 29A may provide a lower resistance, which can result in less noise, but may require more energy, while the shape of the pattern in FIG. 29B may provide a higher resistance, which can result in more noise, but may require less energy.

In use, the sensor 600 may be implanted in the patient as part of the implantable flow restriction system 5, as described above. The sensor 600 will be mounted to or coupled with a portion of the implant 500 with the piezo plate 602 exposed to the blood when implanted. In this arrangement, the diaphragm 610 can deflect due to the pressure in the lumen 513. The sensor 600 can be mounted with the cavity plate 604 in contact with the implant 500 and the piezo plate 602 facing away from the implant 500. When the sensor 600 is electrically coupled to the implantable control unit 100 (e.g., via the wires 114), the implantable control unit 100 receives the measured voltage from the piezoresistors 650A/650B, and the microcontroller 102 can determine the pressure in the lumen 513.

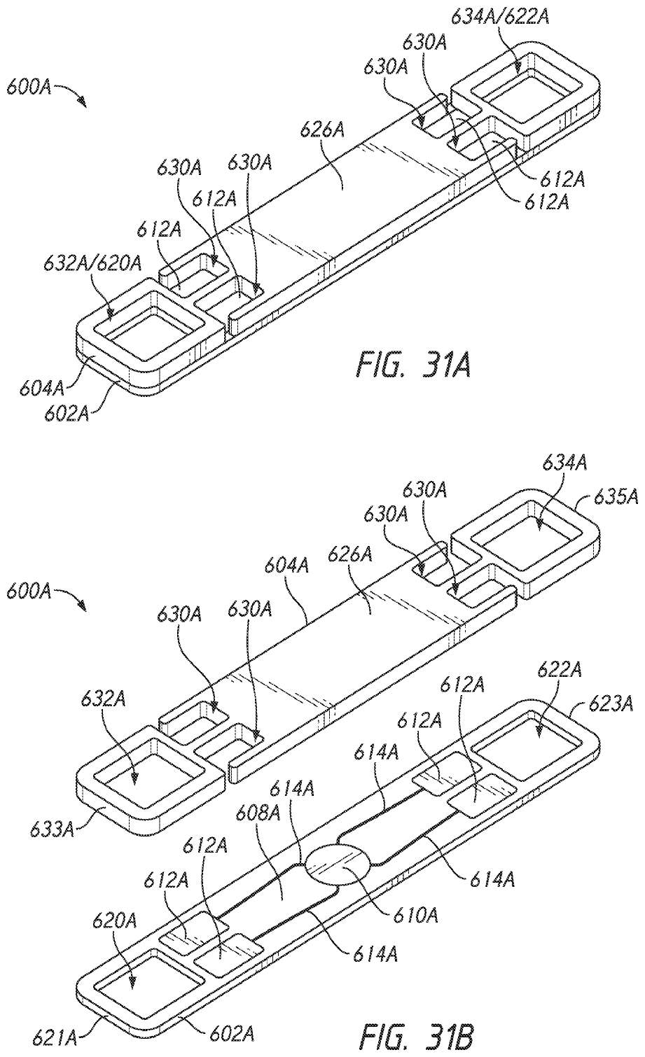

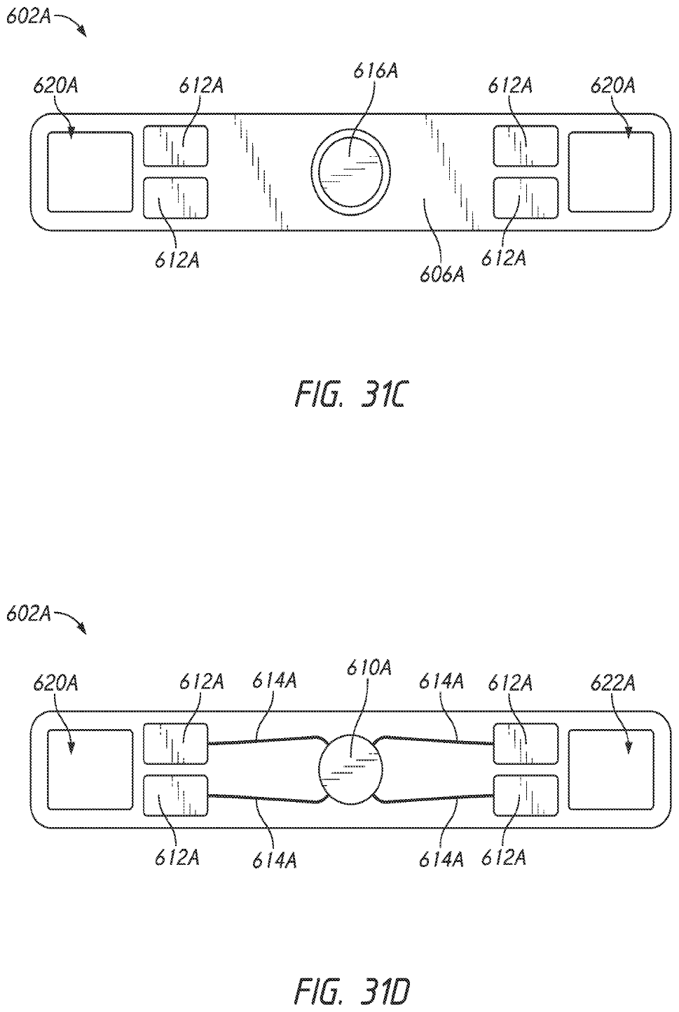

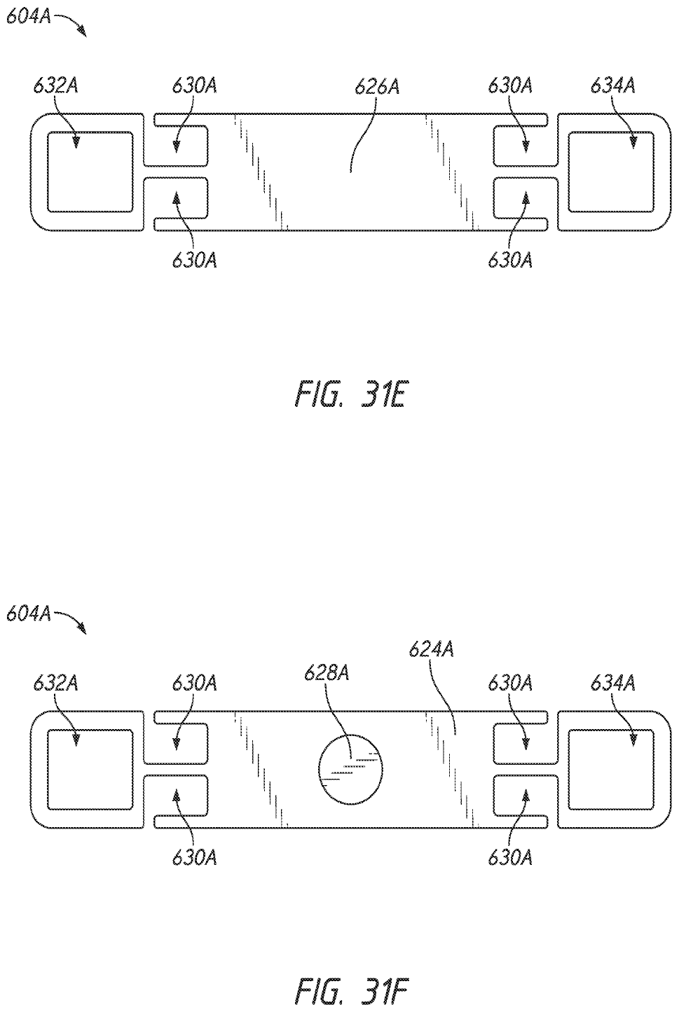

FIGS. 31A-31F illustrate an implementation of a piezoresistive pressure sensor 600A. Some of the features of the sensor 600A are similar to features of the sensor 600 in FIGS. 28A and 28B. Thus, reference numerals used to designate the various features or components of the sensor 600 are identical to those used for identifying the corresponding features of components of the 600A in FIGS. 31A-31E, except that an “A” has been added to the numerical identifier for the sensor 600A. Therefore, the structure and description for the various features of the sensor 600 and how it is operated and manufacture in at least FIGS. 28A-30 are understood to also apply to the corresponding features of the 600A in FIGS. 31A-31E, except as described differently below.

FIG. 31A shows a perspective view of the sensor 600A, FIG. 31B shows an exploded view of the sensor 600A, FIG. 31C shows a front view of the external side 606A of the piezo plate 602A. FIG. 31D shows a front view of the internal side 608 of the piezo plate 602A, FIG. 31E shows a front view of the external side 616A of the cavity plate 604A, and FIG. 31F shows a front view of the internal side 624A of the cavity plate 604A. The sensor 600A differs from the sensor 600 primarily in that the wire bonding slots 630A extend entirely through the cavity plate 604A. Additionally, the wire bonding slots 630A may include an open ended side on the side closest to the ends 633A and 635A. Additionally, the piezo plate 602A may not include wire bonding slots (see e.g., the wire bonding slots 618 of sensor 600).

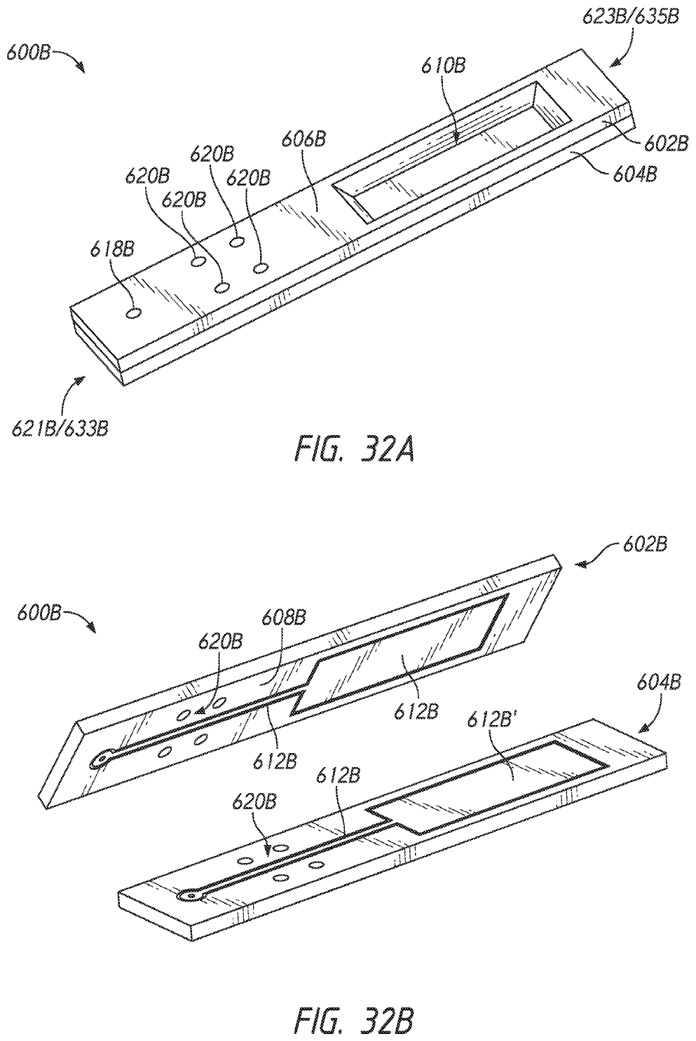

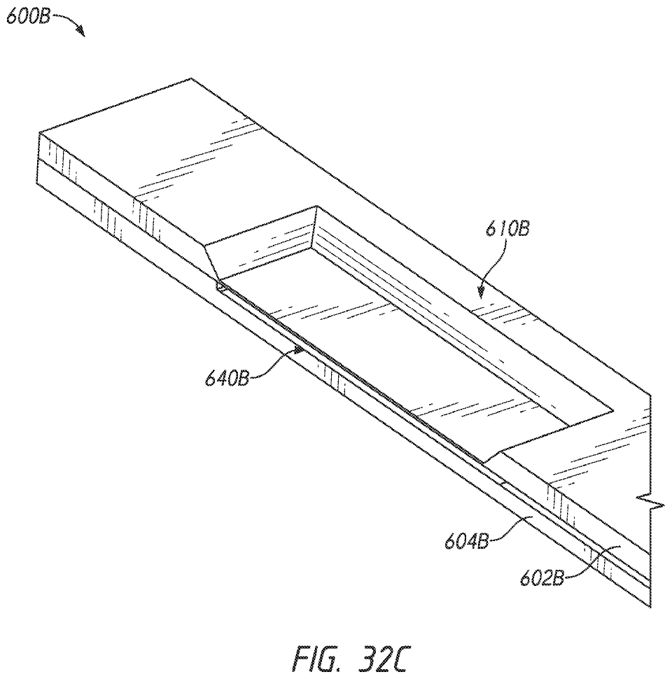

FIGS. 32A-32C illustrate an implementation of a capacitive sensor 600B. Some of the features of the sensor 600B are similar to features of the sensor 600 in FIGS. 28A and 28B. Thus, reference numerals used to designate the various features or components of the sensor 600 are identical to those used for identifying the corresponding features of components of the 600B in FIGS. 32A-32C, except that a “B” has been added to the numerical identifier for the sensor 600B. Therefore, the structure and description for the various features of the sensor 600 and how it is operated and manufacture in at least FIGS. 28A-30 are understood to also apply to the corresponding features of the 600B in FIGS. 32A-32C, except as described differently below.

The capacitive sensor 600B differs from the piezoresistive sensor 600 primarily in the type of sensing provided by the sensors. The piezoresistive sensor 600 is configured to measure changes in electrical resistance correlated to changes in pressure based on changes in the piezoresistive elements 654 when the diaphragm 610 is subjected to pressure. Conversely, the capacitive sensor 600B is configured to measure changes in capacitance caused by the variation in the separation distance between two conductive plates (e.g., between conductive plate 612B and conductive plate 612B′). When a pressure is applied to the capacitive sensor 600B (e.g., when implanted with the system 5), the distance between the conductive plates 612B, 612B′ changes, altering the capacitance between the plates, which can be measured by the implantable control unit 100 to determine the applied pressure.

The capacitive sensor 600B may include a first plate 602B and a second plate 604B. As shown in FIG. 32B, each plate 602B, 604B includes a conductive plate 612B, 612B′ on their internal sides 608B and 624B respectively. The conductive plates 612B, 612B′ may be any conductive material (e.g., gold, silver, copper, titanium, and/or the like). The diaphragm 610B is positioned above the conductive plate 612B on the external side 606B of the first plate 602B. As shown in FIG. 32C, there is an internal gap 640B (e.g., a separation distance) between the conductive plate 612B and the conductive plate 612B′ and below the diaphragm 610B. In some implementations, the internal gap 640B may be between 5-20 micrometers wide in atmospheric pressure. In some implementations, the internal gap 640B may be a vacuum, gas-filed, media-filled, and/or the like. As the pressure applied to the diaphragm 610B changes, so does the size of the internal gap 640B. Each conductive plate 612B, 612B′ may be conductively coupled to a conductive line 614B. The first plate 602B includes a wire exit hole 618B such that a wire from the controller 100 (e.g., the wires 114), can extend into the capacitive sensor 600B and be electrically coupled to the conductive plates 612B, 612B′ via the conductive lines 614B. The capacitive sensor 600B may include a plurality of holes 620B extending through both plates 602B, 604B. The holes 620B can be used to mount the capacitive sensor 600B to a portion of the implant 500 or portion of the system 5, as described above.

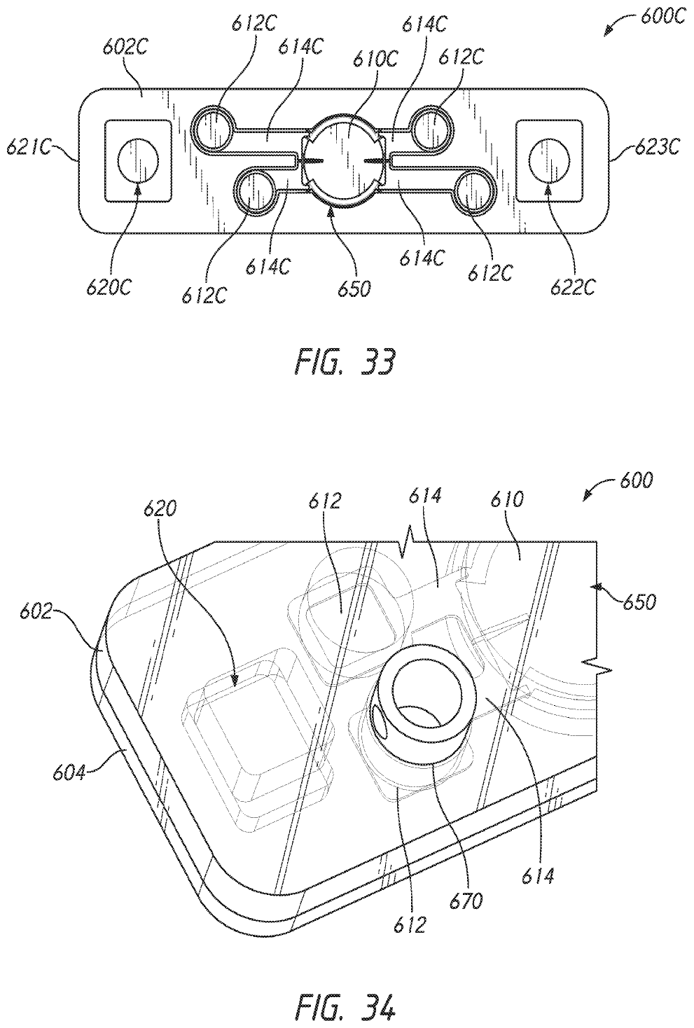

FIG. 33 illustrates an implementation of a piezoresistive pressure sensor 600C. Some of the features of the sensor 600C are similar to features of the sensor 600 in FIGS. 28A and 28B and the sensor 600A in FIGS. 31A-31E. Thus, reference numerals used to designate the various features or components of the sensors 600, 600A are identical to those used for identifying the corresponding features of components of the 600C in FIG. 33, except that a “C” has been added to the numerical identifier for the sensor 600C. Therefore, the structure and description for the various features of the sensor 600 and sensor 600A and how they operated and manufacture in at least FIGS. 28A-30 and in FIGS. 31A-31E respectively are understood to also apply to the corresponding features of the 600C in FIG. 33, except as described differently below.

The sensor 600C differs from the sensor 600 and sensor 600A primarily in the arrangement of the electrical contacts 612C on the piezo plate 602C. In the sensor 600C, the electrical contacts 612C can be staggered relative to each other. For example, a first array of contacts comprising the electrical contacts 612C closest to the first end 621C can be staggered relative to each other such that one electrical contact 612C is closer to the first end 621C and one electrical contact 612C is closer to the diaphragm 610C. Similarly, a second array of contacts comprising the electrical contacts 612C closest to the second end 623C can be staggered relative to each other such that one electrical contact 612C is closer to the second end 623C and one electrical contact 612C is closer to the diaphragm 610C. This arrangement can increase the amount of material between the electrical contacts 612C, which may reduce the likelihood of the sensor 600C being damaged during manufacturing. In some implementations, the electrical contacts 612C can be circular. In other implementations, the electrical contacts 612C can be any other suitable shape such as square, rectangular, diamond, oval, etc. In some implementations, the electrical contacts 612C can be configured to engage a lead (e.g., the wires 114 of the tubing 570) of the implantable control unit 100 in a plug and socket manner. For example, the sensor 600C can be mounted to the tubing 570 and the tubing 570 can include leads on its outer surface coupled to the wires 114. The sensor 600C can be mounted to the tubing 570 such that the electrical contacts 612C engage and couple to the leads of the tubing 570.

FIG. 34 illustrates a close up view of a contact connector 670 and the sensor 600. The contact connector 670 can be configured to electrically and/or mechanically couple the electrical contacts 612 to leads of the implantable control unit 100 (e.g., wires 114 of the tubing 570). In some implementations, the contact connector 670 can be a projection. In some implementations, the contact connector 670 can be a recess. The contact connector 670 can be soldered to the electrical contact 612 and can be coupled to a controller lead via a snap fit connection. In another example the contact connector 670 can be soldered to the controller lead and coupled to the electrical contact 612 via a snap fit connection. The contact connector 670 can be used with any of the sensors described herein.

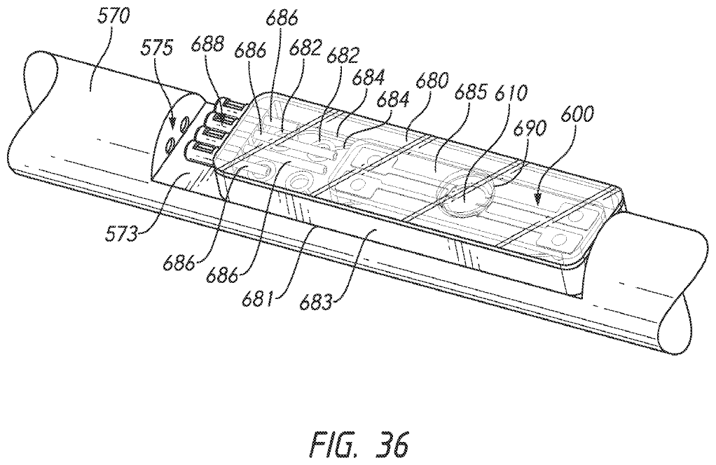

FIG. 36 illustrates an assembly in which a sensor is disposed within an example sensor container assembly 680. The sensor container assembly 680 (also referred to herein at the “sensor case assembly 680”) can be used with any of the sensors described herein. The sensor container assembly 680 can be configured to protect the sensor 600 from damage (e.g., from fluid ingress) and to electrically and/or mechanically couple the sensor 600 to the tubing 570 and the controller leads 114. The sensor container assembly 680 can be a hermetic container. In some implementations, the sensor container assembly 680 can be coupled to the tubing 570 via a cutout portion 573 of the tubing 570.

The sensor container assembly 680 can include a base portion 681, at least one wall 683, and a top portion 685. The at least one wall 683 can extend from the base portion 681 to the top portion 685 to define an internal volume. The sensor container assembly 680 can include or can enclose a plurality of electrical contacts 682, a plurality of conductive lines 684, a plurality of wires 686, a plurality of electrical connectors 688, and/or a diaphragm aperture 690. The plurality of electrical contacts 682 can coupled to or formed on the base portion 681. The plurality of electrical contacts 682 be coupled to the electrical contacts 612 of the sensor 600 via the plurality of conductive lines 684. The plurality of conductive lines 684 can be coupled to or can extend at least partially along the base portion 681. For example, the conductive lines 684 can be at least partially formed on the base portion 681. The plurality of wires 686 can also be coupled to the plurality of electrical contacts 682 (e.g., by extending downward from the elevation illustrated to physically touch the contacts 682 or by providing an intervening material or structure such as solder to provide an electrical connection) for facilitating an electrical connection between the sensor 600 and the implantable control unit 100. The plurality of wires 686 can extend through hermetic wire passthroughs in the sensor container assembly 680 to the plurality of electrical connectors 688. In some cases, the plurality of wires 686 can extend through the at least one wall 683. When the sensor container assembly 680 is coupled to the tubing 570, the plurality of electrical connectors 688 can be inserted into electrical sockets 575 of the tubing 570 that extend to the wires 114. The diaphragm aperture 690 can be a cutout portion extending through the top portion 685. The diaphragm aperture 690 can be aligned with the diaphragm 610 of the sensor 600. The diaphragm aperture 690 can allow the diaphragm 610 to be exposed to the blood flow. The sensor 600 can be coupled to the sensor container assembly 680 to prevent ingress to the sensor container assembly 680 via the diaphragm cutout 690. In some implementations, the glass on the sensor 600 can be metalized by making tracings of metal on the external surface of the sensor 600. These tracings of metal can serve as weld locations, such that the sensor 600 can be welded to the sensor container assembly 680.

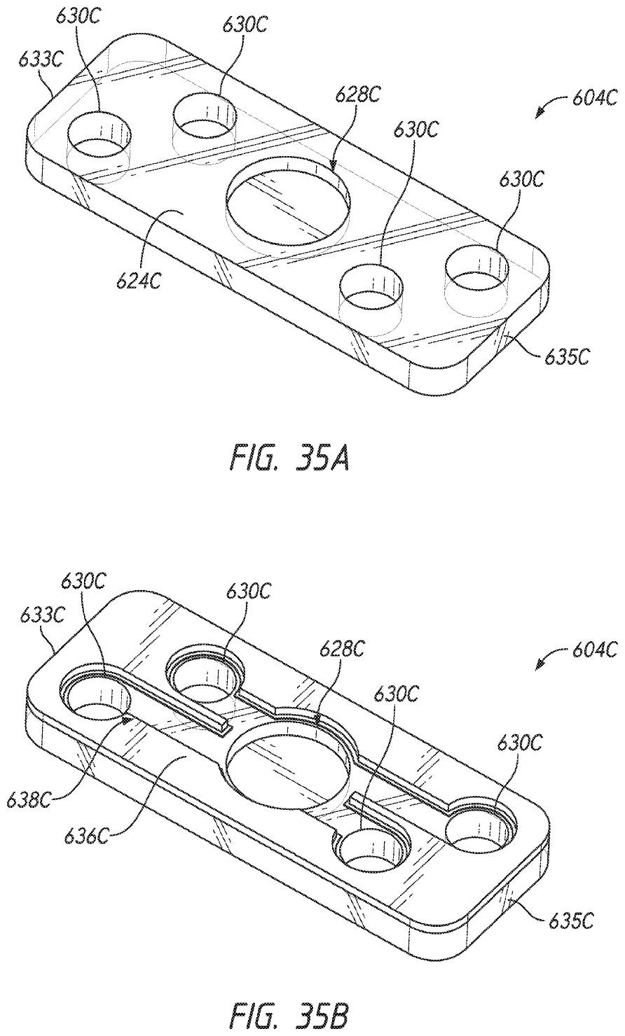

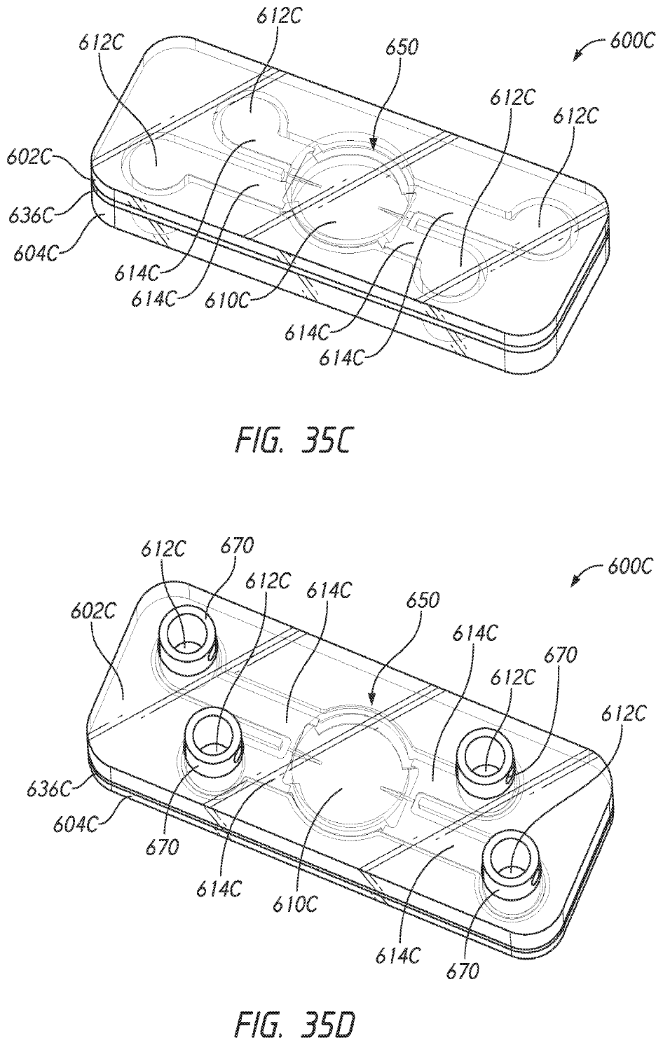

FIGS. 35A-35D illustrate various views of an implementation of the sensor 600C and its components. The sensor 600C in FIGS. 35A-35D can include an intermediate layer 636C to aid in the bonding of the piezo plate 602C to the cavity plate 604C. FIG. 35A shows an isolation view of the cavity plate 604C and FIG. 35B shows the cavity plate 604C with the intermediate layer 636C. The intermediate layer 636C can be metallized to the internal side 624C of the cavity plate 604C. The intermediate layer 636C can include an internal cutout 638C shaped based on the components of the piezo plate 602C. For example, the intermediate layer 636C and the internal cutout 638C can be a negative image of the components of the piezo plate 602C. The internal cutout 638C can be the same shape as the electrical contacts 612C, the conductive lines 614C, and the diaphragm 610C of the piezo plate 602C.

FIG. 35C shows a perspective view of the sensor 600C including the intermediate layer 636C. FIG. 35D shows the sensor 600C with contact connectors 670. As shown, the piezo plate 602C can be bonded to the cavity plate 604C in the assembled sensor 600C with the intermediate layer 636C positioned between the two plates 602C, 604C. Including the intermediate layer 636C can allow the piezo plate 602C to lie flat on the cavity plate 604C, which can improve the glass fusion/anodic bonding between the two components. While the sensor 600C shown in FIGS. 35A-35D does not include the holes 620C, 632C, 622C, 634C, it is recognized that the sensor 600C can include these holes for coupling the sensor 600C to the tubing 570. In some implementations, the contact connectors 670 can be used to couple the sensor 600C to the tubing 570 in addition to or alternatively to the holes 620C, 632C, 622C, 634C.

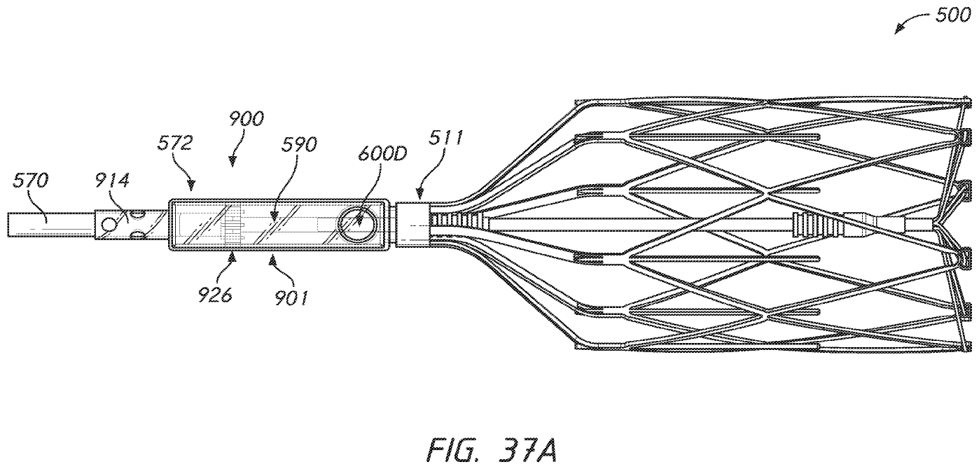

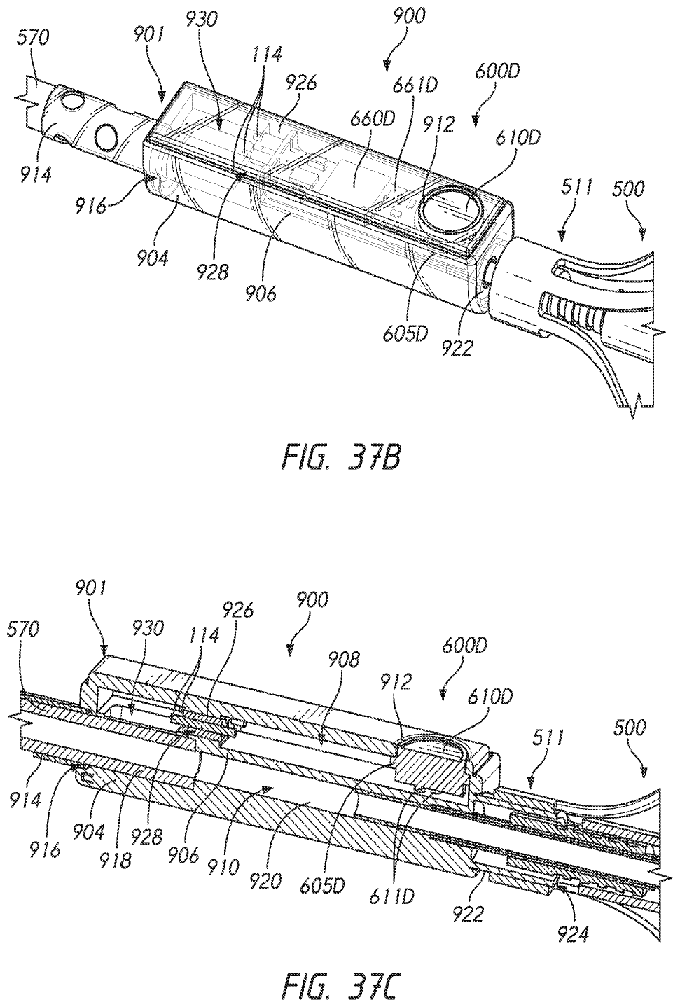

FIGS. 37A-37C illustrate various schematic views of a sensor assembly 900 that can be included in the implantable flow restriction system 5. The sensor assembly 900 can include a sensor container 901, a sensor 600D, and/or a circuit 660D. FIGS. 37A and 37B show a top view and a perspective top view respectively of sensor assembly 900 coupled to the distal end 572 of the tubing 570. In FIGS. 37A and 37B, the sensor container 901 is shown as transparent for illustrative purposes. FIG. 37C shows a perspective section view of the sensor assembly 900 taken along the central axis of the tubing 570.

The sensor assembly 900 can include any of the sensors described herein (e.g., the sensor 600, sensor 600A, capacitive sensor 600B, sensor 600C, and/or the like). In FIGS. 37A-37C, an example pressure sensor 600D is included for illustrative purposes. The sensor 600D can be configured to measure pressure proximal to the implant 500. The sensor 600D can include a sensor body 605D, a diaphragm 610D, and one or more sensor wires 611D. In some cases, the sensor 600D can be an inductive sensor, a capacitive sensor, and/or the like. The diaphragm 610D can be configured to deform when pressure is applied to it, and the sensor 600D can be configured to measure and convert the deformation into an electrical signal corresponding to the pressure level. The sensor wires 611D can be electrically connected to circuit 660D directly or via a printed circuit board 661D, the circuit 660D can be configured to process and/or amplify the signals received from the sensor 600D. The circuit 660D and printed circuit board 661D are not shown in FIGS. 37A and 37C. The circuit 660D can be an application-specific integrated circuit, an amplifier circuit, an analog-to digital convertor circuit, and/or the like. In some cases, the circuit 660D can be configured to transmit the processed and/or amplified data to a microcontroller of the implantable flow restriction system 5 (e.g., the microcontroller 102F). For example, the circuit 660D can be electrically connected to the microcontroller via one or more wires 114 of the tubing 570. In other cases, the sensor assembly 900 can include a microcontroller housed in the sensor container 901 configured to receive the processed and/or amplified signals from the circuit 660D.

The sensor container 901 can be configured to house the circuit 660D and the printed circuit board 661D and/or microcontroller when included in the sensor assembly 900. For example, the sensor container 901 can include one or more walls configured to enclose the circuit 660D. The sensor container 901 can include a cover 902 and a base 904 that define one or more internal volumes. The cover 902 can be coupled to the base 904 and can be sealed to prevent ingress into the one or more internal volumes. In some cases, one or both of the cover 902 and the base 904 can be made of titanium. The various features of the cover 902 and base 904 may be applied to cither the cover 902 or the base 904. The sensor container 901 can further include an internal wall 906 disposed between the cover 902 and the base 904. The internal wall 906 can separate a first internal volume 908 from a second internal volume 910. The first internal volume 908 can be configured to house a majority of the electrical components of the sensor assembly 900. The second internal volume 910 can be configured to receive a portion of the flow restriction system 5, such as the tubing 570 and/or the shaft 590. The sensor 600D can be at least partially disposed within the first internal volume 908. For example, the cover 902 can include an opening 912 for receiving the sensor body 605D. The opening 912 can be sized to allow the diaphragm 610D to be exposed to the environment outside of the sensor container 901. In some cases, the sensor 600D may be coupled to the cover 902. For example, the sensor body 605D may be joined or welded to a bottom side of the cover 902 with the diaphragm 610D positioned in the opening 912.

The sensor container 901 be configured to be coupled to and/or to receive the distal end of a tubular body. For example, the sensor container 901 can be configured to be coupled to and/or to receive the distal end 572 of the tubing 570. The sensor container 901 can include a first mounting portion 914 extending from a side wall of the base 904 that can be used to couple the sensor container 901 to the outside of the tubing 570. The first mounting portion 914 can extend from an opening 916 in a side wall of the base 904. The opening 916 can allow the distal end 572 of the tubing 570 to extend into the second internal volume 910. The second internal volume 910 may include a first channel 918 for receiving the tubing 570. The second internal volume 910 may also include a second channel 920. The first channel 918 may be axially aligned with the second channel 920. The second channel 920 can be configured to receive the shaft 590 of the implantable flow restriction system 5. The shaft 590 is not shown in FIG. 37C for illustrative purposes. The channels 918, 920 can allow the shaft 590 to extend through the sensor container 901 to the implant 500. The sensor container 901 can include a second mounting portion 922 extending from a side wall of the base 904 that can be used to couple the sensor container 901 to the implant 500. The second mounting portion 922 can be opposite the first mounting portion 914. The second mounting portion 922 can include an opening 924 that can allow the shaft 590 to extend to the implant 500. In some cases, a portion of the implant 500 may be positioned within the second mounting portion 922. Additionally or alternatively, the proximal end 511 of the implant 500 can be coupled to an outside surface of the second mounting portion 922.

To facilitate an electrical connection between the wires 114 of the tubing 570 and the circuit 660D, the internal wall 906 can include an opening 930. The opening 930 can be positioned above the first channel 918. The first internal volume 908 can include a second internal wall 926 to isolate the circuit 660D and other electrical components from the opening 930. Accordingly, the second internal wall 926 can partially define a third internal volume in the sensor container 901. The third internal volume can be isolated from the first internal volume 908 via the second internal wall 926. The second internal wall 926 can include one or more hermetic passthroughs 928 configured to allow the wires 114 of the tubing 570 to extend through the second internal wall 926 and be electrically connected to the circuit 660D. In this arrangement, the sensor 600D can be electrically connected to the microcontroller 102F in the implantable control unit 100F.

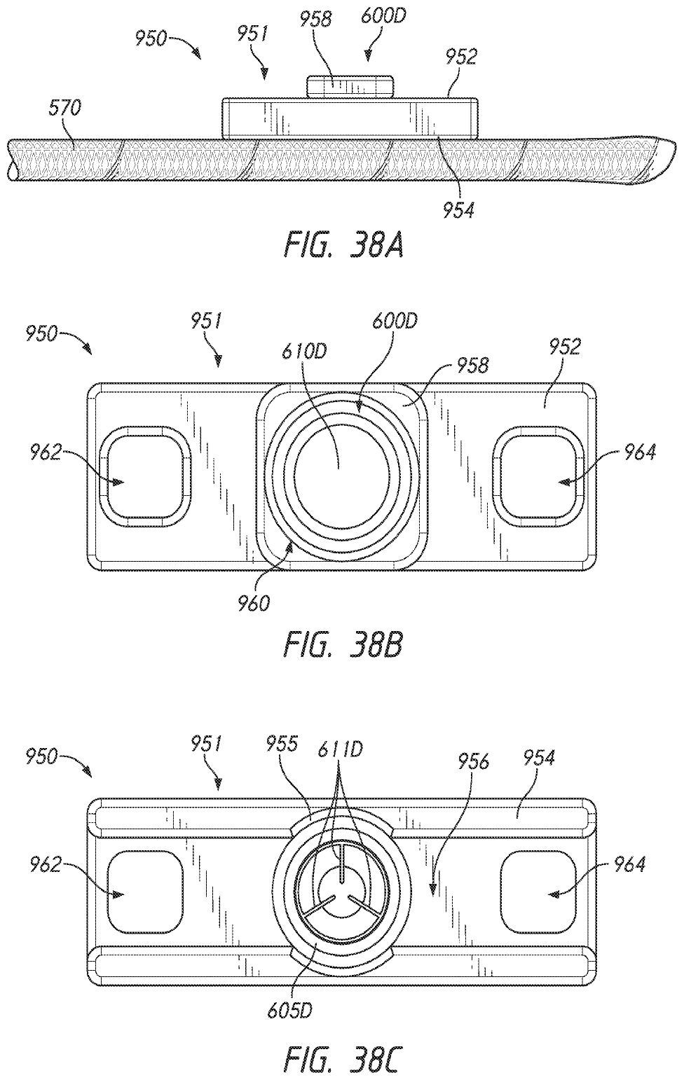

FIGS. 38A-38C illustrate various schematic views of a sensor assembly 950 that can be included in the implantable flow restriction system 5. The sensor assembly 950 can include a sensor mount 951 and a pressure sensor. In the illustrated example, the sensor assembly 950 includes the sensor 600D. However, it is recognized that the sensor assembly 950 can include a sensor of another configuration, such as any of the sensors described herein, such as the sensor 600, sensor 600A, capacitive sensor 600B, sensor 600C, and/or the like.

FIG. 38A shows a side view of the sensor assembly 950 coupled to the tubing 570, FIG. 38B shows a top view of the sensor assembly 950, and FIG. 38C shows a bottom view of the sensor assembly 950. The sensor mount 951 can be configured to house the sensor 600D and to couple the sensor 600D to the tubing 570. The sensor mount 951 can also allow the sensor 600D to be electrically connected to a microcontroller of the implantable flow restriction system 5, such as the microcontroller 102F. The sensor mount 951 can include a top side 952, a bottom side 954, and a channel 955 extending between top side 952 and the bottom side 954. The channel 955 can be configured to receive at least a portion of the sensor 600D. For example, the sensor body 605D can be positioned within the channel 955. In this arrangement, the sensor 600D can be secured to the sensor mount 951. For example, the sensor 600D can be coupled to the top side 952 and/or the channel 955. In one example, the sensor 600D can be coupled to the top side 952 by welding an outside edge of the sensor body 605D to the top side 952.

In some cases, the sensor assembly 950 can include a sensor cover 958. The sensor cover 958 can be configured to protect the sensor 600D. The sensor cover 958 can include a fluid opening 960. The sensor cover 958 can be coupled to the top side 952 of the sensor mount 951 with the fluid opening 960 positioned over the diaphragm 610D. The fluid opening 960 can be configured to allow the diaphragm 610D to be exposed to the environment where implanted. In some cases, the sensor cover 958 may not be in contact with the diaphragm 610D of the sensor 600D.

Referring to FIG. 38C, the bottom side 954 of the sensor mount 951 may include a rounded portion 956. The rounded portion 956 can extend along a length of the bottom side 954. The rounded portion 956 can be configured to allow the sensor assembly 950 to be mounted on a tubular body while reducing the overall profile of the combined sensor assembly 950 and tubular body. The tubular body can comprise the tubing 570, a catheter, and/or the like. While particular reference is made to the tubing 570, it is recognized that the sensor assembly 950 can be used with any other system and may be coupled to any suitable housing, body, implant, and/or the like. To allow the sensor assembly 950 to be coupled to another system, such as the tubing 570, the sensor mount 951 may include one or more securement opening extending from the top side 952 to the bottom side 954. For example, the sensor mount 951 can include a first securement opening 962 and a second securement opening 964. In one example, a reflow process can be used to couple the sensor mount 951 to the 570 by reflowing a reflow material through the openings 962, 964. When the sensor assembly 950 is coupled to the tubing 570, the one or more sensor wires 611D may be electrically connected to the wires 114 of the tubing 570, allowing the sensor 600D to transmit signals to a microcontroller of the flow restriction system 5.

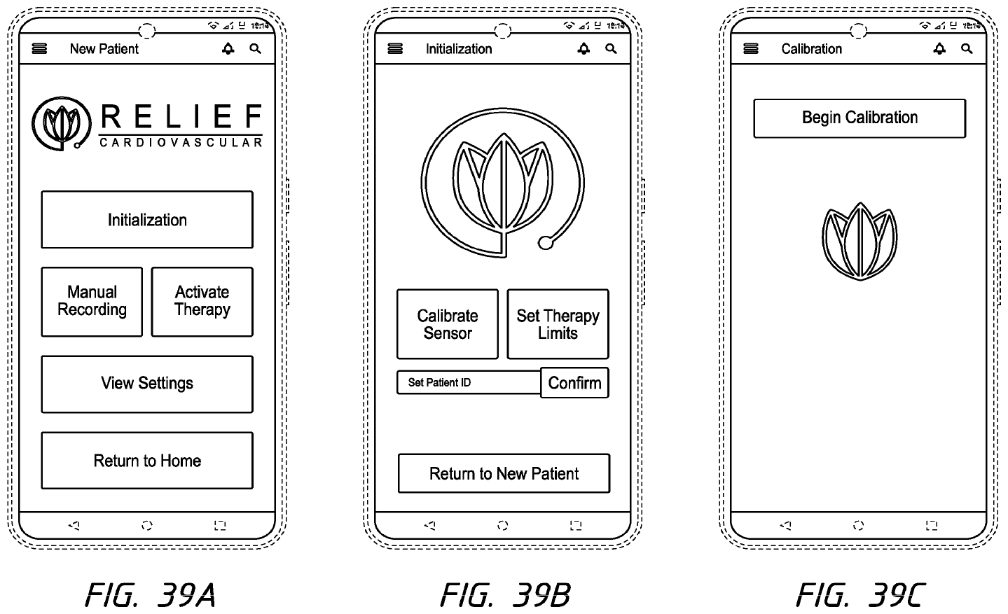

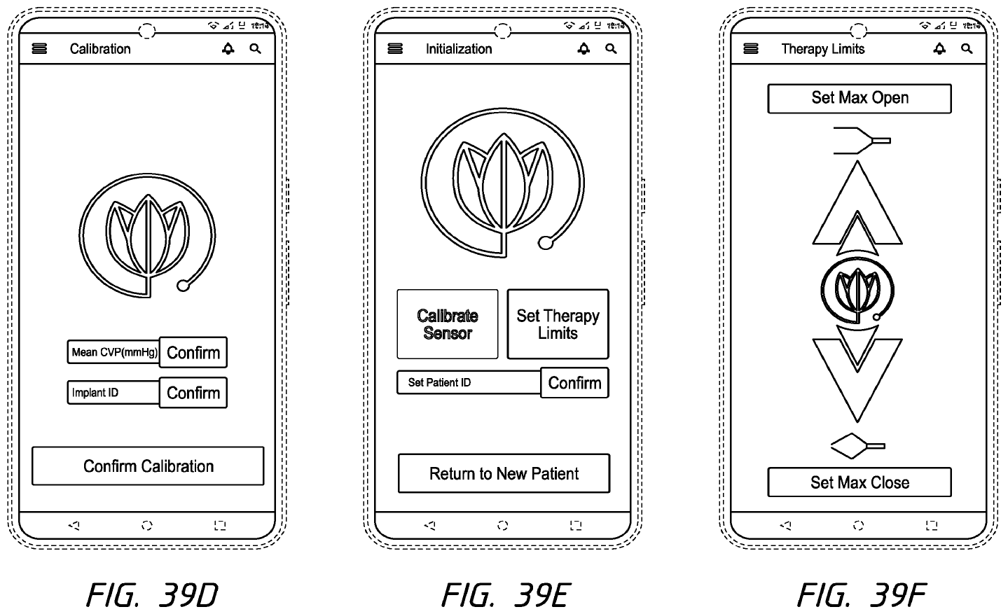

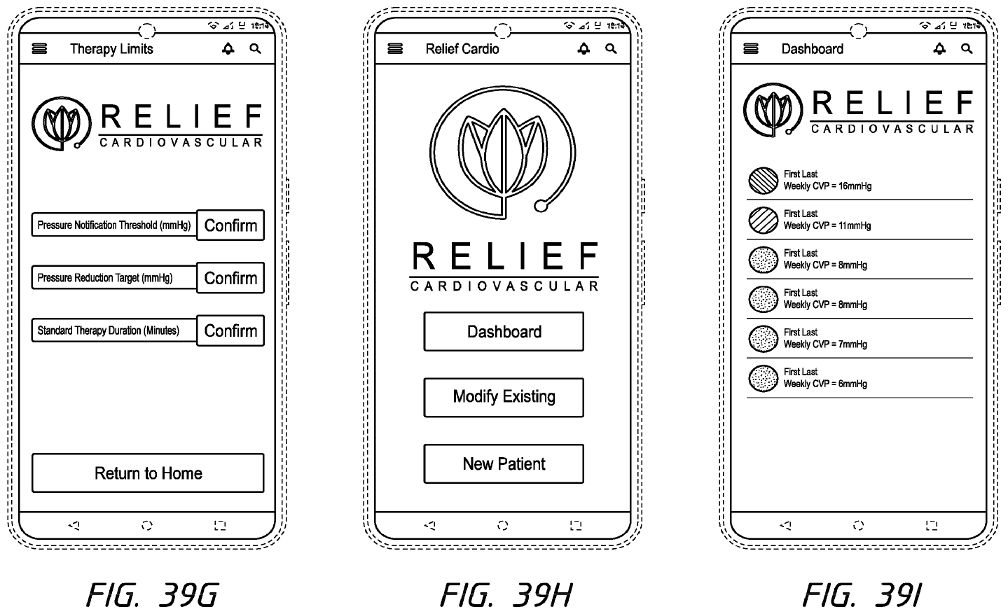

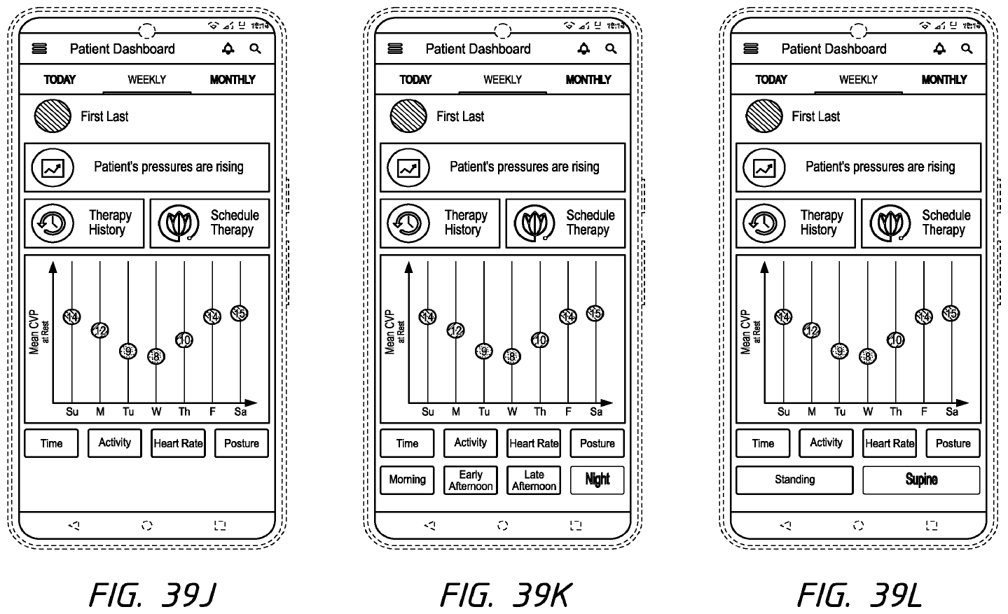

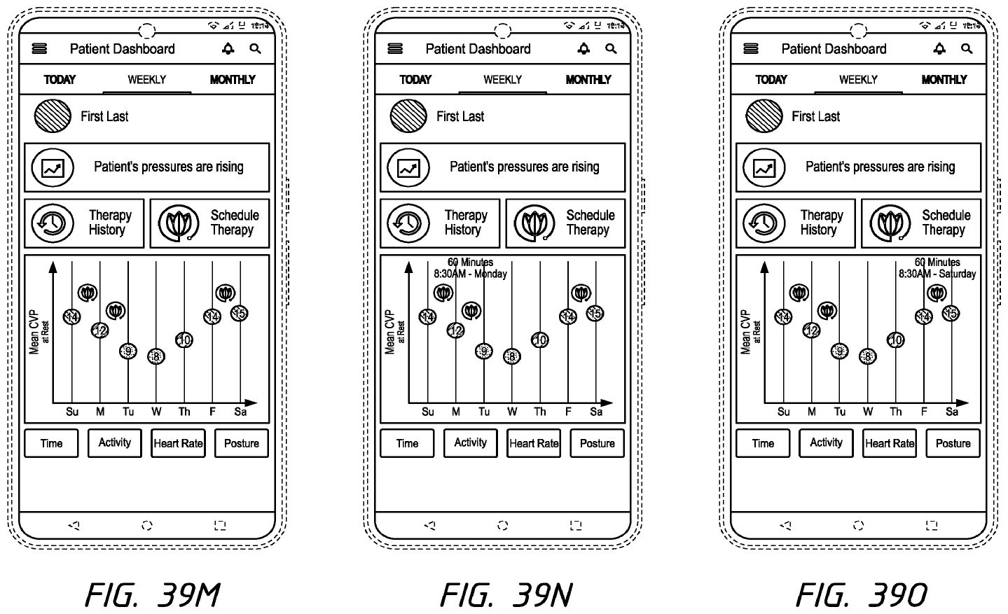

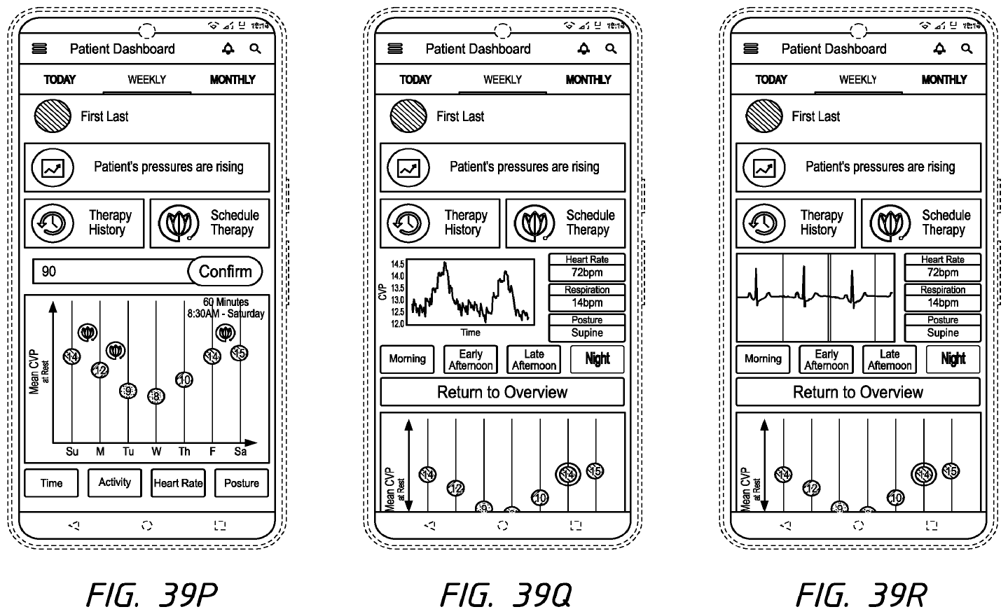

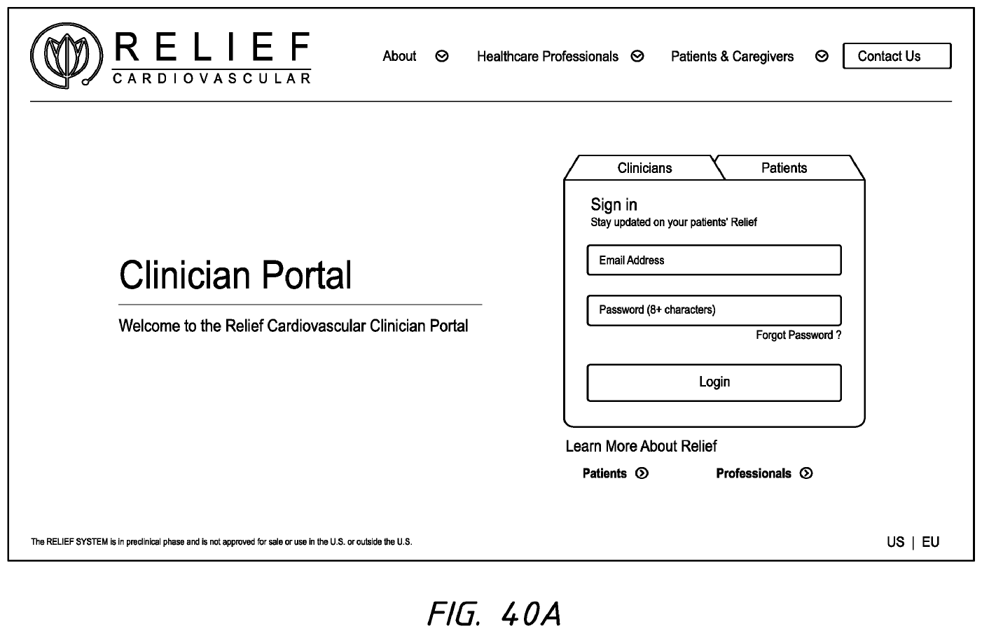

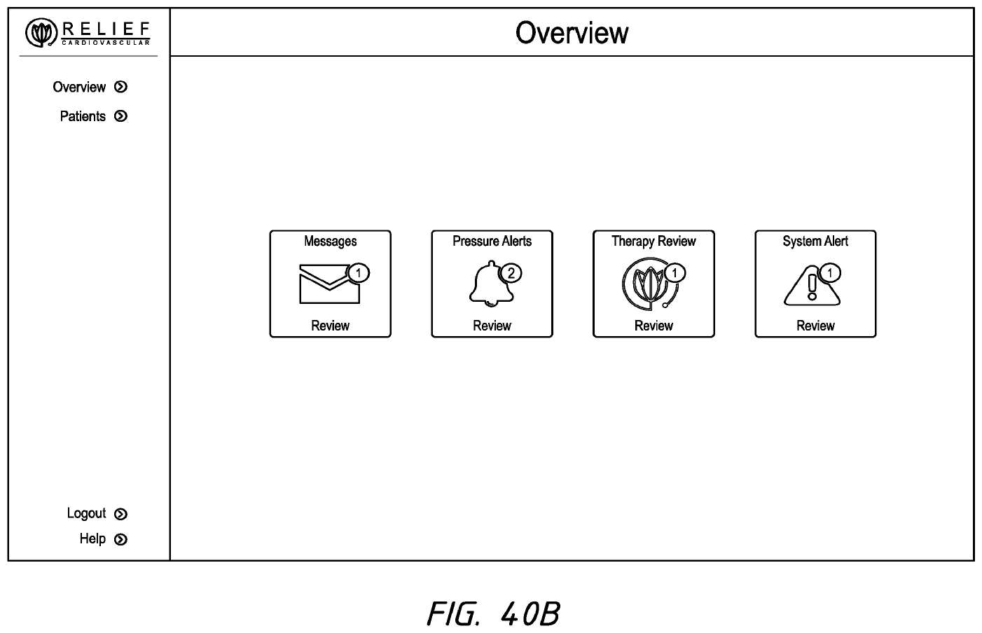

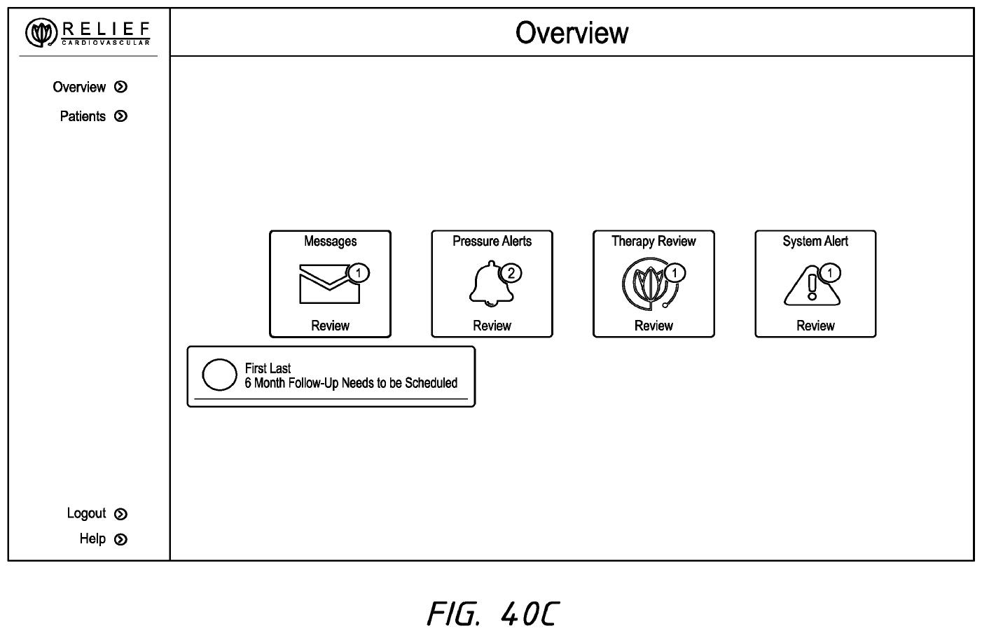

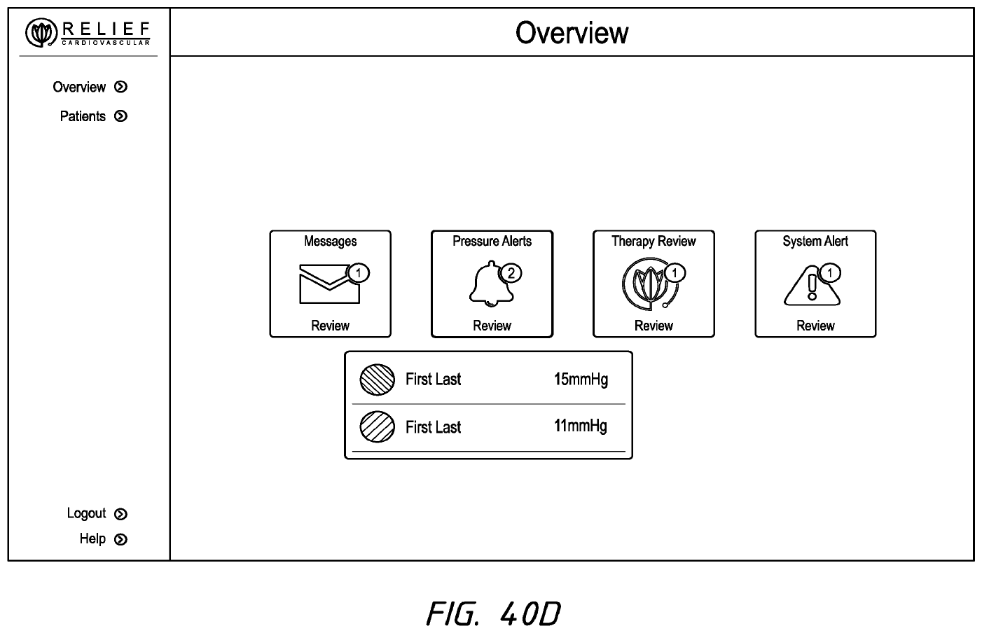

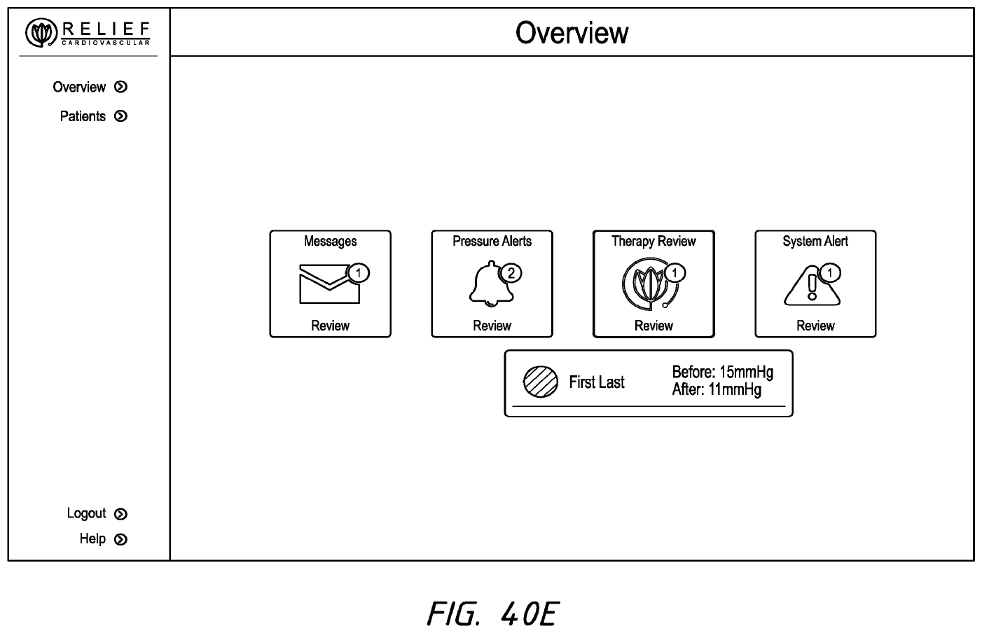

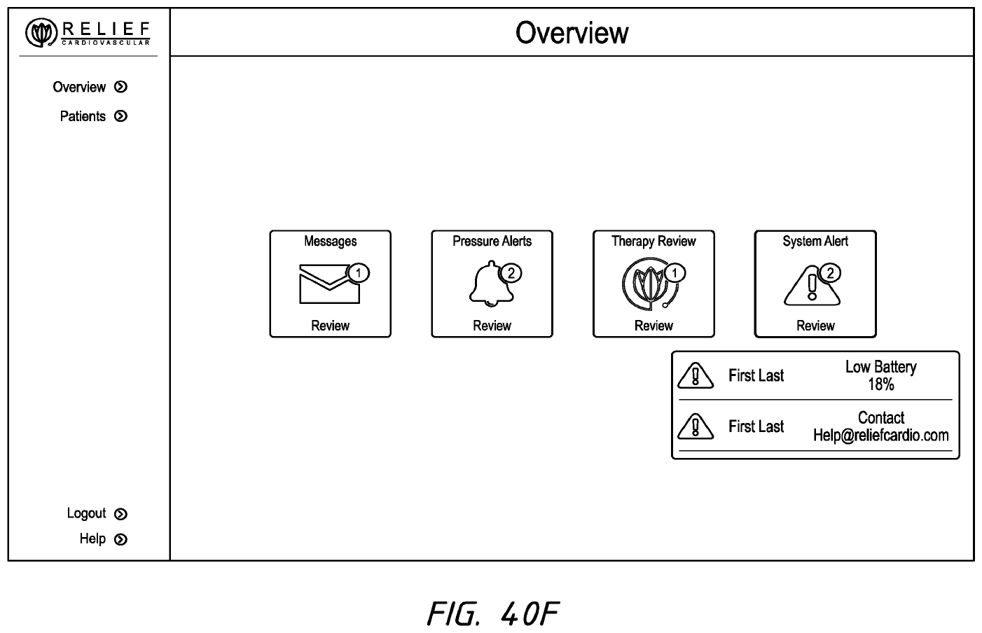

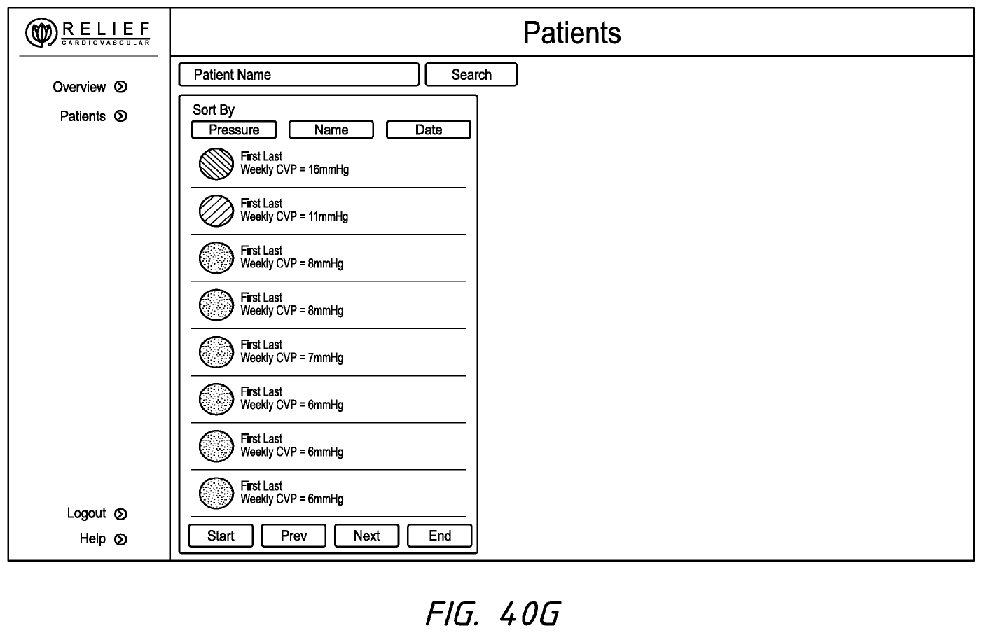

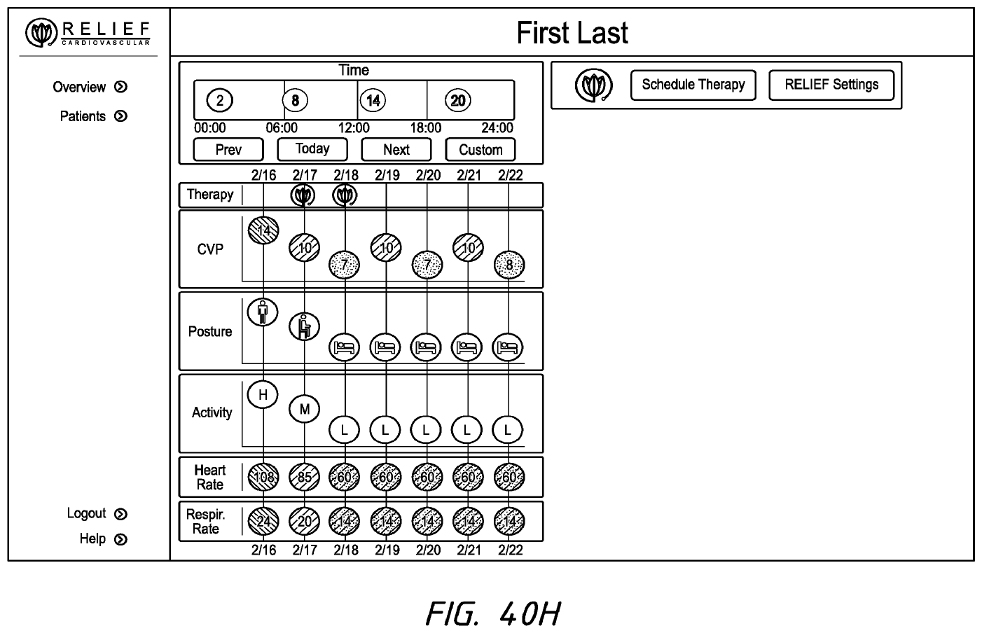

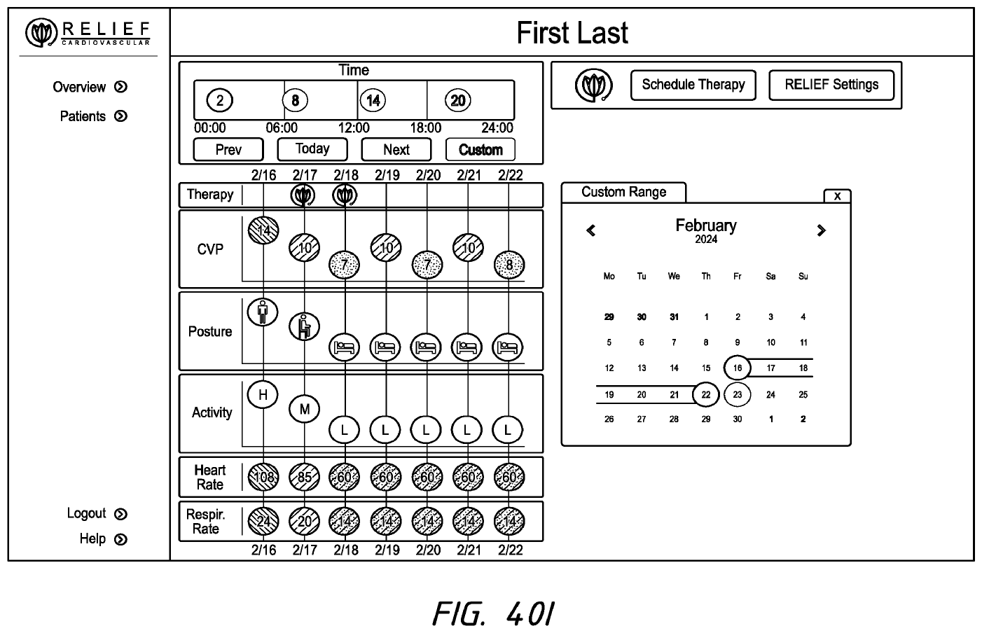

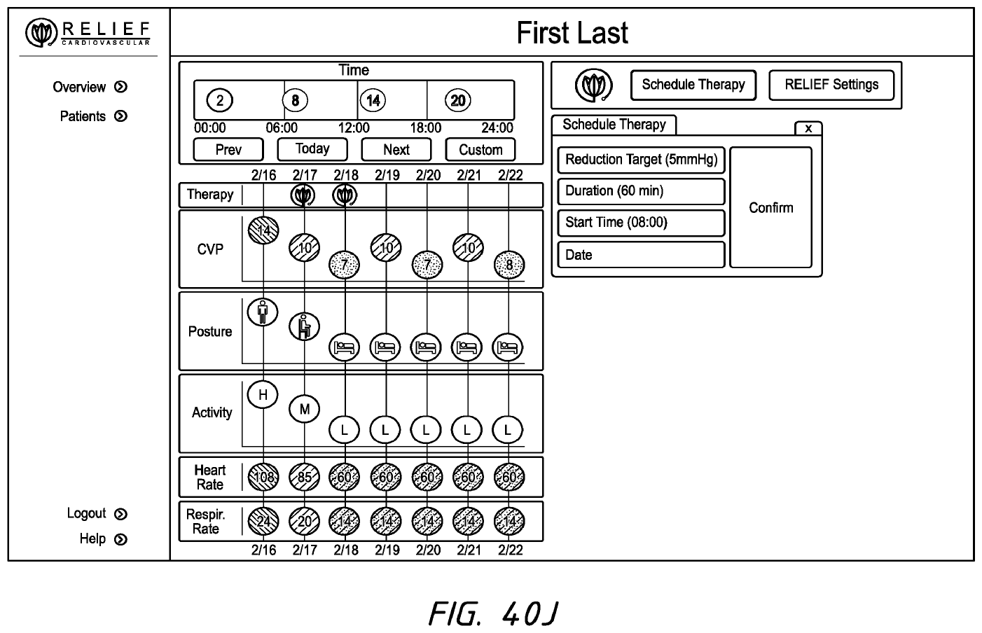

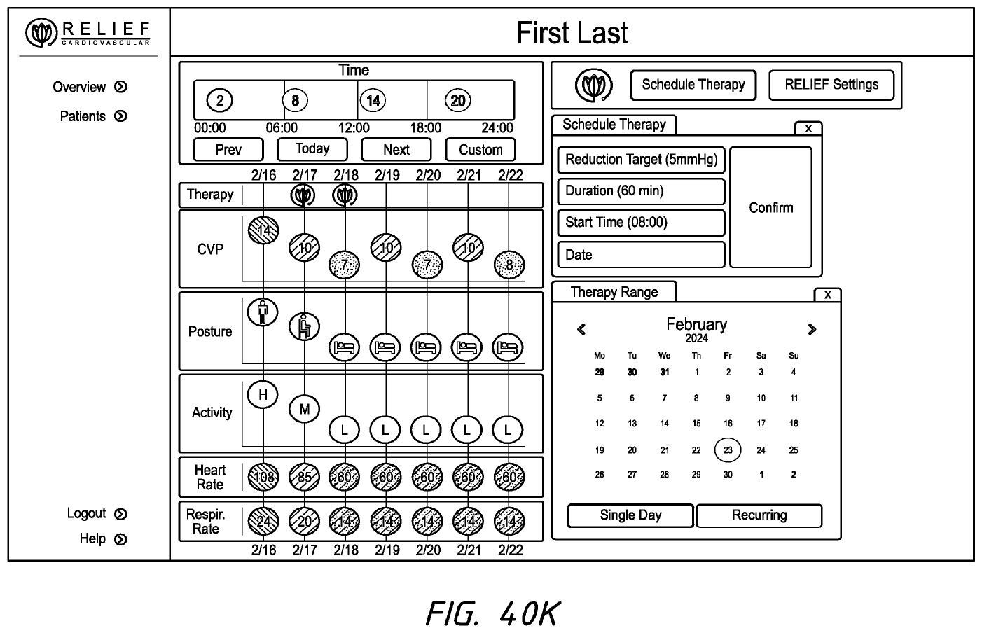

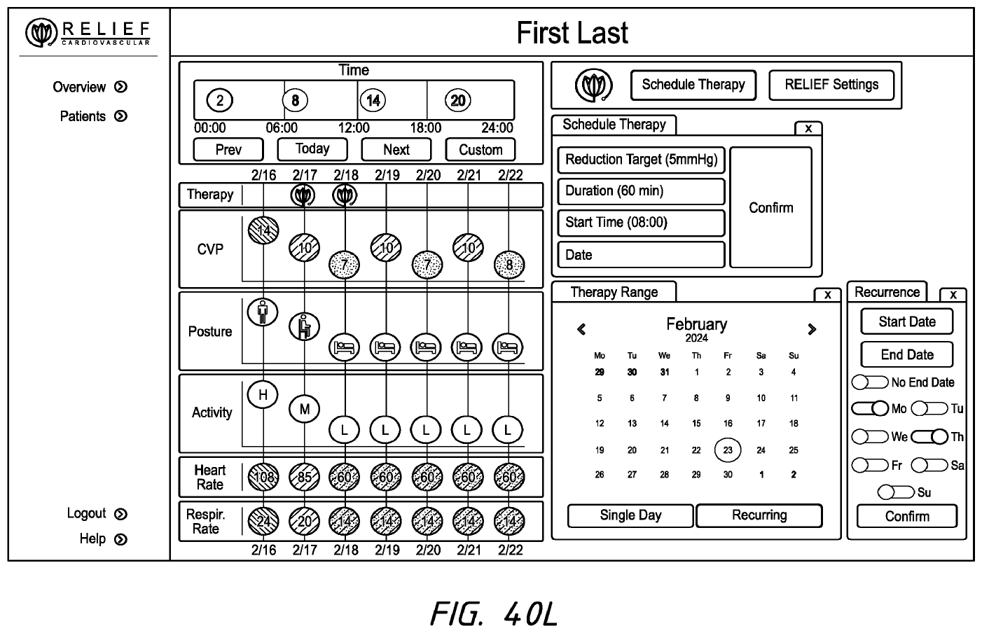

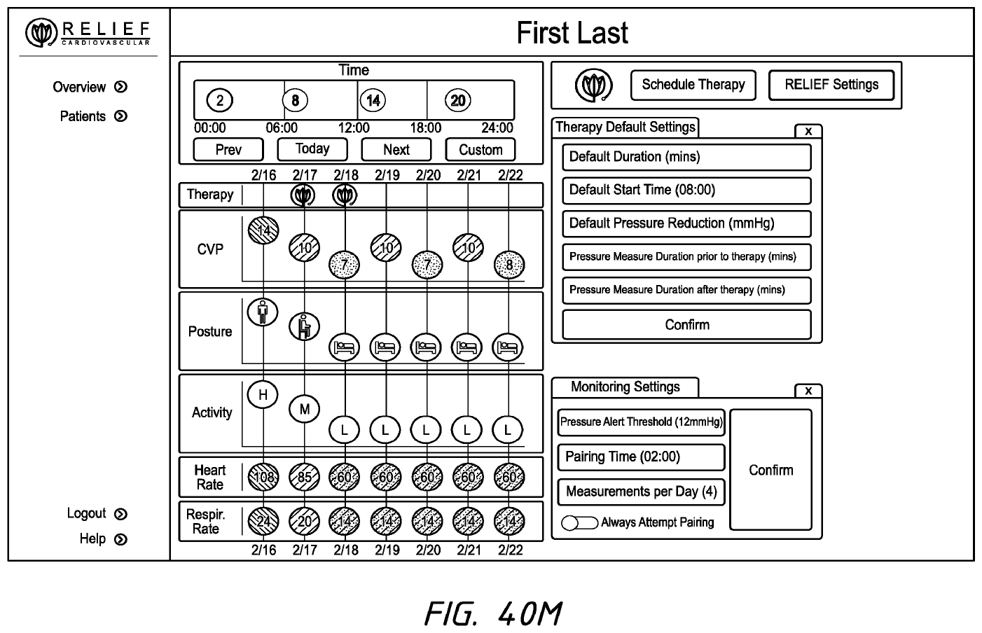

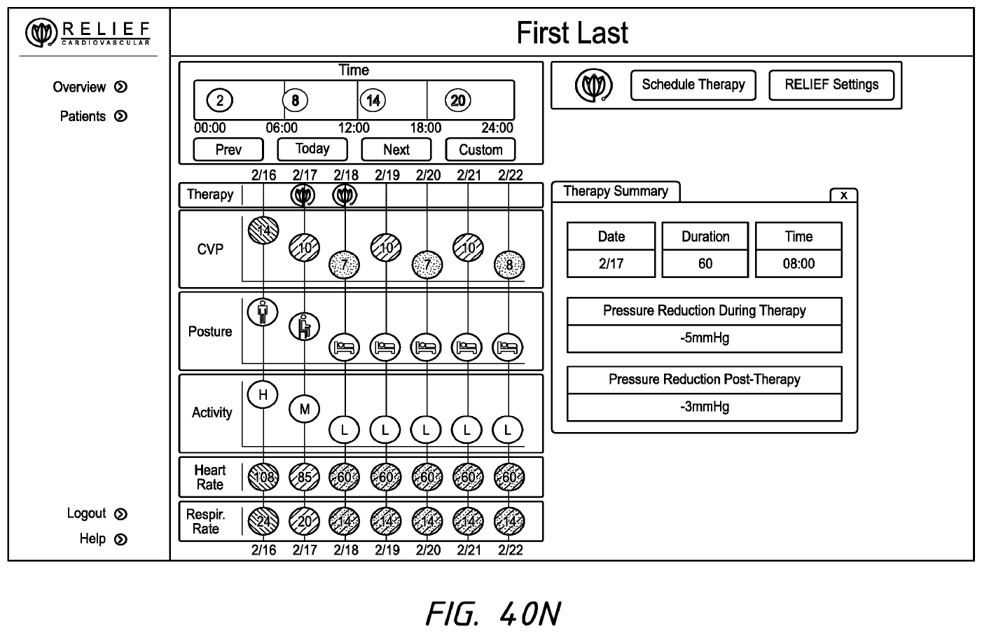

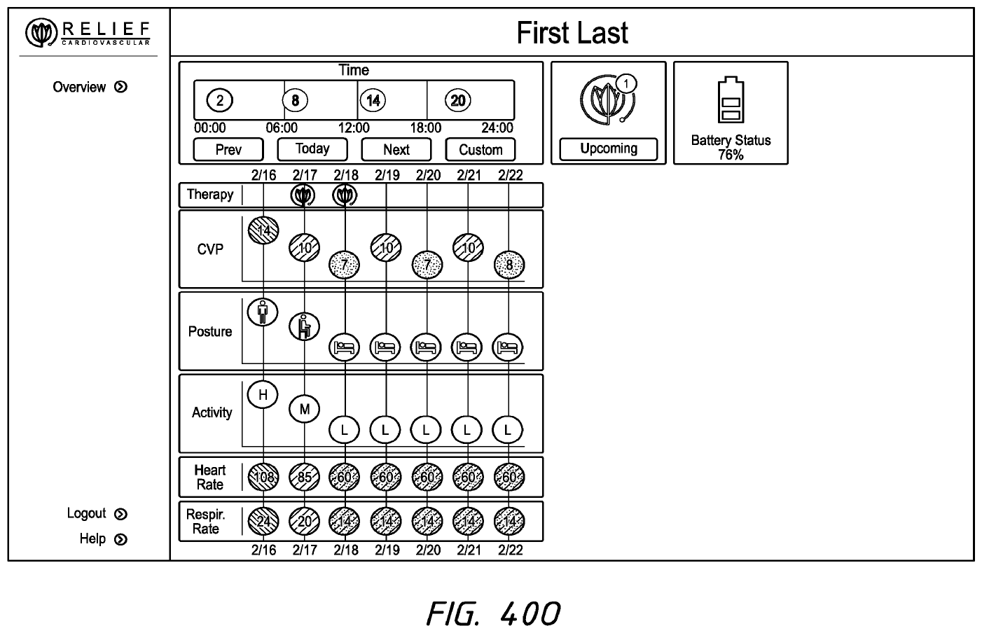

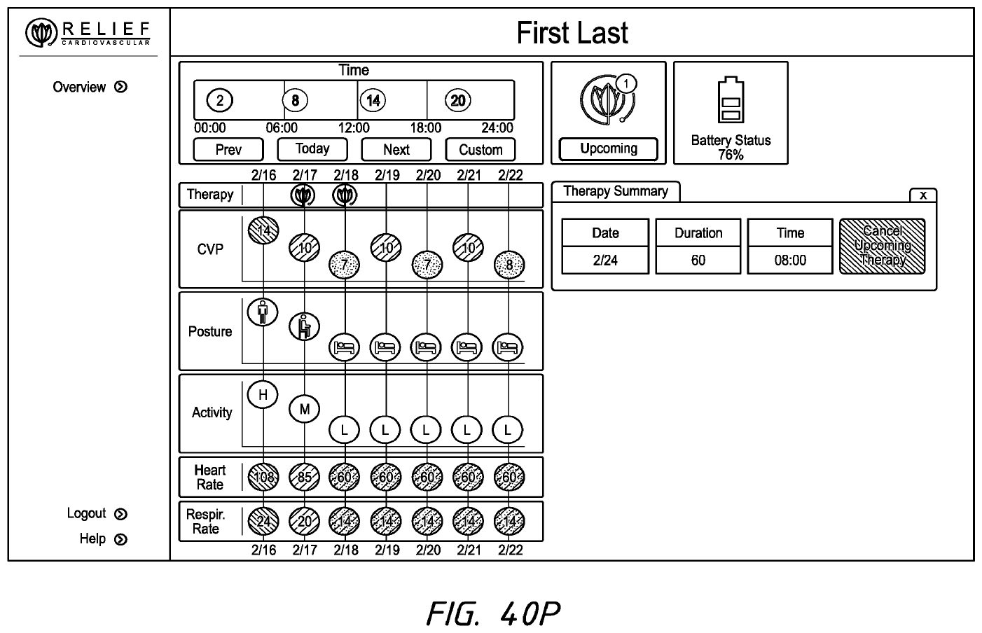

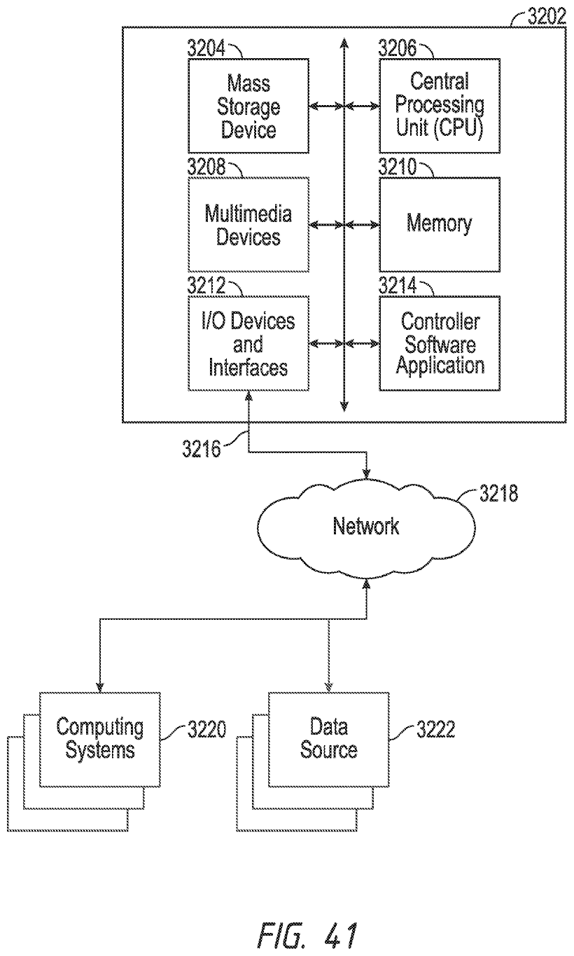

FIGS. 39A-39R illustrate graphical user interfaces presented on a user device and associated with an implantable controller system in accordance with some aspects of this disclosure. FIGS. 40A-40P illustrate graphical user interfaces presented on a user device and associated with an implantable controller system in accordance with some aspects of this disclosure.

Be it known that we have invented new, original and ornamental designs for a DISPLAY SCREEN OR PORTION THEREOF WITH GRAPHICAL USER INTERFACE, of which the following is a specification, reference being had to the accompanying drawings, forming a part thereof.

FIG. 39A is an embodiment of a front view of a display screen or portion thereof with graphical user interface showing our new design.

FIG. 39B is an embodiment of a front view of a display screen or portion thereof with graphical user interface showing our new design.

FIG. 39C is an embodiment of a front view of a display screen or portion thereof with graphical user interface showing our new design.

FIG. 39D is an embodiment of a front view of a display screen or portion thereof with graphical user interface showing our new design.

FIG. 39E is an embodiment of a front view of a display screen or portion thereof with graphical user interface showing our new design.

FIG. 39F is an embodiment of a front view of a display screen or portion thereof with graphical user interface showing our new design.

FIG. 39G is an embodiment of a front view of a display screen or portion thereof with graphical user interface showing our new design.

FIG. 39H is an embodiment of a front view of a display screen or portion thereof with graphical user interface showing our new design.

FIG. 39I is an embodiment of a front view of a display screen or portion thereof with graphical user interface showing our new design.

FIG. 39J is an embodiment of a front view of a display screen or portion thereof with graphical user interface showing our new design.

FIG. 39K is an embodiment of a front view of a display screen or portion thereof with graphical user interface showing our new design.

FIG. 39L is an embodiment of a front view of a display screen or portion thereof with graphical user interface showing our new design.

FIG. 39M is an embodiment of a front view of a display screen or portion thereof with graphical user interface showing our new design.

FIG. 39N is an embodiment of a front view of a display screen or portion thereof with graphical user interface showing our new design.

FIG. 39O is an embodiment of a front view of a display screen or portion thereof with graphical user interface showing our new design.

FIG. 39P is an embodiment of a front view of a display screen or portion thereof with graphical user interface showing our new design.

FIG. 39Q is an embodiment of a front view of a display screen or portion thereof with graphical user interface showing our new design.

FIG. 39R is an embodiment of a front view of a display screen or portion thereof with graphical user interface showing our new design.

FIG. 40A is an embodiment of a front view of a display screen or portion thereof with graphical user interface showing our new design.

FIG. 40B is an embodiment of a front view of a display screen or portion thereof with graphical user interface showing our new design.

FIG. 40C is an embodiment of a front view of a display screen or portion thereof with graphical user interface showing our new design.

FIG. 40D is an embodiment of a front view of a display screen or portion thereof with graphical user interface showing our new design.

FIG. 40E is an embodiment of a front view of a display screen or portion thereof with graphical user interface showing our new design.

FIG. 40F is an embodiment of a front view of a display screen or portion thereof with graphical user interface showing our new design.

FIG. 40G is an embodiment of a front view of a display screen or portion thereof with graphical user interface showing our new design.

FIG. 40H is an embodiment of a front view of a display screen or portion thereof with graphical user interface showing our new design.

FIG. 40I is an embodiment of a front view of a display screen or portion thereof with graphical user interface showing our new design.

FIG. 40J is an embodiment of a front view of a display screen or portion thereof with graphical user interface showing our new design.

FIG. 40K is an embodiment of a front view of a display screen or portion thereof with graphical user interface showing our new design.

FIG. 40L is an embodiment of a front view of a display screen or portion thereof with graphical user interface showing our new design.

FIG. 40M is an embodiment of a front view of a display screen or portion thereof with graphical user interface showing our new design.

FIG. 40N is an embodiment of a front view of a display screen or portion thereof with graphical user interface showing our new design.

FIG. 40O is an embodiment of a front view of a display screen or portion thereof with graphical user interface showing our new design.

FIG. 40P is an embodiment of a front view of a display screen or portion thereof with graphical user interface showing our new design.

The outermost broken lines in FIGS. 39A-39R illustrate a display screen or portion thereof upon which the graphical user interface can be displayed. The broken lines of the outermost rectangle illustrate portions of the display screen or portion thereof with graphical user interface. None of the broken lines form any part of the claimed design.

I claim: The ornamental design for a DISPLAY SCREEN OR PORTION THEREOF WITH GRAPHICAL USER INTERFACE, substantially as shown and described in FIGS. 39A-40P.

Any portions of the implants described herein (e.g., filter portion(s) 520, radial support portion(s) 540, and flow restrictor portion(s) 550) can be omitted, duplicated, or connected to one another in different orders. Furthermore, while the flow restrictor portions 550 and/or flow restrictors 560 have been described as having certain orientations with regard to aspects of the implants 500 and/or the flow of blood traveling therethrough, such flow restrictor portions 550 and/or flow restrictors 560 can be oriented in a reverse manner or in other ways than shown. Furthermore, features of the implants described herein can be implemented in any of the implants described herein. Additionally, while some implants described herein are shown and described as having components for their actuation that are substantially centrally located within their associated lumen (e.g., tubing 570, shaft 590), such implants can be adapted such that such components are located along a circumference or side of the implant to produce an implant having a lumen substantially free of such components.

Although systems, devices, and/or components thereof have been described as having particular orientations and/or locations when implanted within a patient, such orientations and/or locations are not intended to be limiting. For example, while systems, devices, and/or components thereof have been described as extending from the superior vena cava or veins branching therefrom to the inferior vena cava, such systems, devices, and/or components thereof can extend from a femoral vein to the inferior vena cava. For example, while the system 5 has been described as having an implantable controller (e.g., the implantable control unit 100) implanted in an infraclavicular subcutaneous pocket with other portions of the system extending through the superior vena cava and into the inferior vena cava, the implantable controller of system 5 can be adapted for implantation in a subcutaneous pocket in or near the groin of the patient with other portions of the system extending through a femoral vein and into the inferior vena cava. In such implementations, venous access can be through a femoral vein of the patient. Furthermore, in such implementations, the flow restrictor of an implant of such system can be configured similar to or in a reverse manner to the flow restrictor of the implant 500 shown in FIG. 2.

Although systems, devices, and/or components thereof have been described and/or configured for chronic use, any of the systems, devices, and/or components thereof can be configured for acute use and/or used for acutely. For example, in some implementations an implantable controller or actuator as described herein can be positioned outside a patient's body while an implant operably connected thereto is implanted within the patient's vasculature as described herein. In such implementations, an external device may not be required to operate the system, for example, the patient or a user can operate the system via interaction with the controller that resides outside the patient.

Some of the features or advantages encompassed by one or more of the above implementations, or other aspects of the present application, include, but are not limited to, one or more of the following:
an implant configured to controllably and selectively occlude, restrict and/or divert flow within a patient's vasculature
a source of actuation configured to actuate the implant
the implant can be configured to adjustably occlude blood flow in the vasculature in a range of 0 to 100 percent
the implant can be configured for percutaneous delivery
the implant can be configured for surgical implantation
the implant can be positioned intravenously
   the implant can be biased open and actuated to close
   the implant can be biased closed and actuated to open
   the implant can be biased partially closed and configured to open fully when positioned intravenously due to blood flow in the vasculature and actuated to close
   the implant can comprise an expandable body and a flow restrictor, the expandable body configured to engage an interior wall of a vessel of the patient and position the flow restrictor in a blood flow path of the vessel
   the implant can comprise an expandable body with a flow restrictor integrally formed therewith
   the source of actuation can comprise an actuator configured to actuate the implant mechanically
   the implant can be configured to be positioned in an IVC of a patient upstream of the renal veins
      the implant can be configured to adjustably enhance renal circulation and/or improve diuresis
   the implant can be configured to be positioned in an IVC of a patient upstream of the hepatic veins
      the implant can be configured to adjustably enhance hepatic circulation and/or improve liver function
   the implant can be configured to be positioned in an SVC of a patient upstream of the right atrium
   the implant can be configured to adjustably decrease cardiac preload, decrease central venous pressure and/or pressure of other veins disclosed herein, and/or increase cardiac output
the implant can be configured to be positioned extravenously
   the implant can be configured to be positioned adjacent an outer wall of the IVC of the patient
   the implant can adjustably compress a portion of an outer wall of the IVC to adjustably occlude blood flow within the IVC

### ADDITIONAL EMBODIMENTS

Clause 1. A system comprising: an implantable controller system for controlling a blood flow modulator, the implantable controller system comprising a housing enclosing: a microcontroller comprising: one or more computer readable storage devices configured to store a plurality of computer-executable instructions; one or more hardware computer processors in communication with the one or more computer readable storage devices; and a first communication module; an actuator comprising: a control member having a first end coupled with the blood flow modulator and a second end configured to be disposed adjacent to or in the housing; and a motor coupled to the second end of the control member to actuate the control member, wherein actuation of the control member causes the blood flow modulator to move between a low profile state and a high profile pressure modulating state; and a first power source disposed in the housing and configured to generate current for the actuator.

Clause 2. The system of clause 1, further comprising: a pressure sensor configured to generate a pressure signal to be transmitted to the microcontroller as an input for changing states of the blood flow modulator.

Clause 3. The system of clause 1 or clause 2, further comprising: an external device comprising a second housing enclosing: a second power source; and a second communication module, the second communication module configured to communicate with the first communication module.

Clause 4. The system of clause 3, wherein the computer-executable instructions, when executed, configure the one or more hardware computer processors to: receive, from the pressure sensor, a first pressure signal; and generate and transmit, to the external device via the first communication module, an electronic communication including a first pressure measurement corresponding to the first pressure signal.

Clause 5. The system of clause 4, wherein the external device is configured to display, via a graphical user interface, an indicator related to the first pressure measurement.

Clause 6. The system of clause 5, wherein the indicator comprises a first notification when the first pressure measurement is within a threshold or a second notification when the first pressure measurement is above the threshold.

Clause 7. The system of clause 6, wherein the second notification includes a treatment option or an option to cancel treatment.

Clause 8. The system of clause 7, wherein the computer-executable instructions, when executed, further configure the one or more hardware computer processors to: receive, from the external device via the second communication module, treatment instructions based on the treatment option, the treatment instructions comprising a treatment duration and a target state for the blood flow modulator; and activate the actuator such that a traveler translates a first distance in a first direction, the first distance corresponding to the target state.

Clause 9. The system of clause 8, wherein the external device provides the first power source with power via a receiver to activate the actuator or charge a third power source electrically coupled to the microcontroller.

Clause 10. The system of clause 8 or clause 9, wherein the treatment instructions further comprise a request for a second pressure measurement during or after the traveler translates to the target state.

Clause 11. The system of clause 10, wherein the computer-executable instructions, when executed, further configure the one or more hardware computer processors to: receive, from the pressure sensor, a second pressure signal; and generate and transmit, to the external device via the first communication module, an electronic communication including the second pressure measurement corresponding to the second pressure signal.

Clause 12. The system of any of clauses 8 to 11, wherein the computer-executable instructions, when executed, further configure the one or more hardware computer processors to: determine, that the treatment duration has expired; and activate the actuator such that the traveler translates the first distance in a second direction, the second direction opposite the first direction.

Clause 13. The system of clause 12, wherein the blood flow modulator returns to an original state once the traveler translates the first distance in the second direction.

Clause 14. The system of any of clauses 1 to 13, wherein the implantable controller system further comprises a third power source electrically coupled to the microcontroller.

Clause 15. The system of any of clauses 3 to 14, wherein the external device comprises a mobile device.

Clause 16. The system of clause 15, wherein the mobile device is configured to communicate with a remote server configured to receive and store data from the pressure sensor and treatment details used by and transmitted from the implantable controller system to the mobile device.

Clause 17. The system of any of clauses 8 to 16, wherein the traveler further comprises a magnet and the implantable controller system further comprises a plurality of hall effect sensors positioned along a length of the control member, the plurality of hall effect sensors configured to detect a magnetic field produced by the magnet and transmit a corresponding output voltage to the microcontroller.

Clause 18. The system of clause 17, wherein the computer-executable instructions, when executed, configure the one or more hardware computer processors to: receive, from the plurality of hall effect sensors, output voltages; and determine, a relative position of the traveler within the housing.

Clause 19. The system of clause 18, wherein the relative position of the traveler corresponds to a state of the blood flow modulator.

Clause 20. The system of any of clauses 17 to 19, wherein the plurality of hall effect sensors comprises three hall effect sensors equally spaced along the length of the control member.

Clause 21. The system of any of clauses 1 to 20, wherein at least a portion of the actuator is enclosed within an ingress protection system configured to prevent fluid received within the ingress protection system from entering a main portion of the housing.

Clause 22. The system of clause 21, wherein the housing is an external housing and the ingress protection system comprises an internal housing, the internal housing positioned within the external housing and hermetically sealed relative to the housing.

Clause 23. The system of clause 22, wherein the internal housing comprises a different material than the external housing.

Clause 24. The system of clause 22 or clause 23, wherein the external housing further encloses an insulating material positioned between an interior of the external housing and the internal housing, the insulating material configured to cover at least one of the microcontroller and the motor.

Clause 25. The system of Clause 24, wherein the insulating material comprises a resin.

Clause 26. The system of clause 21, wherein the ingress protection system comprises a compartment with an internally threaded hole, the compartment configured to receive the second end of the control member.

Clause 27. The system of clause 26, wherein the second end of the control member is threaded such that the second end engages the internally threaded hole of the compartment.

Clause 28. The system of clause 21, wherein the ingress protection system comprises a sheath, a first end of the sheath coupled to a traveler coupled to the second end of the control member, the second end of the control member positioned within the sheath, wherein the sheath is configured to move between an elongated configuration and a compressed configuration with movement of the traveler.

Clause 29. The system of any of clauses 1 to 28, wherein the implantable controller system further comprises a tube having a first end coupled with the blood flow modulator and a second end configured to be disposed adjacent to or in the housing, the control member extending through the tube such that the control member is not exposed to a patient in use at least along a portion of a length thereof, the control member configured to move relatively to the tube.

Clause 30. The system of clause 29, wherein the housing further comprises an extension portion defining a passage into the housing, wherein the second end of the control member extends through the passage and the second end of the tube extends at least partially through the passage, wherein the tube can be selectively fixed to the housing by applying a compressive force on the second end of the tube.

Clause 31. The system of clause 30, wherein the housing further comprises a nut and wherein an outer surface of the extension portion is threaded, wherein the nut is in a secured configuration when threaded on the extension portion, the nut applying the compressive force on the second end of the tube in the secured configuration.

Clause 32. The system of clause 31, wherein a plurality of controller leads are positioned within the extension portion, each controller lead positioned within a spring, wherein a plurality of sensor leads extend from the pressure sensor and through the tube to a plurality of electrical contacts disposed at the second end of the tube, wherein when the nut is in the secured configuration, the plurality of electrical contacts are engaged with the plurality of controller leads, each controller lead connect to a bus electrically connected to the microcontroller such that the pressure signal can be transmitted to the microcontroller.

Clause 33. The system of clause 32, wherein each controller lead of the plurality of controller leads are positioned within a lead housing, each lead housing comprising a tubular member with a hole such that the tube can extend through the holes of the lead housings.

Clause 34. The system of clause 32 or clause 33, wherein the plurality of controller leads and springs comprise an open ended rectangular shape.

Clause 35. The system of clause 1, further comprising: the blood flow modulator, the blood flow modulator comprising: a flow restrictor coupled with a distal end of a tubing and coupled with a distal end of a shaft extending through a lumen of the tubing, a proximal length of the lumen disposed through a proximal portion of the tubing; a first connector disposed at the proximal portion of the tubing; and a second connector disposed at a proximal end of the shaft, the second connector at least partially disposed within the first connector; wherein the actuator is configured to cause the second connector to translate within the first connector, the actuator having a first configuration corresponding to the low profile state of the blood flow modulator and a second configuration corresponding to the high profile pressure modulating state of the blood flow modulator.

Clause 36. The system of clause 35, wherein the second connector comprises a tube comprising an internally threaded surface and an external surface comprising one or more projections, wherein the first connector comprises a tube comprising an inner surface with one or more recesses, the one or more recesses configured to receive the one or more projections.

Clause 37. The system of clause 36, wherein the one or more projections comprise one or more splines and the one or more recesses comprise one or more spline guides.

Clause 38. The system of clause 36 or clause 37, wherein the control member comprises an externally threaded shaft engaged with the internally threaded surface of the second connector, the externally threaded shaft configured to be rotated by the motor, rotation of the externally threaded shaft causing movement of the second connector relative to the first connector.

Clause 39. The system of clause 38, wherein engagement between the one or more projections and the one or more recesses allow the second connector to translate within the first connector when driven by the control member.

Clause 40. The system of any of clauses 35 to 39, wherein the proximal end of the shaft is located within the proximal length of the lumen of the tubing in the first configuration and in the second configuration.

Clause 41. The system of any of clauses 38 to 40, wherein the motor comprises an output shaft rotatable about a first axis, the output shaft configured to drive rotation of the externally threaded shaft about a second axis, the first axis parallel to the second axis.

Clause 42. The system of any of clauses 35 to 41, wherein the tubing comprises a plurality of first leads positioned radially outward of the lumen and radially inward from an outside surface of the tubing.

Clause 43. The system of clause 42, further comprising an array of electrical contacts coupled to an outside surface of the first connector, the array of electrical contacts coupled to a plurality of second leads.

Clause 44. The system of clause 43, wherein the outside surface of the first connector comprises a plurality of grooves, the plurality of first leads and/or the plurality of second leads configured to be aligned with and/or at least partially recessed in the plurality of grooves.

Clause 45. The system of clause 43 or clause 44, wherein each first lead is electrically connected to a second lead via a crimp sleeve.

Clause 46. The system of clause 45, wherein the crimp sleeves are configured to provide strain relief for the plurality of first leads and the plurality of second leads.

Clause 47. The system of clause 45 or clause 46, wherein the first connector is coupled to the proximal portion of the tubing to secure the crimp sleeves to the first connector.

Clause 48. The system of any of clauses 45 to 47, wherein the first connector is coupled to the proximal portion of the tubing using one or more of solder, an adhesive, and a weld to secure the crimp sleeves to the first connector.

Clause 49. The system of clauses 45 to 47, wherein the first connector is coupled to the proximal portion of the tubing using a reflow process to secure the crimp sleeves to the first connector.

Clause 50. The system of any of clauses 42 to 49, wherein the plurality of first leads are electrically coupled to a pressure sensor of the blood flow modulator.

Clause 51. The system of clause 35, wherein the blood flow modulator further comprises a first sleeve, a second sleeve, and a third sleeve, the first sleeve coupled to the proximal end of the shaft, the third sleeve coupled to the shaft at a location distal to the first sleeve, the second sleeve positioned between the first sleeve and the third sleeve.

Clause 52. The system of clause 51, wherein the second sleeve is not rotationally fixed to the shaft.

Clause 53. The system of clause 51 or clause 52, wherein the second sleeve is coupled to the second connector.

Clause 54. The system of clause 53, wherein the shaft is configured to rotate relative to the second connector.

Clause 55. The system of any of clauses 51 to 54, wherein the first sleeve, the second sleeve, and the third sleeve have inner diameters that are substantially a same size, wherein an outer diameter of the second sleeve is greater than an outer diameter of the first sleeve and an outer diameter of the third sleeve such that the second sleeve cannot translate past the first sleeve or the third sleeve on the shaft.

Clause 56. The system of any of clauses 51 to 55, wherein at least one of the first sleeve and the third sleeve is crimped to the shaft.

Clause 57. The system of any of clauses 51 to 56, wherein at least one of the first sleeve and the third sleeve is welded to the shaft.

Clause 58. The system of any of clauses 35 to 57, wherein the housing comprises an outer surface and one or more isolated compartments recessed into the outer surface, the one or more isolated compartments configured to receive wireless components.

Clause 59. The system of any of clauses 35 to 58, wherein the implantable controller system further comprises an induction receiver configured to activate the actuator or charge the first power source housed in an internal volume of the housing.

Clause 60. The system of clause 59, wherein the induction receiver is disposed in one of the one or more isolated compartments, the induction receiver electrically connected to the actuator or the first power source via leads extending through hermetic passthroughs in the housing and into the internal volume.

Clause 61. The system of any of clauses 35 to 60, wherein the microcontroller is configured to control movement of the actuator between the first configuration and the second configuration.

Clause 62. The system of clause 61, wherein the microcontroller is disposed in a first compartment of the internal volume and the actuator is disposed in a second compartment of the internal volume, the first compartment isolated from the second compartment.

Clause 63. The system of clause 61 or clause 62, wherein the implantable controller system further comprises an antenna electrically connected to the microcontroller, the antenna configured to transmit and receive electronic communications with an external device.

Clause 64. The system of clause 63, wherein the antenna is disposed in one of the one or more isolated compartments, the antenna electrically connected to the microcontroller via leads extending through one or more hermetic passthroughs in the housing and into the internal volume.

Clause 65. The system of any of clauses 61 to 64 wherein the implantable controller system further comprises a magnet and one or more hall effect sensors electrically connected to the microcontroller, wherein the magnet is configured to travel with the second connector as the actuator moves between the first configuration and the second configuration, the microcontroller configured to determine a relative position of the magnet within the housing based on electrical signals received from the one or more hall effect sensors.

Clause 66. The system of clause 65, wherein the relative position of the magnet corresponds to a state of the blood flow modulator.

Clause 67. The system of clause 65 or clause 66, wherein the magnet includes a central hole, the externally threaded shaft extending through the central hole.

Clause 68. The system of clause 67, wherein the implantable controller system is configured to bias the magnet against the second connector.

Clause 69. The system of any of clauses 61 to 68, wherein the implantable controller system further comprises one or more electrocardiogram sensors disposed on the outer surface of the housing and electrically connected to the microcontroller, the one or more electrocardiogram sensors configured to detect electrical activity of a patient's heart.

Clause 70. A system comprising: an implant comprising a flow restrictor coupled with a distal end of a tubing and coupled with a distal end of a shaft extending through a lumen of the tubing, a proximal length of the lumen disposed through a proximal portion of the tubing; a first connector disposed at the proximal portion of the tubing; a second connector disposed at a proximal end of the shaft, the second connector at least partially disposed within the first connector; and an implantable controller system for controlling the flow restrictor between a low profile state and a high profile flow restricting state, the implantable controller system comprising: an implantable housing; and an actuator positioned within the implantable housing, the actuator configured to cause the second connector to translate within the first connector, the actuator having a first configuration corresponding to the flow restrictor being in the low profile state and a second configuration corresponding to the flow restrictor being in the high profile flow restricting state.

Clause 71. The system of clause 70, wherein the second connector comprises a tube comprising an internally threaded surface and an external surface comprising one or more projections, wherein the first connector comprises a tube comprising an inner surface with one or more recesses, the one or more recesses configured to receive the one or more projections.

Clause 72. The system of clause 71, wherein the one or more projections comprise one or more splines and the one or more recesses comprise one or more spline guides.

Clause 73. The system of clause 71 or clause 72, wherein the actuator comprises an externally threaded shaft engaged with the internally threaded surface of the second connector, the externally threaded shaft configured to be rotated by a motor disposed in the implantable housing, rotation of the externally threaded shaft causing movement of the second connector relative to the first connector.

Clause 74. The system of clause 73, wherein engagement between the one or more projections and the one or more recesses allow the second connector to translate within the first connector when driven by the actuator.

Clause 75. The system of any of clauses 70 to 74, wherein the proximal end of the shaft is located within the proximal length of the lumen of the tubing in the first configuration and in the second configuration.

Clause 76. The system of any of clauses 73 to 75, wherein the motor comprises an output shaft rotatable about a first axis, the output shaft configured to drive rotation of the externally threaded shaft about a second axis, the first axis parallel to the second axis.

Clause 77. The system of any of clauses 70 to 76, wherein the tubing comprises a plurality of first leads positioned radially outward of the lumen and radially inward from an outside surface of the tubing.

Clause 78. The system of clause 77, further comprising an array of electrical contacts coupled to an outside surface of the first connector, the array of electrical contacts coupled to a plurality of second leads.

Clause 79. The system of clause 78, wherein the outside surface of the first connector comprises a plurality of grooves, the plurality of first leads and/or the plurality of second leads configured to be aligned with and/or at least partially recessed in the plurality of grooves.

Clause 80. The system of clause 78 or clause 79, wherein each first lead is electrically connected to a second lead via a crimp sleeve.

Clause 81. The system of clause 80, wherein the crimp sleeves are configured to provide strain relief for the plurality of first leads and the plurality of second leads.

Clause 82. The system of clause 80 or clause 81, wherein the first connector is coupled to the proximal portion of the tubing to secure the crimp sleeves to the first connector.

Clause 83. The system of any of clauses 80 to 82, wherein the first connector is coupled to the proximal portion of the tubing using one or more of solder, an adhesive, and a weld to secure the crimp sleeves to the first connector.

Clause 84. The system of clauses 80 to 82, wherein the first connector is coupled to the proximal portion of the tubing using a reflow process to secure the crimp sleeves to the first connector.

Clause 85. The system of any of clauses 77 to 84, wherein the plurality of first leads are electrically coupled to a pressure sensor of the flow restrictor.

Clause 86. The system of clause 70, wherein the implant further comprises a first sleeve, a second sleeve, and a third sleeve, the first sleeve coupled to the proximal end of the shaft, the third sleeve coupled to the shaft at a location distal to the first sleeve, the second sleeve positioned between the first sleeve and the third sleeve.

Clause 87. The system of clause 86, wherein the second sleeve is not rotationally fixed to the shaft.

Clause 88. The system of clause 86 or clause 87, wherein the second sleeve is coupled to the second connector.

Clause 89. The system of clause 88, wherein the shaft is configured to rotate relative to the second connector.

Clause 90. The system of any of clauses 86 to 89, wherein the first sleeve, the second sleeve, and the third sleeve have inner diameters that are substantially a same size, wherein an outer diameter of the second sleeve is greater than an outer diameter of the first sleeve and an outer diameter of the third sleeve such that the second sleeve cannot translate past the first sleeve or the third sleeve on the shaft.

Clause 91. The system of any of clauses 86 to 90, wherein at least one of the first sleeve and the third sleeve is crimped to the shaft.

Clause 92. The system of any of clauses 86 to 90, wherein at least one of the first sleeve and the third sleeve is welded to the shaft.

Clause 93. The system of any of clauses 70 to 92, wherein the implantable housing comprises an outer surface and one or more isolated compartments recessed into the outer surface, the one or more isolated compartments configured to receive wireless components.

Clause 94. The system of any of clauses 70 to 93, wherein the implantable controller system further comprises an induction receiver configured to activate the actuator or charge a power source housed in an internal volume of the implantable housing.

Clause 95. The system of clause 94, wherein the induction receiver is disposed in one of the one or more isolated compartments, the induction receiver electrically connected to the actuator or the power source via leads extending through hermetic passthroughs in the implantable housing and into the internal volume.

Clause 96. The system of any of clauses 70 to 95, wherein the implantable controller system further comprises a microcontroller configured to control movement of the actuator between the first configuration and the second configuration.

Clause 97. The system of clause 96, wherein the microcontroller is disposed in a first compartment of the internal volume and the actuator is disposed in a second compartment of the internal volume, the first compartment isolated from the second compartment.

Clause 98. The system of clause 96 or clause 97, wherein the implantable controller system further comprises an antenna electrically connected to the microcontroller, the antenna configured to transmit and receive electronic communications with an external device.

Clause 99. The system of clause 98, wherein the antenna is disposed in one of the one or more isolated compartments, the antenna electrically connected to the microcontroller via leads extending through one or more hermetic passthroughs in the implantable housing and into the internal volume.

Clause 100. The system of any of clauses 96 to 99, wherein the implantable controller system further comprises a magnet and one or more hall effect sensors electrically connected to the microcontroller, wherein the magnet is configured to travel with the second connector as the actuator moves between the first configuration and the second configuration, the microcontroller configured to determine a relative position of the magnet within the implantable housing based on electrical signals received from the one or more hall effect sensors.

Clause 101. The system of clause 100, wherein the relative position of the magnet corresponds to a state of the flow restrictor.

Clause 102. The system of clause 100 or clause 101, wherein the magnet includes a central hole, the externally threaded shaft extending through the central hole.

Clause 103. The system of clause 102, wherein the implantable controller system is configured to bias the magnet against the second connector.

Clause 104. The system of any of clauses 96 to 103, wherein the implantable controller system further comprises one or more electrocardiogram sensors disposed on the outer surface of the implantable housing and electrically connected to the microcontroller, the one or more electrocardiogram sensors configured to detect electrical activity of a patient's heart.

Clause 105. A system comprising: a blood flow modulator configured to move between a low profile state and a high profile flow restricting state; an implantable controller system for implantation in a patient and configured to control the blood flow modulator, the implantable controller system comprising a microcontroller; and a pressure sensor configured to generate a pressure signal to be transmitted to the microcontroller.

Clause 106. The system of clause 105, wherein the microcontroller is configured to cause data to be transmitted to and/or received from an external device.

Clause 107. The system of clause 106, wherein the implantable controller system is configured to move the blood flow modulator between the low profile state and the high profile flow restricting state in response to input instructions.

Clause 108. The system of clause 106, wherein the pressure sensor is configured to generate a first pressure signal, the microcontroller configured to provide the first pressure signal to the external device in response to a first request received from the external device.

Clause 109. The system of clause 106, wherein the pressure sensor is configured to generate pressure signals at scheduled times, at scheduled intervals, and/or based on instructions received from the external device.

Clause 110. The system of clause 106, wherein the external device is configured to receive response data from the patient, the response data indicating one or more patient events and/or one or more patient conditions.

Clause 111. The system of clause 110, wherein the one or more patient events include changes in the patient's medical condition.

Clause 112. The system of clause 105, wherein the implantable controller system further comprises an accelerometer configured to generate an accelerometer signal to be transmitted to the microcontroller or to an external device configured to receive data from the implantable controller system, the accelerometer signal indicating a patient condition and/or activity level.

Clause 113. The system of clause 112, wherein the patient condition includes lying down, sitting, standing, remaining stationary and/or moving.

Clause 114. The system of clause 112, wherein the activity level includes low activity level, medium activity level, and high activity level.

Clause 115. The system of clause 105, wherein the pressure sensor is configured to generate one or more first pressure signals before a therapy routine, one or more second pressure signals during the therapy routine, and one or more third pressure signals after completion of a therapy routine, wherein the therapy routine comprises moving the blood flow modulator from the low profile state towards the high profile flow restricting state for a duration of time and back to the low profile state.

Clause 116. The system of clause 105, wherein the microcontroller is configured to generate and transmit or to cause a transceiver to transmit a notification when the pressure signal indicates a pressure above a threshold and/or a pressure remaining above the threshold for a first time period.

Clause 117. The system of clause 105, wherein the microcontroller is configured to generate and transmit or cause a transceiver to transmit a notification when the pressure signal indicates a pressure above a threshold when the patient is resting.

Clause 118. The system of clause 105, wherein the system is configured to compare patient pressure measurements generated based on the pressure signal to or to collate such patient pressure measurements with atmospheric pressure measurements.

Clause 119. The system of clause 118, wherein microcontroller is configured to transmit or cause a transceiver to transmit the patient pressure measurements to an external device, wherein the external device is configured to compare the patient pressure measurements to or to collate such patient pressure measurements with the atmospheric pressure measurements in a batch process based on time stamps.

Clause 120. The system of clause 105, wherein the microcontroller is configured: receive, one or more atmospheric pressure measurements from an external device; compare, a first patient pressure measurement generated based on the pressure signal to a corresponding atmospheric pressure measurement; generate, a patient gauge pressure based on the comparison; and transmit, a notification to the external device when the patient gauge pressure exceeds a threshold.

Clause 121. The system of clause 105, wherein the microcontroller is configured to calibrate the pressure sensor based on a comparison of the pressure signal to a second pressure signal generated by a non-invasive pressure measurement device.

Clause 122. The system of any of clauses 105 to 121, wherein the system is configured to receive instructions remotely and/or locally from a physician that causes the system to apply a therapy routine that comprises moving the blood flow modulator from the low profile state towards the high profile flow restricting state.

Clause 123. The system of clause 122, wherein the instructions cause the system to apply the therapy routine for one or more specific durations.

Clause 124. The system of clause 122 or clause 123, wherein the instructions cause the system to apply the therapy routine at a specific magnitude of occlusion.

Clause 125. The system of any of clause 122 to 124, wherein the instructions cause the system to apply the therapy routine at one or more specific times.

Clause 126. A system comprising: an implant comprising a flow restrictor coupled with a distal end of a tubing and coupled with a distal end of a shaft extending through a lumen of the tubing, a proximal portion of the tubing having an outside surface comprising an array of electrical contacts, a proximal length of the lumen disposed through the proximal portion of the tubing; and an implantable controller system for controlling the flow restrictor between a low profile state and a high profile flow restricting state, the implantable controller system comprising: an implantable housing; and an actuator at least partially disposed within the implantable housing, the actuator having a first configuration corresponding to the flow restrictor being in the low profile state and a second configuration corresponding to the flow restrictor being in the high profile flow restricting state, wherein the actuator is at least partially located within the proximal length of the lumen of the tubing.

Clause 127. The system of clause 126, wherein the actuator comprises a tube comprising an internally threaded surface and a traveler having an externally threaded surface engaged with the internally threaded surface of the tube, the tube configured to be rotated by a motor disposed in the implantable housing, rotation of the tube causing movement of the traveler within the proximal length of the lumen of the tubing.

Clause 128. The system of clause 127, wherein the externally threaded surface of the traveler extends over a semicircular periphery, the traveler comprising a connecting structure opposite to the externally threaded surface.

Clause 129. The system of clause 128, wherein the connecting structure comprises a surface spanning a portion of a diameter of the semicircular periphery and a portion coupled to a proximal end of the shaft.

Clause 130. The system of clause 128 or clause 129, wherein the actuator comprises a support member configured to support the connecting structure for linear motion.

Clause 131. The system of clause 130, wherein the support member comprises a cylindrical portion having a semicircular periphery configured to extend into the proximal length of the lumen of the tubing and a support portion configured to slidably support the traveler on the connecting structure opposite to the externally threaded surface.

Clause 132. The system of clause 130 or clause 131, wherein the portion of the connecting structure of the traveler coupled to the proximal end of the shaft comprises a projection and the support member comprises a channel, the projection being disposed in the channel to provide for linear motion of the traveler along the support member.

Clause 133. The system of clause 132, wherein the projection in cross section comprises a circular periphery and the channel in cross section comprises a circular periphery.

Clause 134. The system of any of clauses 126 to 133, wherein the proximal end of the shaft is located within the proximal length of the lumen of the tubing in the first configuration and in the second configuration.

Clause 135. The system of any of clauses 126 to 134, wherein the implantable housing is configured to support the tubing surrounding the proximal length of the lumen along a first axis and the actuator comprises a motor having an output shaft rotatable about a second axis, the second axis being parallel to the first axis.

Clause 136. The system of clause 135, wherein a body of the motor is supported in the implantable housing along the tubing surrounding the proximal length of the lumen.

Clause 137. A system comprising: a controller comprising: an implantable housing comprising an outside surface configured to be placed under skin of a patient, a connection port being located on the outside surface of the implantable housing; an actuator at least partially disposed within the implantable housing, the actuator having a first configuration corresponding to a flow restrictor being in a low profile state and a second configuration corresponding to the flow restrictor being in a high profile flow restricting state; and a first connector accessible through the connection port; and an implant comprising: a tubing having a distal end and a proximal end; the flow restrictor coupled with the distal end of the tubing; a shaft having a proximal end and a distal end coupled with the flow restrictor; and a second connector disposed on the proximal end of the shaft; wherein the second connector is configured to be engaged with the first connector in a locked position by insertion of the second connector into the connection port by a distance along a first direction.

Clause 138. The system of clause 137, wherein the second connector is configured to disengage the locked position with the first connector by insertion of the second connector into the connection port by a further distance in the first direction.

Clause 139. The system of clause 137 or clause 138, wherein the first connector comprises a bore extending from the connection port and a retention spring disposed in a channel adjacent to the bore and the second connector comprises a groove adjacent to a free end of the second connector.

Clause 140. The system of clause 139, wherein a square edge is provided on a side of the groove closest to the free end of the second connector.

Clause 141. The system of clause 140, the second connector comprises a spring compression surface adjacent to the side of the groove farthest from the free end of the second connector, the spring compression surface configured to compress the retention spring to a profile radially outward of the square edge.

Clause 142. The system of clause 141, wherein an angled feature is provided on a side of the groove farthest from the free end of the second connector, the angled feature connecting the groove to the spring compression surface.

Clause 143. The system of any of clauses 139 to 142, wherein the groove is a first groove and further comprising a second groove configured to receive a retention spring following insertion into the connection port by the further distance in the first direction.

Clause 144. The system of clause 143, wherein a side of the second groove closest to the free end of the second connector comprises an angled surface configured to gradually compress the retention spring upon movement of the second connector in a second direction opposite to the first direction.

Clause 145. A system comprising: an implantable controller system for controlling an implantable flow restrictor between a low profile state and a high profile flow restricting state, the implantable controller system comprising: an implantable housing; an actuator at least partially disposed within the implantable housing, the actuator having a first configuration corresponding to the implantable flow restrictor being in the low profile state and a second configuration corresponding to the implantable flow restrictor being in the high profile flow restricting state; an internal power source configured to supply current to the actuator; and a processor configured to control operation of the actuator using power from the internal power source; and an external device comprising an external housing enclosing: a communication module configured to communicate with the implantable controller system; wherein the external device is configured to process information related to user physiology and/or receive user input for activating the actuator within the implantable controller system.

Clause 146. The system of clause 145, wherein the internal power source further comprising an induction receiver disposed within the implantable housing, the induction receiver configured to generate current by induction when exposed to magnetic fields generated by an induction transmitter disposed within the external housing.

Clause 147. The system of clause 146, wherein the internal power source further comprises a battery and/or a capacitor configured to receive the current generated by the induction receiver and to store potential energy for use by the actuator.

Clause 148. The system of any of clauses 145 to 147, wherein the actuator comprises motor and a control member coupled with the motor such that rotation of the motor causes rotation of the control member to cause the actuator to be in the first configuration or in the second configuration.

Clause 149. The system of clause 148, wherein the control member comprises a rod and further comprising a traveler configured to be moved between a first position within the implantable housing and a second position upon rotation of an output shaft of the motor, movement to the first position causing a corresponding movement of a distal end of the rod coupled with moveable elements of the implantable flow restrictor, the corresponding movement resulting in the moveable elements of the implantable flow restrictor being positioned away from a central portion of a blood vessel such that the implantable flow restrictor is in the low profile state, movement to the second position causing a corresponding movement of the distal end of the rod resulting in the moveable elements of the implantable flow restrictor being positioned toward the central portion of the blood vessel such that the implantable flow restrictor is in the high profile flow restricting state.

Clause 150. The system of clause 149, wherein the traveler includes a connector system configured to releasably connect the rod to the traveler.

Clause 151. The system of clause 150, wherein the connector system comprises: a push-to-lock connector comprising: a bore; and a locking mechanism positioned at least partially within the bore; and a flange actuator, the flange actuator positioned within and moveable relative to the bore between an extended configuration and a retracted configuration in which the flange actuator is at a maximum extension into the bore; wherein the rod is in a locked position to the traveler when the flange actuator is in the extended configuration and the rod in in an unlocked position to the traveler when the flange actuator is in the retracted configuration.

Clause 152. The system of clause 151, wherein movement of the traveler to a third position causes the flange actuator to engage an internal wall of the implantable housing, causing the flange actuator to move to the retracted configuration and the rod to the unlocked position.

Clause 153. The system of clause 148, wherein the control member comprises a rod and further comprises a spooling portion at a distal end of the rod disposed within or adjacent to the implantable flow restrictor, the spooling portion configured to be rotated by the distal end of the rod between a first rotational position and a second rotational position upon rotation of a proximal end of the rod by an output shaft of the motor, movement to the first rotational position unwinding tension members coupled with moveable elements of the implantable flow restrictor to allow the implantable flow restrictor to be in the low profile state, movement to the second rotational position causing winding of the tension members coupled with the moveable elements of the implantable flow restrictor to cause the implantable flow restrictor to be in the high profile flow restricting state.

Clause 154. The system of clause 145, wherein the actuator comprises a piston disposed within a pressure vessel disposed in the implantable housing and a pressure lumen having a first end in pressure communication with the pressure vessel and a second end in pressure communication with a control member disposed at or within the implantable flow restrictor, wherein in the first configuration of the actuator the piston is positioned within the pressure vessel to provide a first pressure level in the pressure lumen to provide a first movement of the control member resulting in moveable elements of the implantable flow restrictor being positioned away from a central portion of a blood vessel to allow the implantable flow restrictor be in the low profile state and wherein in the second configuration of the actuator the piston is positioned within the pressure vessel to provide a second pressure level in the pressure lumen different from the first pressure level to provide a second movement of the control member resulting in the moveable elements of the implantable flow restrictor being positioned toward the central portion of the blood vessel to cause the implantable flow restrictor to be in the high profile flow restricting state.

Clause 155. The system of any of clauses 145 to 154, further comprising a pressure sensor configured to generate a pressure signal to be transmitted to the processor.

Clause 156. The system of clause 155, wherein the implantable flow restrictor is configured to be placed within a vascular system, the pressure sensor being coupled to a distal portion of the implantable flow restrictor and comprising a signal conductor extending to a proximal end of the implantable flow restrictor to be coupled with the processor within the implantable controller system and disposed outside of the vascular system when the system is implanted.

Clause 157. The system of clause 155, wherein the pressure sensor configured to be implanted separately from the implantable flow restrictor and configured to wirelessly transmit a blood pressure signal to the processor.

Clause 158. A pressure sensor comprising: a cavity plate comprising a first external side and a first internal side of the pressure sensor, the first internal side comprising a first cavity; and a piezo plate comprising a second external side and a second internal side of the pressure sensor, the second internal side comprising a piezoresistive diaphragm and a plurality of electrical contacts, the second external side comprising a second cavity, the second cavity aligned with the piezoresistive diaphragm, wherein the first internal side is coupled to the second internal side such that the piezoresistive diaphragm is aligned with the first cavity.

Clause 159. The pressure sensor of clause 158, wherein the first cavity is configured to accommodate deflection of the piezoresistive diaphragm.

Clause 160. The pressure sensor of clause 158 or clause 159, wherein the plurality of electrical contacts comprise four electrical contacts, each electrical contact conductively coupled to the piezoresistive diaphragm.

Clause 161. The pressure sensor of any of clauses 158 to 160, further comprising a first hole on a first end and a second hole on a second end, the first hole and the second hole extending through both the cavity plate and the piezo plate, wherein the pressure sensor is configured to couple to an implant via the first hole and the second hole.

Clause 162. The pressure sensor of clause 161, wherein the pressure sensor is configured to couple to a shaft of the implant such that the first external side of the cavity plate faces the shaft in use.

Clause 163. The pressure sensor of any of clauses 158 to 162, wherein the cavity plate and the piezo plate each comprise a segment of a glass wafer.

Clause 164. The pressure sensor of any of clauses 158 to 163, wherein the piezoresistive diaphragm comprises a circular or a square periphery.

Clause 165. The pressure sensor of any of clauses 158 to 164, wherein the first cavity has approximately a same diameter as the piezoresistive diaphragm.

Clause 166. The pressure sensor of any of clauses 158 to 165, the piezoresistive diaphragm comprises a doped polysilicon.

Clause 167. The pressure sensor of any of clauses 158 to 166, wherein a vacuum exists within the first cavity.

Clause 168. The pressure sensor of any of clauses 158 to 166, wherein a partial vacuum exists within the first cavity.

Clause 169. The pressure sensor of any of clauses 158 to 168, further comprising a metal layer coupled to the first external side.

Clause 170. The pressure sensor of clause 169, wherein the metal layer is configured to allow the pressure sensor to be welded to a sensor container.

Clause 171. A pressure sensor comprising: a cavity plate comprising a first cavity and a reflow passage disposed through the cavity plate; and a sensor plate coupled with the cavity plate, the sensor plate comprising a second cavity, a strain sensitive member aligned with the second cavity, and a signal conveyance connected to the strain sensitive member; wherein the reflow passage in the cavity plate is configured to receive a reflow material to secure the pressure sensor to a catheter body.

Clause 172. The pressure sensor of clause 171, wherein the sensor plate comprises an electrical contact and the strain sensitive member comprises an electrical circuit.

Clause 173. The pressure sensor of clause 171 or clause 172, wherein the sensor plate comprises a first array of electrical contacts at one end of the sensor plate and a second array of electrical contacts at another end of the sensor plate, the first cavity disposed between the first array and the second array.

Clause 174. The pressure sensor of any of clauses 171 to 173 wherein the sensor plate comprises a first reflow passage at one end of the sensor plate and a second reflow passage at another end of the sensor plate.

Clause 175. The pressure sensor of any of clauses 171 to 174, wherein the sensor plate comprises a plurality of reflow passages and the cavity plate comprises a plurality of reflow passages, the reflow passages of the cavity plate being aligned with the reflow passages of the sensor plate, whereby a reflow material can bridge from a first external side of the pressure sensor to a second external side of the pressure sensor to secure the pressure sensor to a catheter body.

Clause 176. The pressure sensor of any of clauses 173 to 175, wherein at least one of the first array of electrical contacts and the second array of electrical contacts are staggered.

Clause 177. The pressure sensor of any of clauses 171 to 176, wherein the pressure sensor comprises an elongate structure extending between first and second lateral edges, the sensor plate comprising a plurality of electrical contacts disposed between the strain sensitive member and the first lateral edge of the pressure sensor, a first electrical contact of the plurality of electrical contacts being located closer to the strain sensitive member than is a second electrical contact of the plurality of electrical contacts.

Clause 178. The pressure sensor of any of clauses 172 to 177, wherein at least one of the cavity plate and the sensor plate comprise a glass structure and the electrical contact comprises a first material comprising strong adherence to the glass structure and a second material comprising low resistance.

Clause 179. The pressure sensor of clause 178, wherein the electrical contact comprises a first layer comprising chrome, a second layer comprising chrome, and a third layer comprising gold disposed between the first layer of chrome and the second layer of chrome.

Clause 180. The pressure sensor of any of clauses 172 to 179, wherein the electrical contact is configured to engage a lead to be coupled with the pressure sensor in a plug and socket manner.

Clause 181. The pressure sensor of any of clauses 172 to 180, wherein the electrical contact comprises a recess or protrusion configured to mechanically and electrically couple to a lead by mechanical connection.

Clause 182. The pressure sensor of any of clause 172 to 181, further comprising a metalized ring extending around an internal periphery of at least one of the cavity plate and the sensor plate, the metalized ring positioned between the cavity plate and the sensor plate.

Clause 183. The pressure sensor of any of clause 172 to 181, further comprising a metalized layer positioned between the cavity plate and the sensor plate.

Clause 184. The pressure sensor of clause 183, wherein the metalized layer includes a cutout to accommodate the second cavity, the first array of electrical contacts, the second array of electrical contacts, and the signal conveyance.

Clause 185. A method of regulating blood pressure and/or flow using a flow restrictor positioned within a first blood vessel of a patient upstream of a second blood vessel branching off of the first blood vessel at an ostium, comprising: receiving, via a controller system implanted within the patient, treatment instructions from an external device, the treatment instructions including a treatment duration and a target state for the flow restrictor; and adjusting, via an actuator of the controller system, the flow restrictor from an original state to the target state, wherein the flow restrictor provides greater flow restriction within the first blood vessel upstream of the ostium of the second blood vessel in the target state than in the original state.

Clause 186. The method of clause 185, further comprising: adjusting, via the actuator of the controller system, the flow restrictor form the target state to the original state after the treatment duration has expired.

Clause 187. The method of clause 185 or clause 186, wherein the controller system comprises a power source comprising an induction receiver configured to provide current to the actuator.

Clause 188. The method of clause 187, wherein the controller system comprises a processor connected to the induction receiver, the induction receiver having a power receiving mode and a communications mode, the processor configured to receive the treatment instructions from signals provided to the induction receiver when the induction receiver is in the communications mode.

Clause 189. A sensor container assembly configured to house a pressure sensor comprising: a base portion; a top portion; at least one wall extending from the base portion to the top portion and defining an internal volume; and a pressure sensor disposed in the internal volume.

Clause 190. The sensor container assembly of clause 189, further comprising a plurality of electrical contacts coupled to the base portion within the internal volume.

Clause 191. The sensor container assembly of clause 190, further comprising: a plurality of leads coupled to the plurality of electrical contacts and extending at least partially along the base portion; and a plurality of wires coupled to the plurality of electrical contacts via first ends of the plurality of wires, the plurality of wires extending through at least one of the base portion, the top portion, and the at least one wall.

Clause 192. The sensor container assembly of clause 191, wherein the plurality of wires extend through hermetic wire passthroughs configured to prevent ingress into the sensor container assembly.

Clause 193. The sensor container assembly of any of clauses 189 to 192, wherein the top portion comprises an aperture, the aperture configured to be aligned with a diaphragm of the pressure sensor such that the diaphragm is exposed to an environment external to the sensor container assembly.

Clause 194. The sensor container assembly of any of clauses 191 to 193, wherein each wire of the plurality of wires includes a second end comprising an electrical connector, the electrical connectors configured to be coupled to a controller system in a plug and socket manner.

Clause 195. The sensor container assembly of any of clauses 189 to 194, wherein the pressure sensor comprises the pressure sensor of any of clauses 158 to 181, wherein the pressure sensor is housed within the internal volume.

Clause 196. A sensor container assembly configured to house a pressure sensor comprising: a base portion; a top portion; at least one wall extending from the base portion to the top portion and defining an internal volume; and a plurality of electrical contacts coupled to at least one of the base portion, the top portion, and the at least one wall.

Clause 197. A method of assembling a catheter product, comprising: providing a catheter body; extending a plurality of electrical wires through peripheral lumens formed within an outside surface of the catheter body; routing distal ends of the electrical wires through a plurality of securement passages formed through a cavity plate and a diaphragm plate of a pressure sensor; coupling the distal ends of the electrical wires with contacts formed on the cavity plate; retracting the electrical wires within the peripheral lumens of the catheter body to position an external side of the pressure sensor against the outside surface of the catheter body; and coupling the pressure sensor to the outside surface of the catheter body through the securement passages.

Clause 198. The method of clause 197, wherein the securement passages comprise reflow passages formed through the cavity plate and the diaphragm plate and coupling the pressure sensor to the outside surface of the catheter body comprises reflowing a material through the reflow passages.

Clause 199. The method of clause 197 or clause 198, wherein coupling the pressure sensor to the outside surface of the catheter body comprises flowing an adhesive through the securement passages to provide a material bridge from the pressure sensor to the outside surface of the catheter body.

Clause 200. A sensor assembly configured to be coupled with a housing, the sensor assembly comprising: a sensor mount comprising: a top side; a bottom side; and a channel extending between the top side to the bottom side; and a pressure sensor disposed within the channel and coupled to the top side.

Clause 201. The sensor assembly of clause 200, wherein the bottom side includes a rounded portion extending along a length of the bottom side, the rounded portion configured to allow the sensor mount to be coupled to the housing, the housing at least partially nesting within the sensor mount.

Clause 202. The sensor assembly of clause 200 or clause 201, wherein the pressure sensor is secured along an outside edge of the pressure sensor to the top side.

Clause 203. The sensor assembly of any of clauses 200 to 203, further comprising a sensor cover configured to be positioned over the pressure sensor and coupled to the top side.

Clause 204. The sensor assembly of clause 203, wherein the sensor cover includes a fluid opening, the fluid opening positioned over a diaphragm of the pressure sensor.

Clause 205. The sensor assembly of clause 204, wherein the sensor cover is not in contact with the diaphragm of the pressure sensor.

Clause 206. The sensor assembly of any of clauses 200 to 205, further comprising one or more securement openings extending between the top side and the bottom side, the one or more securement openings configured to allow the sensor mount to be coupled to the housing.

Clause 207. A sensor assembly comprising: a sensor container configured to be coupled with a tubular body, the sensor container comprising: a base; and a cover configured to be coupled to the base to define an internal volume, the cover including a first opening; a pressure sensor disposed in the internal volume and extending through the first opening; and a circuit positioned within the sensor container and electrically connected to the pressure sensor via a first set of leads, the circuit configured to process and/or amplify signals received from the pressure sensor.

Clause 208. The sensor assembly of clause 207, wherein the circuit is configured to transmit processed data to a microcontroller.

Clause 209. The sensor assembly of clause 207 or clause 208, wherein the circuit is an application-specific integrated circuit, an amplifier circuit, or an analog-to digital convertor circuit.

Clause 210. The sensor assembly of any of clauses 207 to 209, wherein the pressure sensor comprises an inductive sensor or a capacitive sensor.

Clause 211. The sensor assembly of any of clauses 207 to 210, wherein the sensor container comprises titanium.

Clause 212. The sensor assembly of any of clauses 207 to 211, wherein the sensor container further comprises a first internal wall configured to divide the internal volume into a first internal volume and a second internal volume, the first internal volume being separate from the second internal volume.

Clause 213. The sensor assembly of clause 212, wherein the base comprises a second opening configured to allow a distal portion of the tubular body to at least partially extend into the second internal volume.

Clause 214. The sensor assembly of clause 212 or clause 213, wherein the sensor container further comprises a second internal wall separating the first internal volume from a third internal volume, the first internal volume isolated from the third internal volume, the pressure sensor disposed in the first internal volume.

Clause 215. The sensor assembly of clause 214, wherein the circuit is electrically connected to the microcontroller by one or more second leads, the one or more second leads extending through the second internal wall and from the third internal volume into the second internal volume.

Clause 216. A system comprising: a network interface configured to communicate with a plurality of network devices; one or more data stores configured to store: computer-executable instructions; a first data set comprising a plurality of pressure measurements collected from a plurality of controller systems implanted in a plurality of patients; and a second data set comprising a plurality of patient events associated with the plurality of patients; and one or more physical computer processors in communication with the one or more data stores, wherein the computer-executable instructions, when executed, configure the one or more physical computer processors to: access, by the network interface and from the one or more data stores, the first data set and the second data set; and generate a predictive model by training a machine learning algorithm, wherein the training comprises: inputting the first data set and the second data set into the machine learning algorithm; and comparing the first data set to the second data set; wherein the predictive model is configured to a determine a likelihood of heart failure events based on the comparison.

Clause 217. The system of clause 216, wherein the training further comprises: inputting a third data set comprising a plurality of measurements collected from the plurality of controller systems; and comparing at least one of the first data set and the second data set to the third data set.

Clause 218. The system of clause 217, wherein the third data set includes one or more: a plurality of accelerometer signals collected from a plurality of accelerometers associated with the plurality of patients, a plurality of ECG signals collected from the plurality of controller systems, a plurality of impedance signals collected from one or more electrodes or any device used for impedance measurements associated with the plurality of patients, a plurality of sounds signals collected from a microphone or any device used for auscultation for sound associated with the plurality of patients, a plurality of temperature signals collected from a thermistor or any device used for measuring body temperature associated with the plurality of patients, and a plurality of oxygen signals collected from an optical sensor or any device used for measuring oxygen saturation associated with the plurality of patients.

Clause 219. The system of any of clauses 216 to 218, wherein the one or more physical computer processors are further configured to: provide, into the predictive model, a first input comprising a first set of pressure measurements and a first set of ECG signals, accelerometer signals, impedance signals, sound signals, temperature signals, and/or oxygen signals collected from a first implantable controller associated with a first patient and/or one or more additional devices associated with the first patient; receive, from the predictive model, an output that includes a likelihood of a heart failure event associated with the first input; and generate, a first heart failure risk score and/or first recommendations for the first patient based on the output.

Clause 220. The system of clause 219, wherein the one or more physical computer processors are further configured to: based at least in part on the first heart failure risk score, generate and transmit, to a first user device associated with the first patient, display instructions configured to present a user interface comprising at least one of the first heart failure risk score and the first recommendations.

Clause 221. The system of clause 219 or 220, wherein the one or more physical computer processors are further configured to: provide, into the predictive model, a second input comprising a patient-reported outcome measure data set associated with the first patient.

Clause 222. The system of clause 221, wherein the patient-reported outcome measure data set includes first patient responses to the Kansas City Cardiomyopathy Questionnaire and/or the Minnesota Living with Heart Failure Questionnaire.

Clause 223. The system of any of clauses 216 to 222, wherein the plurality of patient events include hospitalization events and hospitalization dates for the plurality of patients.

Clause 224. The system of any of clauses 220 to 223, wherein the first recommendations include a recommendation for the first patient to check into a hospital.

Clause 225. The system of any of clauses 220 to 224, wherein the first recommendations include a recommended therapy routine to be administered to the first patient via a blood flow modulator associated with a first controller system implanted in the first patient.

Clause 226. A computer-implement method, comprising: accessing, by a network interface and from one or more data stores: a first data set comprising a plurality of pressure measurements collected from a plurality of controller systems implanted in a plurality of patients; and a second data set comprising a plurality of patient events associated with the plurality of patients; and generating a predictive model by training a machine learning algorithm, wherein the training comprises: inputting the first data set and the second data set into the machine learning algorithm; and comparing the first data set to the second data set; wherein the predictive model is configured to a determine a likelihood of heart failure events based on the comparison.

Clause 227. The computer-implement method of clause 226, wherein the training further comprises: inputting a third data set comprising a plurality of measurements collected from the plurality of controller systems; and comparing at least one of the first data set and the second data set to the third data set.

Clause 228. The computer-implement method of clause 227, wherein the third data set includes one or more: a plurality of accelerometer signals collected from a plurality of accelerometers associated with the plurality of patients, a plurality of ECG signals collected from the plurality of controller systems, a plurality of impedance signals collected from one or more electrodes or any device used for impedance measurements associated with the plurality of patients, a plurality of sounds signals collected from a microphone or any device used for auscultation for sound associated with the plurality of patients, a plurality of temperature signals collected from a thermistor or any device used for measuring body temperature associated with the plurality of patients, and a plurality of oxygen signals collected from an optical sensor or any device used for measuring oxygen saturation associated with the plurality of patients.

Clause 229. The computer-implement method of any of clauses 226 to 228, further comprising: providing, into the predictive model, a first input comprising a first set of pressure measurements and a first set of ECG signals, accelerometer signals, impedance signals, sound signals, temperature signals, and/or oxygen signals collected from a first implantable controller associated with a first patient and/or one or more additional devices associated with the first patient; receiving, from the predictive model, an output that includes a likelihood of a heart failure event associated with the first input; and generating, a first heart failure risk score and/or first recommendations for the first patient based on the output.

Clause 230. The computer-implement method of clause 229, further comprising: based at least in part on the first heart failure risk score, generating and transmitting, to a first user device associated with the first patient, display instructions configured to present a user interface comprising at least one of the first heart failure risk score and the first recommendations.

Clause 231. The computer-implement method of clause 229 or 230, further comprising: providing, into the predictive model, a second input comprising a patient-reported outcome measure data set associated with the first patient.

Clause 232. The computer-implement method of clause 231, wherein the patient-reported outcome measure data set includes first patient responses to the Kansas City Cardiomyopathy Questionnaire and/or the Minnesota Living with Heart Failure Questionnaire.

Clause 233. The computer-implement method of any of clauses 226 to 232, wherein the plurality of patient events include hospitalization events and hospitalization dates for the plurality of patients.

Clause 234. The computer-implement method of any of clauses 230 to 233, wherein the first recommendations include a recommendation for the first patient to check into a hospital.

Clause 235. The computer-implement method of any of clauses 230 to 234, wherein the first recommendations include a recommended therapy routine to be administered to the first patient via a blood flow modulator associated with a first controller system implanted in the first patient.

Clause 236. A non-transitory computer storage medium storing computer-executable instructions that, when executed by a processor, cause the processor to at least: perform the computer-implemented method of any of Clauses 226 to 235.

### Computer Systems

FIG. 41 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 41. The example computer system 3202 is in communication with one or more computing systems 3220 and/or one or more data sources 3222 via one or more networks 3218. While FIG. 41 illustrates an embodiment of a computing system 3202, it is recognized that the functionality provided for in the components and modules of computer system 3202 may be combined into fewer components and modules, or further separated into additional components and modules.

The computer system 3202 can comprise a programming module 3214 that carries out the functions, methods, acts, and/or processes described herein. The programming module 3214 is executed on the computer system 3202 by a central processing unit 3206 discussed further below.

In general, the word “module,” as used herein, refers to logic embodied in hardware or firmware or to a collection of software instructions, having entry and exit points. Modules are written in a program language, such as JAVA. C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 3202 includes one or more processing units (CPU) 3206, which may comprise a microprocessor. The computer system 3202 further includes a physical memory 3210, such as random-access memory (RAM) for temporary storage of information, a read only memory (ROM) for permanent storage of information, and a mass storage device 3204, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 3202 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 3202 includes one or more input/output (I/O) devices and interfaces 3212, such as a keyboard, mouse, touch pad, and printer. The I/O devices and interfaces 3212 can include one or more display devices, such as a monitor, which allows the visual presentation of data to a user. More particularly, a display device provides for the presentation of GUIs as application software data, and multi-media presentations, for example. The I/O devices and interfaces 3212 can also provide a communications interface to various external devices. The computer system 3202 may comprise one or more multi-media devices 3208, such as speakers, video cards, graphics accelerators, and microphones, for example.

The computer system 3202 may run on a variety of computing devices, such as a server, a Windows server, a Structure Query Language server, a Unix Server, a personal computer, a laptop computer, a smart phone, a personal digital assistant, a tablet, and so forth. Servers may include a variety of servers such as database servers (for example, Oracle, DB2, Informix, Microsoft SQL Server, MySQL, or Ingres), application servers, data loader servers, or web servers. In addition, the servers may run a variety of software for data visualization, distributed file systems, distributed processing, web portals, enterprise workflow, form management, and so forth. In other embodiments, the computer system 3202 may run on a cluster computer system, a mainframe computer system and/or other computing system suitable for controlling and/or communicating with large databases, performing high volume transaction processing, and generating reports from large databases. The computing system 3202 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 3202 illustrated in FIG. 41 is coupled to a network 3218, such as a LAN, WAN, or the Internet via a communication link 3216 (wired, wireless, or a combination thereof). Network 3218 communicates with various computing devices and/or other electronic devices. Network 3218 is communicating with one or more computing systems 3220 and one or more data sources 3222. The programming module 3214 may access or may be accessed by computing systems 3220 and/or data sources 3222 through a web-enabled user access point. Connections may be a direct physical connection, a virtual connection, and other connection type. The web-enabled user access point may comprise a browser module that uses text, graphics, audio, video, and other media to present data and to allow interaction with data via the network 3218.

Access to the programming module 3214 of the computer system 3202 by computing systems 3220 and/or by data sources 3222 may be through a web-enabled user access point such as the computing systems' 3220 or data source's 3222 personal computer, cellular phone, smartphone, laptop, tablet computer, e-reader device, audio player, or another device capable of connecting to the network 3218. Such a device may have a browser module that is implemented as a module that uses text, graphics, audio, video, and other media to present data and to allow interaction with data via the network 3218.

The output module may be implemented as a combination of an all-points addressable display such as a cathode ray tube (CRT), a liquid crystal display (LCD), a plasma display, or other types and/or combinations of displays. The output module may be implemented to communicate with input devices 3212 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 3202 may comprise a physical or logical connection established between a remote microprocessor and a mainframe host computer for the express purpose of uploading, downloading, or viewing interactive data and databases online in real time. The remote microprocessor may be operated by an entity operating the computer system 3202, including the client server systems or the main server system, an/or may be operated by one or more of the data sources 3222 and/or one or more of the computing systems 3220. In some embodiments, terminal emulation software may be used on the microprocessor for participating in the micro-mainframe link.

In some embodiments, computing systems 3220 who are internal to an entity operating the computer system 3202 may access the programming module 3214 internally as an application or process run by the CPU 3206.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL ca specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, or the like. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information. HTTP authentication as authentication protocols, IP addresses to track session or identity information, URLs, and the like.

The computing system 3202 may include one or more internal and/or external data sources (for example, data sources 3222). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Caché), a cloud-based database (for example, Amazon RDS, Azure SQL, Microsoft Cosmos DB, Azure Database for MySQL. Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MogoDB Atlas, Amazon Aurora, Amazon DynamoDB, Amazon Redshift, Amazon ElastiCache, Amazon MemoryDB for Redis, Amazon DocumentDB, Amazon Keyspaces, Amazon EKS, Amazon Neptune, Amazon Timestream, or Amazon QLDB), a non-relational database, or a record-based database.

The computer system 3202 may also access one or more databases 3222. The databases 3222 may be stored in a database or data repository. The computer system 3202 may access the one or more databases 3222 through a network 3218 or may directly access the database or data repository through I/O devices and interfaces 3212. The data repository storing the one or more databases 3222 may reside within the computer system 3202.

### Additional Considerations and Terminology

Features, materials, characteristics, or groups described in conjunction with a particular aspect, implementation, or example are to be understood to be applicable to any other aspect, implementation or example described herein unless incompatible therewith. All of the features disclosed in this specification (including any accompanying clauses, abstract and drawings), or all of the steps of any method or process so disclosed, may be combined in any combination, except combinations where at least some of such features or steps are mutually exclusive. The protection is not restricted to the details of any foregoing implementations. The protection extends to any novel one, or any novel combination, of the features disclosed in this specification (including any accompanying claims, abstract and drawings), or to any novel one, or any novel combination, of the steps of any method or process so disclosed.

While certain implementations have been described, these implementations have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some implementations, the actual steps taken in the processes illustrated or disclosed may differ from those shown in the figures. Depending on the implementation, certain of the steps described above may be removed, others may be added. For example, the actual steps or order of steps taken in the disclosed processes may differ from those shown in the figure. Depending on the implementation, certain of the steps described above may be removed, others may be added. Furthermore, the features and attributes of the specific implementations disclosed above may be combined in different ways to form additional implementations, all of which fall within the scope of the present disclosure.

Although the present disclosure includes certain implementations, examples and applications, it will be understood by those skilled in the art that the present disclosure extends beyond the specifically disclosed implementations to other alternative implementations or uses and obvious modifications and equivalents thereof, including implementations which do not provide all of the features and advantages set forth herein. Accordingly, the scope of the present disclosure is not intended to be limited by the described implementations, and may be defined by claims as presented herein or as presented in the future.

Conditional language, such as “can,” “could,” “might,” or “may,” unless specifically stated otherwise, or otherwise understood within the context as used, is generally intended to convey that certain implementations include, while other implementations do not include, certain features, elements, or steps. Thus, such conditional language is not generally intended to imply that features, elements, or steps are in any way required for one or more implementations or that one or more implementations necessarily include logic for deciding, with or without user input or prompting, whether these features, elements, or steps are included or are to be performed in any particular implementation. The terms “comprising.” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

1-184. (canceled)

185. A system comprising:
an implantable controller system for controlling a blood flow modulator, the implantable controller system comprising a housing enclosing:
a microcontroller comprising:
one or more computer readable storage devices configured to store a plurality of computer-executable instructions;
one or more hardware computer processors in communication with the one or more computer readable storage devices; and
a first communication module;
an actuator comprising:
a control member having a first end coupled with the blood flow modulator and a second end configured to be disposed adjacent to or in the housing;
a traveler coupled to the second end of the control member; and
a motor coupled to the traveler, the motor configured to control movement of the traveler relative to the housing, wherein movement of the traveler in a first direction causes the blood flow modulator to move between a low profile state and a high profile pressure modulating state; and
a first power source disposed in the housing and configured to generate current for the actuator.

186. The system of claim 185, further comprising:
a pressure sensor configured to generate a pressure signal to be transmitted to the microcontroller as an input for changing states of the blood flow modulator.

187. The system of claim 185, wherein the computer-executable instructions, when executed, configure the one or more hardware computer processors to:
receive, from a pressure sensor, a pressure signal; and
activate the actuator such that the traveler translates in the first direction until the pressure signal indicates a target pressure.

188. The system of claim 185, wherein the computer-executable instructions, when executed, further configure the one or more hardware computer processors to:
receive, from an external device, treatment instructions comprising a treatment duration and a target state for the blood flow modulator; and
activate the actuator such that the traveler translates a first distance in the first direction, the first distance corresponding to the target state.

189. The system of claim 188, wherein the computer-executable instructions, when executed, further configure the one or more hardware computer processors to:
determine, that the treatment duration has expired; and
activate the actuator such that the traveler translates the first distance in a second direction, the second direction opposite the first direction.

190. The system of claim 185, wherein the traveler further comprises a magnet and the implantable controller system further comprises at least one hall effect sensor positioned near the control member, the at least one hall effect sensor configured to detect a magnetic field produced by the magnet and transmit a corresponding output voltage to the microcontroller.

191. The system of claim 185, wherein at least a portion of the actuator is enclosed within an ingress protection system configured to prevent fluid received within the ingress protection system from entering a main portion of the housing.

192. The system of claim 191, wherein the housing is an external housing and the ingress protection system comprises an internal housing, the internal housing positioned within the external housing and hermetically sealed relative to the housing.

193. The system of claim 185, wherein the implantable controller system further comprises a tube having a first end coupled with the blood flow modulator and a second end configured to be disposed adjacent to or in the housing, the control member extending through the tube such that the control member is not exposed to a patient in use at least along a portion of a length thereof, the control member configured to move relatively to the tube.

194. The system of claim 193, wherein the housing further comprises an extension portion defining a passage into the housing, wherein the second end of the control member extends through the passage and the second end of the tube extends at least partially through the passage, wherein the tube can be selectively fixed to the housing by applying a compressive force on the second end of the tube.

195. A system comprising:
an implant comprising a flow restrictor coupled with a distal end of a tubing and coupled with a distal end of a shaft extending through a lumen of the tubing, a proximal length of the lumen disposed through a proximal portion of the tubing;
a first connector disposed at the proximal portion of the tubing;
a second connector disposed at a proximal end of the shaft, the second connector at least partially disposed within the first connector; and
an implantable controller system for controlling the flow restrictor between a low profile state and a high profile flow restricting state, the implantable controller system comprising:
an implantable housing; and
an actuator positioned within the implantable housing, the actuator configured to cause the second connector to translate within the first connector, the actuator having a first configuration corresponding to the flow restrictor being in the low profile state and a second configuration corresponding to the flow restrictor being in the high profile flow restricting state.

196. The system of claim 195, wherein the second connector comprises a tube comprising an internally threaded surface and an external surface comprising one or more projections, wherein the first connector comprises a tube comprising an inner surface with one or more recesses, the one or more recesses configured to receive the one or more projections.

197. The system of claim 196, wherein the one or more projections comprise one or more splines and the one or more recesses comprise one or more spline guides.

198. The system of claim 196, wherein the actuator comprises an externally threaded shaft engaged with the internally threaded surface of the second connector, the externally threaded shaft configured to be rotated by a motor disposed in the implantable housing, rotation of the externally threaded shaft causing movement of the second connector relative to the first connector.

199. The system of claim 196, wherein engagement between the one or more projections and the one or more recesses allow the second connector to translate within the first connector when driven by the actuator.

200. The system of claim 195, wherein the actuator comprises a motor having an output shaft rotatable about a first axis, the output shaft configured to drive rotation of an externally threaded shaft about a second axis, the first axis parallel to the second axis.

201. The system of claim 195, wherein the implant further comprises a first sleeve, a second sleeve, and a third sleeve, the first sleeve coupled to the proximal end of the shaft, the third sleeve coupled to the shaft at a location distal to the first sleeve, the second sleeve positioned between the first sleeve and the third sleeve.

202. The system of claim 195, wherein the shaft is configured to rotate relative to the second connector.

203. The system of claim 195, wherein the implantable controller system further comprises an induction receiver configured to activate the actuator or charge a power source housed in an internal volume of the implantable housing.

204. A system comprising:
a blood flow modulator configured to move between a low profile state and a high profile flow restricting state;
an implantable controller system for implantation in a patient and configured to control the blood flow modulator, the implantable controller system comprising a microcontroller; and
a pressure sensor configured to generate a pressure signal to be transmitted to the microcontroller.
